(19) United States
(12) Patent Application Publication
(10) Pub. No.: US 2018/0303824 A1
(43) Pub. Date: Oct. 25, 2018

(54) Compositions for deterring abuse of pharmaceutical products and alcohol

(71) Applicant: NOVA SOUTHEASTERN UNIVERSITY, Fort Lauderdale, FL (US)

(72) Inventors: Hossein Omidian, Weston, FL (US); Srinath Muppalaneni, West Palm Beach, FL (US); Yogesh N. Joshi, Tamarac, FL (US); David J. Mastropietro, Davie, FL (US)

(21) Appl. No.: 15/770,193

(22) PCT Filed: Oct. 21, 2016
(86) PCT No.: PCT/US16/58097

§ 371 Date: Apr. 21, 2018

Related U.S. Application Data
(60) Provisional application No. 62/244,637, filed on Oct. 21, 2015.

(51) Int. Cl. A61K 31/485; A61K 31/135; A61K 9/20

(52) U.S. Cl. CPC A61K 31/485; A61K 31/135; A61K 9/2031; A61K 9/2059; A61K 9/205; A61K 9/2054

(57) ABSTRACT

The invention enables the formation of prescription drugs less likely to be abused. Different approaches are employed that can potentially deter abuse by reducing the efficacy of main processes utilized by abusers to speed drug absorption and enhance its effect. Pharmaceutical compositions of the invention incorporate one or more of the following elements: super water-absorbency, alcohol absorption, organic binding agents, inorganic binding agents, adsorption, and tough platforms. These compositions function by preventing the isolation and concentration of drug constituents for misuse and/or preventing excessive intake. Thus, the invention encompasses various compositions and methods for reducing abuse of pharmaceutical products and alcohol.

### CROSS REFERENCE TO RELATED APPLICATIONS

This application claims the benefit of priority to U.S. Provisional Patent Application No. 62/244,637, filed on Oct. 21, 2015, the content of which is hereby incorporated by reference in its entirety.

### FIELD OF THE DISCLOSURE

The invention relates to reducing the incidence of tampering and abuse of pharmaceutical products and alcohol, and more particularly to preventing the isolation and concentration of drug constituents for misuse, and preventing excessive intake.

### BACKGROUND OF THE DISCLOSURE

Prescription drug abuse is at epidemic proportions, and has become a serious problem affecting public health worldwide. Pain medications, CNS depressants, and stimulants are among those commonly abused via different techniques including snorting, injection, and co-ingestion with alcohol.

Tablets, transdermal patches, and nasal sprays are the most commonly abused pharmaceutical products and are frequently tampered by crushing and/or mixing with water and alcohol. The initial step of crushing is needed to abuse drugs by almost all routes such as snorting, injecting, smoking, and orally to achieve rapid absorption of the entire dose at once. It is also very common for abusers to take crushed drug products with alcoholic drinks or other beverages to heighten the effects of the drug and allow quicker entry into the bloodstream.

Abuse of prescription drugs is now a fastest-growing drug problem in the US. In almost 10 years, the number of Americans abusing controlled prescription drugs rose from 7.8 million in 1992 to 15.1 million in 2003. This high number of abusers represents more people than the combined total of those abusing cocaine, hallucinogens, inhalants, and heroin. Recent results from the 2009 National Survey on Drug Use and Health report that an average of 7,000 people each day experiment for the first time with a prescription pain medication, tranquilizer, stimulant, or sedative. The large increase in prescribing and abuse of prescription medications has affected public health in many ways. The number of emergency room visits and unintentional deaths due to controlled prescription drugs has increased sharply over the century from 1998 to 2008. Although these medications are generally safe to take as prescribed, they can be deadly when abused, or and taken inappropriately.

Attributed largely to the misuse and abuse of prescription medications, drug poisonings and overdoses now kill more Americans than car accidents for the first time in history. The prescription pain medications have been most responsible for these deaths; as the number of drug poisoning deaths involving such medications has risen from 4,000 in 1999 to 14,800 in 2008, representing over 40% of drug poisoning deaths in 2008.

As more Americans began abusing prescription drugs, so has the number seeking treatment. Every year, from 1999 to 2008, there has been an increase in the number of individuals seeking treatment for opioid prescription pain medications. Along with the increased abuse and treatment of prescription drugs comes rising medical costs. The overall direct cost to health insurers resulting from the nonmedical use of prescription painkillers has been estimated up to $72.5 billion annually.

The abuse and misuse of prescription medications is not limited to the United States. According to the United Nations 2011 World Drug Report, the demand for cocaine, heroin, and cannabis (each an illicit drug) has declined or stayed the same while the production and abuse of prescription opioid pain medications has grown. There are many factors contributing to this widespread abuse. One incentive type factor is the perception that prescription medications are safe and associated with a low potential for harm and abuse compared to illicit drugs. Another factor is the ease of obtaining prescription medications. Many abusers find that prescription medications are much easier to obtain than illicit (street) drugs. A national survey showed that over 70% of people who abused prescription pain medications obtained them directly from friends or relatives, while only 4.3% acquiring them from drug dealers or strangers.

Even though young adults are those most likely to abuse prescription drugs, young adolescent children and older adults abused them too. The abuse of pain medications among adolescents has increased from 3.3% in 1992 to 9.5% in 2004, and stayed close to this level through 2010. Those aged 50 to 59 also showed an increase in abuse from 2.7% in 2002 to 6.2% in 2009. Serious health risks are associated with abuse of these medications in patients over fifty. The number of emergency room visits involving the misuse and abuse of prescription drugs in those aged over 50 increased 121.1% from 2004 to 2008.

The most commonly prescribed medications by physicians are oral tablets and capsules, and they have become the most commonly abused medications. The National Institute on Drug Abuse lists the top three drug classes abused as opioids, central nervous system (CNS) depressants, and stimulants. Opioids are medications similar to morphine (e.g., oxycodone, hydrocodone, codeine), which commonly produce a sense of well-being or euphoria in the abuser. CNS depressants are medications typically used for sleep or anxiety disorders, which cause drowsiness and a calming effect in users. Stimulants are drugs commonly referred to as “uppers”, because they produce alertness and energy with an overall elevation in mood that makes them top candidate drugs for abuse.

When an oral drug no longer gives the same high or euphoric feeling, abusers may take more (overdose), take it in a different way, or manipulate the medication to produce a greater or more rapid euphoria. Altering the medication from its original form for this purpose can be defined as tampering. Tampering typically results in the drug being absorbed at a faster rate or allows the medication to be given by another route. The most common methods of tampering are as follows:

crushing a tablet medication into a powder so that it can be inhaled through the nose and rapidly enter the bloodstream;

once a tablet medication is reduced to small particles by crushing or chewing, it may be taken orally, smoked, snorted, or mixed with a solution and injected for faster results; and

when swallowed with medications, alcohol causes certain drugs to dissolve more quickly and to be absorbed rapidly, which dangerously intensifies the drug's effect on the body. One approach to address the foregoing is Reformulated Oxycontin® (a powerful pain medication). The original Oxycontin tablet was meant to deliver the drug slowly over 12 hours, but abusers quickly found the effect of alcohol in enhancing the drug solubility and that chewing or crushing the tablet could defeat the slow release mechanism. In response, the manufacturer reformulated the product into a similar looking tablet, resistant to crushing into small pieces, forming a thick viscous fluid upon contact with liquids.

REMOXY is a capsule type product containing thick “taffy” like material inside the capsule shell, which purports to slow down drug release. As of this writing, FDA approval has been delayed due to product inconsistency and unpredictable performance.

Embeda® was approved in the U.S. in 2009, and is a capsule that contains small beads of morphine and a segregated compartment which releases a drug upon crushing that stops morphine from working. In 2011, the product was voluntarily recalled for stability reasons and has yet to return to the marketplace. Reformulated Opana® ER (oxymorphone HCl) utilizes a melt extrusion or a thermal process. Exalgo® (Hydromorphone) has a hard exterior shell and gelling agent. Oxecta® (oxycodone HCl) contains gelling agent and a nasal irritant. Nucynta® ER (Tapentadol) uses an approach similar to the reformulated Opana® ER.

Tampering methods such as crushing, chewing, grating, or grinding a dosage form to obtain smaller particles allows the drug to be taken by alternate routes, and speeds the rate of dissolution. For example, crushing a tablet would allow the abuser to snort or smoke the product, or mix with a suitable liquid to dissolve the drug and inject the resultant solution parenterally after filtration. A great concern to public health is when abusers tamper with extend-release formulations containing a large amount of drug meant to be absorbed slowly over several hours. The ability to easily destroy the controlled release mechanisms of these formulations by crushing or other means allows high levels of drug to be absorbed rapidly and to dangerous levels in the user. Tampering of this nature can occur intentionally as in the case of an abuser seeking to get high, or unintentionally by a legitimate user crushing the tablet for ease of swallowing. Drugs and other excipients soluble in ethanol also have the added danger of “dose-dumping”, meaning release of the entire drug load at once, when taken with an alcoholic beverage.

The development of dosage forms intended to deter, discourage and prevent the non-medical use of highly abused drugs was initially made popular by the incorporation of narcotic antagonist into tablet formulations prone to parenteral abuse. Most of these formulations pertain to oral dosage forms, particularly solid dosage forms. First attempts were the use of opioid antagonist that were not orally bioavailable, but would exert their effect if the dosage form was injected by parenteral routes. In the late 1970's, a combination of the prescription drug pentazocine (Talwin®) along with the antihistamine tripelennamine were being used together parenterally to gain a high similar to heroin. To combat this problem, naloxone was included into the formulation, and marketed in the United States as Talwin®Nx. The naloxone in the reformulated tablet was sufficient to antagonize the effects of pentazocine when administered parenterally yet have limited effects when taken orally. The addition of naloxone to tablets was therefore included to deter intravenous abuse. More recently in 2002, the FDA approved the combination of buprenorphine with naloxone (Suboxone®) as a sublingual tablet for the treatment of opioid dependence outside of a clinic. The naloxone component is added to help deter misuse such as parenteral injection during maintenance therapy. Concerns such as the slow dissolution of the sublingual tablets and unintentional child exposures led to the development of oral films with better mucoadhesion and oral dissolution.

U.S. Pat. No. 7,968,119 describes compositions consisting of an opioid agonist together with a sequestered antagonist agent and an antagonist removal system. U.S. Pat. No. 4,457,933 describes combining the analgesic dose of an opioid with a specific low ratio of naloxone. U.S. Pat. No. 6,228,863 describes oral dosage forms that makes extracting an opioid analgesic from the combined agonist/antagonist mixture at least a two-step process. U.S. Pat. Nos. 6,696,088, 7,658,939, 7,718,192, 7,842,309, and 7,842,311 describe tamper-resistant oral dosage forms having a sequestered antagonist. U.S. Pat. No. 7,914,818 describes both a non-releasable sequestered opioid antagonist along with a releasable opioid antagonist together with the opioid agonist.

U.S. Pat. No. 3,980,766 describes adding ingestible solid materials that have rapid thickening properties in water. Compositions containing aqueous gelling agents are described in U.S. Pat. No. 4,070,494. U.S. Pat. No. 6,309,668 describes tablet compositions having two or more layers, where the gelling agent is in a separate layer from the drug. Abuse deterrent dosage forms containing a gel forming polymer along with an analgesic opioid, nasal tissue irritant, and emetic or inert emesis causing agent are described in U.S. Pat. Nos. 7,201,920, 7,476,402, and 7,510,726. Other patents having deterrent agents include U.S. Pat. No. 4,175,119 describing the use of emetic coating, and U.S. Pat. No. 4,459,278 describing binding the emetic agents to an inert sub stance.

In addition to drug abuse, consumption of alcohol is a major public health concern associated with significant costs and high rates of mortality. Three oral medications, i.e. disulfiram (Antabuse®), naltrexone (Depade®, ReVia®) and acamprosate (Campral®) are currently approved to treat alcohol dependence. In addition, an injectable form of naltrexone (Vivitrol®) is also available.

Carbonaceous adsorbents can be modified to produce micro-porous structures giving the material an extremely large surface area. Activated charcoal is an example of carbonaceous material that first undergoes carbonization, and then an activation step to produce a highly porous material capable of adsorption. Activation refers to the development of surface area by increasing pore volume, pore diameter, and porosity of the material through a physical, chemical, or physiochemical activation process. The activation process usually occurs at high temperatures in an environment of an activating gas (e.g. carbon dioxide, steam) or a chemical activating agent (e.g., phosphoric acid, zinc chloride) or both. The raw material to make activated carbon may start from a variety of sources including animal (animal charcoal), natural gas incomplete combustion (e.g., gas black, furnace black), and burning of fats and oils (e.g., lamp black). However, activated charcoal is derived from wood or vegetable origins.

Activated charcoal is a black porous material that is insoluble in water and organic solvents. Commercially, it is available in many forms such as granular, extruded, pelletized or powdered in varying particle sizes. Activated charcoal for medicinal purposes must meet compendial or similar standards (BP, USP), which includes testing to demonstrate its adsorption power. Additionally, it should have a surface area of at least 900 m²/g to have adequate adsorption potential. The properties of activated charcoal are due largely to its enormous surface area and surface chemistry. The average surface area range of activated charcoal is between 800-1,200 m²/g, and may be modified to as large as 2,800-3,500 m²/g. Although the exact mechanisms of interaction between activated carbon and a substrate are complex, adsorption processes are the most well studied, and may be chemical or physical in nature. For the adsorption process in a liquid, activated charcoal acts as the insoluble adsorbent to which a water soluble adsorbate is adsorbed onto. Adsorption may be dependent on polarity, ionization, and environmental pH, with organic and large poorly water soluble materials adsorbing to a higher degree than polar small molecules. Orally, activated charcoal is most notably used as a gastrointestinal decontamination agent to treat acute overdoses and poisonings.

Prescription drug abuse is now a widespread phenomenon, particularly regarding opioid narcotic analgesics. These medications are having alarming effects to public health as the rate of their abuse increases. According to the CDC, drug overdose deaths in the United States have continuously increased for 11 consecutive years in 2010 with opioids being the driving factor and prescription drugs as a whole involved in 60% of cases. Other abusable analgesics such as tramadol have also increased. For example, visits to the emergency room from tramadol overdoses which cause seizures and repository or CNS depression in patients have recently increased. The use of activated charcoal to treat tramadol overdose was investigated in-vitro and in-vivo, and reported to bound up to 0.05 mg of tramadol for each mg of activated charcoal.

Different physicochemical approaches have been experienced by pharmaceutical industry in developing medications with abuse deterrence capability. These include, for instance, prodrug, agonist/antagonist combinations, enzyme inhibition, aversive, coatings, ion exchange, and viscosity-building agents. Oral analgesics, particularly opioids, have been the first drugs incorporated into such formulations. For example, reformulated OxyContin® (oxycodone HCl) utilizes a mechanically strong tablet matrix which can to some extent resist crushing. Reformulated Opana® ER (oxymorphone HCl) utilizes a melt extrusion or a thermal process to become greatly resistant to crushing. In the meantime, the polymer used in these two medications can build great viscosity in an aqueous medium, which makes other processes such as syringeability, filtration, and overall extraction difficult. Embeda® (morphine sulfate/naltrexone) uses agonist/antagonist approach; Exalgo® (Hydromorphone) has a hard exterior shell and gelling agent; Oxecta® (oxycodone HCl) contains gelling agent and a nasal irritant, and Nucynta® ER (Tapentadol) enjoys the same approach as being utilized in reformulated Opana® ER.

Other approaches include the use of polyvinyl alcohol (PVOH). Both chemically and physically-modified Polyvinyl alcohol (PVOH) structures have found applications in biomedical and pharmaceutical areas. Because of its biocompatibility, drug compatibility, water solubility, film forming, good mechanical and swelling properties, the PVOH polymer itself can be found in a variety of pharmaceutical products, including tablets, ophthalmics, implants, transdermal patches and topical creams. Commercial PVOH polymers and copolymers for industrial and pharmaceutical applications include PVA Emprove (from EMD Millipore, Ph Eur (European Pharmacopoeisa), USP is available in a variety of viscosities and grades of hydrolysis to suit various pharmaceutical applications and uses), Selvol™ (Sekisui Specialty Chemicals), Elvanol™ (DuPont), Gohsenol™ (Nippon Gohsei), and Opadry® (Colorcon Inc., graft copolymers of ethylene glycol and vinyl alcohol).

As shown in the art, PVOH solutions have the ability to form gels under repeated freezing and thawing conditions (cryogelation). An aqueous solution of this polymer (even at low concentrations) can be transformed into a solid rubber-like material via a simple freezing-thawing treatment. Upon freezing a PVOH solution, hydroxyl groups (—OHs) of the adjacent polymer chains interact via intra- and inter-molecular forces, creating an ordered water-insoluble structure. The process is favored when the PVOH material is highly de-acetylated, the aqueous PVOH solution is concentrated (up to 20 wt %), and the PVOH molecular weight is in the range 50,000-130,000. There are several articles reporting the freeze-thaw treatment of PVOH solutions at both low and high concentrations, the effect of added salt on the swelling kinetics, the structure/property relationship and the mechanisms of cryotropic gelation of PVOH. A small number of studies have also been focused on osmotic properties, rheological and thermal properties, amount of sol and gel fractions, influence of low molecular weight polyelectrolytes, applications of PVOH cryogels in cell immobilization, applications for protein delivery, and as a strengthening agent in super-porous hydrogels.

Considering that prescription drug abuse and/or alcohol abuse is prevalent and on the rise worldwide, new compositions capable of dettering this abuse would be valuable.

### SUMMARY OF THE INVENTION

The invention provides various compositions for reducing the incidence of tampering with and abuse of pharmaceutical products and alcohol. These compositions function by preventing the isolation and concentration of drug constituents for misuse and/or preventing excessive intake.

The invention encompasses the use of certain pharmaceutically-acceptable functional polymers, i.e. multifunctional polymers, that are used to make more effective abuse deterrent medications. This disclosure describes different approaches that can potentially deter abuse by reducing the efficacy of main processes utilized by abusers to speed drug absorption and enhance its effect. Pharmaceutical compositions of the disclosure incorporate one or more of the following elements described herein to reduce abuse: super water-absorbency, alcohol absorption, organic binding agents, inorganic binding agents, adsorption, and tough platforms.

As used herein, the term “drug” refers to a pharmaceutically-active ingredient, which is incorporated into a dosage form of the invention. A pharmaceutically-active ingredient is preferably a known drug that is or has the potential to be abused. As used herein, a pharmaceutically-active ingredient is not limited to a drug, but could be any substance capable of being misused and/or abused.

Embodiments disclosed herein are safe and effective if used by regular patients or as prescribed, and are ineffective or less effective in the hand of abusers.

In an embodiment, a pharmaceutical composition of this disclosure is composed of an abusable drug active ingredient, and two primary polymers. The primary polymers utilized in this disclosure are an integral part of the abusable formulation. The first primary polymer, a water-swellable superabsorbent polymer, is a chemically-crosslinked hydrophilic polymer or copolymer, which can at least swell in water to greater than 40 grams per gram of the dry polymer. The water-swellable superabsorbent polymers described herein will change the texture and the flow property of the dosage form in the solution state. Depending on its concentration in the tablet, this polymer significantly reduces the amount of filtrate during the extraction process. The second primary polymer, a plastic agent, is a thermoplastic water-soluble or water-insoluble polymer, which provides a mechanical property to the dosage form in the solid state.

Abusers generally utilize crushing and extraction processes in order to retrieve the high concentration of the active ingredient from the original dosage form. Once crushed, they will either directly abuse it by insufflation, or they add the crushed powder into an aqueous solution or a hydro-alcoholic solution for further extraction of the active ingredient(s).

In one form of abuse, the abuser will use the whole tablet with an ingestion of alcohol. The primary polymers of this disclosure increase the resistance of the tablet to mechanical crushing, and change the solution state of the extraction medium into a solid gel, by which no or minimum drug will be extracted from the abuse-deterred dosage form.

The primary polymers of this disclosure can operate to produce no change, or an insignificant change in the release profile of the active ingredient in the acidic environment of the stomach, when used as intended for a regular patient. Polymers of this disclosure can be physically mixed with the active ingredient to make a matrix tablet, or can be used as a separate layer to make bi- or multiple layer tablets, or can be used in the preparation of other dosage forms.

The invention enables the formation of prescription drugs less likely to be abused by the most common methods of medication tampering. The disclosure addresses each tampering method, and defines a way to lessen its likelihood of occurring. The invention thus targets multiple methods of abuse with the use of one or more polymers that can be incorporated into the current methods of tablet manufacturing.

The following points highlight the theoretical concept and approach for discouraging or preventing each type of tampering method.

CRUSHING: Prospective abusers crush tablets containing potent pharmaceutical ingredients that can directly be snorted into the nose. The active medication is quickly absorbed through the nasal tissue and into the blood stream giving the abuser a quick “high” and a euphoric or desired feeling.

According to an embodiment of the disclosure, primary superabsorbent polymers will be added to tablets, and upon being crushed and inhaled, will swell and form a gel layer when in contact with the wet nasal lining. The changing of dry powder into a gel mass in the nose also “traps” the drug and prevents its quick release into the blood. These two effects are intended to discourage abuse by the nasal route and slow release of the drug into the bloodstream. Moreover the primary plastic agent incorporated into the tablet formulation causes the tablet to be crushed into much larger pieces, and makes the overall crushing process more difficult. As opposed to fine particles, large pieces of crushed tablet with less contact surface area provide a slower drug release into the nasal lining in case of insufflation, and/or act to retard the dissolution and extraction in case of abuse by injection.

INTRAVENOUS (IV) ABUSE: After successfully crushing a tablet containing a drug for abuse, the powder is dissolved in water, alcohol, or other available liquids. The mixture is then filtered to remove any un-dissolved material before being drawn up into a syringe and injected. This results in a large amount of drug entering the body at once and provides the user with a powerful “rush” and euphoric effect.

In accordance with the disclosure, water-swellable superabsorbent polymers can be incorporated into the tablet to deter this type of abuse. After a tablet containing one or more of these polymers is crushed and mixed with an appropriate amount of liquid needed for intravenous injection, the powder in the liquid medium, in a very short period of time turns into a swollen gel that traps the active drug and liquid. The water-swollen mass cannot be filtered using a regular filter paper such as coffee filter paper, or lab filter papers. This approach is therefore designed to impede the ability to abuse a tablet by intravenous injection.

ALCOHOL CO-INGESTION: Swallowing the tablet medication (whole tablet or crushed) with alcohol is commonly experienced to enhance the effect of both drug and alcohol. For those drugs that dissolve in alcohol, this act also gives the user a quicker euphoric feeling since the drug can dissolve and enter the bloodstream more quickly.

In accordance with the disclosure, alcohophilic superabsorbent polymers can be added to the tablet, which when swallowed with alcohol, absorb and trap both alcohol and the dissolved drug so its quick absorption and euphoric effects are less likely to occur.

The inventors have determined that advantageous polymer properties for abuse deterrent applications include characteristics for 1) interacting with moisture in the air when exposed from a crushed tablet, 2) swelling and gelling in water and hydro-alcoholic solutions which are used by abusers to tamper with the medication, and 3) absorbing alcohol and soluble drug when medication is co-ingested with alcoholic beverages.

Polymers with great affinity for water tend to display the least affinity for alcohol, and vice versa. Alternatively stated, a polymer that absorbs significant amounts of water or significantly increases the viscosity of an aqueous solution, will experience a very weak interaction with water if alcohol is added into an aqueous solution. The disclosure identifies specific types of polymers with moderate affinity for both water and alcohol, and/or polymer combinations where one has good affinity for water and the other a good affinity for alcohol.

In accordance with the disclosure, primary superabsorbent polymers advantageously can be: made of very hydrophilic monomers, ionics and non-ionics; chemically crosslinked; absorbent of an aqueous medium rich in water; absorbent of an aqueous medium rich in alcohol; and very hygroscopic. In addition, they can: form an integral part of the formulation; prevent crushed medication particles from becoming free flowing under any abusable action such as snorting; effectively prevent filterability and impede the ability to abuse a tablet by intravenous injection; trap the drug dissolved in the hydroalcoholic solution and prevent its rapid absorption and euphoric effects when swallowed with alcoholic beverages.

Examples of such polymers include crosslinked polymers, copolymers and terpolymers of water-soluble monomers of sodium acrylate, potassium acrylate, sodium methacrylate, potassium methacrylate, potassium sulfopropyl acrylate, acrylamide, 2-acrylamido 2-methyl 1-propane sulfonic acid (AMPS), and methacrylamidopropyltrimethyl ammonium chloride.

Superabsorbent polymers of this disclosure include crosslinked poly(sodium acrylate), crosslinked poly(sulfopropyl acrylate potassium), crosslinked polyacrylamide, crosslinked copolymer of acrylamide and sodium acrylate. Synthetic polymers of this disclosure can be prepared following a general experimental procedure that we previously reported which are incorporated herein by reference, or their purified commercial counterparts can be used instead.

An additional component includes a primary plastic agent, which advantageously: is soluble or insoluble in water; has good thermoplastic properties; and has binding and adhesion properties. Additionally, the plastic agent should be capable of being processed at relatively low temperature in order to avoid drug thermal decomposition. The inventors have found these materials generally have glass transition temperature at around 35-55° C.

Plastic agents used in this disclosure can be blends of polyvinyl acetate and other polymers, or copolymers of vinyl acetate and other monomers.

While the foregoing primary polymers can provide sufficient performance to deter abuse, secondary polymers, which can serve as superviscosifier polymers, can be advantageously used along with the primary polymers to enhance the deterrence capacity of the dosage form. A superviscosifier is a very high molecular weight polymer with great affinity for both water and alcohol. In other words, a superviscosifier can provide significant viscosity in both aqueous and hydroalcoholic (very rich in alcohol) solutions.

Secondary polymers (Superviscosifier polymers) are advantageously made of very hydrophilic monomers, ionic and non-ionics; are not chemically crosslinked; enhance viscosity of the aqueous medium rich in water; and enhance viscosity of the aqueous medium rich in alcohol. Their function can be only to enhance the efficacy of the primary polymers used in the formulation. The secondary polymers contribute to preventing filterability and impeding the ability to abuse a tablet by intravenous injection.

Examples of such polymers include polyethylene oxide, methyl cellulose, hydroxypropyl methylcellulose, carboxymethyl cellulose, hydroxyethyl cellulose, hydroxypropyl cellulose, guar gum, and xanthan.

In the examples, tramadol and dextromethorphan HBr are used a representative of a pharmaceutically-active ingredient. It should be understood that other drugs can be used, as described elsewhere herein.

The terms “a” or “an”, as used herein, are defined as one or more than one. The term plurality, as used herein, is defined as two or more than two. The term another, as used herein, is defined as at least a second or more. The terms “including” and “having,” as used herein, are defined as comprising (i.e., open language). The term “coupled,” as used herein, is defined as “connected,” although not necessarily directly, and not necessarily mechanically.

As used herein, the term “about” means plus or minus ten (10) percent of the stated numerical value.

The phrase “pharmaceutically-acceptable excipient” refers to an inactive and non-toxic substance used in association with an active substance usually to prepare a dosage form.

Other objectives and advantages of this invention will become apparent from the following description taken in conjunction with the accompanying drawings, wherein are set forth, by way of illustration and example, certain embodiments of this invention. The drawings constitute a part of this specification and include exemplary embodiments of the present invention and illustrate various objects and features thereof.

### BRIEF DESCRIPTION OF THE DRAWINGS

A more complete understanding of the present invention may be obtained by references to the accompanying drawings when considered in conjunction with the subsequent detailed description. The embodiments illustrated in the drawings are intended only to exemplify the invention and should not be construed as limiting the invention to the illustrated embodiments.

FIG. 1 is a bar graph showing gel strength of representative polyethylene oxide (PEO) in various solutions. This data shows average gel strength of about 34 mN in water, saline, 20% ethanol (EtOH), and 40% ethanol (EtOH).

FIG. 2 is graph showing sustained release profiles of a 250 mg tablet of high molecular weight polyethylene oxide (PEO) in water and in a 0.1N hydrochloric acid (HCL) solution.

FIG. 3 is a graph showing drug binding capacity of carboxymethylcellulose. Carboxymethylcellulose (250 mg) was dissolved in hydro-alcoholic solutions (60%, 65%, 70%, 90%, and pure ethanol) containing 25 mg tramadol HCL.

FIG. 4 is a bar graph showing gel strength when using aluminum chloride and sodium carboxymethylcellulose (CMC).

FIGS. 5-10 show solvents: water, normal saline, 20% ethanol (EtOH), 40% ethanol, and 0.1 N HCL.

FIG. 5 is a bar graph showing gel strength of carboxymethylcellulose (Akucell®); Akucell®, Akucell® Prosolv®, Akucell® Prosolv AlOH, and Akucell® Prosolv® AlCl₃.

FIG. 6 is a bar graph showing gel strength of carboxymethylcellulose (Ticalose®); Ticalose®, Ticalose® Prosolv®, Ticalose® Prosolv® AlOH₃, and Ticalose® Prosolv® AlCl₃.

FIG. 7 is a bar graph showing gel strength of carboxymethylcellulose Akucell® Prosolv® AlCl₃ and Ticalose® 6000 Prosolv® AlCl₃.

FIG. 8 is a bar graph showing gel strength of carboxymethylcellulose Akucell® Prosolv® AlOH₃ and Ticalose® 6000 Prosolv® AlOH₃.

FIG. 9 is a bar graph showing gel strength of carboxymethylcellulose Akucell® Prosolv® and Ticalose® 6000 Prosolv®.

FIG. 10 is a bar graph showing gel strength of carboxymethylcellulose Akucell® Prosolv® and Ticalose® 6000 Prosolv®. This experiment was conducted in the absence of gelling agent.

FIG. 11 is a graph showing drug release in 0.1 N HCL as a dissolution medium.

FIG. 12 is a graph showing drug release in distilled water as a dissolution medium.

FIG. 13 is a graph showing drug release of a control tablet in 0.1 N HCL and distilled water as dissolution media.

FIG. 14 is a graph showing drug release of a sustained release (SR) tablet AlOH₃ in 0.1 N HCL and distilled water as dissolution media.

FIG. 15 is a graph showing drug release of a sustained release (SR) AlCl₃ tablet in 0.1 N HCL and distilled water as dissolution media.

FIG. 16 is a bar graph showing gel strength when using zinc acetate and sodium carboxymethylcellulose (CMC).

FIG. 17 is a bar graph showing gel strength of a Ticalose® Prosolv® mixture and a Ticalose® Prosolv® with zinc acetate mixture in solvents water, saline, 20% ethanol (EtOH), 40% ethanol, and 0.1 N HCL.

FIG. 18 is a bar graph showing gel strength of polyethylene oxide (PEO Polyox™ WSR), carboxymethylcellulose (CMC) Ticalose®, and carboxymethylcellulose (CMC) AlCl₃.

FIG. 19 is a graph showing dissolution profiles of tramadol (25 mg) in 0.1 N HCL and water. The tablets also included zinc acetate (50 mg), carboxymethylcellulose (250 mg), and Prosolv® (75 mg).

FIG. 20 shows photographs of the formulation Ticalose® 6000 (250 mg) in water (10 ml).

FIG. 21 shows photographs of the formulation Ticalose® 6000 (250 mg) in normal saline (10 ml).

FIG. 22 shows photographs of the formulation Ticalose® 6000 (250 mg) in 20% ethanol, EtOH (10 ml).

FIG. 23 shows photographs of the formulation Ticalose® 6000 (250 mg) in 40% ethanol, EtOH (10 ml).

FIG. 24 shows photographs of the formulation Ticalose® 6000 (250 mg) in 0.1 N HCL (10 ml).

FIG. 25 shows photographs of the formulation Ticalose® 6000 (250 mg) in water (10 ml) and AlCl₃ (25 mg).

FIG. 26 shows photographs of the formulation Ticalose® 6000 (250 mg) in normal saline (10 ml) and AlCl₃ (25 mg).

FIG. 27 shows photographs of the formulation Ticalose® 6000 (250 mg) in 20% ethanol, EtOH (10 ml) and AlCl₃ (25 mg).

FIG. 28 shows photographs of the formulation Ticalose® 6000 (250 mg) in 40% ethanol, EtOH (10 ml) and AlCl₃ (25 mg).

FIG. 29 shows photographs of the formulation Ticalose® 6000 (250 mg) in 0.1 N HCL (10 ml) and AlCl₃ (25 mg).

FIG. 30 shows photographs of the formulation Ticalose® 6000 (250 mg) in water (10 ml) and AlOH₃ (25 mg).

FIG. 31 shows photographs of the formulation Akucell® (250 mg) in water (10 ml) and AlOH₃ (25 mg).

FIG. 32 shows photographs of the formulation Akucell® (250 mg) in normal saline (10 ml) and AlOH₃ (25 mg).

FIG. 33 shows photographs of the formulation Akucell® (250 mg) in 20% ethanol, EtOH (10 ml) and AlOH₃ (25 mg).

FIG. 34 shows photographs of the formulation Akucell® (250 mg) in 40% ethanol, EtOH (10 ml) and AlOH₃ (25 mg).

FIG. 35 shows photographs of the formulation Akucell® (250 mg) in 0.1 N HCL (10 ml) and AlOH₃ (25 mg).

FIG. 36 shows photographs of the formulation Ticalose® 6000 (250 mg), Prosolv® (100 mg), and zinc acetate (50 mg) in water (10 ml).

FIG. 37 shows photographs of the formulation Ticalose® 6000 (250 mg), Prosolv® (100 mg), and zinc acetate (50 mg) in normal saline (10 ml).

FIG. 38 shows photographs of the formulation Ticalose® 6000 (250 mg), Prosolv® (100 mg), and zinc acetate (50 mg) in 20% ethanol, EtOH (10 ml).

FIG. 39 shows photographs of the formulation Ticalose® 6000 (250 mg), Prosolv® (100 mg), and zinc acetate (50 mg) in 40% ethanol, EtOH (10 ml).

FIG. 40 shows photographs of the formulation Ticalose® 6000 (250 mg), Prosolv® (100 mg), and zinc acetate (50 mg) in 0.1 N HCL (10 ml).

FIG. 41 is a photograph of the test apparatus used to test adhesive properties of polyvinyl alcohol-based (PVOH) cryogels.

FIG. 42 is a photograph of the test apparatus used to test hardness properties of polyvinyl alcohol-based (PVOH) cryogels.

FIG. 43 is a graph showing adhesive force of PVOH cryogels at 5% wt, 8% wt, and 10% wt concentration.

FIG. 44 is a graph showing adhesiveness of PVOH cryogels at 5% wt, 8% wt, and 10% wt concentration.

FIG. 45 is a graph showing gumminess of PVOH cryogels at 5% wt, 8% wt, and 10% wt concentration.

FIG. 46 is a graph showing hardness of PVOH cryogels at 5 wt %, 8 wt %, and 10 wt % concentration.

FIG. 47 is a bar graph showing water absorption (swelling) of PVOH cryogels at 5 wt %, 8 wt %, and 10 wt % concentration.

FIG. 48 is a graph showing acetaminophen (APAP) release from PVOH cryogels using water as a dissolution medium; 5 wt % solution concentration.

FIG. 49 is a graph showing acetaminophen (APAP) release from PVOH cryogels using water as a dissolution medium; 10 wt % solution concentration.

FIG. 50 is a graph showing that a tramadol-loaded PVOH cryogel made at 5 wt % solution concentration can release over 80% of tramadol-HCL in approximately 45 minutes in both water and 0.1 N HCL as dissolution media.

FIG. 51 is a bar graph showing that a tramadol-loaded PVOH cryogel made at 5 wt % solution concentration can release over 80% of Ttamadol-HCL in approximately 45 minutes in both water and 0.1 N HCL as dissolution media.

FIG. 52 is a graph showing swelling of croscarmellose sodium PVOH cryogels in water, 0.1 N HCL, and 40% ethanol.

FIG. 53 is a graph showing deformation of croscarmellose sodium PVOH cryogels after swelling in water and 0.1 N HCL.

FIG. 54 is a graph showing hardness of croscarmellose sodium PVOH cryogels.

FIG. 55 is a graph showing tramadol release from tramadol-croscarmellose sodium PVOH cryogel composites using water and 0.1 N HCL as dissolution media.

FIG. 56 is a graph showing tramadol release from shredded tramadol-croscarmellose sodium PVOH cryogel composites using water and 0.1 N HCL as dissolution media.

FIG. 57 is a graph showing tramadol release from tramadol-croscarmellose sodium PVOH cryogel composites using 0.1 N HCL as dissolution medium.

FIG. 58 is a schematic diagram illustrating formation of a multilayer cryogel.

FIG. 59 is a schematic diagram illustrating that layers having different properties can be assembled into multilayer cryogels.

FIG. 60 is a schematic diagram illustrating preparation of a bilayer cryogel dosage form.

FIG. 61 shows photographs illustrating bending in a tablet dosage form.

FIG. 62 is a bar graph showing results of a drug concentration (tramadol) analysis in various solutions including water, saline, 40% ethanol (EtOH 40%), pH3 solution, and 0.1 N HCL. Ac-di-Sol® was used as the deterrent agent in the formulation. The concentrations of drug in solution and drug trapped in the polymers were analyzed.

FIG. 63 is a graph showing drug release (tramadol) using water and 0.1 N HCL as dissolution media.

FIGS. 64-67 show photographs from extraction studies on a tablet formulation including tramadol HCL (100 mg), Ticalose® 6000 (300 mg), and Prosolv® (100 mg) in various dissolution media.

FIG. 64 is a series of photographs of vials of the composition using water as the dissolution media.

FIG. 65 is a series of photographs of vials of the composition using saline as the dissolution media.

FIG. 66 is a series of photographs of vials of the composition using solution of pH 3 as the dissolution media.

FIG. 67 is a series of photographs of vials of the composition using 40% ethanol (EtOH 40%) as the dissolution media.

FIG. 68 is a graph showing sustained drug (tramadol) release using water and 0.1 N HCL as dissolution media.

FIG. 69 is a graph showing sustained drug (tramadol) release of the crush-resistant formulation using water and 0.1 N HCL as dissolution media. The graph also shows the heat treated formulation compared to the untreated formulation. Kollidon® SR 150 mg

FIG. 70 is a graph showing sustained drug (tramadol) release of the crush-resistant formulation using water and 0.1 N HCL as dissolution media. The graph also shows the heat treated formulation compared to the untreated formulation. Kollidon® SR 250 mg

FIG. 71 is a graph showing drug (tramadol) release of a non-heat treated formulation using water and 0.1 N HCL as dissolution media. Kollidon® SR 250 mg, Ac-Di-Sol® 150 mg (croscarmellose)

FIG. 72 is a graph showing drug (tramadol) release of a heat treated formulation using water, 0.1 N HCL, and solution at pH 3 as dissolution media. Kollidon® SR 250 mg, Ac-Di-Sol® 150 mg (croscarmellose)

FIGS. 73A-C show data from crush resistance studies of tablets including 50% Kollidon® SR and 30% Ac-Di-Sol® (croscarmellose)

FIG. 73A shows photographs of the non-heat treated formulation after 10 rotations.

FIG. 73B shows photographs of the heat treated formulation after 10 rotations.

FIG. 73C shows photographs of the heat treated formulation after 50 rotations.

FIGS. 74A-C show data from particle size distribution studies.

FIG. 74A is a bar graph showing results from a sustained release formulation including tramadol HCL 100 mg, Ticalose® 6000 150 mg, and Kollidon® SR 150 mg.

FIG. 74B is a bar graph showing results from a sustained release formulation including tramadol HCL 100 mg, Ticalose® 6000 150 mg, and Kollidon® SR 250 mg.

FIG. 74C is a bar graph showing results from an immediate and sustained release formulation including tramadol HCL 100 mg, Ac-Di-Sol® (croscarmellose) 150 mg, and Kollidon® SR 250 mg.

FIG. 75 is a photograph showing relative particle size.

FIG. 76 is a bar graph showing particle size of heat treated and non-heat treated formulations.

FIGS. 77-80 show the effect of deterrent (superdisintegrant) concentration on tramadol release with no heat treatment. The dissolution media were water and 0.1 N HCL.

FIG. 77 is a graph showing results of release from a formulation including no deterrent.

FIG. 78 is a graph showing results of release from a formulation including 10% deterrent.

FIG. 79 is a graph showing results of release from a formulation including 20% deterrent.

FIG. 80 is a graph showing results of release from a formulation including 30% deterrent.

FIGS. 81-84 show the effect of deterrent (superdisintegrant) concentration on tramadol release with heat treatment. The dissolution media were water and 0.1 N HCL.

FIG. 81 is a graph showing results of release from a formulation including no deterrent.

FIG. 82 is a graph showing results of release from a formulation including 10% deterrent.

FIG. 83 is a graph showing results of release from a formulation including 20% deterrent.

FIG. 84 is a graph showing results of release from a formulation including 30% deterrent.

FIG. 85 is a graph comparing release (tramadol) profiles of heated and non-heat treated formulations using water as a dissolution media. 0/250

FIG. 86 is a graph comparing release (tramadol) profiles of heated and non-heat treated formulations using water as a dissolution media. 50/250

FIG. 87 is a graph comparing release (tramadol) profiles of heated and non-heat treated formulations using water as a dissolution media. 100/250

FIG. 88 is a graph comparing release (tramadol) profiles of heated and non-heat treated formulations using water as a dissolution media. 150/250

FIG. 89 is a graph comparing release (tramadol) profiles of heated and non-heat treated formulations using 0.1 N HCL as a dissolution media. 0/250

FIG. 90 is a graph comparing release (tramadol) profiles of heated and non-heat treated formulations using 0.1 N HCL as a dissolution media. 50/250

FIG. 91 is a graph comparing release (tramadol) profiles of heated and non-heat treated formulations using 0.1 N HCL as a dissolution media. 100/250

FIG. 92 is a graph comparing release (tramadol) profiles of heated and non-heat treated formulations using 0.1 N HCL as a dissolution media. 150/250

FIG. 93 is a photograph of a tablet in a ball mill container with a steel ball. Both heat treated and non-heat (regular) treated tablets were tested for crush resistance using a ball mill apparatus.

FIG. 94 is a bar graph showing particle size distribution of heat treated and non-heat (regular) treated tablets following ball milling.

FIG. 95 is a bar graph showing particle size distribution of heat treated and non-heat (regular) treated tablets following ball milling at greater than >250 μm and less than <250 μm.

FIG. 96 shows photographs illustrating the physical appearance of treated and non-heat (regular) treated tablets following ball milling.

FIG. 97 is a bar graph showing data from extraction studies using a formulation including tramadol HCL, Prosolv®, and Ac-di-Sol®. The percentages of drug trapped are shown using different dissolution media; water, saline, EtOH 40%, solution of pH 3, and 0.1 N HCl.

FIG. 98 is a graph showing drug (tramadol) release from a formulation including tramadol HCL, Prosolv®, and Ac-di-Sol® using water and 0.1 N HCl as dissolution media.

FIGS. 99A-B show data regarding effect of heat treatment on drug release of the formulation including tramadol HCL, Prosolv®, and Ac-di-Sol®.

FIG. 99A is a graph showing that non-heat treated and heat treated tablets at different temperatures were able to release tramadol content in water up to 44-48%.

FIG. 99A is a graph showing that non-heat treated and heat treated tablets at different temperatures were able to release tramadol content in 0.1 N HCl up to 100%.

FIGS. 100-102 show data resulting from ball mill studies.

FIG. 100 is a bar graph showing crush resistance of the formulation at various ratios of A (deterrent agent) to polyethylene oxide (PEO).

FIGS. 101 and 102 are graphs showing crush resistance of the formulation at various ratios of A (deterrent agent) to polyethylene oxide (PEO) obtained from sieve #20.

FIGS. 103-106 show data regarding effect of the molecular weight of PEO on thermal properties of the formulation.

FIG. 103 is a graph showing resulting from a first heating cycle using different grades of PEO.

FIG. 104 is a graph showing resulting from a second heating cycle using different grades of PEO.

FIG. 105 is a graph showing melting points of different grades of PEO.

FIG. 106 is a graph showing heat fusion of different grades of PEO.

FIG. 107 is a bar graph showing mechanical resistance via ball milling of formulations having different grades of PEO.

FIG. 108 is a bar graph showing mechanical resistance via grind milling of formulations having different grades of PEO.

FIG. 109 is a graph showing effect of curing temperatures on crush resistance of the formulation. An amount of particles retained on various sieve numbers is shown.

FIG. 110 is a graph showing effect of curing temperatures on crush resistance of the formulation. Various melting temperatures are shown.

FIG. 111 is a graph showing drug (tramadol) release of a formulation including tramadol 25 mg, Ac-di-Sol® 250 mg, and PEO 250 mg using water and 0.1 N HCl as dissolution media.

FIG. 112 is a bar graph showing crush resistance of heat treated and untreated tablets after processing in a ball mill.

FIG. 113 is a bar graph showing crush resistance of heat treated and untreated tablets after processing in a grind mill.

FIGS. 114-116 are photographs showing results from a pill crusher study.

FIG. 114 shows photographs of untreated tablets after 50 rotations in a pill crusher.

FIG. 115 shows photographs of treated tablets after 50 rotations in a pill crusher.

FIG. 116 shows photographs of a side-by-side comparison of the physical appearance of treated and untreated tablets after pill crushing.

FIGS. 117-118 are photographs showing results from a ball mill study.

FIG. 117 shows photographs of particle distribution, >250 μg and <250 μg, of an untreated tablet (no heat).

FIG. 118 shows photographs of particle distribution, >250 μg and <250 μg, of an treated tablet (heat).

FIGS. 119-120 are photographs showing results from a grind mill study.

FIG. 119 shows photographs of particle distribution, >250 μg and <250 μg, of an untreated tablet (no heat).

FIG. 120 shows photographs of particle distribution, >250 μg and <250 μg, of an treated tablet (heat).

FIG. 121 is a graph showing drug (tramadol) release from PEO (100K)-based tablets using water and 0.1 N HCl as dissolution media.

FIG. 122 is a bar graph showing results of a crush resistance study of heated and untreated PEO (100K)-based tablets using a ball mill.

FIG. 123 is a bar graph showing results of a crush resistance study of heated and untreated PEO (100K)-based tablets using a grind mill.

FIGS. 124-132 show data resulting from tests on tablets prepared using PEO (100K) and tablets prepared using a polyox coagulant.

FIG. 124 shows results of a particle size distribution test, after grind milling, of heated and untreated tablets containing Polyox™ coagulant.

FIG. 125 shows results of a particle size distribution test, after grind milling, of heated and untreated tablets containing PEO (100K).

FIGS. 126-127 are photographs showing results from a pill crusher study.

FIG. 126 shows photographs of untreated tablets after 50 rotations in a pill crusher.

FIG. 127 shows photographs of treated tablets after 50 rotations in a pill crusher.

FIGS. 128-129 are photographs showing results from a ball mill study.

FIG. 128 shows photographs of particle distribution, >250 μg and <250 μg, of an untreated tablet (no heat).

FIG. 129 shows photographs of particle distribution, >250 μg and <250 μg, of an treated tablet (heat).

FIGS. 130-131 are photographs showing results from a grind mill study.

FIG. 130 shows photographs of particle distribution, >250 μg and <250 μg, of an untreated tablet (no heat).

FIG. 131 shows photographs of particle distribution, >250 μg and <250 μg, of an treated tablet (heat).

FIG. 132 is a graph showing drug (tramadol) release from tablets including PEO (100K) and polyox Coagulant™ using water and 0.1 N HCl as dissolution media.

FIGS. 133-134 show data resulting from crush resistance tests on formulations including 12.5 mg, 25 mg, and 50 mg of Carbowax™ polyethylene glycol (PEG) 8000 (a plasticizer). A formulation without PEG 8000 was used as a control.

FIG. 133 is a bar graph showing crush resistance of untreated (no heat) tablets after processing in a ball mill.

FIG. 134 is a bar graph showing crush resistance of heat treated tablets after processing in a ball mill.

FIG. 135 is a bar graph showing particle size distribution after ball milling of the control formulation without PEG 8000. No heat treatment.

FIG. 136 is a bar graph showing particle size distribution after grind milling of the control formulation without PEG 8000. Heat treatment.

FIG. 137 is a bar graph showing particle size distribution after ball milling of the formulation including 25 mg PEG 8000. Heat treatment.

FIG. 138 is a bar graph showing particle size distribution after grind milling of the formulation including 25 mg PEG 8000. Heat treatment.

FIG. 139 is a bar graph showing particle size distribution after ball milling and grind milling of the control formulation without PEG 8000.

FIG. 140 is a bar graph showing particle size distribution after ball milling and grind milling of the formulation including 25 mg PEG 8000.

FIG. 141 is a bar graph showing particle size distribution after ball milling of the formulation including 25 mg PEG 8000 and the control formulation without PEG 8000.

FIG. 142 is a bar graph showing particle size distribution after grind milling of the formulation including 25 mg PEG 8000 and the control formulation without PEG 8000.

FIG. 143 is a bar graph showing particle size distribution of the control formulation without PEG 8000 after heat treatment at 100° C. for 1 hour.

FIG. 144 is a bar graph showing particle size distribution of the formulation including PEG 8000 after heat treatment at 100° C. for 1 hour.

FIG. 145 is a bar graph showing particle size distribution of the control formulation without PEG 8000 after ball milling and grind milling.

FIG. 146 is a bar graph showing particle size distribution of the formulation including PEG 8000 after ball milling and grind milling.

FIG. 147A is a bar graph showing particle size distribution of the control formulation without PEG 8000 after heat treatment at 80° C. for 1 hour.

FIG. 147B is a bar graph showing particle size distribution of the formulation including PEG 8000 after heat treatment at 80° C. for 1 hour.

FIG. 148 is a bar graph showing particle size distribution of the control formulation without PEG 8000 after heat treatment at 80° C. for 1 hour with ball milling and grind milling.

FIG. 149 is a bar graph showing particle size distribution of the formulation including PEG 8000 after heat treatment at 80° C. for 1 hour with ball milling and grind milling.

FIG. 150 is a bar graph showing percentage of particles retained on sieve #20; particles larger than 850 μm.

FIG. 151 is a bar graph showing particle size distribution for both the control formulation without PEG 8000 and the formulation including PEG 8000 using different heat treatments and different time periods of ball milling.

FIG. 152 is a bar graph showing particle size distribution for both the control formulation without PEG 8000 and the formulation including PEG 8000 using different heat treatments and different time periods of grind milling.

FIG. 153 is a graph showing drug release for both the control formulation without PEG 8000 and the formulation including PEG 8000 using water and 0.1 N HCl as dissolution media. No heat treatment.

FIG. 154 is a graph showing drug release for both the control formulation without PEG 8000 and the formulation including PEG 8000 using water and 0.1 N HCl as dissolution media after heat treatment at 90° C. for 1 hour.

FIG. 155 is a graph showing drug release for both the control formulation without PEG 8000 and the formulation including PEG 8000 using water and 0.1 N HCl as dissolution media after heat treatment at 120° C. for 1 hour.

FIG. 156 is a graph showing different amounts of clay as added into 10 ml of 25 μg/ml tramadol aqueous solution. Dispersions were vortexed for 5 seconds and then centrifuged at 1500 rpm for 5 minutes. The supernatant was then analyzed for tramadol concentration.

FIG. 157 is a schematic diagram illustrating coagulation resulting from addition of a coagulable deterrent agent to a drug.

FIG. 158 is a bar graph showing results of drug entrapment by coagulation test. The amount of drug (tramadol) bound to clay particles is shown.

FIG. 159 is a series of three photographs showing that coagulation reduces the volume of filtrate in the coagulation system. The left panel shows 10 ml of starting solution; the middle panel shows residue; and the right panel shows volume of filtrate remaining.

FIG. 160 is a series of three photographs showing that coagulation reduces the volume of filtrate in the coagulation system. These are photographs from a test using a greater amount of clay than that which is shown in FIG. 159. The left panel shows 10 ml of starting solution; the middle panel shows residue; and the right panel shows volume of filtrate remaining.

FIG. 161 is a photograph showing the physical appearance of calcium bentonite (1 g) mixed with different amounts of sodium carbonate after centrifugation at 1500 rpm for 5 minutes. From left to right: 0% Na₂CO₃; 40% Na₂CO₃; 50% Na₂CO₃; 70% Na₂CO₃; and 100% Na₂CO₃.

FIGS. 162-168 are photographs showing the physical appearance of differences observed between sodium bentonite and its calcium form.

FIG. 162 is a photograph of calcium bentonite reaction to polyethylene oxide (PEO).

FIG. 163 is a photograph of sodium bentonite reaction to PEO.

FIG. 164 is a photograph of dispersion stability of calcium bentonite after centrifugation at 1500 rpm for 5 minutes.

FIG. 165 is a photograph of dispersion stability of sodium bentonite after centrifugation at 1500 rpm for 5 minutes.

FIG. 166 is a photograph of dispersion stability of calcium bentonite (left) and sodium bentonite (right) after centrifugation at 4500 rpm for 5 minutes.

FIG. 167 is a photograph of calcium bentonite after filtration through 0.2 μm filter.

FIG. 168 is a photograph of sodium bentonite after filtration through 0.2 μm filter.

FIG. 169 is a bar graph showing filtration times of sodium bentonite and calcium bentonite.

FIG. 170 is a photograph showing sodium bentonite (left) and calcium bentonite (right) retention on a filter membrane following vacuum filtration.

FIGS. 171-180 are a series of photographs demonstrating suspension stability of sodium and calcium bentonite under normal gravity for 12 hours.

FIG. 171 shows the physical appearance of calcium bentonite with no drug added.

FIG. 172 shows the physical appearance of sodium bentonite with no drug added.

FIG. 173 shows the physical appearance of calcium bentonite with 1000 μg/mL drug added.

FIG. 174 shows the physical appearance of sodium bentonite with 1000 μg/mL drug added.

FIG. 175 shows the physical appearance of calcium bentonite with 500 μg/mL drug added.

FIG. 176 shows the physical appearance of sodium bentonite with 500 μg/mL drug added.

FIG. 177 shows the physical appearance of calcium bentonite with 200 μg/mL drug added.

FIG. 178 shows the physical appearance of sodium bentonite with 200 μg/mL drug added.

FIG. 179 shows the physical appearance of calcium bentonite with 100 μg/mL drug added.

FIG. 180 shows the physical appearance of sodium bentonite with 100 μg/mL drug added.

FIG. 181 is a graph of a thermogravimetric analysis used to determine amounts of moisture in various bentonite clays.

FIGS. 182-189 are a series of photographs demonstrating flocculation behavior of bentonite samples after adding 2 mg polyethylene oxide (PEO) in 10 mL of suspension containing 25 mg clay (bentonite).

FIG. 182 shows the physical appearance of calcium clay in water with 1000 μg/mL drug added.

FIG. 183 shows the physical appearance of sodium clay in water with 1000 μg/mL drug added.

FIG. 184 shows the physical appearance of calcium clay in water with 100 μg/mL drug added.

FIG. 185 shows the physical appearance of sodium clay in water with 100 μg/mL drug added.

FIG. 186 shows the physical appearance of calcium clay in 0.1 N HCL with 1000 μg/mL drug added.

FIG. 187 shows the physical appearance of sodium clay in 0.1 N HCL with 1000 μg/mL drug added.

FIG. 188 shows the physical appearance of calcium clay in 0.1 N HCL with 100 μg/mL drug added.

FIG. 189 shows the physical appearance of sodium clay in 0.1 N HCL with 100 μg/mL drug added.

FIG. 190 is a bar graph illustrating particle size distribution of calcium clay and sodium clay.

FIG. 191 is a graph illustrating amounts of drug recovered from the croscarmellose sodium-dextromethorphan HBR complexes (CCS-DEX complexes) after extraction in 0.1 N HCL.

FIG. 192 is a graph showing drug release for a formulation including 250 mg of CCS-DEX complexes using water and 0.1 N HCl as dissolution media. No heat treatment.

FIG. 193 is a graph showing drug release for a heat-treated formulation including 250 mg of CCS-DEX complexes using water and 0.1 N HCl as dissolution media.

FIG. 194 is a photograph showing the physical appearance of the tablets including 250 mg of CCS-DEX complexes in water (left) and 0.1 N HCL (right) after 24 hours.

FIG. 195 is a bar graph showing particle size distribution after crush resistance studies using a high shear grinder. Tablets including 250 mg of CCS-DEX complexes with and without heat treatment were tested.

### DETAILED DESCRIPTION OF THE DISCLOSURE

As required, detailed embodiments are disclosed herein; however, it is to be understood that the disclosed embodiments are merely examples and that the systems and methods described below can be embodied in various forms. Therefore, specific structural and functional details disclosed herein are not to be interpreted as limiting, but merely as a basis for the claims and as a representative basis for teaching one skilled in the art to variously employ the present subject matter in virtually any appropriately detailed structure and function. Further, the terms and phrases used herein are not intended to be limiting, but rather, to provide an understandable description of the concepts.

The examples serve the purpose of promoting an understanding of the principles of the invention. It will nevertheless be understood that no limitation of the scope of the invention is thereby intended. Any alterations and further modification in the described compositions, tablets, formulations, and methods and any further application of the principles of the invention as described herein, are contemplated as would normally occur to one skilled in the art to which the invention relates.

### Example 1: Poly(Ethylene Oxide) as Deterrent Agent

Crush resistance and viscosity building are the two most common physicochemical characteristics that have been utilized in formulating abuse-deterrent medications, and to achieve both, very high molecular weight polyethylene oxide (PEO) has been used. It has a very simple structure, potentially least interaction with drug and other excipient, it builds great viscosity in water and hydro-alcoholic solutions, and it melts at relatively low temperature in its solid state. However, low melting temperature of its solid state, shear-dependent viscosity of its solution state, salt and light sensitivity are among disadvantages that need to be taken into consideration when polyethylene oxide is used as deterrent. Moreover, it has very slow dissolution kinetics in aqueous systems; in fact its complete dissolution in water may take hours if no provision is taken to expedite the process.

PEO concentration in tablets with deterrence potential can be as high as 200-300 mg per tablet, and the amount of liquid that abusers use to extract the drug from the tablet can be as average as 10 mL. At such high concentration of PEO in an aqueous medium (2-3 wt. %), the PEO solution turns into a PEO gel for which gel strength can alternatively be measured.

### Gel Strength of Representative PEO Solutions:

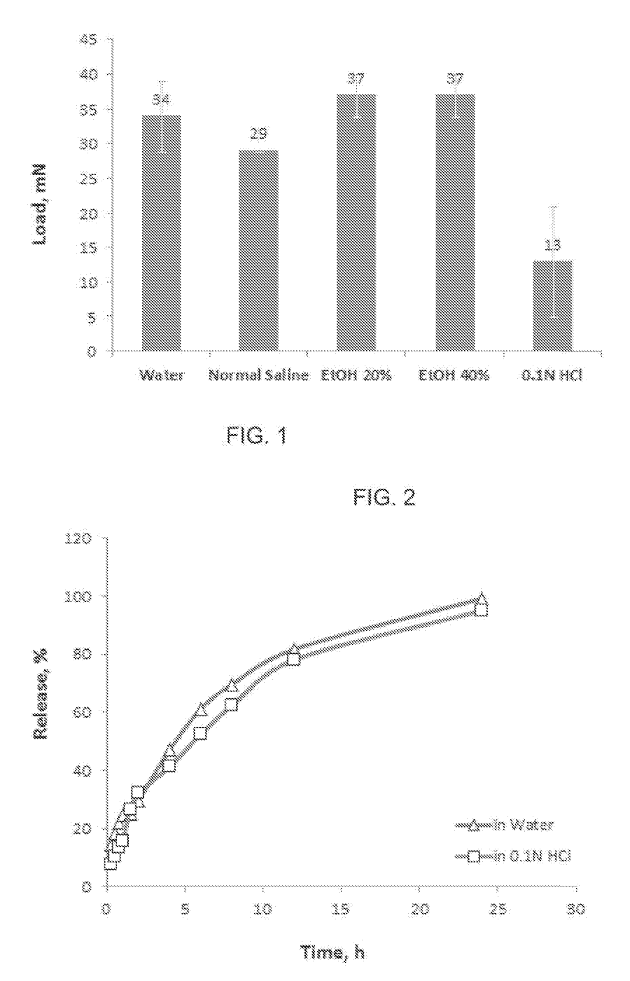

250 mg of PEO WSR coagulant was dissolved in respective solutions (water, normal saline, 20% ethanol or EtOH 20%, and 40% ethanol or EtOH 40%) and kept overnight for complete and homogeneous dissolution. For the hydrocholoric acid (HCl) study, the solution pH of PEO in water was changed to 1 using concentrated HCl. The gel strengths were measured on Texture Analyzer (CT3, Brookfield Engineering). A spherical probe was attached to the shaft and was moved into the sample at the speed of 2 mm/sec. Once it reached the trigger load, the probe moved to a distance of 4 mm into the gel at the speed of 0.5 mm/sec, and the magnitude of load at the target was measured. The gel strength data shows average gel strength of about 34 mN in water, saline, EtOH 20% and EtOH 40%. The PEO gel strength in 0.1N HCl medium was found insignificant. Results are illustrated in FIG. 1

Drug Release from Representative PEO Tablets:

Tablets containing 25 mg of tramadol HCl, 250 of PEO (WSR coagulant), and Prosolv® SMCC were prepared using a Carver press at 1000 pound force. The tablets prepared were studied for dissolution profile in distilled water and 0.1N HCl at 370 C @ 50 rpm. Samples were drawn at specific time points, and drug concentration was determined using a UV-spectrophotometer @271 nm.

Based on the dissolution data, a tablet containing 250 mg of high molecular weight PEO would offer very similar sustained release profiles in water and 0.1N HCl solutions (ANOVA p value of 0.674). Results are illustrated in FIG. 2.

The data presented for PEO drug release and gel strength show that hydrochloric acid plays a major role in gel strength with lesser effect on drug release. The instant invention offers an alternative method of abuse deterrence where the hydrochloric acid plays a major role in drug release from abuse-deterrent compositions.

### Example 2: Carboxymethylcellulose as Deterrent Agent

Materials: Materials examined in this disclosure are tramadol HCl, sodium carboxymethyl cellulose (Ticalose® 6000 from TIC Gums, and Akucell® from AkzoNobel), aluminum chloride hexahydrate, aluminum hydroxide, zinc acetate, talc, and Prosolv® SMCC (silicified microcrystalline cellulose, JRS Pharma).

### Drug Binding Capacity of Carboxymethylcellulose:

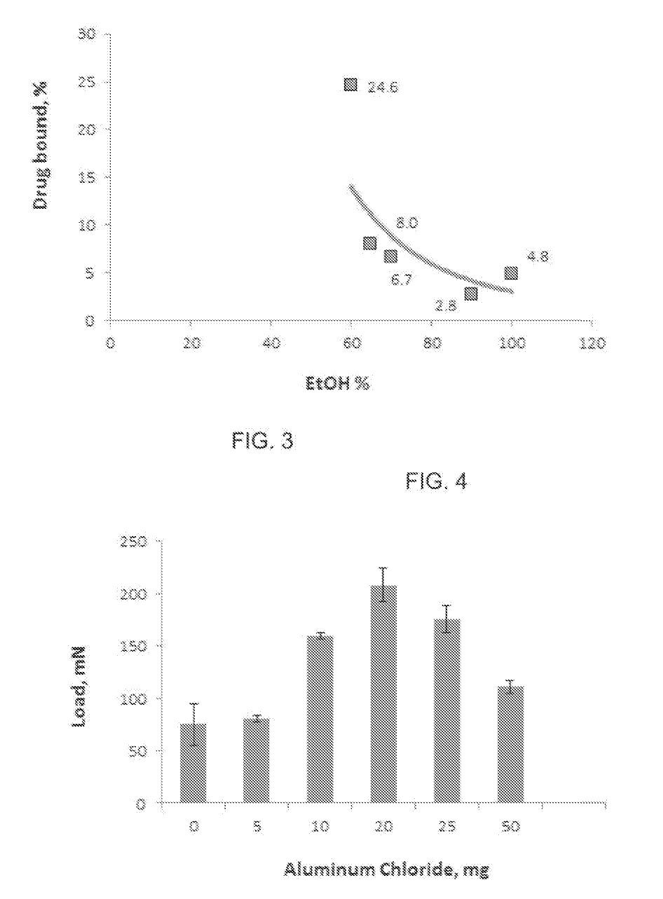

Carboxymethylcellulose (250 mg) was dissolved in hydro-alcoholic solutions (60%, 65%, 70%, 90% and pure ethanol) containing 25 mg of tramadol HCl. The solution was centrifuged @1500 rpm for 5 min, then filtered through a 0.2 μm syringe filter; 0.5 ml of the supernatant solution was diluted with 10 ml of solvent, and then analyzed for drug concentration by UV spectroscopy @271 nm. Results are illustrated in FIG. 3.

### Gel Strength Studies Using Three-Valent Salts:

Aluminum chloride and sodium carboxymethylcellulose (CMC) were weighed and mixed in a glass mortar. 10 mL of distilled water was added to physical mixture. Rapid gelation was observed within a few seconds. Gels were transferred to 20 mL glass vial and gel strengths were measured on Texture Analyzer (CT3, Brookfield Engineering). A spherical probe was attached to the shaft and moved into the sample at the speed of 2 mm/sec. Once it reached the trigger load, the probe moved to a distance of 4 mm into the gel at the speed of 0.5 mm/sec, and load at the target was measured. Results are illustrated in FIG. 4.

### Optimum Concentration of Aluminum Chloride (CMC Gel Strength in Water):

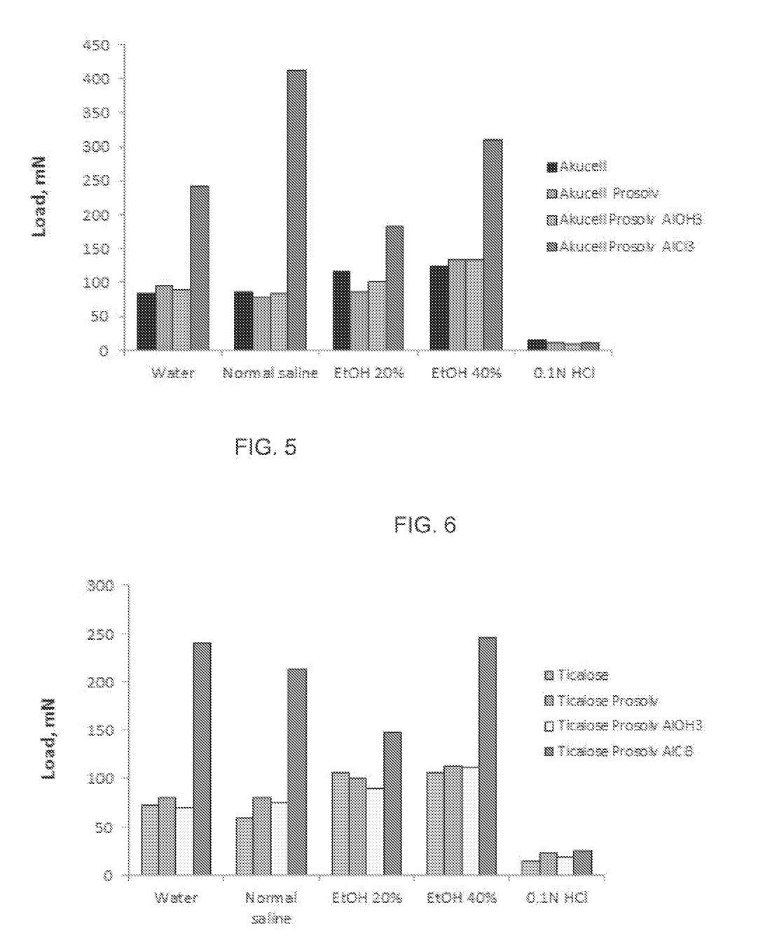

As shown in Table 1, and as illustrated in FIG. 5, a certain amount of aluminum chloride was added to 250 mg sodium CMC dissolved in 10 mL of deionized water. Gel strength of the CMC composition in water was increased with increase in aluminum chloride concentration, and reached the peak at about 20 mg per 250 mg of CMC. However, gel was separated from water as aluminum chloride concentration increased to 100 mg. In fact, addition of 20 mg of aluminum chloride significantly increased the gel strength of the 25 mg/ml aqueous solution of CMC up to 3 folds.

TABLE 1

| AlCl₃: 6H₂O, | Sodium CMC | Load, |
|---|---|---|
| mg | AF 0305 (mg) | mN |
| 0 | 250 | 75 |
| 5 | 250 | 80 |
| 10 | 250 | 159 |
| 20 | 250 | 208 |
| 25 | 250 | 176 |
| 50 | 250 | 111 |

Two different grades of Sodium CMC (Akucell® AF0305 and Ticalose® 6000) were studied for their gel strength in different solvents including water, normal saline, 20% ethanol (EtOH 20%), 40% ethanol (EtOH 40%), and 0.1N HCl, as shown in Tables 2 and 3.

TABLE 2
Gel strength data for sodium CMC (Akucell ® AF0305)
in different media.

| | | Amount, |
|---|---|---|
| Composition | Ingredients | mg |
| Blank | Sodium CMC (Akucell ® AF0305) | 250 |
| Control | Sodium CMC (Akucell ® AF0305) | 250 |
| | Prosolv ® SMCC 90 | 125 |
| Al(OH)₃ gel | Sodium CMC (Akucell ® AF0305) | 250 |
| | Prosolv ® SMCC 90 | 125 |
| | Aluminum hydroxide | 25 |
| AlCl₃ gel | Sodium CMC (Akucell ® AF0305) | 250 |
| | Prosolv ® SMCC 90 | 125 |
| | Aluminum chloride | 25 |

TABLE 3

| | | | |
|---|---|---|---|
| Blank | Control | Al(OH)3 gel | AlCl₃ gel |

| | |
|---|---|
| Medium | Gel Strength, mN |

| | | | | |
|---|---|---|---|---|
| Water | 83 | 95 | 90 | 242 |
| Normal saline | 86 | 78 | 83 | 412 |
| EtOH 20% | 116 | 85 | 101 | 183 |
| EtOH 40% | 123 | 134 | 134 | 311 |
| 0.1N HCl | 15 | 12 | 10 | 12 |

Further in accordance with the disclosure, and with reference to FIG. 5:
In all solutions except in 0.1N HCl, the composition of CMC and aluminum chloride remained intact and no sign of water and gel separation observed even in solutions rich in alcohol. Phase separation occurred only in 0.1N HCl solution.
Gel strength of all compositions is insignificant in 0.1N HCl, averaged at around 13 mN similar to the PEO solution gel strength in 0.1N HCl.
Gel strength of Akucell® dissolved in water, normal saline, EtOH 20% and EtOH 40% is not affected by the addition of Prosolv® or aluminum hydroxide.
Except with aluminum chloride, gel strength of the composition in water is averaged at 89 mN.
Except with aluminum chloride, gel strength of the composition in normal saline is averaged at 82 mN.
Except with aluminum chloride, gel strength of the composition in aqueous EtOH 20% is averaged at 100 mN.
Except with aluminum chloride, gel strength of the composition in aqueous EtOH 40% is averaged at 130 mN.
Gel strength in water, saline, EtOH 20%, and EtOH 40% increased 2.7, 5.0, 1.83, and 2.4 folds respectively for AlCl3 Akucell® gels compared to the gels with no aluminum chloride treatment.
Gel strength in water, saline, EtOH 20% and EtOH 40% increased 2.6, 2.8, 2.7, and 3.5 folds respectively for untreated Akucell® gels compared to corresponding PEO gel strength data.
Gel strength in water, saline, EtOH 20% and EtOH 40% increased 7.1, 14, 5, and 8.4 folds respectively for AlCl3 Akucell® gels compared to corresponding PEO gel strength data.

Tables 4 and 5 illustrate gel strength data of sodium CMC (Ticalose 6000) in different media.

TABLE 4

| | | Amount, |
|---|---|---|
| Composition | Ingredients | mg |
| Blank | Sodium CMC (Ticalose ® 6000) | 250 |
| Control | Sodium CMC (Ticalose ® 6000) | 250 |
| | Prosolv ® SMCC 90 | 125 |
| Al(OH)₃ gel | Sodium CMC (Ticalose ® 6000) | 250 |
| | Prosolv ® SMCC 90 | 125 |
| | Aluminum hydroxide | 25 |
| AlCl₃ gel | Sodium CMC (Ticalose ® 6000) | 250 |
| | Prosolv ® SMCC 90 | 125 |
| | Aluminum chloride | 25 |

TABLE 5

| | | | |
|---|---|---|---|
| Blank | Control | Al(OH)₃ gel | AlCl₃ gel |

| | |
|---|---|
| Medium | Gel Strength, mN |

| | | | | |
|---|---|---|---|---|
| Water | 72 | 80 | 70 | 240 |
| Normal saline | 59 | 80 | 75 | 213 |
| EtOH 20% | 106 | 101 | 90 | 147 |
| EtOH 40% | 106 | 113 | 111 | 245 |
| 0.1N HCl | 15 | 23 | 18 | 25 |

With reference to Tables 4 and 5, and FIG. 6:
Gel strength of all compositions is insignificant in 0.1N HCl, averaged at around 18 mN almost similar to the PEO solution gel strength in 0.1N HCl.
Gel strength of Ticalose® dissolved in water, normal saline, EtOH 20% and EtOH 40% is not affected by the addition of Prosolv® or aluminum hydroxide.
Except with aluminum chloride, gel strength of the composition in water is averaged at 74 mN.
Except with aluminum chloride, gel strength of the composition in normal saline is averaged at 71 mN.
Except with aluminum chloride, gel strength of the composition in EtOH 20% is averaged at 99 mN.
Except with aluminum chloride, gel strength of the composition in EtOH 40% is averaged at 110 mN.
Gel strength in water, normal saline, EtOH 20% and EtOH 40% increased 3.2, 3.0, 1.5, 2.2 folds respectively for AlCl3 Ticalose® gels compared to the gels with no aluminum chloride treatment.
Gel strength in water, saline, EtOH 20% and EtOH 40% increased 2.2, 2.4, 2.7, and 2.9 folds respectively for untreated Ticalose® gels compared to corresponding PEO gel strength data.
Gel strength in water, saline, EtOH 20% and EtOH 40% increased 7.1, 7.3, 3.9, and 6.6 folds respectively for AlCl3 Ticalose® gels compared to corresponding PEO gel strength data.

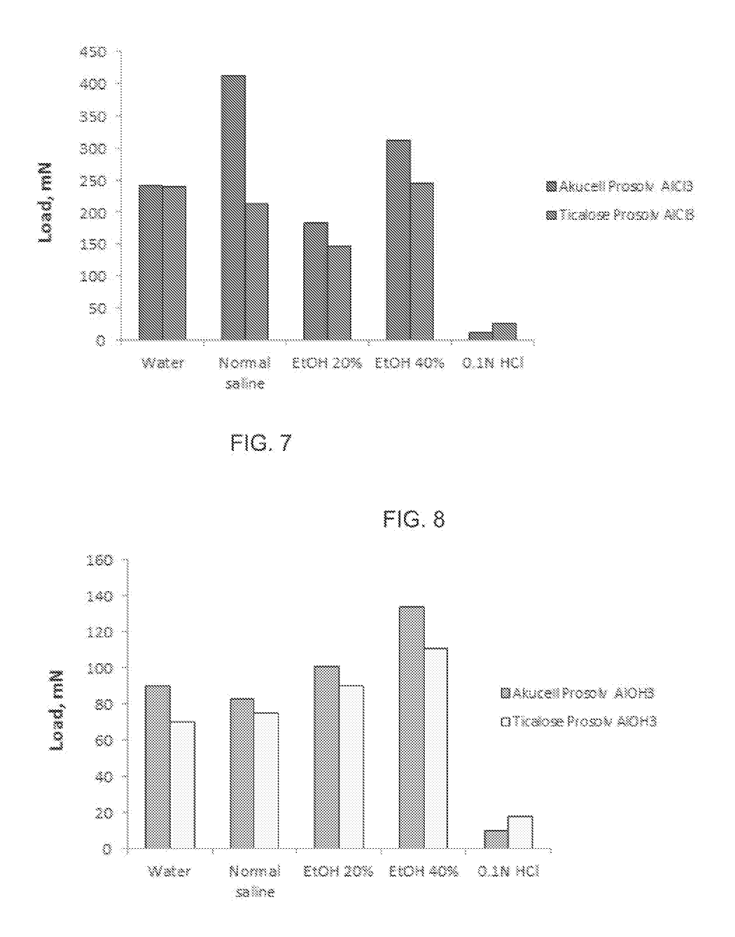

With reference to FIG. 7, two grades of CMC (Akucell® and Ticalose® 6000) displayed almost the same gel strength in all solutions when treated with the same amount of aluminum chloride.

With reference to FIG. 8, two grades of CMC (Akucell® and Ticalose® 6000) displayed almost the same gel strength in all solutions when treated with the same amount of aluminum hydroxide.

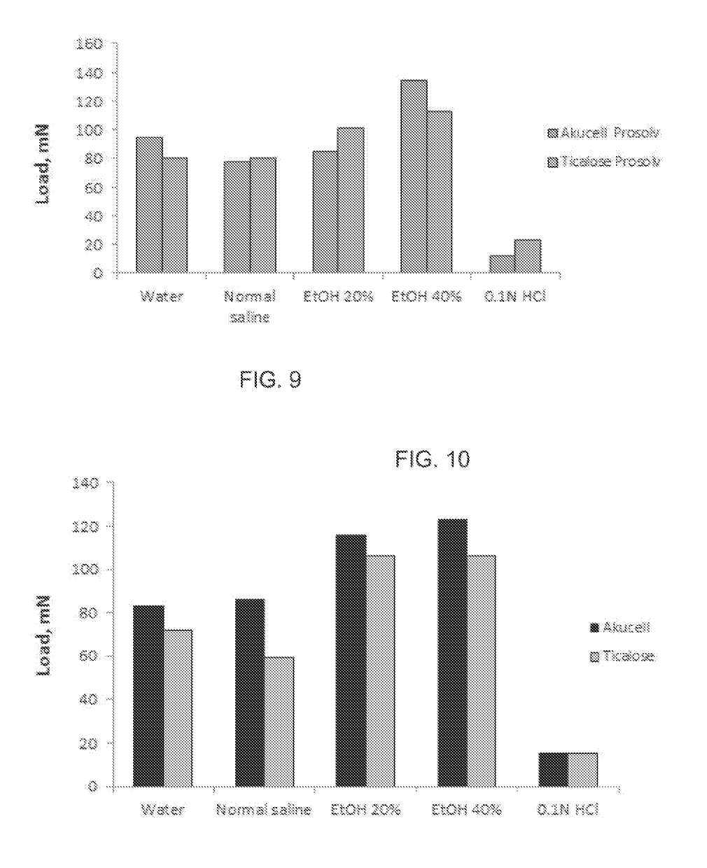

With reference to FIG. 9, two grades of CMC (Akucell® and Ticalose® 6000) displayed almost the same gel strength in all solutions when mixed with Prosolv. FIG. 10 illustrates that in the absence of gelling agent, two grades of CMC (Akucell® and Ticalose® 6000) displayed almost the same gel strength in all solutions.

### Drug Release Studies

Four formulations were prepared to represent immediate and sustained drug release compositions. Individual components were weighed and mixed using a glass mortar and pestle. The mixture was compressed on a Carver press at a 1000 pound force. The tablets prepared were studied for dissolution profile in distilled water and 0.1NHCl at 37° C. @ 50 rpm. Samples were drawn at specific time points and drug concentration was determined using a UV-spectrophotometer @271 nm, as shown in Table 6.

TABLE 6

| | Tramadol | | Ticalose ® | Aluminum |
|---|---|---|---|---|
| | HCl, | Prosolv ®, | 6000, | compound, |
| | mg | mg | mg | mg |
| Immediate release | 25 | 375 | — | — |
| Sustained release, control | 25 | 125 | 250 | — |
| Sustained release, Al(OH)₃ | 25 | 100 | 250 | 25 |
| Sustained release, AlCl₃ | 25 | 100 | 250 | 25 |

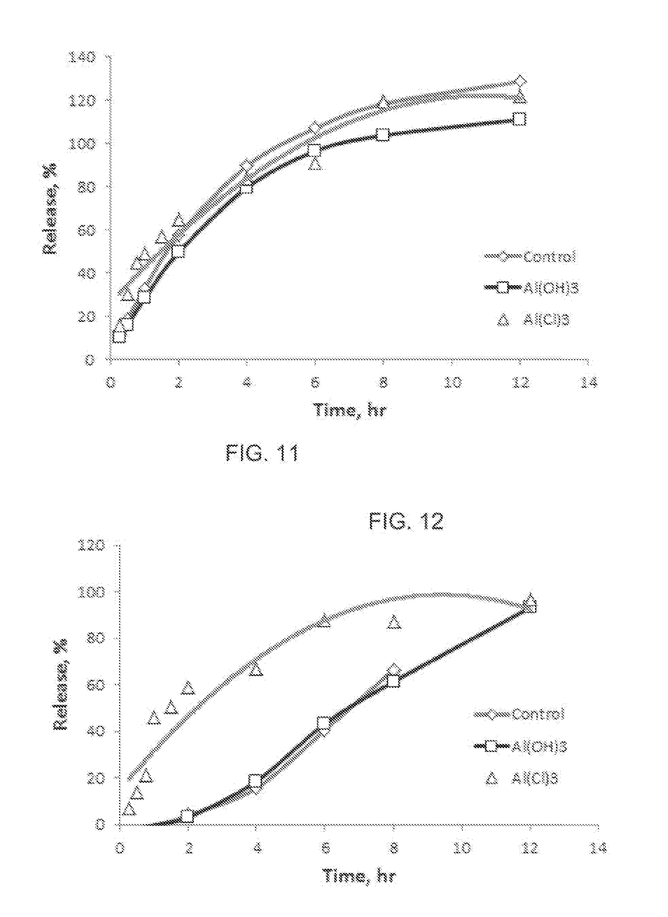

Tables 7-10 illustrate the foregoing formulations in 0.1N HCl as a dissolution medium. The results are illustrated in FIG. 11.

TABLE 7
Immediate release tablet

| Time, | Absorption @271 nm | % |
|---|---|---|
| h | (average of 3 readings) | released |
| 0.25 | 0.173767 | 112.9 |
| 0.5 | 0.173267 | 112.6 |
| 1 | 0.174167 | 113.1 |
| 8 | 0.174033 | 113.1 |

TABLE 8
Sustained release tablet (Control)

| Time, | Absorption @271 nm | % |
|---|---|---|
| h | (average of 3 readings) | released |
| 0.25 | 0.0189 | 11.5 |
| 0.5 | 0.029933 | 18.7 |
| 1 | 0.051667 | 32.9 |
| 2 | 0.089667 | 57.8 |
| 4 | 0.1383 | 89.7 |
| 6 | 0.164967 | 107.1 |
| 8 | 0.182167 | 118.4 |
| 12 | 0.197733 | 128.6 |

TABLE 9
Sustained release tablet, using Al(OH)₃

| Time, | Absorption @271 nm | % |
|---|---|---|
| h | (average of 3 readings) | released |
| 0.25 | 0.017533 | 10.6 |
| 0.5 | 0.025867 | 16.1 |
| 1 | 0.044567 | 28.3 |
| 2 | 0.077467 | 49.9 |
| 4 | 0.1229 | 79.6 |
| 6 | 0.148733 | 96.5 |
| 8 | 0.1599 | 103.8 |
| 12 | 0.171167 | 111.2 |

TABLE 10
Sustained release tablet, using AlCl₃

| Time, | Absorption @271 nm | % |
|---|---|---|
| h | (average of 3 readings) | released |
| 0.25 | 0.0258 | 16.0 |
| 0.5 | 0.0475 | 30.2 |
| 0.75 | 0.0698 | 44.8 |
| 1 | 0.0767 | 49.4 |
| 1.5 | 0.0883 | 56.9 |
| 2 | 0.1002 | 64.7 |
| 4 | 0.1292 | 83.7 |
| 6 | 0.1404 | 91.0 |
| 8 | 0.1838 | 119.5 |
| 12 | 0.1874 | 121.8 |

Tables 10-13 illustrate the foregoing formulations in distilled water as a dissolution medium. The results are illustrated in FIG. 12.

TABLE 10
Immediate release tablets:

| Time, | Absorption @271 nm | % |
|---|---|---|
| h | (average of 3 readings) | released |
| 0.25 | 0.157933 | 90.3 |
| 0.5 | 0.161933 | 92.6 |
| 8 | 0.161633 | 92.4 |

TABLE 11
Sustained release tablet (Control):

| | | |
|---|---|---|
| Time, | Absorption @271 nm | % |
| h | (average of 3 readings) | released |

Trial 1:

| | | |
|---|---|---|
| 0.25 | 0.0029 | −1.24 |
| 0.5 | 0.0025 | −1.5 |
| 1 | 0.005733 | 0.4 |
| 2 | 0.014267 | 5.5 |
| 4 | 0.0393 | 20.2 |
| 6 | 0.078 | 43.1 |
| 8 | 0.1239 | 70.2 |

Trial 2:

| | | |
|---|---|---|
| 0.25 | 0.001067 | −2.3 |
| 0.5 | −0.00097 | −3.6 |
| 1 | 0.001867 | −1.8 |
| 2 | 0.009633 | 2.7 |
| 4 | 0.024033 | 11.2 |
| 6 | 0.069633 | 38.1 |
| 8 | 0.1113 | 62.7 |

TABLE 12
Sustained release tablet, using Al(OH)₃

| | | |
|---|---|---|
| Time, | Absorption @271 nm | % |
| h | (average of 3 readings) | released |

Trial 1:

| | | |
|---|---|---|
| 0.5 | 0.005533 | 0.3 |
| 1 | 0.0077 | 1.6 |
| 2 | 0.015533 | 6.2 |
| 4 | 0.039933 | 20.6 |
| 6 | 0.0737 | 40.5 |
| 8 | 0.093433 | 52.2 |
| 12 | 0.151 | 86.2 |

Trial 2:

| | | |
|---|---|---|
| 0.25 | −0.00087 | −3.5 |
| 0.5 | 0.0017 | −1.9 |
| 1 | 0.0055 | 0.3 |
| 4 | 0.0331 | 16.6 |
| 6 | 0.08275 | 45.9 |
| 8 | 0.1242 | 70.3 |
| 12 | 0.1746 | 100.1 |

TABLE 13
Sustained release tablet, using AlCl₃

| Time, | Absorption @271 nm | % |
|---|---|---|
| h | (average of 2 readings) | released |
| 0.25 | 0.01705 | 7.1 |
| 0.5 | 0.02875 | 14.0 |
| 0.75 | 0.04125 | 21.4 |
| 1 | 0.0827 | 45.9 |
| 1.5 | 0.0911 | 50.8 |
| 2 | 0.1049 | 58.9 |
| 4 | 0.11875 | 67.1 |
| 6 | 0.1536 | 87.7 |
| 8 | 0.153 | 87.3 |
| 12 | 0.1687 | 96.6 |

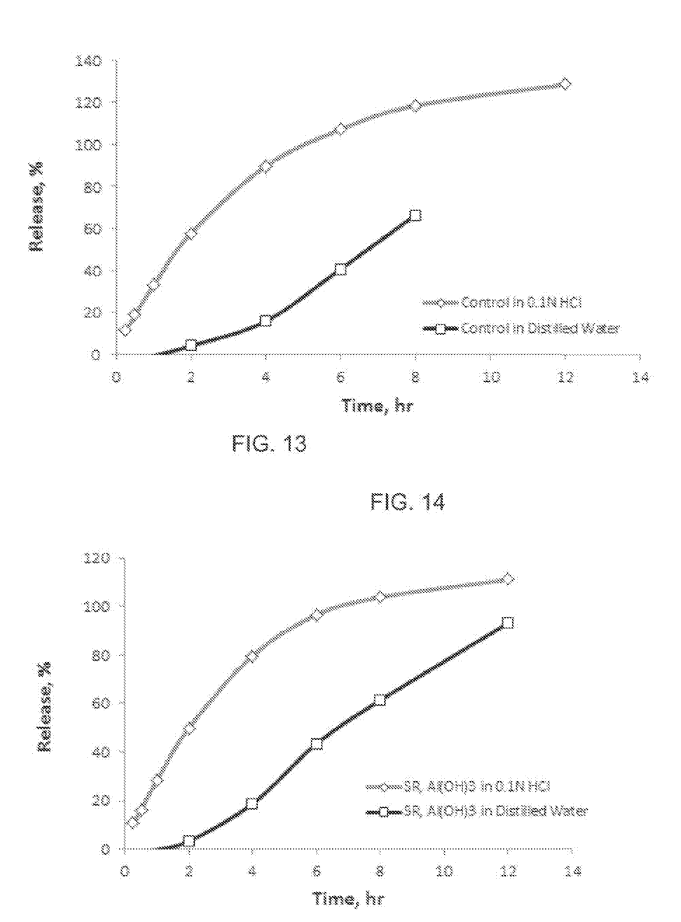

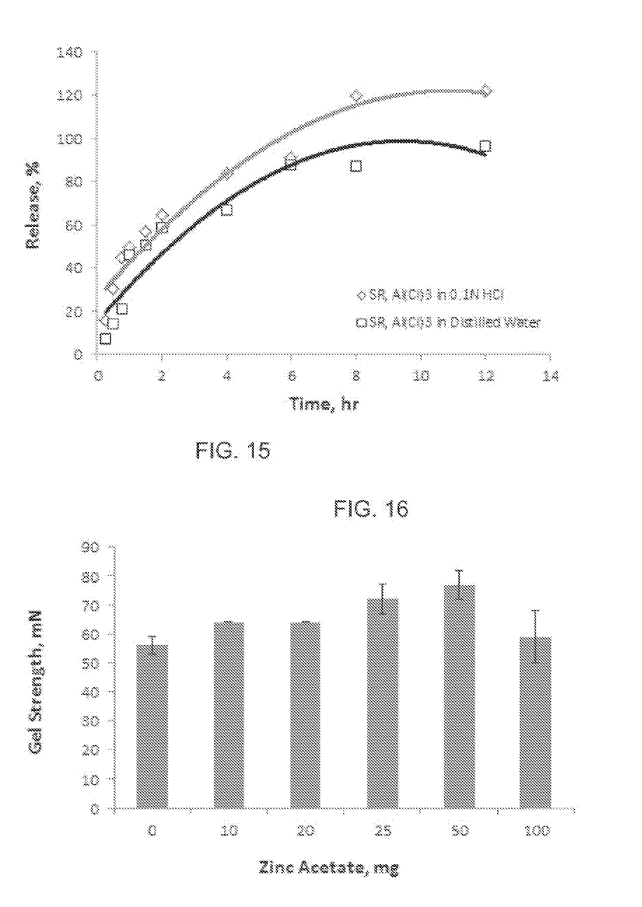

FIGS. 13-15 illustrate the same formulations in distilled water and 01.N HCl dissolution media.

### Gel Studies Using a Two-Valent Cation:

In accordance with the disclosure, an advantageous ratio of zinc acetate to sodium carboxymethylcellulose, in order to achieve maximum gel strength, is accomplished as follows.

Method: Zinc acetate and sodium carboxymethylcellulose (CMC) were weighed and mixed in a glass mortar, and 10 mL of distilled water was then added to physical mixture. Rapid gelation was observed within a few seconds. Gels were transferred to a 20 mL glass vial, and gel strength was measured on Texture Analyzer (CT3, Brookfield Engineering). A spherical probe (TA18) was attached to the shaft and was moved towards the sample at a speed of 2 mm/sec. Once it reached the trigger load of 44 mN, the probe moved to a distance of 4 mm into the gel at a speed of 0.5 mm/sec, and the load at the target was measured. Different compositions of gels were prepared and studied for their gel strength in a similar manner. The zinc/CMC ratio of 1:5 displayed the maximum gel strength in water, as shown in Table 14, and illustrated in FIG. 16.

TABLE 14

| Zinc acetate, | CMC, | Gel Strength, |
|---|---|---|
| mg | mg | mN |
| 0 | 250 | 56 |
| 10 | 250 | 64 |
| 20 | 250 | 64 |
| 25 | 250 | 72 |
| 50 | 250 | 77 |
| 100 | 250 | 59 |

### Gelation in Other Solutions:

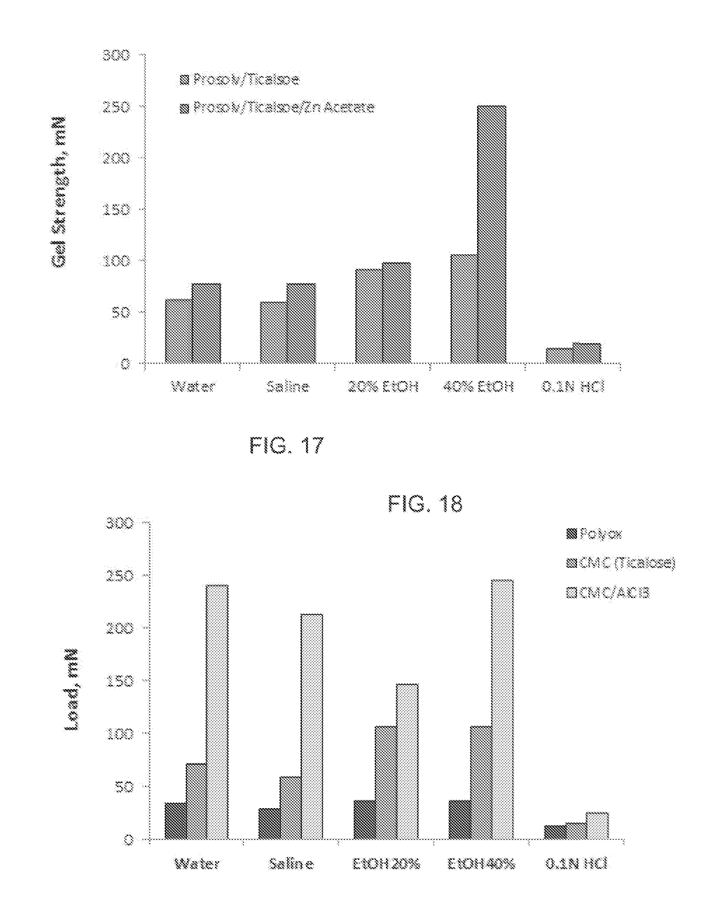

Once the optimal ratio of zinc acetate and sodium CMC was determined, studies were conducted to evaluate the effect of Prosolv® and solvents on gelation. Initially physical mixtures of Prosolv SMCC 90 (100 mg) and Ticalose® 6000 (250 mg) were weighed and mixed using a glass mortar with pestle. Different solvents (ultra-pure water, normal saline, hydro-alcoholic solutions and 0.1N HCl) of 10 mL volume were added to physical mixture. Gels formed were transferred to 20 mL glass vial and their strength was measured on a CT3 Texture analyzer. Same study was repeated for physical mixtures of zinc acetate (50 mg), Prosolv® SMCC 90(100 mg) and Ticalose® 6000 (250 mg), as shown in Table 15, and as illustrated in FIG. 17. It is noted that in 40% Ethanol solution, gel separated out of the solution.

TABLE 15

| | Prosolv/Ticalose | Prosolv ®/Ticalose ®/Zinc acetate |
|---|---|---|
| | Gel strength, mN | |
| Water | 62 | 77 |
| Saline | 59 | 77 |
| EtOH 20% | 91 | 98 |
| EtOH 40% | 106 | 250 |
| 0.1N HCl | 15 | 20 |

Table 16 and FIG. 18 show gel strength data for PEO, CMC without gel enhancer, and CMC with aluminum chloride as gel enhancer.

TABLE 16

| | Polyox ® WSR | CMC (Ticalose ®) | CMC/AlCl₃ |
|---|---|---|---|
| Medium | Gel strength, mN | | |
| Water | 34 | 72 | 240 |
| Saline | 29 | 59 | 213 |
| EtOH 20% | 37 | 106 | 147 |
| EtOH 40% | 37 | 106 | 245 |
| 0.1N HCl | 13 | 15 | 25 |

### Drug Release Studies:

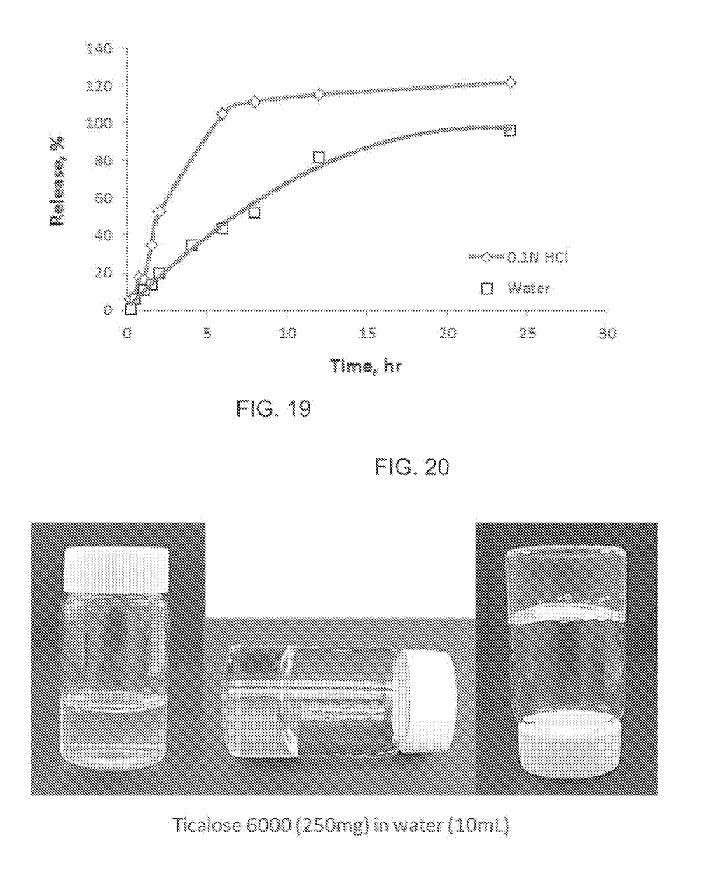

Tramadol HCl (25 mg), zinc acetate (50 mg), CMC (250 mg), and Prosolv® (75 mg) were mixed using a glass mortar and pestle. 400 mg of the mixture was weighed and then compressed on Carver press at 1000 pound force. The tablets prepared were studied for dissolution profile in distilled water and 0.1N HCl at 37° C. @ 50 rpm. Samples were drawn at specific time points and drug concentration was determined using a UV-spectrophotometer @271 nm. Results are shown in Tables 17-18, and are illustrated in FIG. 19.

TABLE 17
Drug release in 0.1N HCl:

| Time, | Absorption @271 nm | % |
|---|---|---|
| h | (average of 3 readings) | released |
| 0.25 | 0.029 | 6.0 |
| 0.5 | 0.031 | 7.2 |
| 0.75 | 0.044 | 17.9 |
| 1 | 0.050 | 16.0 |
| 1.5 | 0.067 | 34.7 |
| 2 | 0.096 | 52.4 |
| 4 | 0.118 | 66.9 |
| 6 | 0.180 | 104.9 |
| 8 | 0.193 | 111.4 |
| 12 | 0.199 | 115.2 |
| 24 | 0.217 | 121.6 |

TABLE 18
Drug release in water:

| Time, | Absorption @271 nm | % |
|---|---|---|
| h | (average of 2 readings) | released |
| 0.25 | 0.006 | 0.6 |
| 0.45 | 0.019 | 6.2 |
| 1 | 0.027 | 10.8 |
| 1.5 | 0.032 | 13.4 |
| 2 | 0.040 | 19.7 |
| 4 | 0.069 | 34.6 |
| 6 | 0.081 | 43.5 |
| 8 | 0.098 | 52.1 |
| 12 | 0.150 | 81.4 |
| 24 | 0.179 | 96.4 |

### Drug Binding Capacity of Multifunction Fillers:

Talc:

Into 10 mL of drug solution (containing 25 mg of tramadol HCl), 50 mg of talc was added and vortexed for 5 seconds followed by centrifugation @1500 rpm for 5 minutes. The solution was filtered through a 0.2 μm syringe filter; 0.5 ml of the supernatant solution was diluted with 10 ml of solvent, and then analyzed for drug concentration by UV spectroscopy @271 nm. Results are shown in Table 19.

TABLE 19

| | Absorption @271 nm | % |
|---|---|---|
| Solvent | (average of 3 readings) | Bound |
| Water | 0.5634 | 27.6 |
| 0.1N HCl | 0.730267 | 2.6 |
| pH 3 | 0.695333 | 4.4 |
| Normal saline | 0.646133 | 15.3 |
| EtOH 40% | 0.7137 | 5.6 |

Zinc Acetate:

Into 10 mL of drug solution (containing 25 mg of tramadol HCl), 50 mg of zinc acetate was added and vortexed for 5 seconds followed by centrifugation @1500 rpm for 5 minutes. The solution was filtered through a 0.2 μm syringe filter; 0.5 ml of the supernatant solution was diluted with 10 ml of solvent, and then analyzed for drug concentration by UV spectroscopy @271 nm. Results are shown in Table 20.

TABLE 20

| | Absorption @271 nm | % |
|---|---|---|
| Solvent | (average of 3 readings) | Bound |
| Water | 0.697133 | 10.7 |
| 0.1N HCl | 0.697433 | 7.0 |
| pH 3 | 0.7063 | 2.9 |
| Normal saline | 0.706833 | 7.4 |
| EtOH 40% | 0.6615 | 12.5 |

Aluminum Hydroxide:

Into 10 mL of drug solution (containing 25 mg of tramadol HCl), 10 mg of aluminum hydroxide was added and vortexed for 5 seconds followed by centrifugation @1500 rpm for 5 minutes. The solution was filtered through a 0.2 μm syringe filter; 0.5 ml of the supernatant solution was diluted with 10 ml of solvent, and then analyzed for drug concentration by UV spectroscopy @271 nm. Results are shown in Table 21.

TABLE 21

| | Absorption @271 nm | % |
|---|---|---|
| Solvent | (average of 3 readings) | Bound |
| Water | 0.693533 | 11.1 |
| 0.1N HCl | 0.716433 | 4.5 |
| pH 3 | 0.7207 | 0.9 |
| Normal saline | 0.728367 | 4.6 |
| EtOH 40% | 0.7137 | 5.6 |

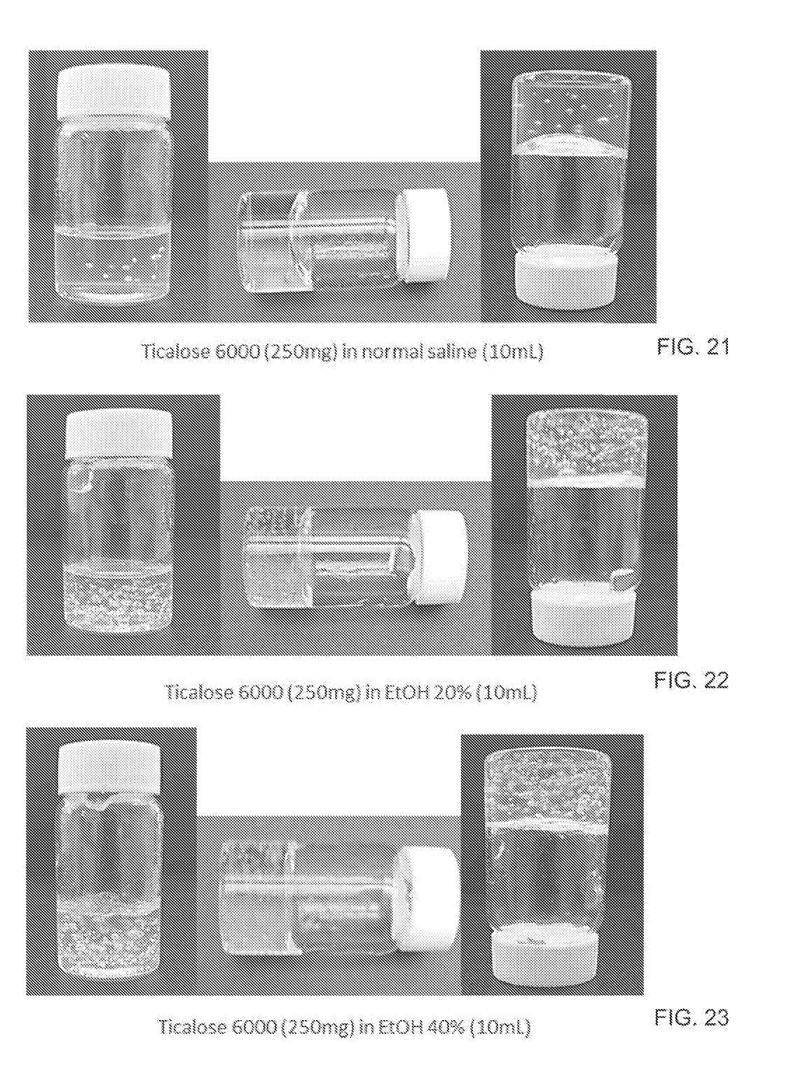

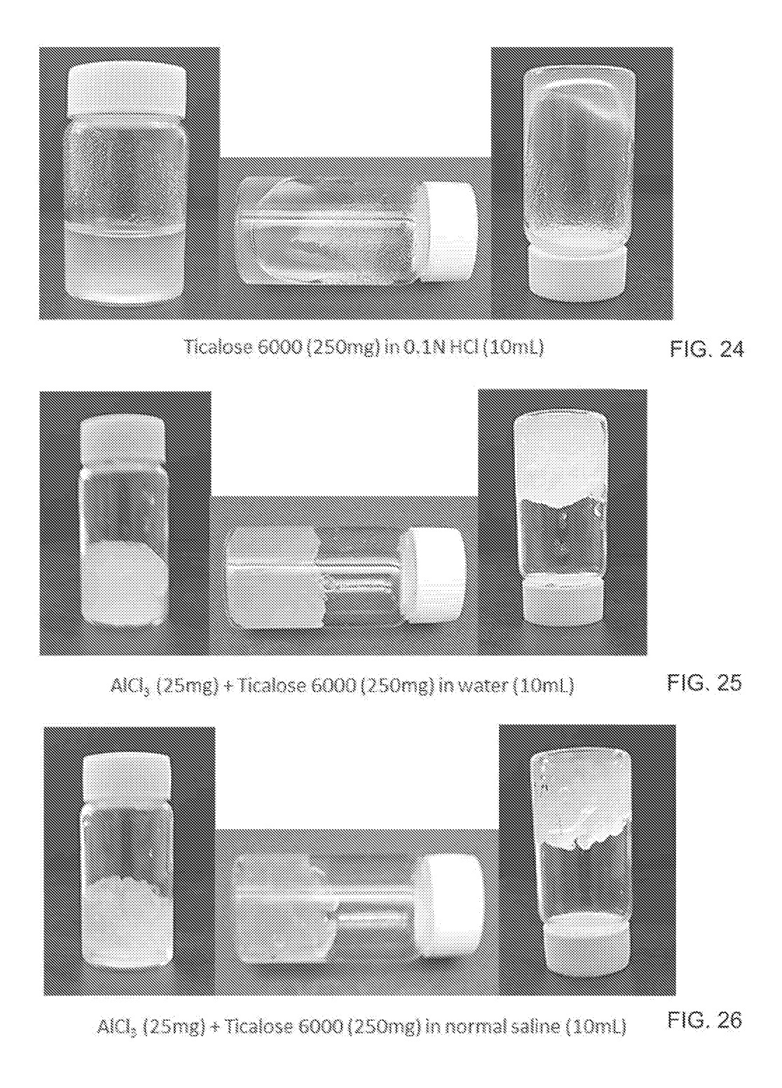

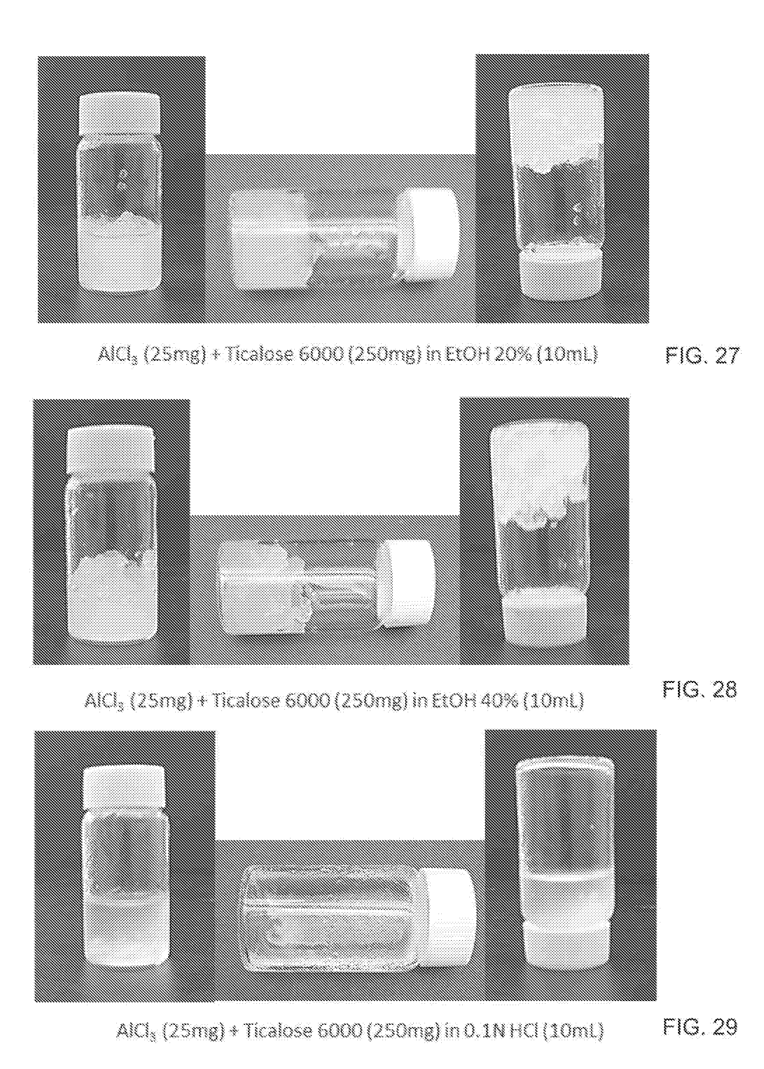

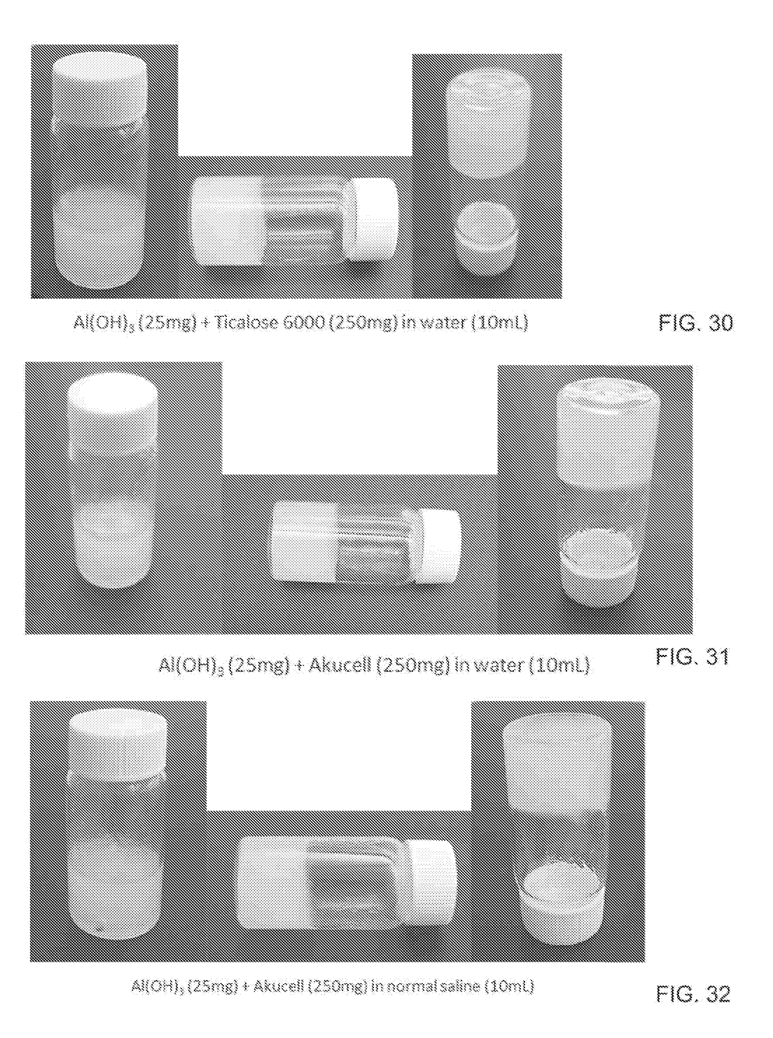

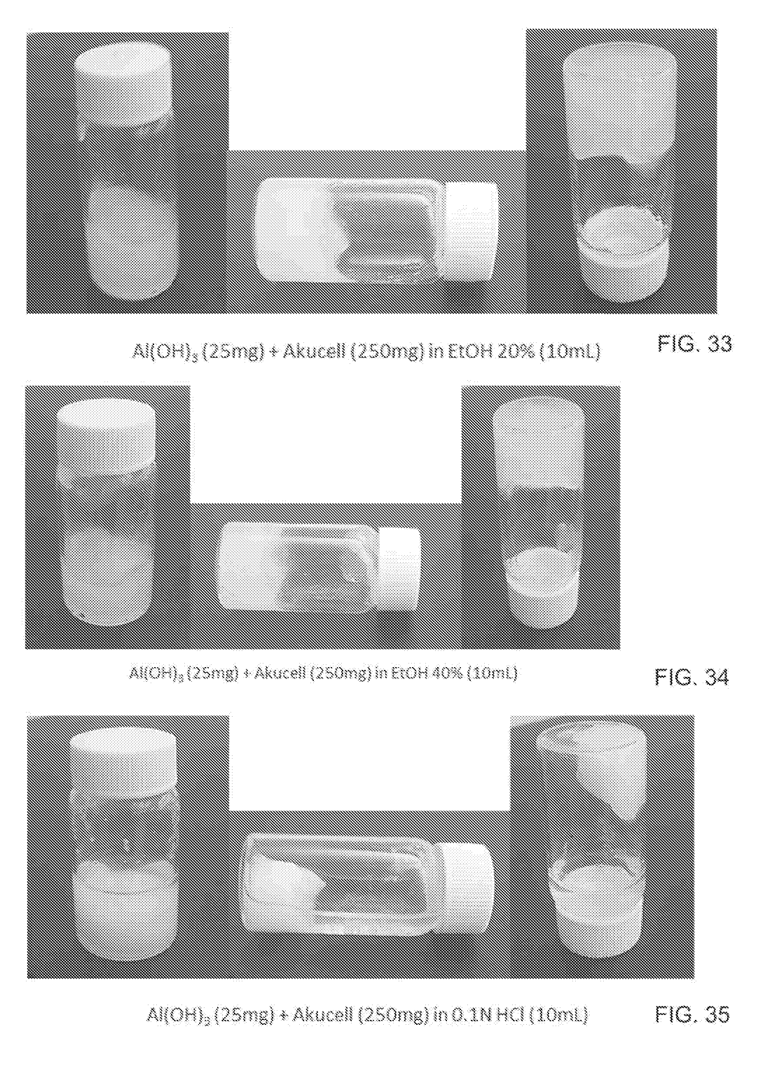

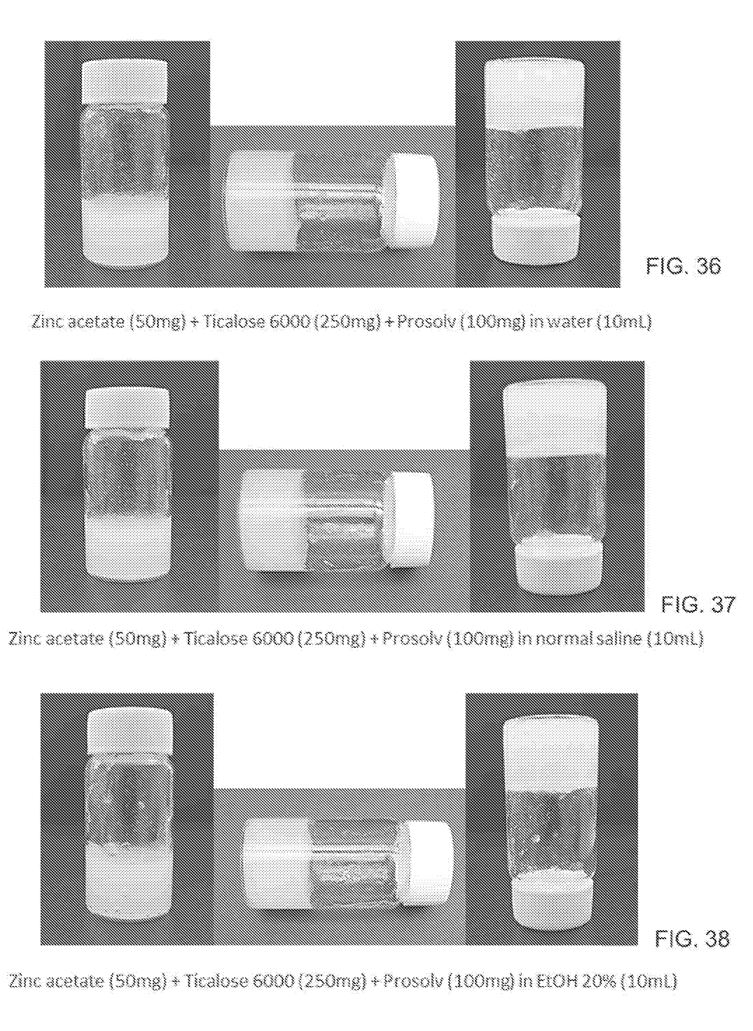

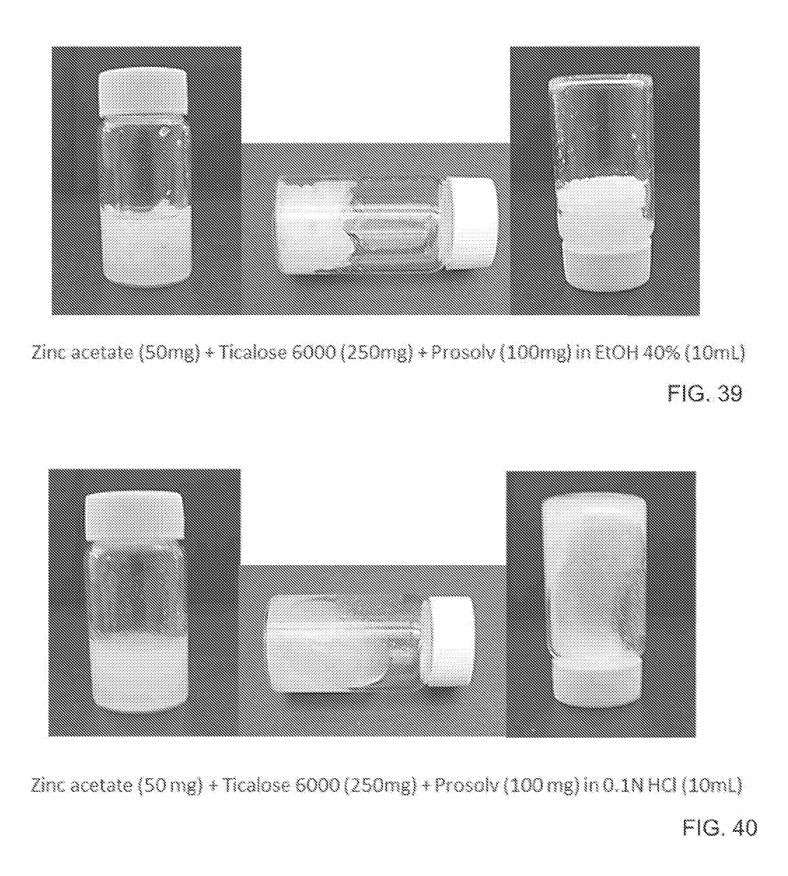

The following photos depict various formulations as described herein, in various solutions as indicated. FIGS. 20-24 are Ticalose® 6000 (250 mg) gels in different solutions (10 mL); FIGS. 25-29 are Ticalose® 6000 (250 mg) AlCl3 (25 mg) gels; FIG. 30 is Ticalose® 6000 (250 mg) Al(OH)3 (25 mg) gel in water; FIGS. 31-35 are Akucell® (250 mg) Al(OH)3 (25 mg) gels; and FIGS. 36-40 are Ticalose® 6000 (250 mg) Zinc Acetate (50 mg) Prosolv® (100 mg) gels.

The inventive composition described above includes the following:

1. A multifunction polymer:

a. That can be added into the tablet at high concentration, provides gel strength up to 5-10 folds higher than what can be achieved with the PEO polymers while offering a similar release profile in 0.1N HCl.

b. The multifunction polymer of this disclosure is capable of binding with basic molecules such as tramadol HCl.

c. The multifunction polymer of this disclosure can provide different release profiles in water and 0.1N HCl, an almost a zero order in water, while first order in 0.1N HCl. In other words, polymers of this disclosure hardly release the drug over short term in abuse solutions (primarily in water), while offering easy release in-vivo or under in-vitro dissolution testing using 0.1N HCl.

d. The multifunction polymer of this disclosure is capable of binding to other excipients used in the tablet intended to modulate the gel strength and drug release profile.

2. A multifunction gel enhancer:

a. That strongly binds to the multifunction polymer in solution, and enhances its gelling property in respective solution.

b. That moderately binds to the multifunction polymer in solution, and modulates the drug release.

c. That partially binds to the basic active drug in solution.

3. A multifunction filler:

a. That remains non-reactive to the multifunction polymer of this disclosure; it however partially binds to the basic drug molecule in solution.

### Example 3: Polyvinyl Alcohol Based Cryogels

In accordance with the invention, a therapeutic crush-resistant cryogel composite with abuse-deterrent capability is formulated using Polyvinyl Alcohol (PVOH), which is a synthetic hydrophilic linear polymer produced as varied copolymer of vinyl alcohol with vinyl acetate, vinyl amine, vinyl pyrrolidone, or ethylene glycol. Due to reactive functional groups on its structure, PVOH undergoes chemical changes such as esterification and etherification, as well as physical changes such as crystallization and ion-polymer complexation. At a given molecular weight and degree of hydrolysis, the mechanical property of the final PVOH cryogel product is essentially determined by its original solution concentration, and the number of freezing and thawing cycles. Lower and higher temperature extremes and duration at which the platform is treated over specific temperature would also affect the cryogel mechanical properties to a lesser extent. The higher the concentration and the greater the number of cycles, the greater would become the cryogel mechanical property. Lower and higher extremes of mechanical properties would be undesirable due to lack of mechanical integrity and the higher PVOH concentration, respectively. However, a cryogel with optimum and desirable mechanical properties can be designed by adjusting the solution concentration and the factors influencing the cryogelation process.

When polymers are used as controlled delivery medium, their concentration in the dosage form should be kept to a minimum while offering desirable properties. Lower concentration of the polymer would minimize the polymer-drug interaction, instability of the dosage form, and facilitate its processing. PVOH cryogels generally consist of water and the PVOH polymer, and the cryogel can feasibly be made at PVOH concentration ranging 10-20 wt %. Over such range, the cryogels can become mechanically very strong like a tough rubber, which may be difficult to remove from the GI tract in that state, and disintegration of the material could be a challenge. Such concentrations can also cause excessive crystallization of the polymer, which causes instability. Moreover, it will increase the chance of unwanted interaction between the polymer and the drug, and can physically hinder the effective release of the drug from the cryogel platform.

The cryogel polymers of this disclosure would be desirable in areas where a controlled delivery vehicle with certain mechanical properties would be very advantageous. For instance, cryogels of this disclosure can be used as a vehicle for immediate or controlled release of abusable medications, whereas the vehicle possesses certain mechanical strength and viscoelastic properties that prevent the dosage form from being abused by crushing or grinding.

This invention provides a cryogel vehicle, which is manufactured at the lowest PVOH concentration yet possessing reasonable mechanical property, resembling a soft rubber. In one embodiment, a cryogel with desirable physical mechanical properties is disclosed having desirable adhesive force, adhesiveness, gumminess, and hardness making the PVOH-based delivery platform crush-resistant. At solution concentrations of about 5 wt %, a PVOH cryogel possesses tackiness, and adheres to variety of surfaces including endothelial cells. The maximum tackiness can be achieved at concentrations at around 2-3 wt % while a cryogel can still be formed. Drugs (water soluble or water-insoluble) can be dispersed into the aqueous solution of PVOH at such concentration as solid powder, solution, or dispersion. The drug-loaded mix can then undergo freezing-thawing cycle(s), by which the drug would be entrapped into the PVOH cryogel structure. Drug release would then be governed by either a diffusion mechanism if the gel is mechanically strong, or by a combined diffusion-erosion mechanism if the gel is structurally weak. A cryogel formed at such concentrations, loaded with the drug, can be housed into, for example, a soft or hard gelatin capsule for oral administration. In another embodiment, a crush resistant abuse-deterrent cryogel composite can be simply formed in any desirable shape for faster disintegration and guaranteed emptying from the stomach. This, for instance, can simply be achieved by shredding the cryogel or cryogel composite, and molding the material into desired dimensions or shapes. These cryogels can also be easily prepared in the form of a thick slab, thin film, extruded, or molded product, to suitably address the service needs.

In another embodiment, a cryogel composite containing an abuse-deterrent agent is disclosed, where drug is entrapped within its structure, and then released in a controlled manner under regular oral administration in the acidic stomach.

The PVOH cryogels of this invention possess advantages such as ease of manufacturing (one step preparation, no need for purification), ease of sterilization, simple and stable polymer structure, minimum drug-polymer interaction, absence of other chemicals or excipients, no residual processing chemicals, ease of drug loading by any form, drug loading at room temperature, and feasible processing such as molding and cutting.

### Examples of Cryogels

### Preparation

Polyvinyl alcohol (PVOH) aqueous solutions were prepared by dissolving PVOH in ultrapure water at 90° C. for two hours with constant stirring. Solutions were then allowed to cool at room temperature before being used. After being cooled, a specific weight of the solution was transferred into plastic molds (dimensions 4.1×4.1×0.8 cm) and placed into a bench-top cooler (VWR Boekel Mini Fridge II 260009). The solutions were then subjected to two freeze-thaw cycles consisting of four hours of freezing at −10° C. followed by two hours of thawing at 25° C.

### Adhesive Properties

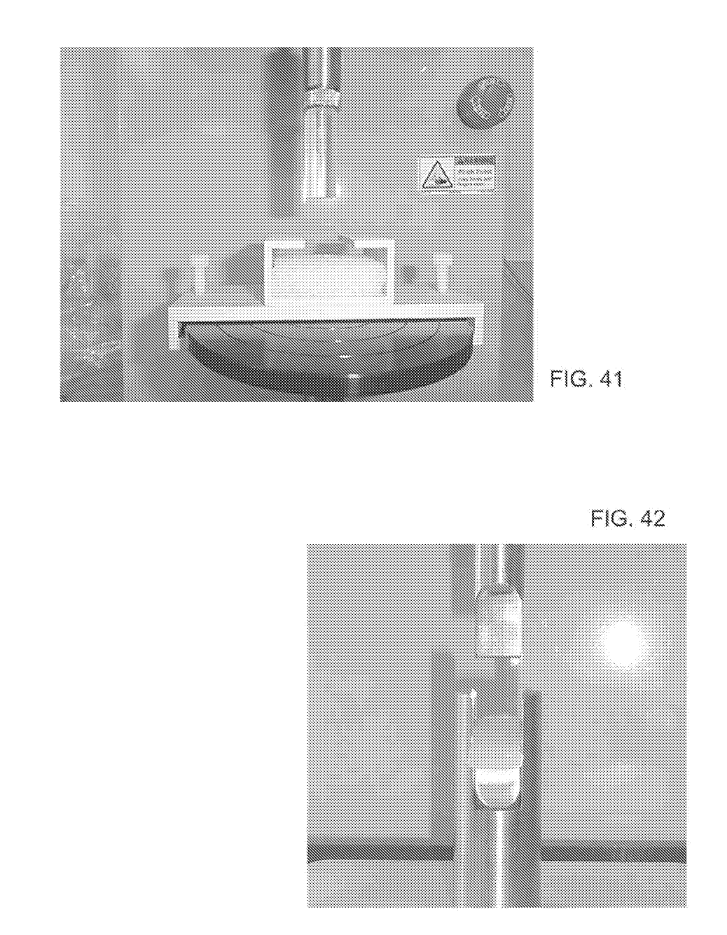

PVOH cryogels (5%, 8% and 10% w/w) still in their plastic molds were subjected to adhesive tests using a texture analyzer (CT3, Brookfield Engineering). Tests were performed using an acrylic probe of 12.7 mm diameter and 35 mm length (TA10) attached to the arm of the texture analyzer. The probe was lowered toward the sample until a trigger load of 67 mN was achieved. After this trigger, the probe was moved into the sample at a rate of 0.5 mm/sec to a distance of 4 mm. This was repeated for 2 cycles on each sample and a plot of load (mN) versus time (sec) was used by the instrument software (TexturePro CT) to achieve the desired parameters of adhesive force, adhesiveness, and gumminess. All tests were performed in triplicates at room temperature. Adhesive force (N or mN) is the maximum force required to pull the probe away from the sample. Adhesiveness (mJ) is the work required overcoming attractive forces between the sample and the surface of the probe. Gumminess is the energy required converting a semi-solid to a state ready to swallow. Gumminess is calculated by multiplying hardness of the substance with cohesiveness of the substance. The test apparatus is shown in FIG. 41, and test parameters are shown in Table 22.

TABLE 22
Test type : TPA

| | |
|---|---|
| Target: 4.0 mm | Recovery time: 0 |
| Hold time: 0 s | Same trigger: true |
| Trigger load: 67 mN | Pretest speed: 2 mm/s |
| Test speed: 0.5 mm/s | Probe: TA10 |
| Return speed: 0.5 mm/s | Fixture: TA-ATT |
| # of cycles: 2 | Load cell: 4500 g |

### Hardness

PVOH cryogels (5%, 8% and 10% w/w) still in their plastic molds were subjected to hardness testing using a texture analyzer (CT3, Brookfield Engineering). Testing was performed using Volodkevich Bite Jaws, (TA-VBJ, Brookfield Engineering) an accessory attached to the arm of the texture analyzer. The probe was lowered toward the sample until a trigger load of 67 mN was achieved. After this trigger, the probe was moved at a rate of 0.5 mm/sec to a distance of 4 mm into each sample to calculate the hardness work (mJ) done by the instrument software (TexturePro CT). All tests were performed in triplicates at room temperature. The test apparatus is shown in FIG. 42, and test parameters are shown in Table 23.

TABLE 23
Test type: Compression

| | |
|---|---|
| Target: 4.0 mm | Recovery time: 0 |
| Hold time: 0 s | Same trigger: False |
| Trigger load: 67 mN | Pretest speed: 2 mm/s |
| Test speed: 0.5 mm/s | Probe: TA-VBJ |
| Return speed: 0.5 mm/s | Fixture: TA-BT-KIT |
| # of cycles: 1 | Load cell: 4500 g |

### Swelling

Prepared PVOH cryogels (3%, 5%, 8% and 10% w/w) were trimmed into square dimensions weighing 1 gm. The square samples were then placed into 50 ml of ultrapure water at room temperature and allowed to swell freely. At specific time points, the samples were removed from the swelling medium, surface dried with a Kim Wipe, and weighed.

### Results

### Adhesive Force Data

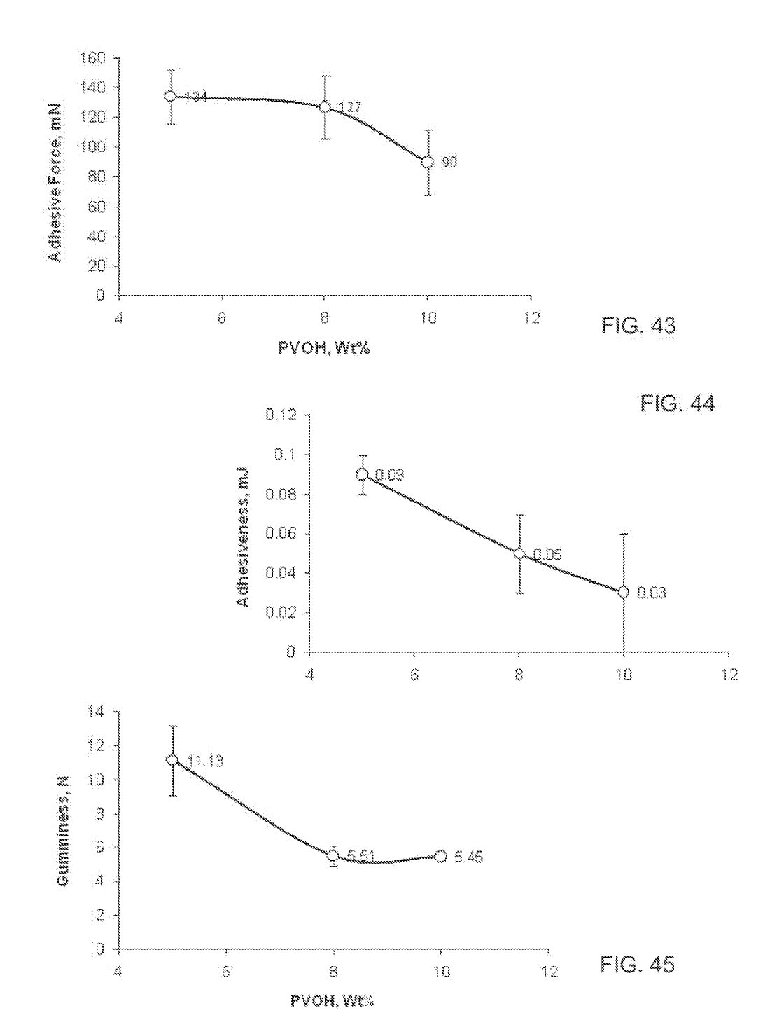

Table 24 and FIG. 43 shows adhesive force for cryogels at 5 and 8 wt % solution concentrations are similar, however the adhesive force drops down at a fast rate for cryogel made at 10% PVOH concentration.

TABLE 24

| [PVOH], | Adhesive force, mN | | | | |
|---|---|---|---|---|---|
| Wt % | Sample 1 | Sample 2 | Sample 3 | Mean | SD |
| 5 | 142 | 147 | 113 | 134 | 18 |
| 8 | 142 | 103 | 137 | 127 | 21 |
| 10 | 88 | 69 | 113 | 90 | 22 |

### Adhesiveness Data

Table 25 and FIG. 44 show that adhesiveness of cryogels made at 5, 8 and 10 wt % is reduced in a linear trend as the PVOH concentration increases over the range of solution concentrations studied.

TABLE 25

| [PVOH], | Adhesiveness, mJ | | | | |
|---|---|---|---|---|---|
| Wt % | Sample 1 | Sample 2 | Sample 3 | Mean | SD |
| 5 | 0.1 | 0.09 | 0.09 | 0.09 | 0.01 |
| 8 | 0.03 | 0.07 | 0.05 | 0.05 | 0.02 |
| 10 | 0.04 | 0 | 0.06 | 0.03 | 0.03 |

### Gumminess Data

Table 26 and FIG. 45 show that cryogel made at 5% offers more gummy properties whereas those made at 8 and 10 wt % possesses lower gumminess (or higher rigidity).

TABLE 26

| | Gumminess, N | | | | |
|---|---|---|---|---|---|
| [PVOH], Wt % | Sample 1 | Sample 2 | Sample 3 | Mean | SD |
| 5 | 10.996 | 9.123 | 13.267 | 11.13 | 2.08 |
| 8 | 5.388 | 4.939 | 6.19 | 5.51 | 0.63 |
| 10 | 5.561 | 5.192 | 5.585 | 5.45 | 0.22 |

### Hardness Data

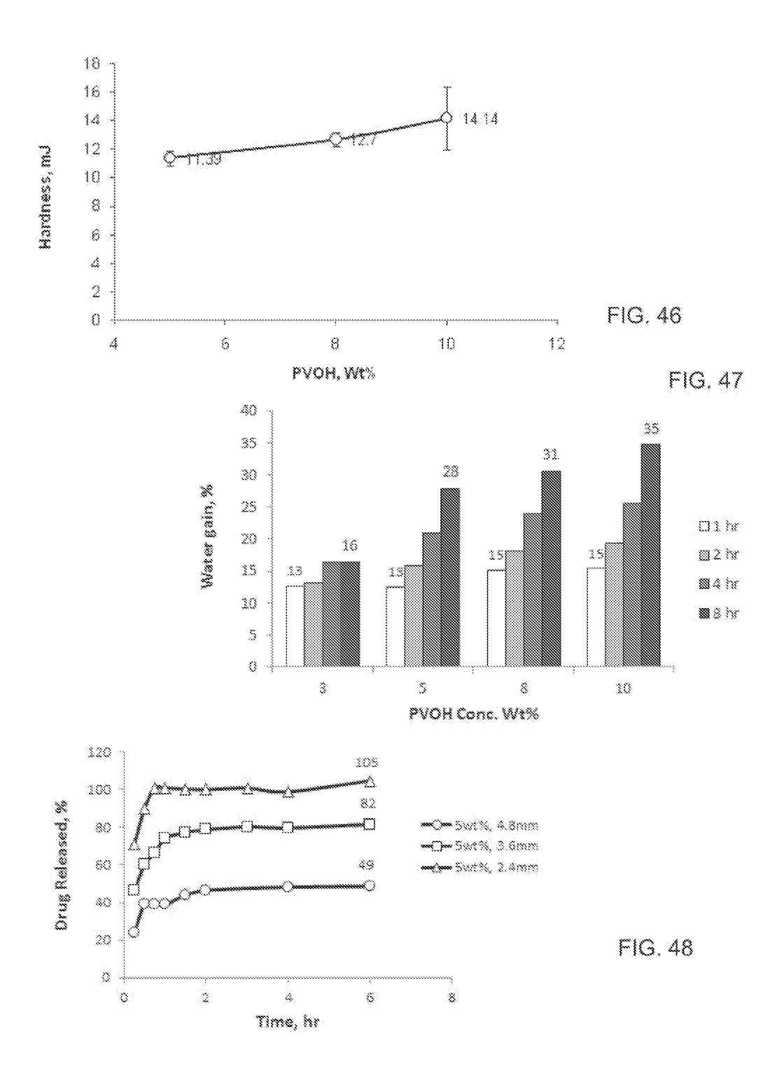

Table 27 and FIG. 46 show there is no significant difference in hardness of the cryogels made at 5, 8, and 10 wt % PVOH solution concentrations.

TABLE 27

| | Hardness, mJ | | | | |
|---|---|---|---|---|---|
| [PVOH], Wt % | Sample 1 | Sample 2 | Sample 3 | Mean | SD |
| 5 | 11.62 | 11.75 | 10.81 | 11.39 | 0.51 |
| 8 | 12.55 | 13.25 | 12.31 | 12.7 | 0.49 |
| 10 | 11.58 | 15.41 | 15.42 | 14.14 | 2.21 |

### Water Absorption (Swelling) Data

Table 28 and FIG. 47 show that cryogels made at higher PVOH concentrations can absorb more water than those made at lower PVOH concentrations.

TABLE 28

| | [PVOH] Wt % | | | | | | | |
|---|---|---|---|---|---|---|---|---|
| | 3 | | 5 | | 8 | | 10 | |
| | | Weight | | Weight | | Weight | | Weight |
| Time, | Weight | increase | Weight | increase | Weight | increase | Weight | increase |
| hr. | (g) | (%) | (g) | (%) | (g) | (%) | (g) | (%) |
| 0 | 1.0 | 0 | 1.0 | 0 | 1.0 | 0 | 1.0 | 0 |
| 0.5 | 1.105 | 10.5 | 1.095 | 9.5 | 1.103 | 10.3 | 1.109 | 10.9 |
| 1 | 1.127 | 12.7 | 1.125 | 12.5 | 1.151 | 15.1 | 1.154 | 15.4 |
| 2 | 1.132 | 13.2 | 1.158 | 15.8 | 1.181 | 18.1 | 1.194 | 19.4 |
| 4 | 1.164 | 16.4 | 1.209 | 20.9 | 1.239 | 23.9 | 1.256 | 25.6 |
| 8 | 1.164 | 16.4 | 1.279 | 27.9 | 1.306 | 30.6 | 1.347 | 34.7 |
| 12 | N/a | N/a | 1.314 | 31.4 | 1.356 | 35.6 | 1.405 | 40.5 |
| 24 | N/a | N/a | 1.364 | 36.4 | 1.412 | 41.2 | 1.514 | 51.4 |

### Acetaminophen (APAP) Loaded Cryogels

### Preparation

APAP-PVOH cryogels were prepared by first making PVOH aqueous solutions in ultrapure water at a concentration of 5 and 10% w/w. After solutions were cooled to room temperature, 10 mg of acetaminophen powder (Sigma-Aldrich) per gram of PVOH solution was added under constant stirring until completely dissolved. A specific weight (2, 3.5, 5 g) of the drug loaded solution was then cast into plastic molds (dimensions 4.1×4.1×0.8 cm) and placed into a bench-top cooler (VWR Boekel Mini Fridge II 260009). The solutions were subjected to two freeze-thaw cycles consisting of four hours of freezing at −10° C. followed by two hours of thawing at 25° C.

### Drug Release

APAP-PVOH cryogels were released from their molds and average thickness determined by digital caliper. The cryogels were then subjected to dissolution studies using a USP 2 paddle method (Distek dissolution system 2100A) in 900 ml of ultrapure water at 37.5° C. at a paddle rotational speed of 50 rpm. APAP concentration in the dissolution medium was analyzed using UV-Visible spectroscopy at 243 nm (UV-1700, Shimadzu) over time.

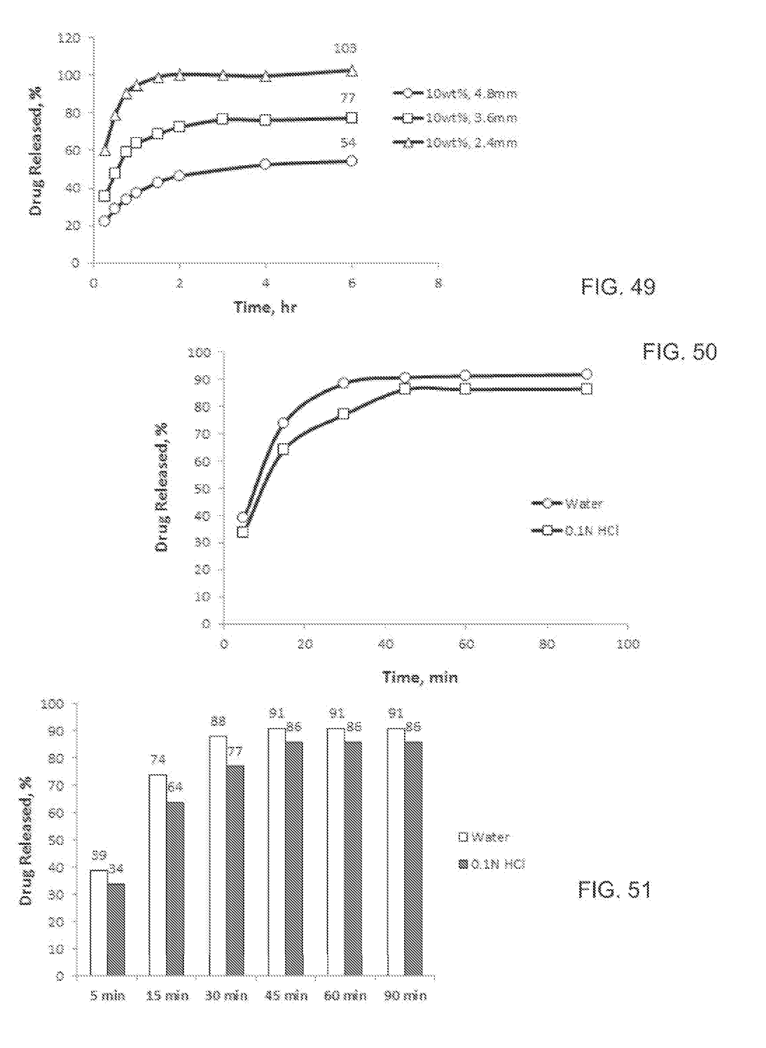

FIGS. 48-49 show overall data from the acetaminophen study, and that the desirable drug release can be controlled by the thickness of the cryogel platform.

### Tramadol Hydrochloride-Loaded Cryogels

### Preparation

Tramadol-PVOH cryogels were prepared by first making a PVOH aqueous solution in ultrapure water at a concentration of 5% w/w. After the solution was cooled to room temperature, each cryogel was made by adding 20 mg of tramadol hydrochloride (Medisca, Inc.) to 2 gm of PVOH solution. Each mixture was mixed until tramadol was completely dissolved. The drug loaded solution was then cast into plastic molds (dimensions 4.1×4.1×0.8 cm) and placed into a bench-top cooler (VWR Boekel Mini Fridge II 260009). The solutions were subjected to two freeze-thaw cycles consisting of four hours of freezing at −10° C. followed by two hours of thawing at 25° C.

### Drug Release

Tramadol-PVOH cryogels were released from their molds and subjected to dissolution studies using a USP 2 paddle method (Distek dissolution system 2100A) at a paddle rotational speed of 50 rpm. The dissolution media were 900 ml of ultrapure water, and 0.1N HCl, all at 37.5° C. Tramadol concentration in the dissolution medium was analyzed over time using UV-Visible spectroscopy at 271 nm (UV-1700, Shimadzu).

TABLE 29

| Dissolution | Time | | [Tramadol HCl] | % of drug |
|---|---|---|---|---|
| medium | (min) | abs @ 271 nm | μg/ml | released |
| Water | 5 | 0.0503 | 8.68 | 39.04 |
| Water | 15 | 0.0951 | 16.40 | 73.80 |
| Water | 30 | 0.1139 | 19.64 | 88.39 |
| Water | 45 | 0.1167 | 20.12 | 90.53 |
| Water | 60 | 0.1174 | 20.25 | 91.10 |
| Water | 90 | 0.1181 | 20.36 | 91.62 |
| 0.1N HCl | 5 | 0.0499 | 7.51 | 33.78 |
| 0.1N HCl | 15 | 0.0903 | 14.24 | 64.08 |
| 0.1N HCl | 30 | 0.1075 | 17.10 | 76.95 |
| 0.1N HCl | 45 | 0.1197 | 19.14 | 86.13 |
| 0.1N HCl | 60 | 0.1198 | 19.16 | 86.20 |
| 0.1N HCl | 90 | 0.1200 | 19.19 | 86.35 |

Table 29 and FIGS. 50-51 show that a tramadol-loaded PVOH cryogel made at 5 wt % solution concentration can release over 80% of tramadol HCl in about 45 minutes in both water and 0.1N HCl as dissolution medium.

### Croscarmellose Sodium Cryogel Composite

### Preparation

Croscarmellose sodium PVOH cryogel composites were prepared by first making a PVOH aqueous solution in ultrapure water at a concentration of 5% w/w. After the solution was cooled to room temperature, 10% of croscarmellose sodium (Ac-di-Sol®) was added and uniformly mixed. The composite mixture was then cast into plastic molds (dimensions 4.1×4.1×0.8 cm) and placed into a bench-top cooler (VWR Boekel Mini Fridge II 260009). The solutions were subjected to two freeze-thaw cycles consisting of four hours of freezing at −10° C. followed by two hours of thawing at 25° C.

### Swelling

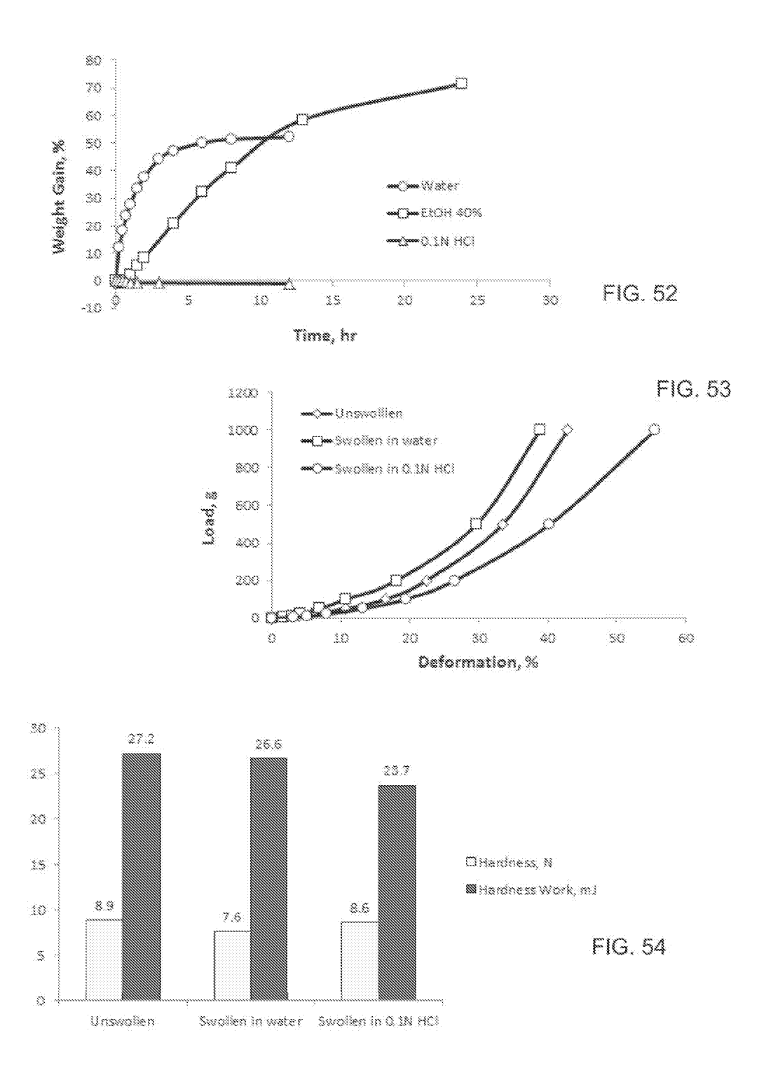

PVOH cryogels were released from their molds and allowed to swell in either ultrapure water, 0.1N HCl, or 40% w/v ethanol at room temperature. At different time intervals, the cryogels were removed from the swelling medium, and surface-dried using a Kim wipe before being weighed. The reported values, shown in Table 30 and FIG. 52 are the average of three samples.

TABLE 30

| Swelling medium | Time, hr. | % weigh gain |
|---|---|---|
| Water | 0 | 0.00 |
| Water | 0.25 | 12.20 |
| Water | 0.5 | 18.21 |
| Water | 0.75 | 23.89 |
| Water | 1 | 27.82 |
| Water | 1.5 | 33.61 |
| Water | 2 | 37.83 |
| Water | 3 | 44.24 |
| Water | 4 | 47.33 |
| Water | 6 | 50.08 |
| Water | 8 | 51.38 |
| Water | 12 | 52.11 |
| 0.1N HCl | 0 | 0.00 |
| 0.1N HCl | 0.25 | 0.00 |
| 0.1N HCl | 0.5 | 0.00 |
| 0.1N HCl | 0.75 | −0.26 |
| 0.1N HCl | 1 | −0.61 |
| 0.1N HCl | 1.5 | −0.69 |
| 0.1N HCl | 3 | −0.69 |
| 0.1N HCl | 12 | −0.95 |
| 40% ethanol in water | 0 | 0.00 |
| 40% ethanol in water | 0.25 | −1.00 |
| 40% ethanol in water | 0.5 | −0.44 |
| 40% ethanol in water | 0.75 | 0.10 |
| 40% ethanol in water | 1 | 2.46 |
| 40% ethanol in water | 1.5 | 5.38 |
| 40% ethanol in water | 2 | 8.59 |
| 40% ethanol in water | 4 | 20.86 |
| 40% ethanol in water | 6 | 32.21 |
| 40% ethanol in water | 8 | 41.03 |
| 40% ethanol in water | 13 | 58.45 |
| 40% ethanol in water | 24 | 71.57 |

### Mechanical Properties

Load-Deformation measured by a bench-top comparator:

PVOH cryogels were released from their molds and allowed to swell in ultrapure water or in 0.1N HCl. Unswollen cryogels were used as a comparison. After being allowed to swell for 12 hours, the cryogels were removed from the swelling medium and surface-dried using a Kim wipe. The cryogels were then placed on the working surface of a bench-top comparator (2W, B.C.Ames). The thickness of each cryogel was measured under increasing load and the percent of deformation measured. Each test was performed on two samples at room temperature and the average reported in Table 32 and FIG. 53.

TABLE 32

% Deformation

| Load (g) | Un-swollen | Water swollen | 0.1N HCl swollen |
|---|---|---|---|
| 0 | 0.00 | 0.00 | 0.00 |
| 5 | 1.94 | 1.71 | 3.05 |
| 10 | 3.87 | 2.89 | 5.10 |
| 20 | 5.81 | 4.06 | 7.84 |
| 50 | 10.69 | 6.95 | 13.26 |
| 100 | 16.50 | 10.74 | 19.41 |
| 200 | 22.39 | 18.05 | 26.51 |
| 500 | 33.46 | 29.69 | 40.32 |
| 1000 | 42.92 | 38.90 | 55.62 |

### Hardness Measured by Texture Analyzer:

Testing was performed using Volodkevich Bite Jaws, (TA-VBJ, Brookfield Engineering) an accessory (TA-VBJ, Brookfield Engineering) attached to the arm of the texture analyzer. The probe was lowered toward the sample until a trigger load of 67 mN was achieved. After this trigger, the probe was moved at a rate of 0.5 mm/sec to a distance of 4 mm into each sample to calculate hardness work (mJ) done by the instrument software (TexturePro CT). Results are illustrated in FIG. 54.

### Tramadol-Croscarmellose Sodium PVOH Cryogel Composites

### Preparation

Tramadol-PVOH composite cryogels were prepared by first making PVOH aqueous solution in ultrapure water at a concentration of 5% w/w. After the solution was cooled to room temperature, each cryogel was made by adding 20 mg of tramadol hydrochloride (Medisca, Inc.) to 2 gm of PVOH solution. Each solution was mixed until tramadol HCl was completely dissolved. Next, 10% of croscarmellose sodium (Ac-di-Sol®) was added and uniformly mixed. The drug loaded mixture was then cast into plastic molds (dimensions 4.1×4.1×0.8 cm), and placed into a bench-top cooler (VWR Boekel Mini Fridge II 260009). The solutions were subjected to two freeze-thaw cycles consisting of four hours of freezing at −10° C. followed by two hours of thawing at 25° C.

### Drug Release

Drug Release from Cryogel Slab

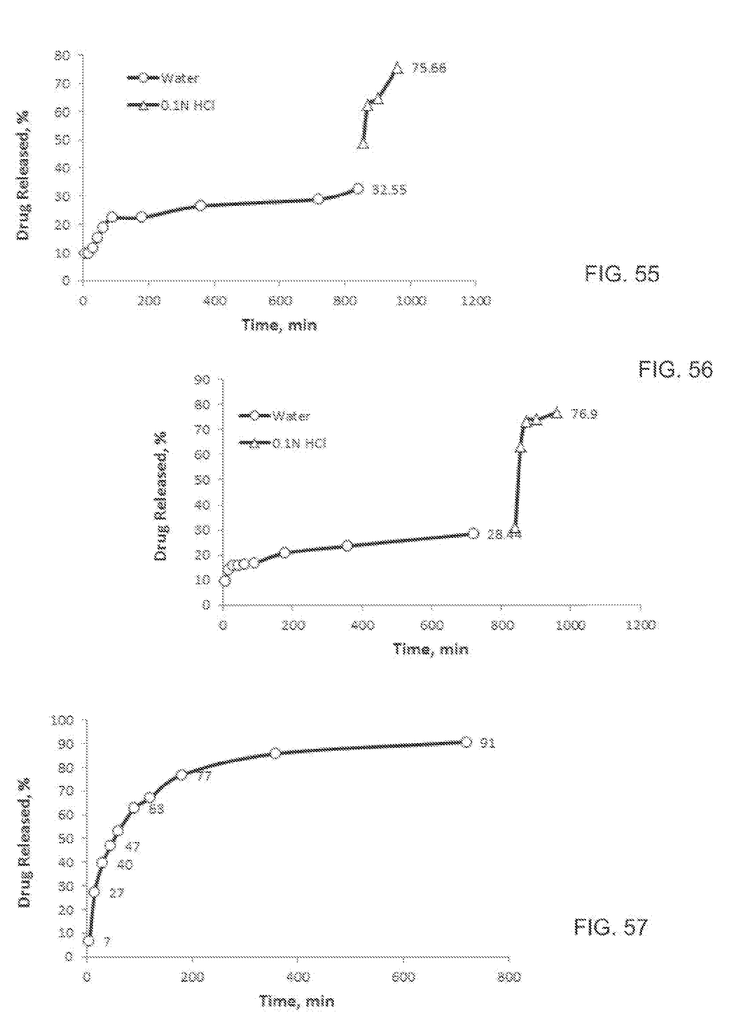

Tramadol-PVOH composite cryogels were released from their molds and subjected to dissolution studies using a USP 2 paddle method (Distek dissolution system 2100A) in 900 ml of ultrapure water at 37.5° C. at a paddle rotational speed of 50 rpm. After 850 minutes, the dissolution medium was changed to 0.1N HCl by adding concentrated hydrochloric acid into the dissolution medium. Tramadol concentration in the dissolution medium was analyzed using UV-Visible spectroscopy at 271 nm (UV-1700, Shimadzu) over time. Results are shown in Table 33 and FIG. 55.

TABLE 33

| Dissolution | Time | | [Tramadol HCl] | % of drug |
|---|---|---|---|---|
| medium | (min) | abs @ 271 nm | μg/ml | released |
| Water | 5 | 0.0080 | 2.03 | 9.44 |
| Water | 15 | 0.0079 | 2.02 | 9.40 |
| Water | 30 | 0.0106 | 2.47 | 11.53 |
| Water | 45 | 0.0153 | 3.29 | 15.35 |
| Water | 60 | 0.0197 | 4.05 | 18.88 |
| Water | 90 | 0.0239 | 4.78 | 22.26 |
| Water | 180 | 0.0241 | 4.81 | 22.42 |
| Water | 360 | 0.0292 | 5.69 | 26.52 |
| Water | 720 | 0.0323 | 6.22 | 28.97 |
| Water | 840 | 0.0367 | 6.98 | 32.55 |
| 0.1N HCl | 855 | 0.0679 | 10.50 | 48.94 |
| 0.1N HCl | 870 | 0.0853 | 13.39 | 62.42 |
| 0.1N HCl | 900 | 0.0882 | 13.88 | 64.71 |
| 0.1N HCl | 960 | 0.1023 | 16.23 | 75.66 |

### Drug Release for Shredded Cryogel

Tramadol-PVOH composite cryogels were released from their molds and placed into a micro-mill grinder (Bel-Art Products) for 5 seconds. The shredded pieces were then subjected to dissolution studies using a USP 2 paddle method (Distek dissolution system 2100A) in 900 ml of ultrapure water at 37.5° C. at a paddle rotational speed of 50 rpm. After 840 minutes, the dissolution medium was changed to 0.1N HCl by adding concentrated hydrochloric acid into the dissolution medium. Tramadol concentration in the dissolution medium was analyzed using UV-Visible spectroscopy at 271 nm (UV-1700, Shimadzu) over time. Results are shown in Table 34 and FIG. 56.

TABLE 34

| Dissolution | Time | | [Tramadol HCl] | % of drug |
|---|---|---|---|---|
| medium | (min) | abs @ 271 nm | μg/ml | released |
| Water | 5 | 0.0083 | 2.08 | 9.49 |
| Water | 15 | 0.0140 | 3.07 | 14.02 |
| Water | 30 | 0.0160 | 3.41 | 15.60 |
| Water | 45 | 0.0160 | 3.41 | 15.60 |
| Water | 60 | 0.0170 | 3.59 | 16.38 |
| Water | 90 | 0.0175 | 3.66 | 16.74 |
| Water | 180 | 0.0226 | 4.54 | 20.76 |
| Water | 360 | 0.0262 | 5.16 | 23.59 |
| Water | 720 | 0.0323 | 6.22 | 28.44 |
| 0.1N HCl | 840 | 0.0360 | 6.86 | 31.35 |
| 0.1N HCl | 855 | 0.0882 | 13.88 | 63.39 |
| 0.1N HCl | 870 | 0.1015 | 16.09 | 73.52 |
| 0.1N HCl | 900 | 0.1022 | 16.21 | 74.05 |
| 0.1N HCl | 960 | 0.1059 | 16.83 | 76.90 |

### Drug Release in 0.1N HCl

Tramadol-PVOH composite cryogels were released from their molds and subjected to dissolution studies using a USP 2 paddle method (Distek dissolution system 2100A) in 900 ml of 0.1N HCl at 37.5° C. at a paddle rotational speed of 50 rpm. Tramadol concentration in the dissolution medium was analyzed using UV-Visible spectroscopy at 271 nm (UV-1700, Shimadzu) over time. Results are shown in Table 35 and FIG. 57.

TABLE 35

| Dissolution | Time | | [Tramadol HCl] | % of drug |
|---|---|---|---|---|
| medium | (min) | abs @ 271 nm | μg/ml | released |
| 0.1N HCl | 5 | 0.0142 | 1.54 | 6.72 |
| 0.1N HCl | 15 | 0.0325 | 6.25 | 27.23 |
| 0.1N HCl | 30 | 0.0489 | 9.09 | 39.58 |
| 0.1N HCl | 45 | 0.0586 | 10.75 | 46.83 |
| 0.1N HCl | 60 | 0.0669 | 12.18 | 53.06 |
| 0.1N HCl | 90 | 0.0799 | 14.43 | 62.87 |
| 0.1N HCl | 120 | 0.0853 | 15.36 | 66.92 |
| 0.1N HCl | 180 | 0.0984 | 17.62 | 76.76 |
| 0.1N HCl | 360 | 0.1104 | 19.69 | 85.77 |
| 0.1N HCl | 720 | 0.1170 | 20.83 | 90.73 |

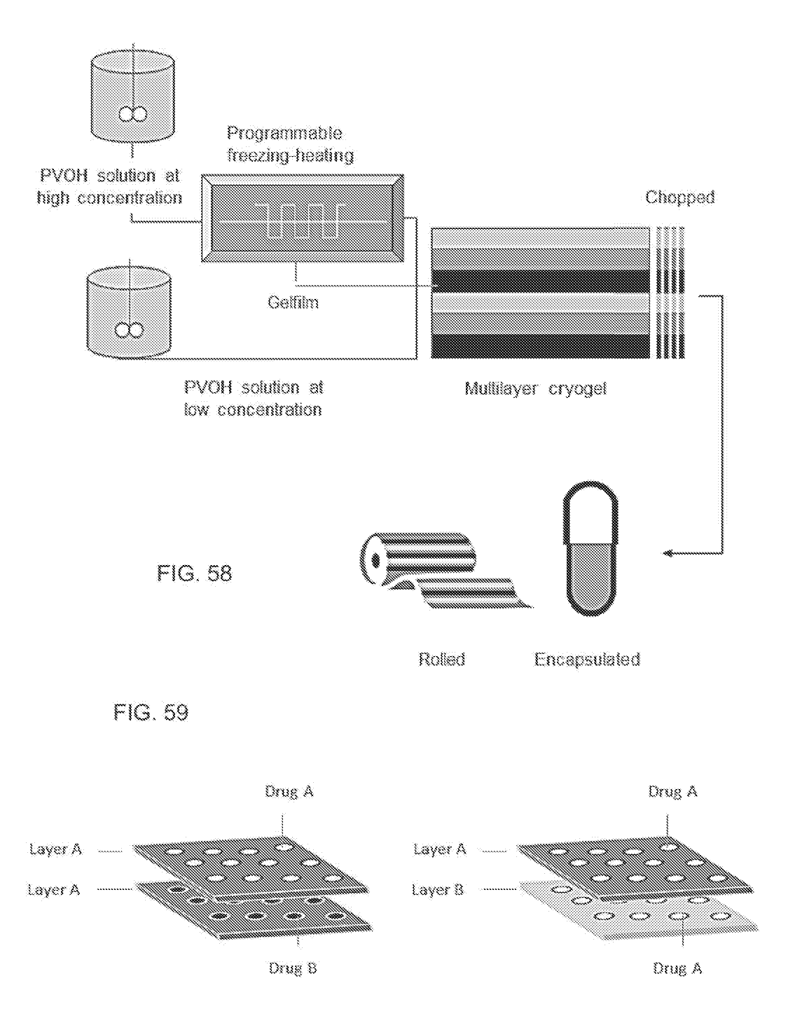

FIG. 58 illustrates a multilayer cryogel formed according to the process herein, that, after cryogelation, can be chopped, rolled or encapsulated. The cryogel layers may contain different drugs, possess different physical and mechanical properties, and contain different excipients and additives such as salts, solvents, plasticizers, surfactants, air and pore-forming agent.

FIG. 59 shows different layers that can be assembled into a multi-layer cryogel structure. Layers A and B are different in adhesive force, adhesiveness, gumminess, hardness, swelling capacity, and swelling rate. Layers A and B can have same or different dimensions and may contain same or different medications. Layers A and B may also contain same or different deterrent agents at any concentrations as described in this disclosure.

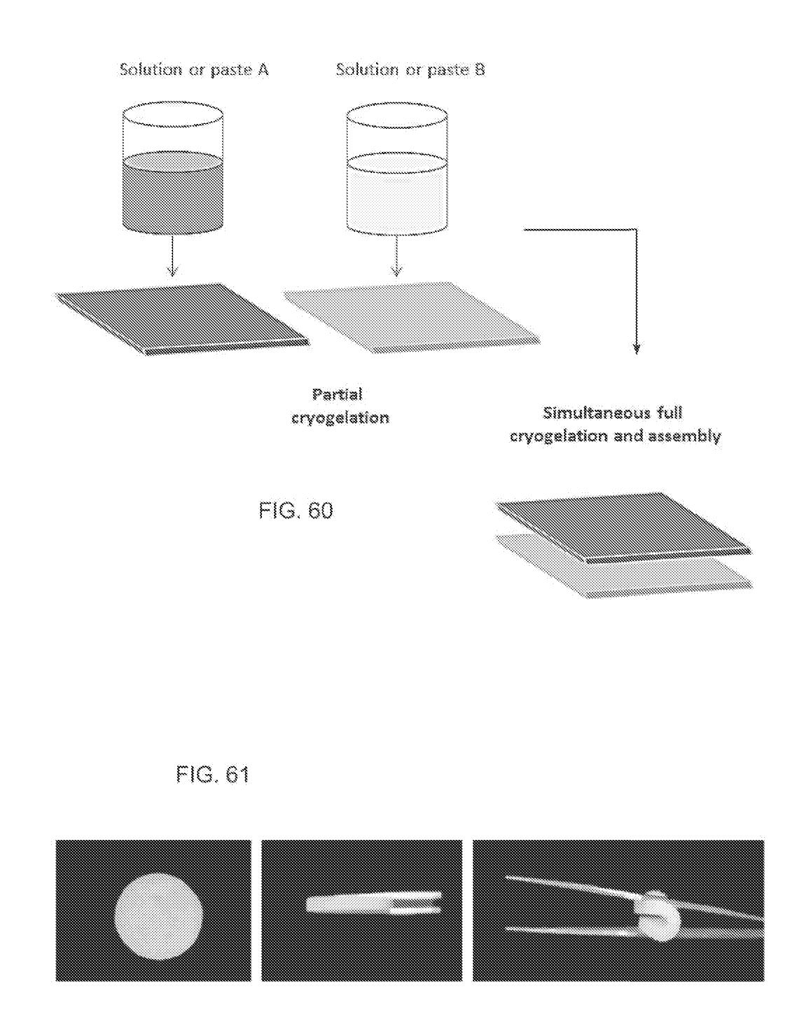

FIG. 60 shows a method to prepare a bilayer cryogel dosage form. For example, a paste “A” can be formed from a deterrent agent of the disclosure, in an amount of 10 wt % is added to a 5 wt % PVOH solution containing an abusable drug. A different paste “B” can be made using another formulation herein. Pastes A or B, after complete homogenization, undergo partial cryogelation, i.e., freezing and thawing to an extent that the cryogel is being formed but not reached its final physical mechanical property. This can be achieved by incomplete freezing or thawing or conducting the two processes at fewer cycles and shorter periods of time. Partially-cryogelled A and B layers will then be assembled and undergo full cryogelation process until desirable adhesive force, adhesiveness, gumminess, hardness and swelling are attained.

FIG. 61 shows a tablet shape dosage form cut from a cylindrical molded part (left and middle), bent at 180° displaying non-crushable rubber-like property; the composition is made of 5 wt % polyvinyl alcohol cryogel containing 10 wt % crosslinked carboxymethyl cellulose (Croscarmellose Na) as deterrent agent.

### Example 4: A Deterrent Cage for Immediate and Sustained Release

In accordance with the invention, a deterrent cage can be formulated with an abusable medication, providing a crush-resistant platform having the ability to release its drug in a sustained manner. In another embodiment, the same platform can be used to release the drug in an immediate fashion. The deterrent cage is composed of three different polymers including a water-swellable polymer, a water-soluble polymer, and a water-insoluble polymer, that can optimally be prepared by mixing individual polymers at certain ratio, or by using pre-mixed commercially available products, as shown in Table 36.

### Immediate Release Formulations

The example formulation tested is shown in Table 36.

TABLE 36

| | |
|---|---|
| Tramadol HCl | 25 mg |
| Prosolv ® | 170 mg |
| Ac-di-Sol ® | 305 mg |
| Total | 500 mg |

Extraction Studies:

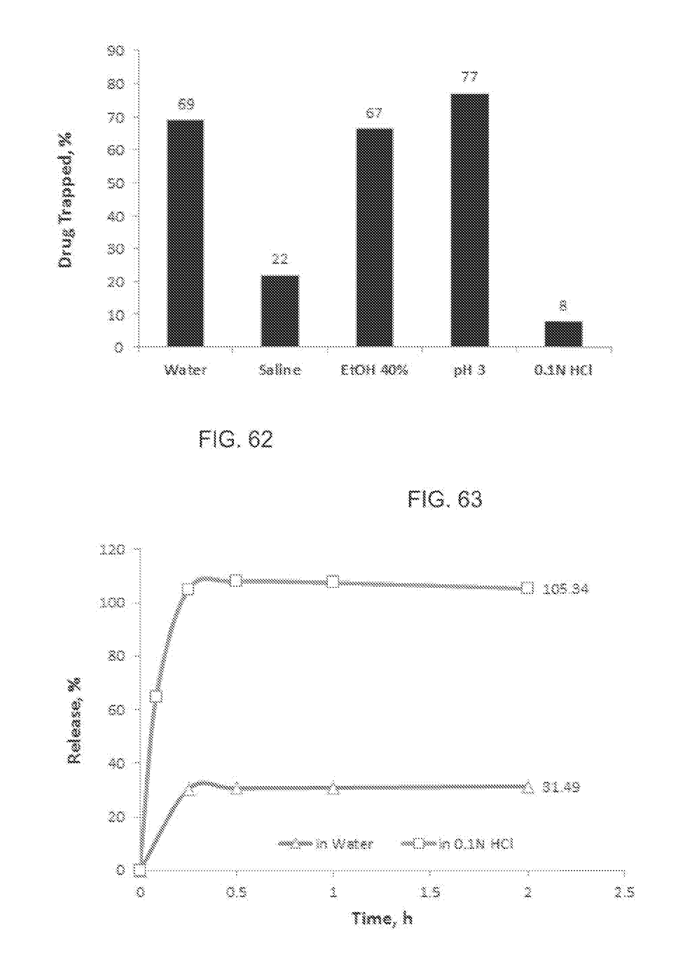

Physical mixture was transferred to 20 mL glass vial. 10 mL of water was added and vortexed for 5 seconds followed by centrifugation@1500 rpm for 5 minutes. The dispersion was filtered through 0.25 μm syringe filter, and 0.5 mL of the filtrate was diluted to 10 mL. The solution was analyzed for drug concentration by UV spectroscopy @271 nm. The results are shown in Table 37 and FIG. 62.

TABLE 37

| | | | | | Conc., | Amount, | % in | % |
|---|---|---|---|---|---|---|---|---|
| Medium | Abs 1 | Abs 2 | Abs 3 | Avg | μg/mL | mg | solution | Trapped |
| Water | 0.2346 | 0.2389 | 0.2415 | 0.238333 | 38.86 | 7.77 | 31.09 | 68.91 |
| Saline | 0.5974 | 0.5946 | 0.5947 | 0.595567 | 97.54 | 19.51 | 78.03 | 21.97 |
| EtOH 40% | 0.2554 | 0.2573 | 0.2559 | 0.2562 | 41.82 | 8.36 | 33.45 | 66.55 |
| pH 3 | 0.1683 | 0.1685 | 0.1681 | 0.1683 | 28.58621 | 5.72 | 22.87 | 77.13 |
| 0.1N HCl | 0.6897 | 0.6893 | 0.6901 | 0.6897 | 114.98 | 23.00 | 91.99 | 8.01 |

Drug Release Studies:

Prepared tablets were studied for their drug release profile in ultrapure water and 0.1N HCl @ 50 rpm and 37±2° C. (USP II). Samples were pulled at different time points and the % drug released was plotted versus time. The first 5 hours, the release was studied in ultrapure water, and then the medium was changed to 0.1N HCl by adding concentrated HCl. The results are shown in Table 38 and FIG. 63.

TABLE 38

| | | | | | | |
|---|---|---|---|---|---|---|
| Time, | | | | Conc., | Amount, | Release, |
| h | Abs 1 | Abs 2 | Avg | μg/mL | mg | % |

In water

| | | | | | | |
|---|---|---|---|---|---|---|
| 0.25 | 0.0458 | 0.0473 | 0.04655 | 8.42 | 7.58 | 30.31 |
| 0.5 | 0.0458 | 0.0487 | 0.04725 | 8.53 | 7.68 | 30.71 |
| 1 | 0.0471 | 0.0482 | 0.04765 | 8.60 | 7.74 | 30.94 |
| 2 | 0.0479 | 0.0493 | 0.0486 | 8.75 | 7.87 | 31.49 |

In 0.1N HCl

| | | | | | | |
|---|---|---|---|---|---|---|
| 0.083 | 0.1049 | 0.1096 | 0.10725 | 17.91 | 16.12 | 64.47 |
| 0.25 | 0.177 | 0.1776 | 0.1773 | 29.58 | 26.63 | 106.50 |
| 0.5 | 0.182 | 0.1827 | 0.18235 | 30.43 | 27.38 | 109.53 |
| 1 | 0.1826 | 0.1805 | 0.18155 | 30.29 | 27.26 | 109.05 |
| 2 | 0.1783 | 0.1774 | 0.17785 | 29.68 | 26.71 | 106.83 |

The formulation is composed of only one deterrent agent (croscarmellose, Ac-di-Sol®). Trapping capacity of this formulation is 69%, 22%, 67%, 77% in water, saline, EtOH40%, and pH 3 medium respectively. Such composition can release about 92% of its contents in 0.1N HCl providing an immediate release profile.

### Sustained Release Formulations

### Regular Sustained Release Formulation (Trial 1):

Formulation:

The formulation tested is shown in Table 39.

TABLE 39

| | |
|---|---|
| Tramadol HCl | 100 mg |
| Ticalose ® 6000 (CMC) | 300 mg |
| Prosolv ® | 100 mg |
| Total | 500 mg |

Preparation of Tablets:

Each ingredient was weighed and added to glass mortar where they were mixed by geometric dilution. Physical mixture was transferred to tablet press and compressed@2000 pounds force.

Extraction Studies:

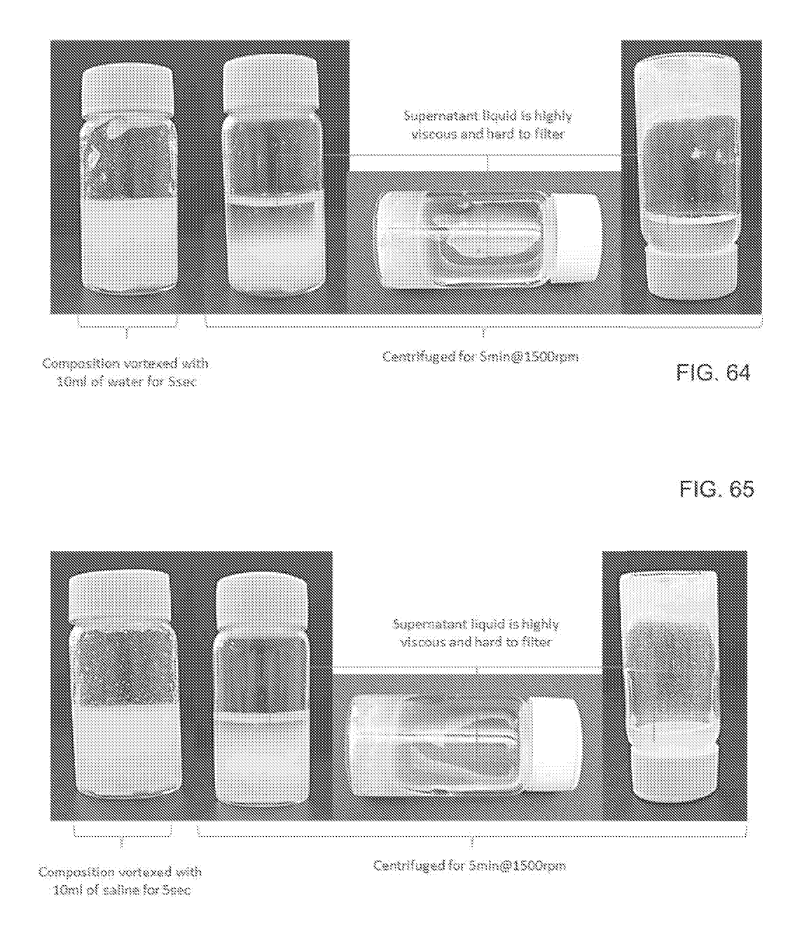

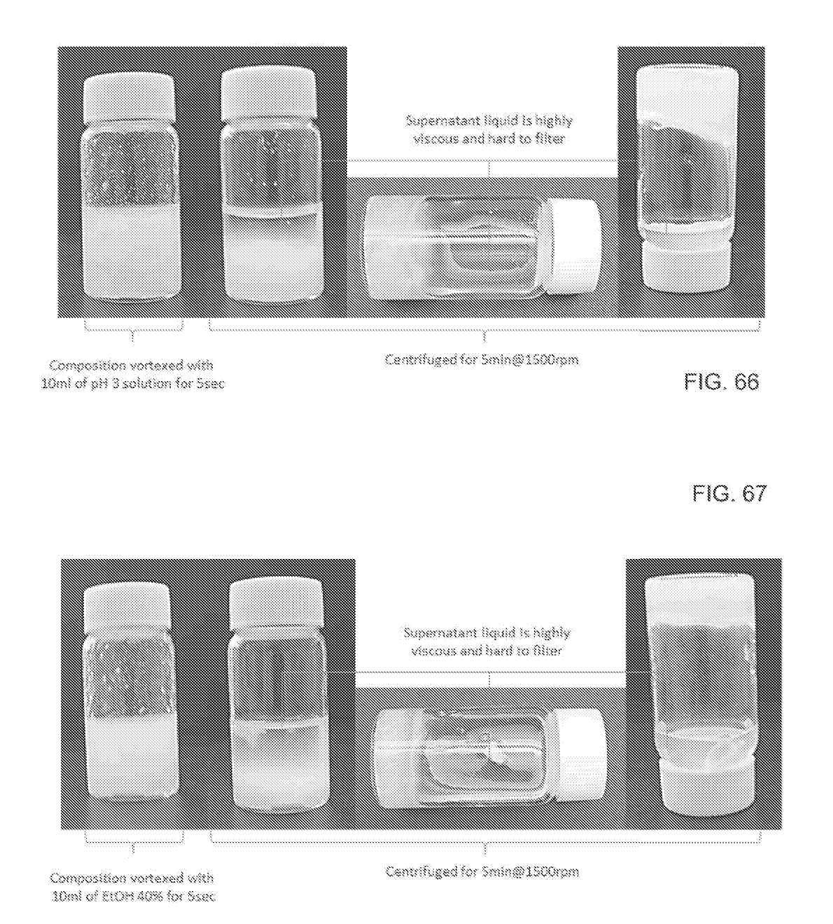

Physical mixture was transferred to 20 mL glass vial. 10 mL of water was added and vortexed for 5 seconds followed by centrifugation@1500 rpm for 5 minutes. As can be seen in FIGS. 64-67, the mixtures are extremely viscous, and no filtration or extraction is possible in any of the mediums, including water, pH3, pH1, saline, and EtOH 40%.

Drug Release Studies:

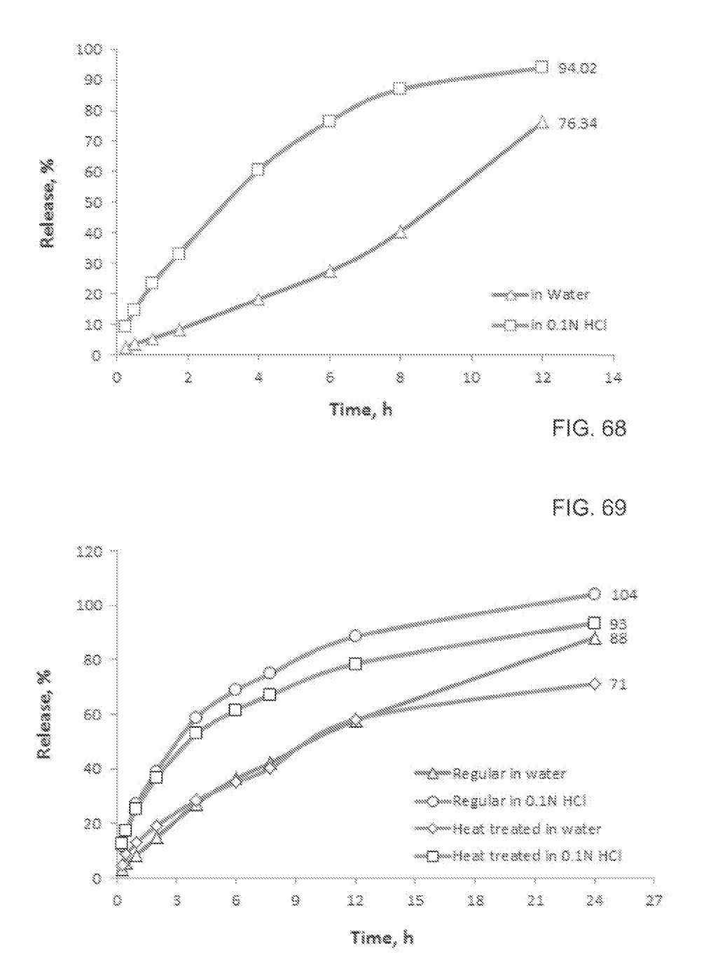

Prepared tablets were studied for their drug release profile in ultrapure water and 0.1N HCl @ 50 rpm and 37±2° C. Samples were pulled at different time points and graphs were plotted with % drug released vs time. Results are shown in Table 40 and FIG. 68.

TABLE 40

| | | | | | | |
|---|---|---|---|---|---|---|
| Time, | | | | Conc., | Amount, | Release, |
| h | Abs 1 | Abs 2 | Avg | μg/mL | mg | % |

In water

| | | | | | | |
|---|---|---|---|---|---|---|
| 0.25 | 0.0117 | 0.0121 | 0.0119 | 2.92 | 2.63 | 2.63 |
| 0.5 | 0.0183 | 0.0194 | 0.01885 | 4.02 | 3.62 | 3.62 |
| 1 | 0.0309 | 0.0314 | 0.03115 | 5.98 | 5.38 | 5.38 |
| 1.75 | 0.0516 | 0.0521 | 0.05185 | 9.26 | 8.34 | 8.34 |
| 4 | 0.1205 | 0.1224 | 0.12145 | 20.31 | 18.28 | 18.28 |
| 6 | 0.1853 | n/a | 0.1853 | 30.44 | 27.40 | 27.40 |
| 8 | 0.277 | 0.2771 | 0.27705 | 45.01 | 40.51 | 40.51 |
| 12 | 0.5299 | 0.5259 | 0.5279 | 84.83 | 76.34 | 76.34 |

In 0.1N HCl

| | | | | | | |
|---|---|---|---|---|---|---|
| 0.25 | 0.0601 | 0.0609 | 0.0605 | 10.12 | 9.11 | 9.11 |
| 0.5 | 0.0975 | 0.0969 | 0.0972 | 16.23 | 14.61 | 14.61 |
| 1 | 0.1553 | 0.1547 | 0.155 | 25.87 | 23.28 | 23.28 |
| 1.75 | 0.2203 | 0.2217 | 0.221 | 36.87 | 33.18 | 33.18 |
| 4 | 0.4036 | 0.4039 | 0.40375 | 67.33 | 60.59 | 60.59 |
| 6 | 0.5096 | 0.5115 | 0.51055 | 85.13 | 76.61 | 76.61 |
| 8 | 0.5786 | 0.5801 | 0.57935 | 96.59 | 86.93 | 86.93 |
| 12 | 0.6266 | 0.6266 | 0.6266 | 104.47 | 94.02 | 94.02 |

The sustained release formulation in trial 1 is composed of Ticalose® 6000 as a viscosifying agent. This composition provides a sustained release profile with different release profiles in water and in 0.1N HCl. As shown in the graph (FIG. 68), the release in water is significantly hindered over the first 8-10 hrs (maximum release of 50%) in the dissolution medium. However the release of a same drug in 0.1N HCl is reached to a plateau value of about 95% over the same period of time.

### Crush Resistant Sustained Release Formulation (Trial 2)

### Formulation:

The formulation tested is shown in Table 41.

TABLE 41

| | |
|---|---|
| Tramadol HCl | 100 mg |
| Ticalose ® 6000 | 150 mg |
| Kollidon ® SR | 150 mg |
| Total | 400 mg |

Preparation of tablets: Each ingredient was weighed and added to glass mortar where they were mixed by geometric dilution. Physical mixture was transferred to a tablet press and compressed @2000 pounds force. Results are shown in Tables 42 and 43, and FIG. 69.

TABLE 42

| | | | | | | | |
|---|---|---|---|---|---|---|---|
| Time, | | | | | Conc., | Amount, | Release, |
| h | Abs 1 | Abs 2 | Abs 3 | Avg | μg/mL | mg | % |

Regular tablet in water

| | | | | | | | |
|---|---|---|---|---|---|---|---|
| 0.25 | 0.0128 | 0.0155 | 0.0182 | 0.0155 | 3.49 | 3.14 | 3.14 |
| 0.5 | 0.0311 | 0.0311 | 0.0313 | 0.031167 | 5.98 | 5.38 | 5.38 |
| 1 | 0.0537 | 0.0531 | 0.0538 | 0.053533 | 9.53 | 8.58 | 8.58 |
| 2 | 0.0982 | 0.0968 | 0.0974 | 0.097467 | 16.50 | 14.85 | 14.85 |
| 4 | 0.1836 | 0.1846 | 0.1847 | 0.1843 | 30.29 | 27.26 | 27.26 |
| 6 | 0.2496 | 0.246 | 0.2498 | 0.248467 | 40.47 | 36.42 | 36.42 |
| 7.75 | 0.2878 | 0.2899 | 0.2886 | 0.288767 | 46.87 | 42.18 | 42.18 |
| 12 | 0.3961 | 0.3967 | 0.3978 | 0.396867 | 64.03 | 57.62 | 57.62 |
| 24 | 0.6084 | 0.6119 | 0.6073 | 0.6092 | 97.73 | 87.96 | 87.96 |

Regular tablet in 0.1N HCl

| | | | | | | | |
|---|---|---|---|---|---|---|---|
| 0.25 | 0.0759 | 0.0767 | 0.077 | 0.0763 | 12.75 | 11.48 | 11.48 |
| 0.5 | 0.1154 | 0.1158 | 0.1164 | 0.1156 | 19.30 | 17.37 | 17.37 |
| 1 | 0.1788 | 0.1804 | 0.1802 | 0.1796 | 29.97 | 26.97 | 26.97 |
| 1.75 | 0.2594 | 0.2606 | 0.2611 | 0.26 | 43.37 | 39.03 | 39.03 |
| 4 | 0.391 | 0.3918 | 0.3922 | 0.3914 | 65.27 | 58.74 | 58.74 |
| 6 | 0.4584 | 0.4586 | 0.4594 | 0.4585 | 76.45 | 68.81 | 68.81 |
| 7.75 | 0.4995 | 0.4987 | 0.4988 | 0.4991 | 83.22 | 74.90 | 74.90 |
| 12 | 0.589 | 0.5894 | 0.5889 | 0.5892 | 98.23 | 88.41 | 88.41 |
| 24 | 0.693 | 0.694 | 0.6947 | 0.6935 | 115.62 | 104.06 | 104.06 |

TABLE 43

| | | | | | | | |
|---|---|---|---|---|---|---|---|
| Time, | | | | | Conc., | Amount, | Release, |
| h | Abs 1 | Abs 2 | Abs 3 | Avg | μg/mL | mg | % |

Heat treated tablet in water

| | | | | | | | |
|---|---|---|---|---|---|---|---|
| 0.25 | 0.0264 | 0.0269 | 0.0266 | 0.026633 | 5.26 | 4.73 | 4.73 |
| 0.5 | 0.0532 | 0.0542 | 0.0538 | 0.0537 | 9.56 | 8.60 | 8.60 |
| 1 | 0.0834 | 0.0839 | 0.0842 | 0.08365 | 14.31 | 12.88 | 12.88 |
| 2 | 0.1273 | 0.1279 | 0.1301 | 0.1276 | 21.29 | 19.16 | 19.16 |
| 4 | 0.1932 | 0.1926 | 0.194 | 0.1929 | 31.65 | 28.49 | 28.49 |
| 6 | 0.2388 | 0.2371 | 0.2401 | 0.23795 | 38.80 | 34.92 | 34.92 |
| 7.75 | 0.272 | 0.2782 | 0.2795 | 0.2751 | 44.70 | 40.23 | 40.23 |
| 12 | 0.3978 | 0.4022 | | 0.4 | 64.52 | 58.07 | 58.07 |
| 24 | 0.4952 | 0.4885 | | 0.49185 | 79.10 | 71.19 | 71.19 |

Heat treated tablet in 0.1N HCl

| | | | | | | | |
|---|---|---|---|---|---|---|---|
| 0.25 | 0.0845 | 0.0837 | 0.082 | 0.0834 | 13.93 | 12.54 | 12.54 |
| 0.5 | 0.1149 | 0.1144 | 0.1144 | 0.114567 | 19.13 | 17.22 | 17.22 |
| 1 | 0.1681 | 0.1676 | 0.168 | 0.1679 | 28.02 | 25.22 | 25.22 |
| 2 | 0.2443 | 0.2439 | 0.2443 | 0.244167 | 40.73 | 36.66 | 36.66 |
| 4 | 0.3511 | 0.351 | 0.3513 | 0.351133 | 58.56 | 52.70 | 52.70 |
| 6 | 0.4095 | 0.4097 | 0.4104 | 0.409867 | 68.34 | 61.51 | 61.51 |
| 7.75 | 0.4468 | 0.4464 | 0.4468 | 0.446667 | 74.48 | 67.03 | 67.03 |
| 12 | 0.5227 | 0.5226 | 0.5232 | 0.522833 | 87.17 | 78.46 | 78.46 |
| 24 | 0.623 | 0.6223 | 0.6227 | 0.622667 | 103.81 | 93.43 | 93.43 |

The formulation in trial 2 is composed of Ticalose® 6000 as a viscosifier and Kollidon® SR as a plastic agent providing crush resistance when heated at 120° C. for 1 hr.

### Crush Resistant Sustained Release Formulation (Trial 3)

Formulation:

The example formulation tested is shown in Table 44.

TABLE 44

| | |
|---|---|
| Tramadol HCl | 100 mg |
| Ticalose ® 6000 | 150 mg |
| Kollidon ® SR | 250 mg |
| Total | 500 mg |

Preparation of Tablets:

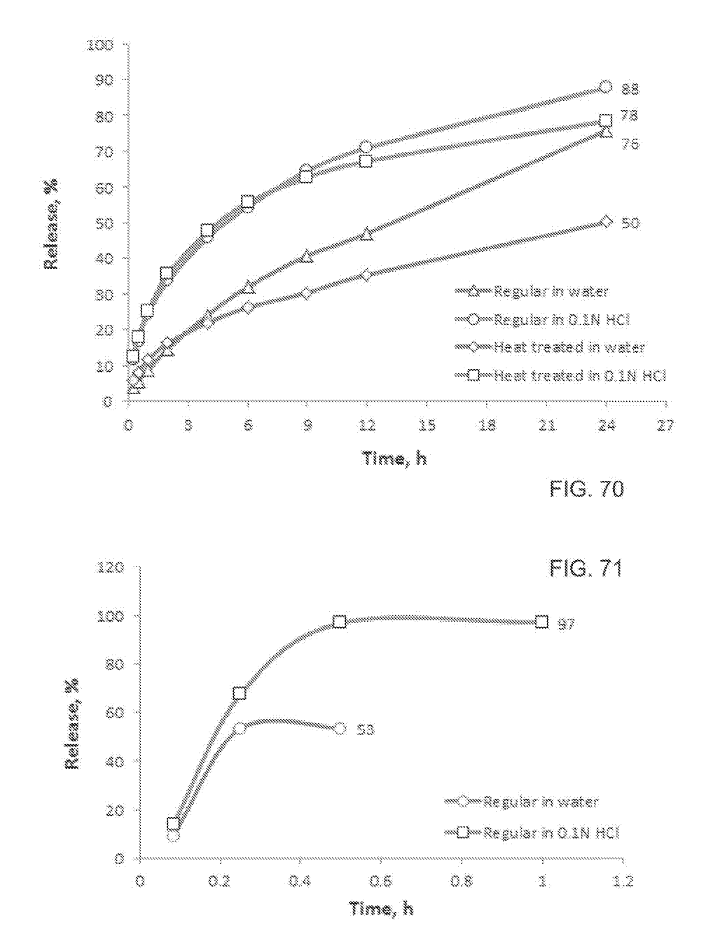

Each ingredient was weighed and added to a glass mortar where they were mixed by geometric dilution. Physical mixture was transferred to tablet press and compressed @2000 pounds force. Results are shown in Tables 45 and 46, and FIG. 70.

TABLE 45

| | | | | | | | |
|---|---|---|---|---|---|---|---|
| Time, | | | | | Conc., | Amount, | Release, |
| h | Abs 1 | Abs 2 | Abs 3 | Avg | μg/mL | mg | % |

Regular tablet in water

| | | | | | | | |
|---|---|---|---|---|---|---|---|
| 0.25 | 0.0212 | 0.0212 | 0.0219 | 0.021433 | 4.43 | 3.99 | 3.99 |
| 0.5 | 0.0327 | 0.0331 | 0.0331 | 0.032967 | 6.26 | 5.64 | 5.64 |
| 1 | 0.0544 | 0.0553 | 0.0552 | 0.054967 | 9.76 | 8.78 | 8.78 |
| 2 | 0.0964 | 0.0959 | 0.097 | 0.096433 | 16.34 | 14.70 | 14.70 |
| 4 | 0.163 | 0.1635 | 0.1624 | 0.162967 | 26.90 | 24.21 | 24.21 |
| 6 | 0.217 | 0.2172 | 0.2172 | 0.217133 | 35.50 | 31.95 | 31.95 |
| 9 | 0.2786 | 0.2786 | 0.2803 | 0.279167 | 45.34 | 40.81 | 40.81 |
| 12 | 0.3225 | 0.3228 | 0.3232 | 0.322833 | 52.28 | 47.05 | 47.05 |
| 24 | 0.5236 | 0.5267 | 0.5219 | 0.524067 | 84.22 | 75.80 | 75.80 |

Regular tablet in 0.1N HCl

| | | | | | | | |
|---|---|---|---|---|---|---|---|
| 0.25 | 0.0769 | 0.0773 | | 0.0771 | 12.88 | 11.60 | 11.60 |
| 0.5 | 0.1113 | 0.1121 | 0.1119 | 0.1117 | 18.65 | 16.79 | 16.79 |
| 1 | 0.1626 | 0.1644 | 0.1631 | 0.1635 | 27.28 | 24.56 | 24.56 |
| 1.75 | 0.2251 | 0.2251 | 0.2244 | 0.2251 | 37.55 | 33.80 | 33.80 |
| 4 | 0.3053 | 0.3066 | 0.3059 | 0.30595 | 51.03 | 45.92 | 45.92 |
| 6 | 0.3619 | 0.3627 | 0.3628 | 0.3623 | 60.42 | 54.38 | 54.38 |
| 9 | 0.4301 | 0.4297 | 0.4298 | 0.4299 | 71.68 | 64.52 | 64.52 |
| 12 | 0.4727 | 0.4727 | 0.4727 | 0.4727 | 78.82 | 70.94 | 70.94 |
| 24 | 0.5842 | 0.5845 | 0.5845 | 0.5844 | 97.43 | 87.69 | 87.69 |

TABLE 46

| | | | | | | | |
|---|---|---|---|---|---|---|---|
| Time, | | | | | Conc., | Amount, | Release, |
| h | Abs 1 | Abs 2 | Abs 3 | Avg | μg/mL | mg | % |

Heat treated tablet in water

| | | | | | | | |
|---|---|---|---|---|---|---|---|
| 0.25 | 0.0355 | 0.0359 | 0.0352 | 0.035533 | 6.67 | 6.00 | 6.00 |
| 0.5 | 0.0497 | 0.0513 | 0.0516 | 0.050867 | 9.11 | 8.20 | 8.20 |
| 1 | 0.0747 | 0.0751 | 0.0756 | 0.075133 | 12.96 | 11.66 | 11.66 |
| 2 | 0.1078 | 0.1075 | 0.1094 | 0.108233 | 18.21 | 16.39 | 16.39 |
| 4 | 0.1471 | 0.1476 | 0.1476 | 0.147433 | 24.43 | 21.99 | 21.99 |
| 6 | 0.1769 | 0.1771 | 0.1779 | 0.1773 | 29.17 | 26.26 | 26.26 |
| 9 | 0.2054 | 0.2069 | 0.2087 | 0.207 | 33.89 | 30.50 | 30.50 |
| 12 | 0.243 | 0.2407 | 0.2389 | 0.240867 | 39.26 | 35.34 | 35.34 |
| 24 | 0.3436 | 0.3464 | 0.3433 | 0.344433 | 55.70 | 50.13 | 50.13 |

Heat treated tablet in 0.1N HCl

| | | | | | | | |
|---|---|---|---|---|---|---|---|
| 0.25 | 0.0824 | 0.0824 | 0.0824 | 0.0824 | 13.77 | 12.39 | 12.39 |
| 0.5 | 0.1187 | 0.1191 | 0.1193 | 0.119033 | 19.87 | 17.89 | 17.89 |
| 1 | 0.1688 | 0.1689 | 0.1688 | 0.168833 | 28.17 | 25.36 | 25.36 |
| 1.75 | 0.2372 | 0.2374 | 0.2378 | 0.237467 | 39.61 | 35.65 | 35.65 |
| 4 | 0.3164 | 0.317 | 0.3175 | 0.316967 | 52.86 | 47.58 | 47.58 |
| 6 | 0.3715 | 0.3702 | 0.3704 | 0.3707 | 61.82 | 55.64 | 55.64 |
| 9 | 0.4174 | 0.418 | 0.4178 | 0.417733 | 69.66 | 62.69 | 62.69 |
| 12 | 0.4482 | 0.4478 | 0.4479 | 0.447967 | 74.69 | 67.23 | 67.23 |
| 24 | 0.5225 | 0.5234 | 0.5226 | 0.522833 | 87.17 | 78.46 | 78.46 |

The formulation in trial 3 is composed of Ticalose® 6000 as a viscosifier and Kollidon® SR as a plastic agent at higher concentration of Kollidon® SR providing crush resistance when heated at 120° C. for 1 hr. Addition of more SR into composition reduces the ultimate amount of release from (93-100%) down to (78-88%) in 0.1N HCl. The heated tablet provides more hindrance to release.

### One Platform for Both Immediate and Sustained Release Formulations

Formulation (Trial 1):

The formulation tested is shown in Table 47.

TABLE 47

| | |
|---|---|
| Tramadol ® HCl | 100 mg |
| Ac-Di-Sol ® | 150 mg |
| Kollidon ® SR | 250 mg |
| Total | 500 mg |

Preparation of Tablets:

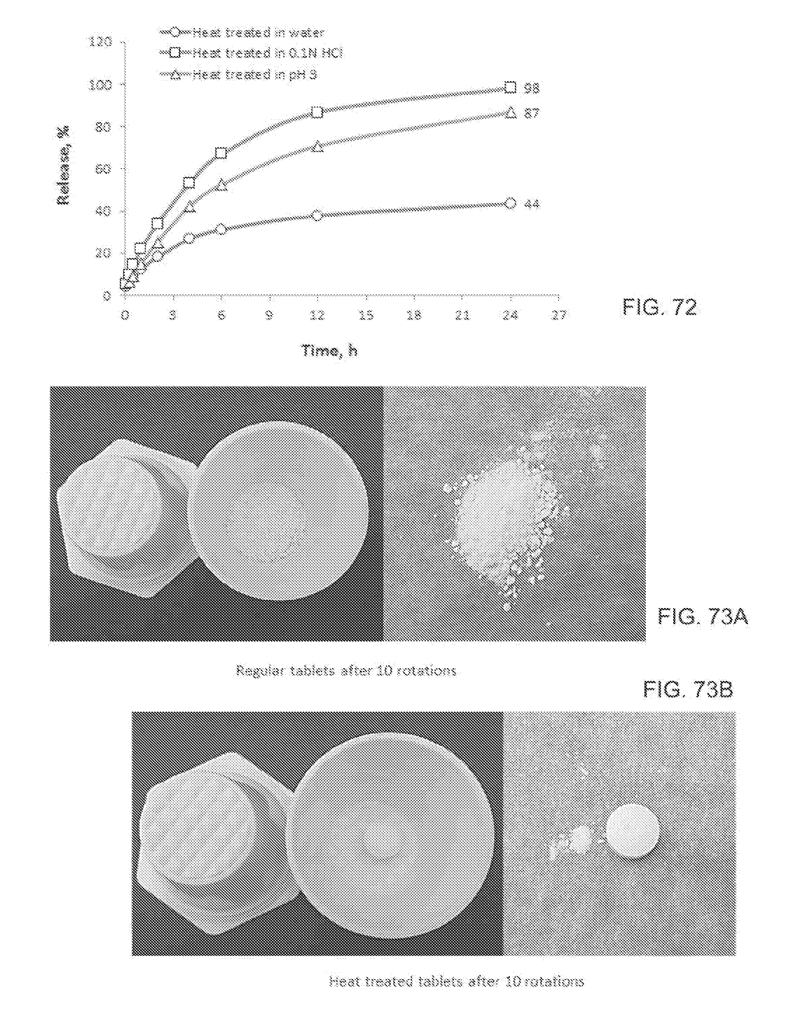

Each ingredient was weighed and added to glass mortar where they were mixed by geometric dilution. Physical mixture was transferred to tablet press and compressed @2000 pounds force. Results are shown in Tables 48-49 and FIGS. 71-72.

TABLE 48

| | | | | | | | |
|---|---|---|---|---|---|---|---|
| Time, | | | | | Conc., | Amount, | Release, |
| h | Abs 1 | Abs 2 | Abs 3 | Avg | μg/mL | mg | % |

Regular tablet in water

| | | | | | | | |
|---|---|---|---|---|---|---|---|
| 0.083 | 0.058 | 0.0599 | 0.0604 | 0.059433 | 10.47 | 9.42 | 9.42 |
| 0.25 | 0.3683 | 0.3667 | 0.3669 | 0.3673 | 59.33 | 53.40 | 53.40 |
| 0.5 | 0.3669 | 0.3672 | | 0.36705 | 59.29 | 53.36 | 53.36 |
| 9 | 0.3647 | 0.3661 | | 0.3654 | 59.03 | 53.13 | 53.13 |

Regular tablet in 0.1N HCl

| | | | | | | | |
|---|---|---|---|---|---|---|---|
| 0.083 | 0.0933 | 0.0931 | 0.0919 | 0.092767 | 15.49 | 13.95 | 13.95 |
| 0.25 | 0.4504 | 0.4478 | 0.4494 | 0.4492 | 74.90 | 67.41 | 67.41 |
| 0.5 | 0.6458 | 0.6428 | 0.6476 | 0.6454 | 107.60 | 96.84 | 96.84 |
| 1 | 0.6472 | 0.6478 | 0.6464 | 0.647133 | 107.89 | 97.10 | 97.10 |
| 9 | 0.6281 | 0.6276 | 0.6281 | 0.627933 | 104.69 | 94.22 | 94.22 |

Regular tablets (non-heated) provide immediate but hindered release in water up to 50%, whereas the same formulation in 0.1N HCl releases almost all its contents in an immediate fashion. Contrary to other compositions containing Kollidon® SR with sustained release property, the composition containing Kollidon® SR and a superdisintegrant provides immediate release in both water and 0.1N HCl if not heat-treated.

TABLE 49

| | | | | | | | |
|---|---|---|---|---|---|---|---|
| Time, | | | | | Conc., | Amount, | Release, |
| h | Abs 1 | Abs 2 | Abs 3 | Avg | μg/mL | mg | % |

Heat treated tablet in water

| | | | | | | | |
|---|---|---|---|---|---|---|---|
| 0.083 | 0.0221 | 0.0234 | 0.0233 | 0.022933 | 4.67 | 4.20 | 4.20 |
| 0.25 | 0.0365 | 0.0381 | 0.0388 | 0.0378 | 7.03 | 6.33 | 6.33 |
| 0.5 | 0.0551 | 0.0567 | 0.0568 | 0.0562 | 9.95 | 8.96 | 8.96 |
| 1 | 0.0802 | 0.0809 | 0.0815 | 0.080867 | 13.87 | 12.48 | 12.48 |
| 2 | 0.1191 | 0.1201 | 0.1205 | 0.1199 | 20.06 | 18.06 | 18.06 |
| 4 | 0.1812 | 0.1799 | 0.1818 | 0.180967 | 29.76 | 26.78 | 26.78 |
| 6 | 0.2112 | 0.212 | 0.2122 | 0.2118 | 34.65 | 31.19 | 31.19 |
| 9 | 0.2405 | 0.2417 | 0.2424 | 0.241533 | 39.37 | 35.43 | 35.43 |
| 12 | 0.2567 | 0.2588 | 0.2594 | 0.2583 | 42.03 | 37.83 | 37.83 |
| 24 | 0.2956 | 0.2997 | 0.3005 | 0.2986 | 48.43 | 43.59 | 43.59 |

Heat treated tablet in 0.1N HCl

| | | | | | | | |
|---|---|---|---|---|---|---|---|
| 0.083 | 0.035 | 0.0355 | 0.0356 | 0.035367 | 5.93 | 5.34 | 5.34 |
| 0.25 | 0.0624 | 0.0629 | 0.063 | 0.062767 | 10.49 | 9.45 | 9.45 |
| 0.5 | 0.095 | 0.0955 | 0.0963 | 0.0956 | 15.97 | 14.37 | 14.37 |
| 1 | 0.1469 | 0.1478 | 0.1458 | 0.146833 | 24.51 | 22.06 | 22.06 |
| 2 | 0.2268 | 0.2256 | 0.2256 | 0.226 | 37.70 | 33.93 | 33.93 |
| 4 | 0.353 | 0.3538 | 0.3553 | 0.354033 | 59.04 | 53.14 | 53.14 |
| 6 | 0.4462 | 0.4462 | 0.4462 | 0.4462 | 74.40 | 66.96 | 66.96 |
| 9 | 0.5299 | 0.5284 | 0.5291 | 0.529133 | 88.22 | 79.40 | 79.40 |
| 12 | 0.5784 | 0.5784 | 0.5779 | 0.578233 | 96.41 | 86.77 | 86.77 |
| 24 | 0.6553 | 0.6543 | 0.6565 | 0.655367 | 109.26 | 98.34 | 98.34 |

Heat treated tablet in pH 3 solution

| | | | | | | | |
|---|---|---|---|---|---|---|---|
| 0.25 | 0.0425 | 0.0427 | 0.0428 | 0.042667 | 6.93 | 6.23 | 6.23 |
| 0.5 | 0.0614 | 0.0614 | 0.0615 | 0.061433 | 10.16 | 9.14 | 9.14 |
| 1 | 0.0981 | 0.0984 | 0.0986 | 0.098367 | 16.53 | 14.88 | 14.88 |
| 2 | 0.165 | 0.1655 | 0.1658 | 0.165433 | 28.09 | 25.28 | 25.28 |
| 4 | 0.2747 | 0.2753 | 0.2747 | 0.2749 | 46.97 | 42.27 | 42.27 |
| 6 | 0.3409 | 0.3409 | 0.3409 | 0.3409 | 58.34 | 52.51 | 52.51 |
| 8 | 0.3899 | 0.3906 | 0.3901 | 0.3902 | 66.84 | 60.16 | 60.16 |
| 12 | 0.4583 | 0.4586 | 0.4591 | 0.458667 | 78.65 | 70.78 | 70.78 |
| 24 | 0.5623 | 0.5626 | 0.5629 | 0.5626 | 96.57 | 86.91 | 86.91 |

As opposed to non-heated tablets which provided immediate release profile, same heated tablets containing Kollidon® SR and superdisintegrant provided a 24 hr sustained release in 0.1N HCl. Same composition displayed a hindered sustained release of maximum 44% in water. The amount of drug release from same heated composition in pH 3 medium was reached to 87%.

### Crush Resistance Studies:

Tablet Crusher

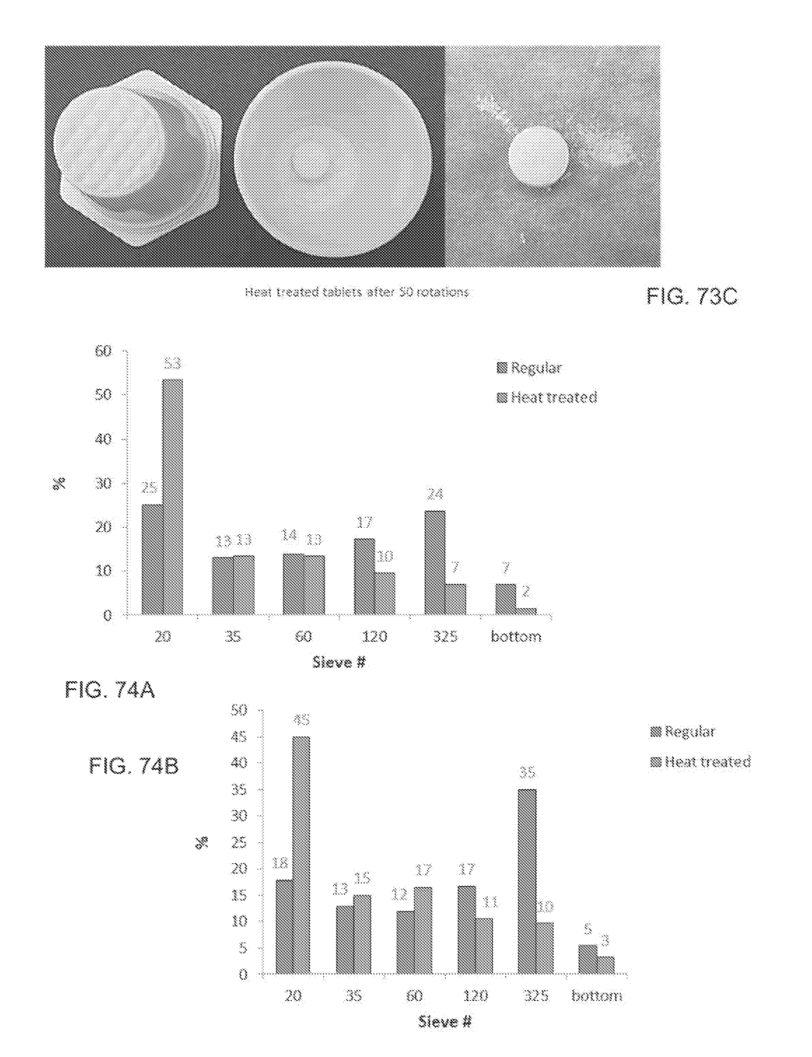

Tablets prepared as in Trial 1 (immediate and sustained release) were tested using a regular tablet crusher. Composition containing 30% of Ac-di-Sol® and 50% of Kollidon® SR displayed different resistance to crushing when tested in a tablet crusher. As can be seen in FIGS. 73-75, regular tablets were easily crushed to fine powders, whereas the heated tablets resisted crushing to an almost full extent.

### Particle Size Distribution Studies

Tablets were heat treated for 1 h@120° C. Non-treated and heat treated tablets of a same composition were then placed in a heavy-duty grinder (MicroMill®II by Science Ware) for 10 sec. Crushed tablets were collected and characterized for particle size distribution. Sieve analysis was carried out by placing sieves (#20, 35, 60, 120, 325 and bottom) on sifter (SS-3CP by Cole Palmer). Tapping was done for one minute at 60 taps per minute. Results are shown in FIGS. 74A-74C.

### Formulation (Sustained Release Trial 2):

The formulation tested is shown in Table 50 and results are shown in FIG. 74A.

TABLE 50

| | |
|---|---|
| Tramadol HCl | 100 mg |
| Ticalose ® 6000 | 150 mg |
| Kollidon ® SR | 150 mg |
| Total | 400 mg |

### Formulation (Sustained Release Trial 3):

The formulation tested is shown in Table 51 and results are shown in FIG. 74B.

TABLE 51

| | |
|---|---|
| Tramadol HCl | 100 mg |
| Ticalose ® 6000 | 150 mg |
| Kollidon ® SR | 250 mg |
| Total | 500 mg |

### Formulation (Immediate and Sustained Release Trial 1):

The formulation tested is shown in Table 52 and results are shown in FIG. 74C.

TABLE 52

| | |
|---|---|
| Tramadol HCl | 100 mg |
| Ac-Di-Sol ® | 150 mg |
| Kollidon ® SR | 250 mg |
| Total | 500 mg |

### Overall Effect of Heat Treatment on Tablet Crushability

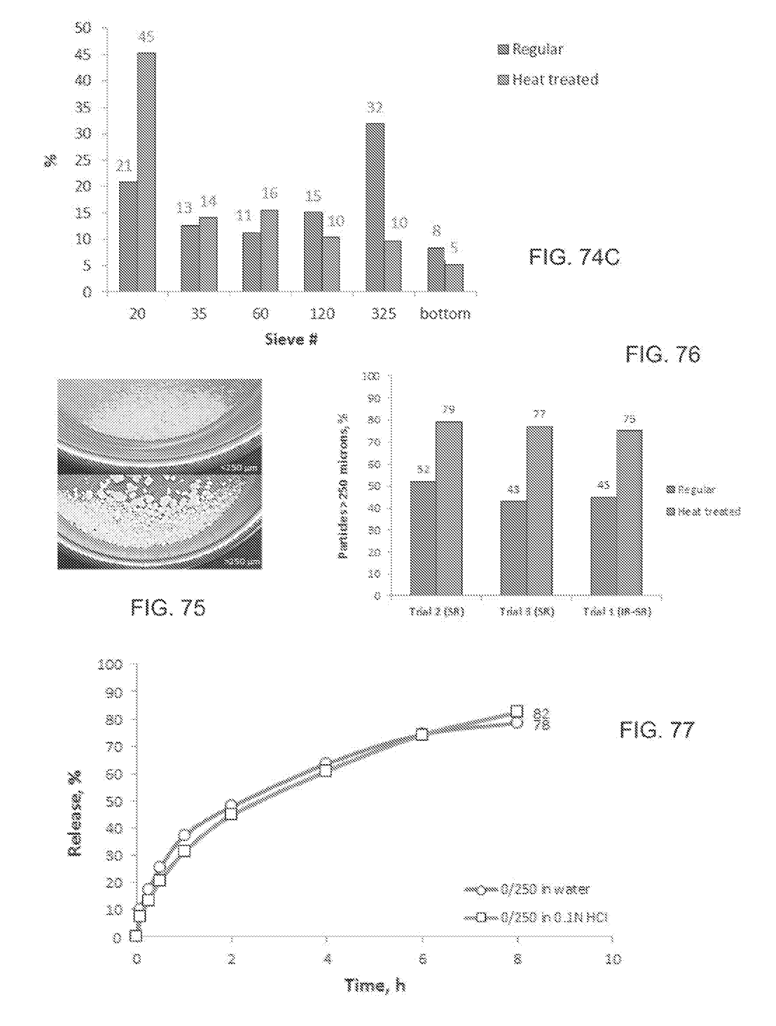

Relative particle size is shown in FIG. 75. Regardless of the sustained release formulation, the % of particles >250 μm remained at the level of 43-52% for regular tablets, whereas the % of particles >250 μm was in the range of 75-79% for heat-treated tablets, as illustrated in FIG. 76.

### More Studies on Deterrent Cage (Trial 1 Immediate and Sustained Release Platform)

Effect of Deterrent Concentration on Tramadol Release from Deterrent Cage

Regular Tramadol Tablets with No Heat-Treatment

The formulation tested is shown in Table 52, tested in water, as shown in Table 54, and in HCl, as shown in Table 55.

TABLE 53

| | | |
|---|---|---|
| Tramadol HCl | 100 | mg |
| Ac-Di-Sol ® | 0-150 | mg |
| Kollidon ® SR | 250 | mg |
| Total | 500 | mg |

TABLE 54
In Water

| Time, h | 0/250 | 50/250 | 100/250 | 150/250 |
|---|---|---|---|---|
| 0 | 0 | 0 | 0 | 0 |
| 0.083 | 9.92 | 7.28 | 11.65 | 9.42 |
| 0.25 | 17.60 | 27.43 | 42.58 | 53.40 |
| 0.5 | 25.52 | 61.40 | 59.98 | 53.36 |
| 1 | 37.32 | 70.90 | 60.63 | 53.36 |
| 2 | 47.96 | 70.52 | 61.63 | 53.36 |
| 4 | 63.55 | 74.26 | 63.51 | 53.36 |
| 6 | 74.35 | 74.26 | 63.51 | 53.36 |
| 8 | 78.42 | 74.26 | 63.51 | 53.36 |

TABLE 55
In 0.1N HCl

| Time, h | 0/250 | 50/250 | 100/250 | 150/250 |
|---|---|---|---|---|
| 0 | 0 | 0 | 0 | 0 |
| 0.083 | 7.34 | 8.75 | 25.61 | 13.95 |
| 0.25 | 13.44 | 32.21 | 69.86 | 67.41 |
| 0.5 | 20.67 | 62.71 | 97.77 | 96.84 |
| 1 | 31.22 | 96.43 | 97.82 | 96.84 |
| 2 | 44.78 | 96.95 | 97.82 | 96.84 |
| 4 | 60.85 | 96.95 | 97.82 | 96.84 |
| 6 | 74.07 | 96.95 | 97.82 | 96.84 |
| 8 | 82.29 | 96.95 | 97.82 | 96.84 |

As can be seen in FIG. 77, non-heated tablet composition containing 0% superdisintegrant provided same sustained release profiles in water and in 0.1N HCl. This composition released the drug up to 80%.

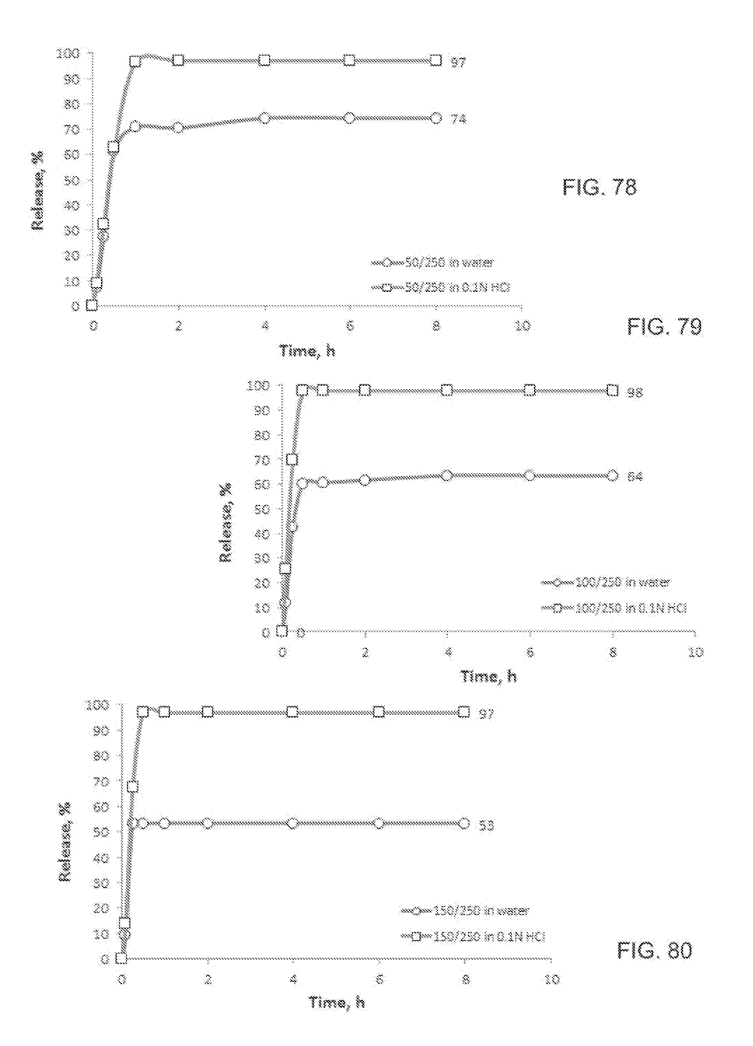

As can be seen in FIG. 78, non-heated tablet composition containing 10% superdisintegrant provided an immediate release profile in both water and 0.1N HCl. However, such composition displayed a hindered release of up to 74% in water.

As can be seen in FIG. 79, non-heated tablet composition containing 20% superdisintegrant provided an immediate release profiles in both water and in 0.1N HCl. However, such composition displayed a hindered release of up to 64% in water.

As can be seen in FIG. 80, non-heated tablet composition containing 30% superdisintegrant provided an immediate release profiles in both water and in 0.1N HCl. However, such composition displayed a hindered release of up to 53% in water.

### Heat-Treated Tramadol Tablets

Release data for heat-treated tramadol tablets in water are shown in Table 56, and for HCl in Table 57.

TABLE 56
In water

| Time, h | 0/250 | 50/250 | 100/250 | 150/250 |
|---|---|---|---|---|
| 0.083 | 12.23 | 8.14 | 6.90 | 4.20 |
| 0.25 | 16.69 | 10.76 | 9.80 | 6.33 |
| 0.5 | 20.95 | 13.44 | 13.30 | 8.96 |
| 1 | 25.97 | 19.00 | 18.27 | 12.48 |
| 2 | 33.34 | 26.60 | 25.49 | 18.06 |
| 4 | 39.54 | 35.28 | 33.06 | 26.78 |
| 6 | 42.95 | 40.57 | 38.25 | 31.19 |
| 12 | 47.63 | 52.23 | 46.79 | 37.83 |
| 24 | 61.15 | 61.99 | 51.56 | 43.59 |

TABLE 57
In 0.1N HCl

| Time, h | 0/250 | 50/250 | 100/250 | 150/250 |
|---|---|---|---|---|
| 0.083 | 9.21 | 9.50 | 7.62 | 5.34 |
| 0.25 | 12.78 | 13.82 | 10.55 | 9.45 |
| 0.5 | 17.53 | 18.50 | 15.12 | 14.37 |
| 1 | 26.28 | 25.27 | 22.40 | 22.06 |
| 2 | 33.85 | 33.95 | 32.96 | 33.93 |
| 4 | 40.31 | 44.66 | 46.61 | 53.14 |
| 6 | 44.18 | 50.89 | 55.88 | 66.96 |
| 12 | 50.98 | 62.86 | 74.27 | 86.77 |
| 24 | 62.47 | 78.58 | 88.34 | 98.34 |

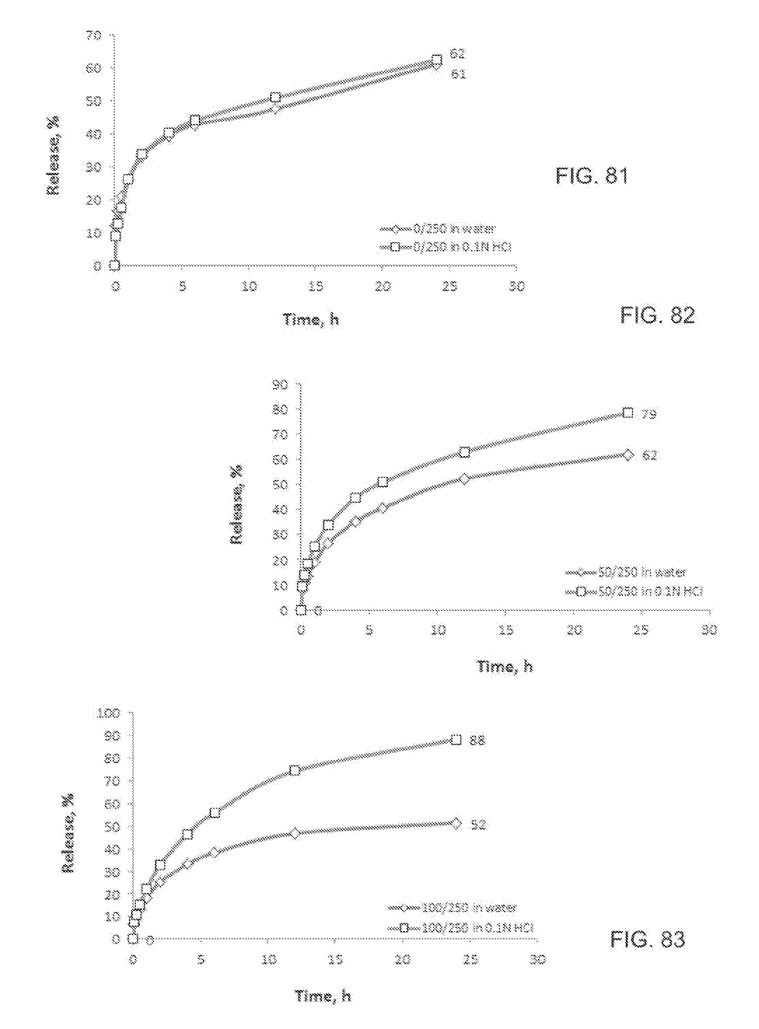

As can be seen in FIG. 81, a heat-treated tablet composition containing 0% superdisintegrant provided a similar sustained release profile in both water and in 0.1N HCl. This composition released the drug up to 60%.

As can be seen in FIG. 82, a heat-treated tablet composition containing 10% superdisintegrant provided different sustained release profiles in water (up to 62% in 24 hr) and in 0.1N HCl (up to 79% in 24 hr).

As can be seen in FIG. 83, a heat-treated tablet composition containing 20% superdisintegrant provided different sustained release profiles in water (up to 52% in 24 hr) and in 0.1N HCl (up to 88% in 24 hr).

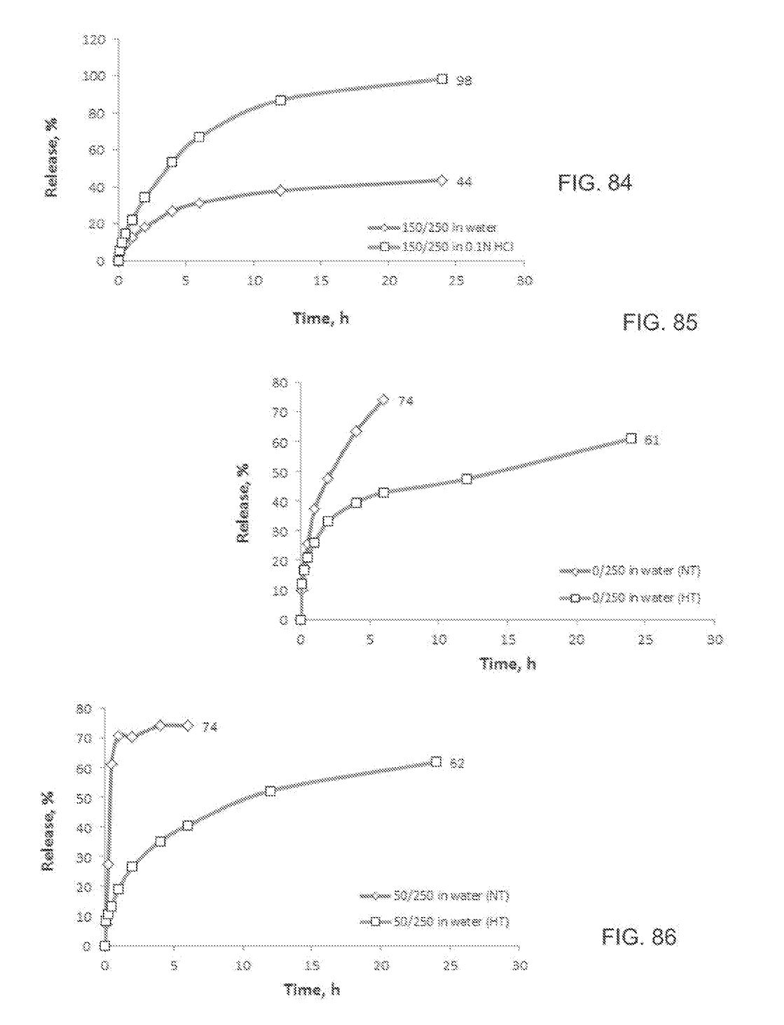

As can be seen in FIG. 84, a heat-treated tablet composition containing 30% superdisintegrant provided different sustained release profiles in water (up to 44% in 24 hr) and in 0.1N HCl (up to 98% in 24 hr).

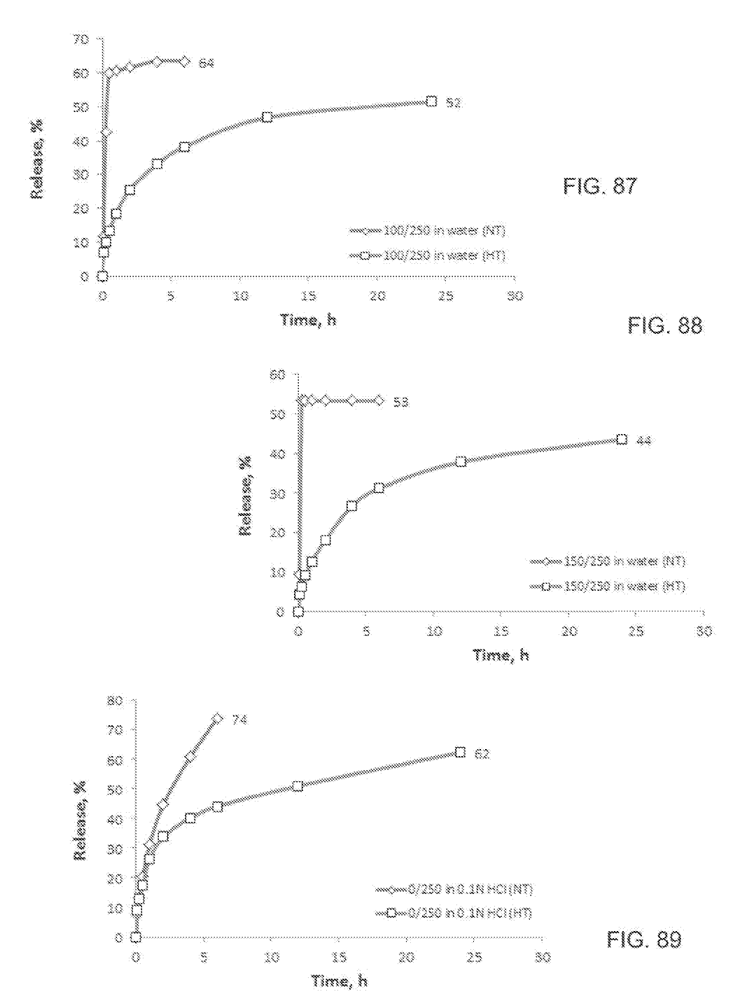

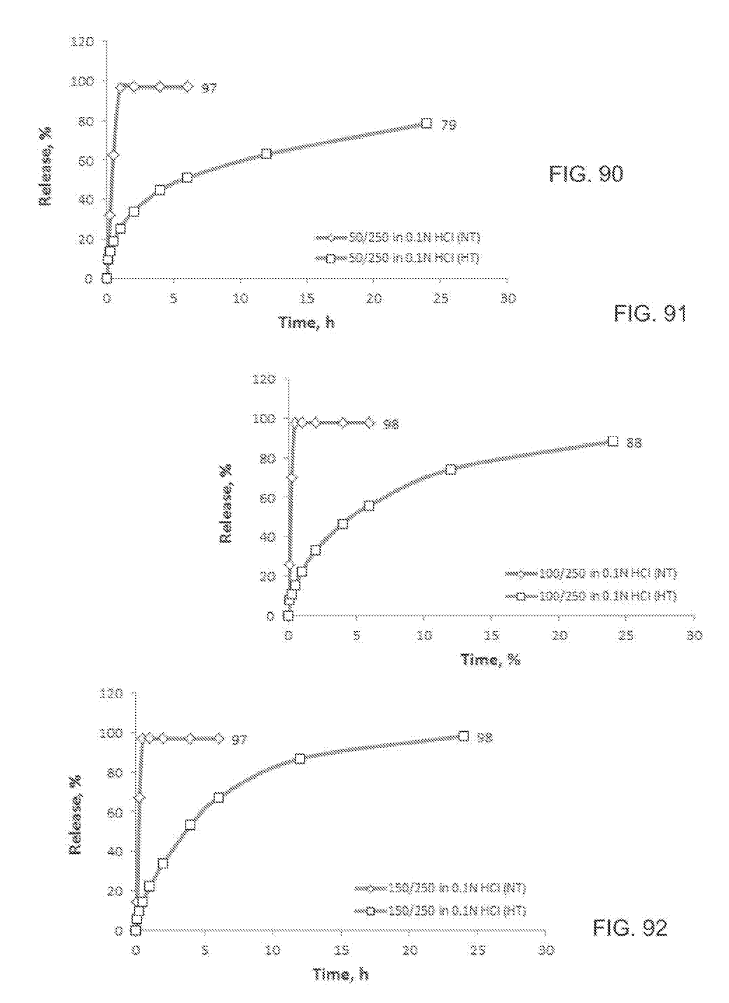

FIGS. 85-92 illustrate a comparison of the release profiles from regular and heat-treated tablets, for the formulations indicated in the figures (blue/light lines are regular, red/dark lines are heat treated), and in Table 58.

TABLE 58

| | Tramadol release | | Tramadol release | |
|---|---|---|---|---|
| | in Water | | in 0.1N HCl | |
| Ac-di-Sol ®, % | % | Release | % | Release |
| REGULAR TABLETS | | | | |
| 0 | 78 | Sustained | 74 | Sustained |
| 10 | 74 | Immediate | 97 | Immediate |
| 20 | 64 | Immediate | 98 | Immediate |
| 30 | 53 | Immediate | 97 | Immediate |
| HEAT-TREATED | | | | |
| TABLETS | | | | |
| 0 | 61 | Sustained | 62 | Sustained |
| 10 | 62 | Sustained | 79 | Sustained |
| 20 | 52 | Sustained | 88 | Sustained |
| 30 | 44 | Sustained | 98 | Sustained |

Crush Resistance of the Deterrent Cage (Immediate and Sustained Release Trial 1) by Ball Mill

The formulation tested is shown in Table 59.

TABLE 59

| | |
|---|---|
| Tramadol HCl | 100 mg |
| Ac-Di-Sol ® | 150 mg |
| Kollidon ® SR | 250 mg |
| Total | 500 mg |

Tablet preparation: Ingredients were mixed in glass mortar with pestle and compressed on Carver press @ 2000 pounds force. The prepared tablets were heat treated @120° C. for 1 hour.

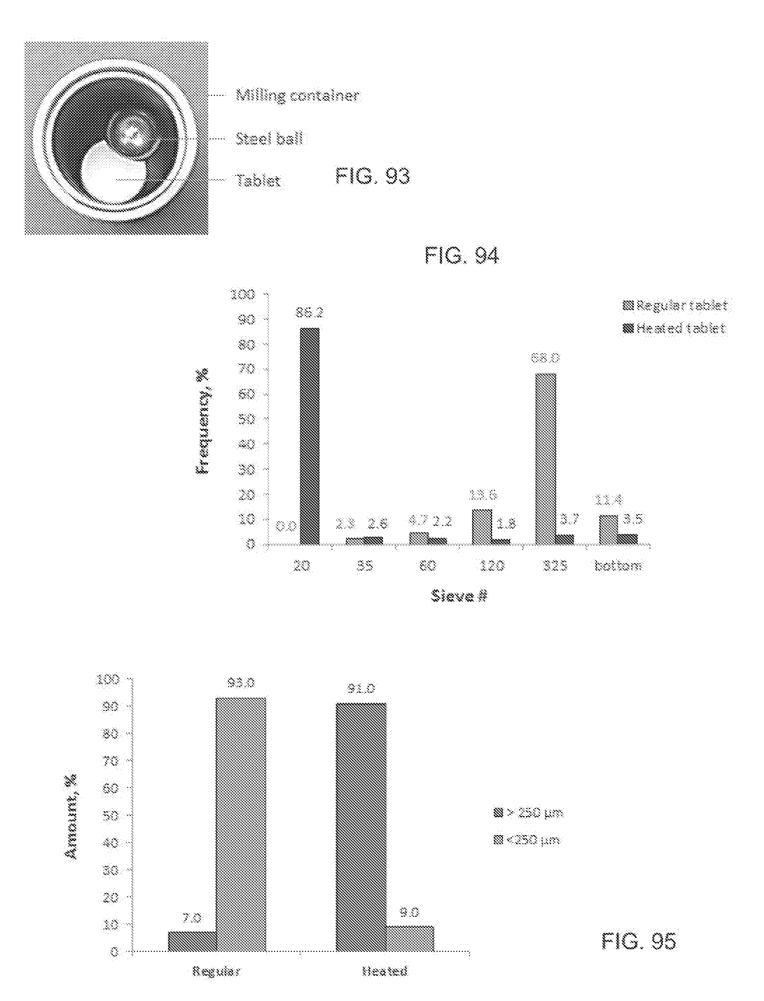

Crushing tablets using ball-mill: Both heat treated and regular tablets were crushed using a ball-mill for 5 minutes at a frequency of 25 Hz. In this test, the configuration included a Retsch Mixer mill MM200, # of balls 2, ball diameter 10 mm, with both balls and the mill container made of stainless steel, as shown in FIG. 93. Initial and final weights of the tablets were measured. The crushed powder was transferred to the sieves and particle size distribution was determined. Results are shown in Tables 60 and 61, with frequency and powder weight illustrated in FIGS. 94 and 95, respectively. Visual appearance is shown in FIG. 96.

TABLE 60
Sieve analysis of crushed regular tablets:

| | Initial | Final | powder | |
|---|---|---|---|---|
| Sieve # | weight, g | weight, g | weight, g | % frequency |
| 20 | 96.092 | 96.092 | 0 | 0 |
| 35 | 94.839 | 94.85 | 0.011 | 2.330508 |
| 60 | 90.09 | 90.112 | 0.022 | 4.661017 |
| 120 | 89.716 | 89.78 | 0.064 | 13.55932 |
| 325 | 85.405 | 85.726 | 0.321 | 68.00847 |
| bottom | 98.524 | 98.578 | 0.054 | 11.44068 |

TABLE 61
Sieve analysis of crushed heated tablets:

| | initial | Final | powder | |
|---|---|---|---|---|
| Sieve # | weight, g | weight, g | weight, g | % frequency |
| 20 | 96.21 | 96.602 | 0.392 | 86.15385 |
| 35 | 94.836 | 94.848 | 0.012 | 2.637363 |
| 60 | 90.12 | 90.13 | 0.01 | 2.197802 |
| 120 | 89.756 | 89.764 | 0.008 | 1.758242 |
| 325 | 85.434 | 85.451 | 0.017 | 3.736264 |
| bottom | 98.507 | 98.523 | 0.016 | 3.516484 |

### Example 5: Crush Resistant Abuse Deterrent Formulations Using PEO

Polyethylene oxide (PEO) is the most common polymer used in the preparation of Nucynta® ER, reformulated OxyContin, reformulated Opana® ER, as well as a number of other abuse-deterrent formulations. Some formulations benefit from its solution properties while others benefit from both of the solution and solid properties of PEO. Major advantages of this polymer is its low melting point in solid state, and high viscosity build up in solution state. Moreover, the polymer is non-toxic, can be used at high concentration, and has least interactions with the active within the formulation. Due to its excellent film forming capability at low temperatures, and ability to build high viscosity in water and hydroalcoholic solutions, this polymer can provide crush and extraction resistance in solid and solution states respectively. Out of different grades, the high molecular weight PEOs have practically been used to achieve crush and extraction resistance as PEOs at higher molecular weight (POLYOX™ Coagulant) can provide superior mechanical properties and solution viscosity. On the other hand, the polymer has certain weaknesses, in particular in its solution state. The solutions of PEO are extremely sensitive to boiling temperatures, high shear rates (rate of mixing), and salts. In other words, the high viscosity built up by PEO in water or hydroalcoholic solutions would be significantly lost if the temperature of the PEO solution is brought up to high temperatures, agitated at high speed, and mixed with salts.

Various deterrent agents capable of binding, absorbing, and adsorbing a basic drug such as Tramadol HCl are described. However, the deterrence efficiency of some deterrents can be sensitive to the medium that abusers use to extract the drug, and this is attributed to the mechanism by which these deterrents function. Those based on organics (such as crosslinked carboxymethylcellulose) are more effective at low ion and alcohol concentrations due to their purely anionic structure. Moreover, their degree of carboxyl substitution can significantly affect the deterrence capacity of this deterrent. Those based on inorganics (such as bentonite clay) tend to bind to the drug effectively even in the presence of 0.1N HCl. Others functioning via adsorption mechanism (such as charcoal) are sensitive to the presence of alcohol while they can provide excellent binding even in the presence of hydrochloric acid. Abusers, in practice, utilize any effective liquid, individually or in combination, to maximize the yield of drug extraction. Therefore, a deterrent blend or composition is envisioned within the disclosure in order to maximize the deterrence capacity when two or more of these extracting medium (water, alcohol, salt, juices, etc.) are used.

The instant invention offers a very effective formulation strategy to provide crush and extraction resistance to abusable medications. Methods to achieve effective crush resistance properties of PEO together with extraction resistance of the deterrent agents are discussed. It is shown that it's not necessary to use a high molecular weight PEO to achieve crush resistance; in fact the data shows that even the lowest molecular weight PEO grade (100K, Sigma Aldrich) could provide a level of crush resistance similar to that of the highest molecular weight PEO grade (Polyox™ WSR 303). This is primarily due to lower film forming temperature of the lower grades which enhances the adhesiveness and hence the integrity of the tablet composition. The lower film forming temperature of the lower grades also means that the tablets containing PEO can thermally be processed at lower temperature or for shorter period of time at higher temperatures, reducing the risk of drug degradation or interaction with other excipients. The instant invention discloses a tablet composition containing tramadol HCl, lower molecular weight PEOs, and crosslinked carboxymethylcellulose (AcDiSol®) at 1/1 weight ratio, heat treated at temperatures lower than 100° C. for 1 hr. Such composition can provide two completely different release profiles in water and in 0.1N HCl. In other words, contrary to compositions containing PEO providing almost same extended release profiles in both solutions, this composition displays very limited release in water and completed release in 0.1N HCl.

Moreover, such composition is extremely resistant to mechanical crushing utilizing pill crusher, ball mill (at different frequency and time), and grind mill.

### EXAMPLES

Immediate Release Formulations with No Crush Resistance Feature Containing a Deterrent Agent

The deterrence capacity of a composition containing high amounts of crosslinked carboxymethyl cellulose has previously been reported. In consideration of this, a test formula is provided in Table 62.

TABLE 62

| | |
|---|---|
| Tramadol HCl | 25 mg |
| Prosolv ® | 170 mg |
| Ac-di-Sol ® | 305 mg |
| Total | 500 mg |

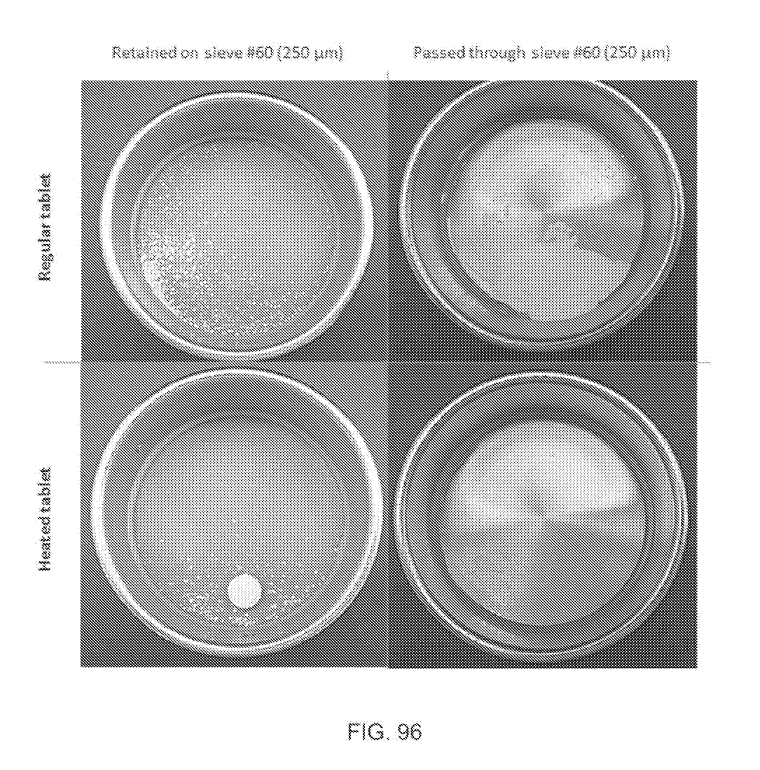

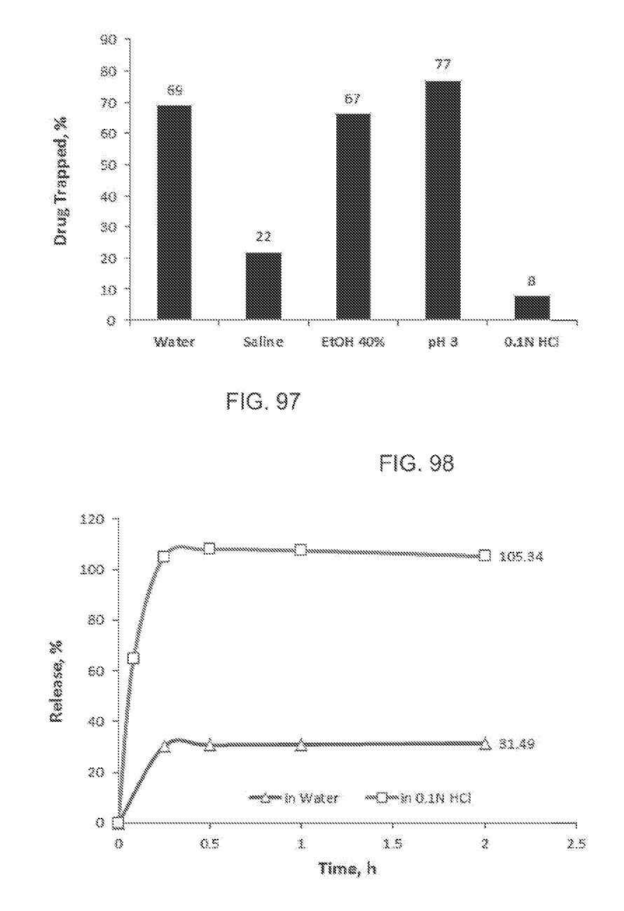

Extraction studies: The physical mixture was transferred to 20 mL glass vial. 10 mL of solvent was added and vortexed for 5 seconds followed by centrifugation@1500 rpm for 5 minutes. The dispersion was filtered through 0.25 μm syringe filter, and 0.5 mL of the filtrate was diluted to 10 mL. The solution was analyzed for drug concentration by UV spectroscopy @271 nm. Results are shown in FIG. 97.

Drug Release Studies: Prepared tablets were studied for their drug release in ultrapure water and 0.1N HCl @ 50 rpm and 37±20 C (USP II). Samples were pulled at different time points and the % drug released was plotted versus time. The first 5 hours, the release was studied in ultrapure water, and then the medium was changed to 0.1N HCl by adding concentrated HCl. Results are illustrated in FIG. 98.

The formulation is composed of only one deterrent agent (Ac-di-Sol®). Trapping capacity of this formulation is 69%, 22%, 67%, 77% in water, saline, EtOH40%, and pH 3 medium, respectively. Such composition can release about 92% of its contents in 0.1N HCl providing an immediate release profile. However, this formulation is not resistant to any mechanical forces and simply grinds into powder.

### Approaches to Improve Crush Resistance

Heat Treatment

The following examples show how binding property of polyethylene oxide can be utilized in preparation of crush resistant compositions containing a deterrent agent. The tablets containing PEO were heat treated at different temperatures.

Formulation: Tramadol HCl (100 mg), AcDiSol® (250 mg) and PEO WSR Coagulant (150 mg). Tablets were prepared by mixing the ingredients in a glass pestle and mortar, compressed at 20001b force on a Carver press. Data are shown in Tables 63-68.

Regular Tablets Undergone No Heat Treatment

TABLE 63
Drug release in water

| Time, | | | | | Conc. | Amount, | % |
|---|---|---|---|---|---|---|---|
| h | abs 1 | abs 2 | abs 3 | avg abs | μg/mL | mg | Release |
| 0 | 0 | 0 | 0 | 0 | 0 | 0 | 0 |
| 0.083 | 0.0378 | 0.0396 | 0.0396 | 0.039 | 7.22 | 6.50 | 6.50 |
| 0.25 | 0.094 | 0.0913 | 0.0925 | 0.093 | 15.73 | 14.16 | 14.16 |
| 0.5 | 0.1954 | 0.1954 | 0.1979 | 0.196 | 32.18 | 28.96 | 28.96 |
| 1 | 0.2834 | 0.2886 | 0.2885 | 0.287 | 46.56 | 41.90 | 41.90 |
| 2 | 0.2997 | 0.2993 | 0.2987 | 0.299233 | 48.53 | 43.68 | 43.68 |
| 4 | 0.3053 | 0.3053 | 0.3052 | 0.305267 | 49.49 | 44.54 | 44.54 |

TABLE 64
Drug release in 0.1N HCl

| Time, | | | | | Conc. | Amount, | % |
|---|---|---|---|---|---|---|---|
| h | abs 1 | abs 2 | abs 3 | avg abs | μg/mL | mg | Release |
| 0 | 0 | 0 | 0 | 0 | 0 | 0 | 0 |
| 0.083 | 0.0739 | 0.074 | 0.073 | 0.0737 | 12.31 | 11.08 | 11.08 |
| 0.25 | 0.1957 | 0.1954 | 0.1965 | 0.1959 | 32.68 | 29.41 | 29.41 |
| 0.5 | 0.4766 | 0.4763 | 0.4768 | 0.4766 | 79.46 | 71.52 | 71.52 |
| 1 | 0.6458 | 0.6464 | 0.6465 | 0.6462 | 107.74 | 96.97 | 96.97 |
| 2 | 0.6499 | 0.6493 | 0.65 | 0.6498 | 108.32 | 97.49 | 97.49 |
| 4 | 0.6371 | 0.6371 | 0.6368 | 0.637 | 106.20 | 95.58 | 95.58 |

Tablets Undergone Heat Treatment at 65° C./1 hr

TABLE 65
Drug release in water

| Time, | | | | | Conc. | Amount, | % |
|---|---|---|---|---|---|---|---|
| h | abs 1 | abs 2 | abs 3 | avg abs | μg/mL | mg | Release |
| 0 | 0 | 0 | 0 | 0 | 0 | 0 | 0 |
| 0.083 | 0.0405 | 0.04 | 0.0402 | 0.0402 | 7.42 | 6.68 | 6.68 |
| 0.25 | 0.099 | 0.0994 | 0.1003 | 0.0996 | 16.84 | 15.15 | 15.15 |
| 0.5 | 0.1904 | 0.1884 | 0.1893 | 0.1894 | 31.09 | 27.98 | 27.98 |
| 1 | 0.3009 | 0.307 | 0.3064 | 0.3048 | 49.41 | 44.47 | 44.47 |
| 2 | 0.3195 | 0.3237 | 0.324 | 0.3224 | 52.21 | 46.99 | 46.99 |
| 4 | 0.3224 | 0.3236 | | 0.323 | 52.30 | 47.07 | 47.07 |

TABLE 66
Drug release in 0.1N HCl

| Time, | | | | | Conc. | Amount, | % |
|---|---|---|---|---|---|---|---|
| h | abs 1 | abs 2 | abs 3 | avg abs | μg/mL | mg | Release |
| 0 | 0 | 0 | 0 | 0 | 0 | 0 | 0 |
| 0.083 | 0.1018 | 0.1019 | 0.1021 | 0.1019 | 17.02 | 15.32 | 15.32 |
| 0.25 | 0.2599 | 0.2616 | 0.2606 | 0.2607 | 43.48 | 39.14 | 39.14 |
| 0.5 | 0.42 | 0.4202 | 0.42 | 0.4200 | 70.04 | 63.04 | 63.04 |
| 1 | 0.5889 | 0.592 | 0.5917 | 0.5909 | 98.51 | 88.66 | 88.66 |
| 2 | 0.6355 | 0.6362 | 0.635 | 0.6356 | 105.96 | 95.37 | 95.37 |
| 4 | 0.6428 | 0.6421 | 0.642 | 0.6423 | 107.08 | 96.38 | 96.38 |

Tablets Undergone Heat Treatment at 120° C./1 hr:

TABLE 67
Drug release in water

| Time, | | | | | Conc. | Amount, | % |
|---|---|---|---|---|---|---|---|
| h | abs 1 | abs 2 | abs 3 | avg abs | μg/mL | mg | Release |
| 0 | 0 | 0 | 0 | 0 | 0 | 0 | 0 |
| 0.083 | 0.0566 | 0.0568 | 0.0574 | 0.0569 | 10.07 | 9.06 | 9.06 |
| 0.25 | 0.126 | 0.125 | 0.1251 | 0.1254 | 20.93 | 18.84 | 18.84 |
| 0.5 | 0.228 | 0.2285 | 0.2289 | 0.2285 | 37.30 | 33.57 | 33.57 |
| 1 | 0.3137 | 0.3147 | 0.3145 | 0.3143 | 50.92 | 45.83 | 45.83 |
| 2 | 0.3182 | 0.3165 | 0.3165 | 0.3171 | 51.36 | 46.22 | 46.22 |
| 4 | 0.3354 | 0.3352 | 0.335 | 0.3352 | 54.24 | 48.81 | 48.81 |

TABLE 68
Drug release in 0.1N HCl

| Time, | | | | | Conc. | Amount, | % |
|---|---|---|---|---|---|---|---|
| h | abs 1 | abs 2 | abs 3 | avg abs | μg/mL | mg | Release |
| 0 | 0 | 0 | 0 | 0 | 0 | 0 | 0 |
| 0.083 | 0.1005 | 0.1003 | 0.1 | 0.1003 | 16.74 | 15.07 | 15.07 |
| 0.25 | 0.2562 | 0.2562 | 0.2559 | 0.2561 | 42.72 | 38.45 | 38.45 |
| 0.5 | 0.4832 | 0.4845 | 0.4842 | 0.4839 | 80.69 | 72.63 | 72.63 |
| 1 | 0.6449 | 0.6444 | 0.6442 | 0.6445 | 107.45 | 96.71 | 96.71 |
| 2 | 0.653 | 0.653 | 0.6531 | 0.6530 | 108.87 | 97.99 | 97.99 |
| 4 | 0.668 | 0.6682 | 0.6682 | 0.6681 | 111.39 | 100.25 | 100.25 |

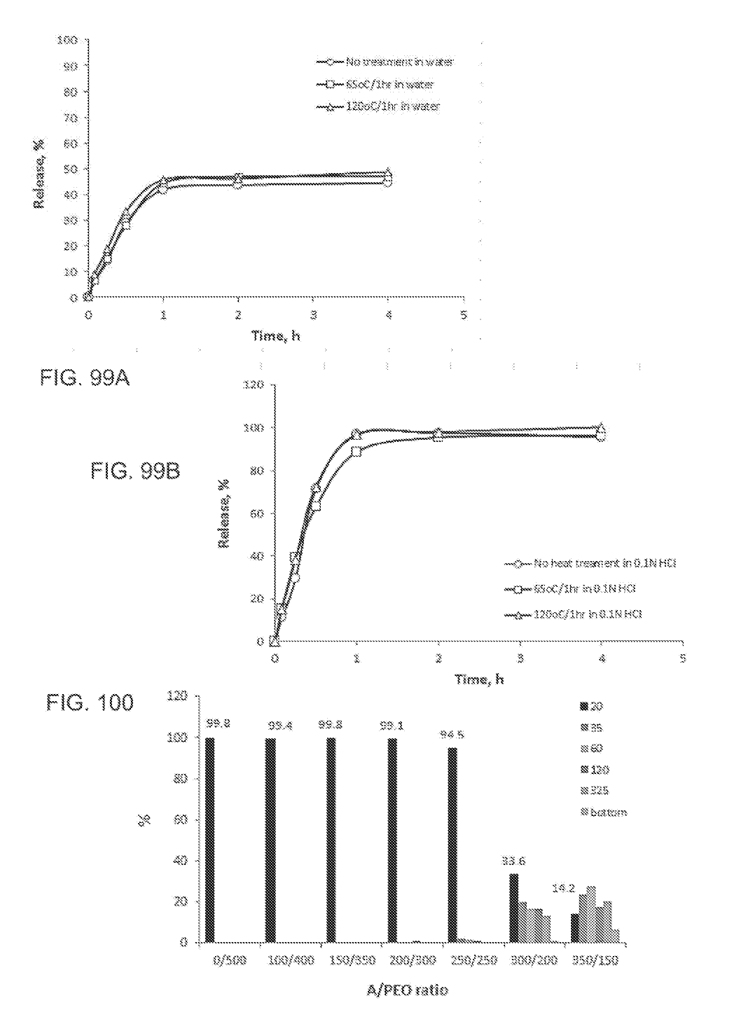

FIG. 99A shows non-treated and heat treated tablets at different temperatures were able to release their tramadol content in water up to about 44-48%. In other words, the amount of drug release in water was found to be independent of heat treatment.

FIG. 99B shows non-treated and heat treated tablets at different temperatures were able to release their tramadol content in 0.1NHCl up to 100%. In other words, the drug release in 0.1N HCl was found to be independent of heat treatment.

### Optimizing the A/PEO Ratio to Achieve Maximum Deterrence and Binding:

Ball Mill Studies:

Tablets were crushed using a ball mill (Retsch Mixer Mill MM200). Two steel balls (1 cm in diameter) were used at frequency of 25/sec for 5 minutes. The crushed powder was transferred to sieves and particle size distribution was determined. Sieves were placed in a column by their sieve number (20, 35, 60, 120 and 325) and receiver at the bottom. Top sieve was loaded with the sample and sieves were placed on a sifter (Sieve sifter SS-3CP by Cole Parmer). Tapping was run for one minute @ 60 tapping per minute. Particles retained on each sieve were separated and weighed.

TABLE 69
% retained on sieve#: (weight of retained powder
on sieve#/total weight of powder)*100

| | A/PEO Ratio | | | | | | |
|---|---|---|---|---|---|---|---|
| Sieve # | 0/500 | 100/400 | 150/350 | 200/300 | 250/250 | 300/200 | 350/150 |
| 20 | 99.8 | 99.4 | 99.78 | 99.13 | 94.53 | 33.63 | 14.16 |
| 35 | 0 | 0 | 0 | 0.43 | 1.97 | 20.04 | 23.74 |
| 60 | 0 | 0 | 0 | 0.65 | 1.53 | 16.48 | 27.4 |
| 120 | 0.4 | 0 | 0 | 0.87 | 1.09 | 16.48 | 17.58 |
| 325 | 0.2 | 0.213 | 0 | 0.43 | 0.22 | 13.36 | 20.32 |
| bottom | 0 | 0.213 | 0 | 0.43 | 0 | 1.34 | 6.4 |

With reference to FIG. 100, this study shows A-PEO compositions containing up to 50 wt % of A would still be comparable to higher PEO compositions as far as their resistance to crushing is concerned.

After ball milling for 5 min, same tablets were treated in a grind mill for 10 sec. Compositions containing 0/500, 100/400, 150/350, 200/300, and 250/250 completely resisted the grind milling.

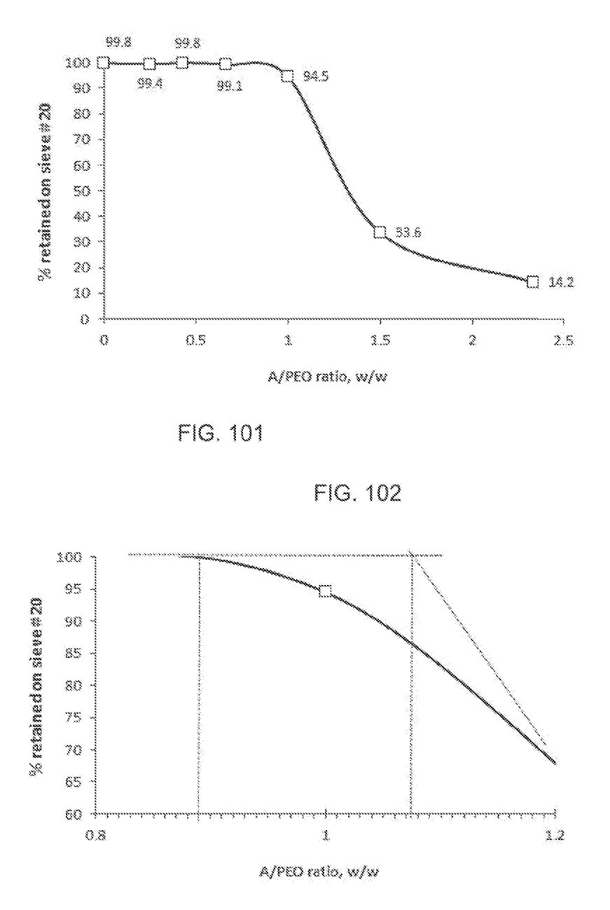

FIGS. 101 and 102 show that A/PEO ratio of 0.89 would be the most ideal composition where the composition benefits maximum crush resistance offered by PEO and maximum deterrence offered by “A”. Data shows that the composition will begin to significantly lose its crush resistance at an A/PEO ratio of 1.075.

### Effect of Molecular Weight on Thermal Properties:

DSC Studies:

Different grades of PEO were weighed and transferred to aluminum pans and sealed hermetically. The first heating cycle was as follows: 1 min at 25° C., heating the sample to 150° C. @ 10° C./min, held for one min at 150° C., and cooled back to 25° C. The first cycle was conducted to erase the thermal history. Second cycle was performed similar to the first cycle to obtain actual softening or melting data.

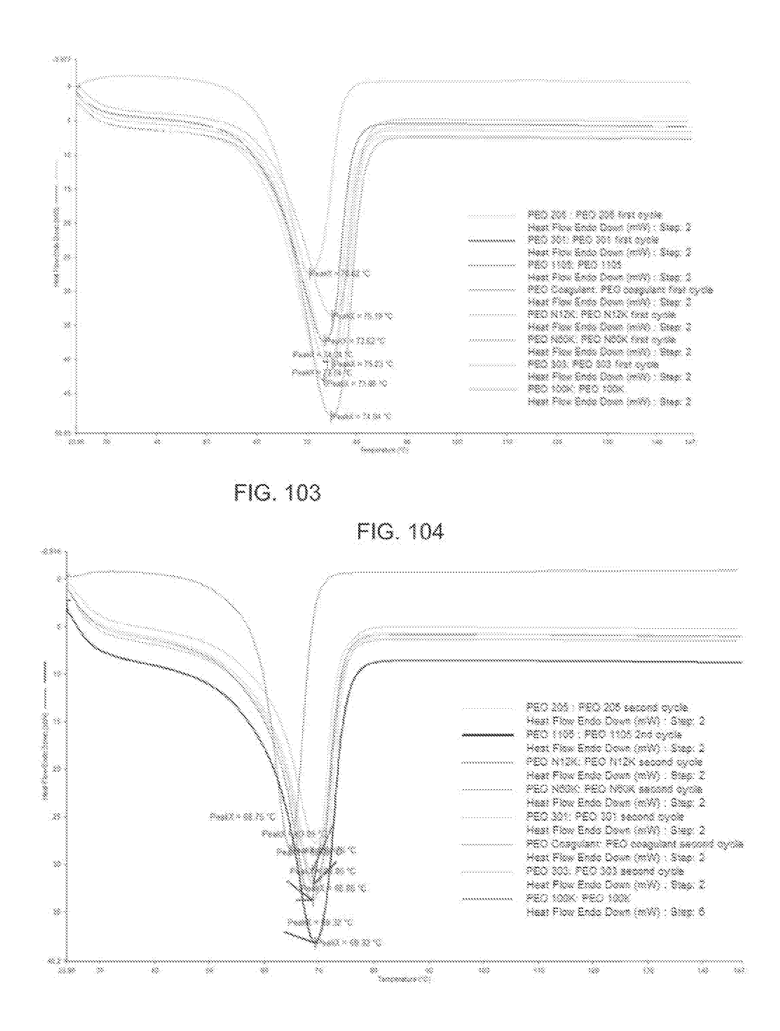

The first cycle is illustrated in FIG. 103, and the second cycle in FIG. 104, with corresponding data shown in Table 70.

TABLE 70

| | | 1^{st} cycle | 1^{st} Heat of | 2^{nd} cycle | 2^{nd} Heat of |
|---|---|---|---|---|---|
| PEO | MW | MP, ° C. | Fusion, J/g | MP, ° C. | Fusion, J/g |
| Sigma | 100,000 | 70.62 | 181.94 | 64.76 | 132.89 |
| 205 | 600,000 | 73.86 | 180.87 | 68.52 | 141.55 |
| 1105 | 900,000 | 74.94 | 195.53 | 69.32 | 142.94 |
| N12K | 1,000,000 | 73.54 | 188.87 | 68.86 | 141.86 |
| N60K | 2,000,000 | 74.24 | 198.36 | 69.05 | 144.97 |
| 301 | 4,000,000 | 73.62 | 187.35 | 68.75 | 139.16 |
| Coag- | 5,000,000 | 75.19 | 180.54 | 69.47 | 134.12 |
| ulant | | | | | |
| 303 | 7,000,000 | 75.23 | 208.06 | 69.71 | 153.14 |

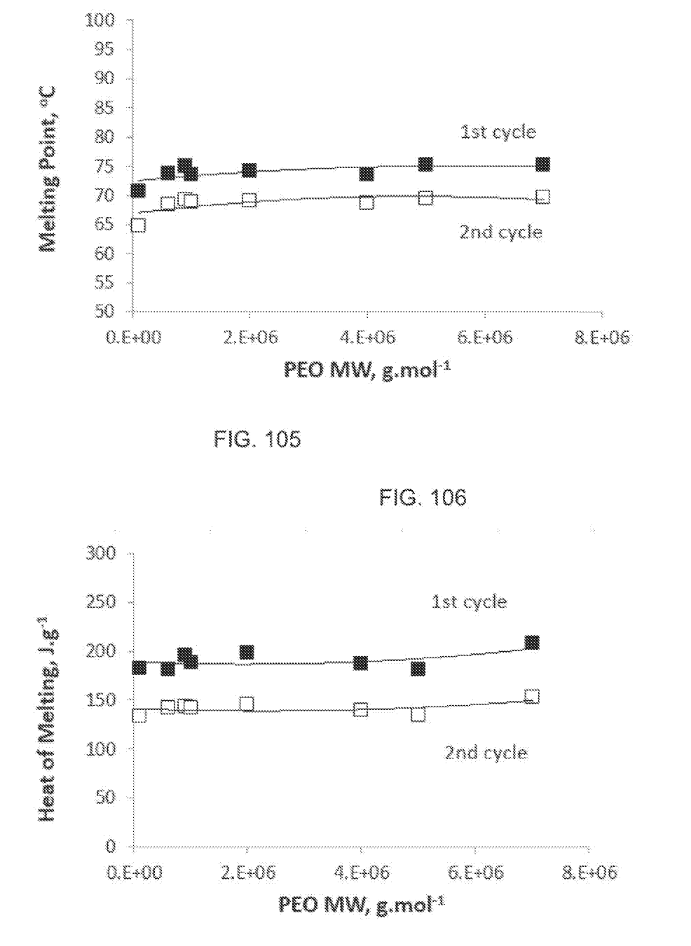

With reference to FIGS. 105 and 106, DSC studies show that PEOs after erasing their thermal history show almost the same melting point at around 69° C. and heat fusion of about 142 J/g regardless of their molecular weight. In other words, intermolecular interactions within PEO grades are independent of the PEO molecular weight. This also means that the effect of PEO molecular weight on mechanical properties should be negligible as shown below.

### Effect of Molecular Weight on Mechanical Properties

At A/PEO ratio of 1, the effect of PEO molecular weight on crush resistance was studied, as shown in Tables 71.

TABLE 71

| | Molecular Weight, | |
|---|---|---|
| PEO Grade | g/mol | Viscosity (cP) |
| 205 | 600,000 | 4500-8800(5%) |
| 1105 | 900,000 | 8800-17,600(5%) |
| N 12K | 1,000,000 | 400-800(2%) |
| N60K | 2,000,000 | 2000-4000(2%) |
| 301 | 4,000,000 | 1650-5500(1%) |
| Coagulant | 5,000,000 | 5500-7500(1%) |
| 303 | 7,000,000 | 7500-10000(1%) |

Tablet preparation: 250 mg of AcDiSol® and 250 mg of each PEO grade were mixed in a pestle and mortar, compressed into tablet at 2000 lb on a Carver press, and then heated at 120° C. for 1 hr.

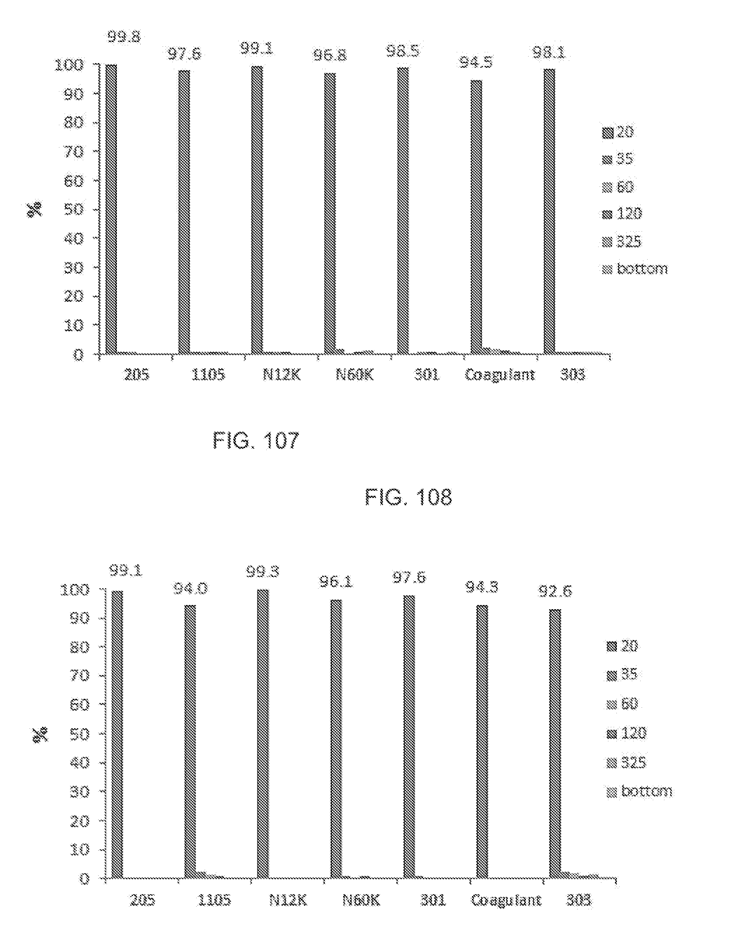

With both ball mill and grind mill studies, the PEOs of different grades predominantly produce particles larger than 850 μm (retained on sieve #20). With reference to FIGS. 107 and 108, mechanical strength study shows that the tablet composed of 50% of AcDiSol® and 50% of PEO205 could resist ball milling (5 min) and grind milling (10 sec) to the exact same extent as very high molecular weight PEOs do. In fact, the lowest molecular weight could offer better mechanical strength in general, which might be due to better flow and film forming under similar thermal conditions.

### Effect of Curing Temperature

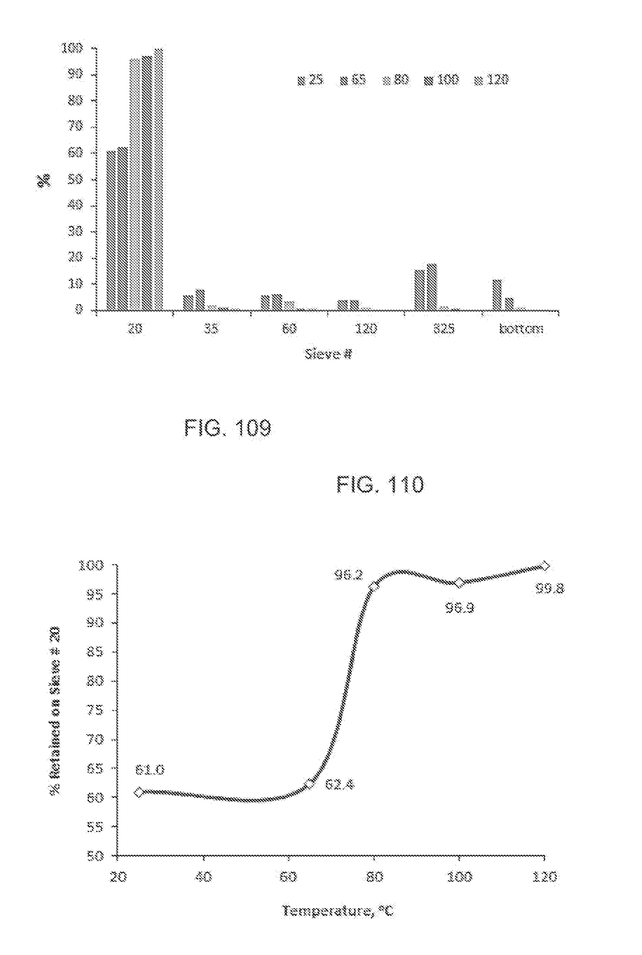

Since PEO205 was found to be as effective as high molecular weight PEOs including PEO coagulant and 303, we performed additional studies on tablets composed of 50% AcDiSol® and 50% PEO205, to evaluate the effect of curing temperature on crush resistance. The the graphs in FIGS. 109 and 110 show that curing the tablet at 80, 100, and 120° C. for 1 hr would have almost a same effect on the percentage of particles retained on sieve #20 (particles larger than 850 μm).

### Drug Release and Crush Resistance Studies on PEO 205-Based Tablets

Tablet composition: Tramadol HCl (25 mg), AcDiSol® (250 mg) and PEO 205 (250 mg). Tablets were prepared by mixing the ingredients in a glass pestle and mortar. Physical mixture was compressed at 2000 lb pressure on a Carver press, and then heat treated at 90° C. for 1 hr. Results are shown in Tables 72 and 73.

Drug Release Studies

TABLE 72
In water

| Time, | | | | | Conc., | Amount, | % |
|---|---|---|---|---|---|---|---|
| h | abs 1 | abs 2 | abs 3 | avg abs | μg/mL | mg | Release |
| 0.083 | 0.0134 | 0.0128 | 0.0122 | 0.0128 | 3.06 | 2.76 | 11.03 |
| 0.25 | 0.0223 | 0.0234 | 0.0236 | 0.0231 | 4.70 | 4.23 | 16.91 |
| 0.5 | 0.0278 | 0.0278 | 0.0283 | 0.027967 | 5.47 | 4.92 | 19.70 |
| 1 | 0.043 | 0.0433 | 0.044 | 0.043433 | 7.93 | 7.13 | 28.53 |
| 2 | 0.0671 | 0.0667 | 0.0658 | 0.066533 | 11.59 | 10.43 | 41.73 |
| 4 | 0.0681 | 0.0698 | 0.0701 | 0.069333 | 12.04 | 10.83 | 43.33 |

TABLE 73
In 0.1N HCl

| Time, | | | | | Conc., | Amount, | % |
|---|---|---|---|---|---|---|---|
| h | abs 1 | abs 2 | abs 3 | avg abs | μg/mL | mg | Release |
| 0.083 | 0.0292 | 0.0291 | 0.0283 | 0.028867 | 4.84 | 4.36 | 17.44 |
| 0.25 | 0.063 | 0.063 | 0.0624 | 0.0628 | 10.50 | 9.45 | 37.80 |
| 0.5 | 0.0928 | 0.0925 | 0.0925 | 0.0926 | 15.47 | 13.92 | 55.68 |
| 1 | 0.1298 | 0.1299 | 0.1298 | 0.129833 | 21.67 | 19.51 | 78.02 |
| 2 | 0.1698 | 0.1699 | 0.1702 | 0.169967 | 28.36 | 25.53 | 102.10 |
| 4 | 0.1832 | 0.1836 | 0.1908 | 0.185867 | 31.01 | 27.91 | 111.64 |

As shown in FIG. 111, a tablet containing 250 mg AcDiSol® and 250 mg of the WSR 205, heat treated at 90° C. for 1 hr could release up to 40% of its 25 mg tramadol content. On the other hand, same tablet released all its tramadol content in 0.1N HCl. Tramadol release in both water and 0.1N HCl reach plateau after about 2 hrs.

Crush Resistance Studies

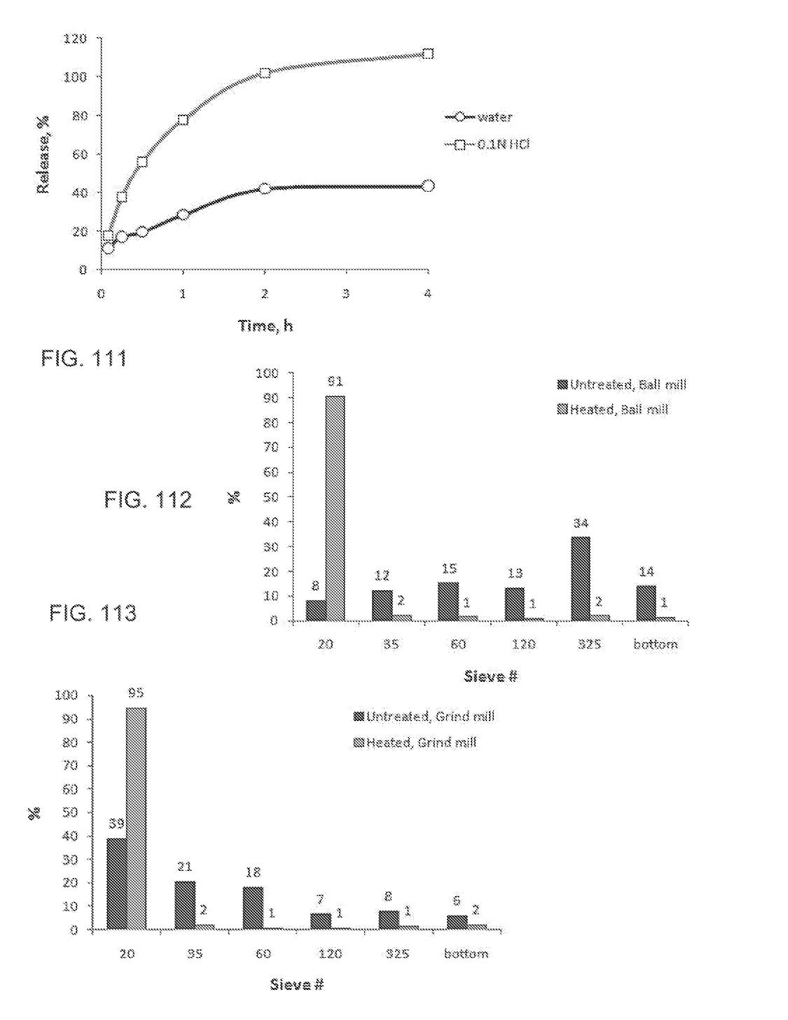

Untreated and heat-treated tablets were processed using a ball mill for 5 min, and then with a grind mill for 10 sec, with results illustrated in FIGS. 112-113.

Pill Crusher Study

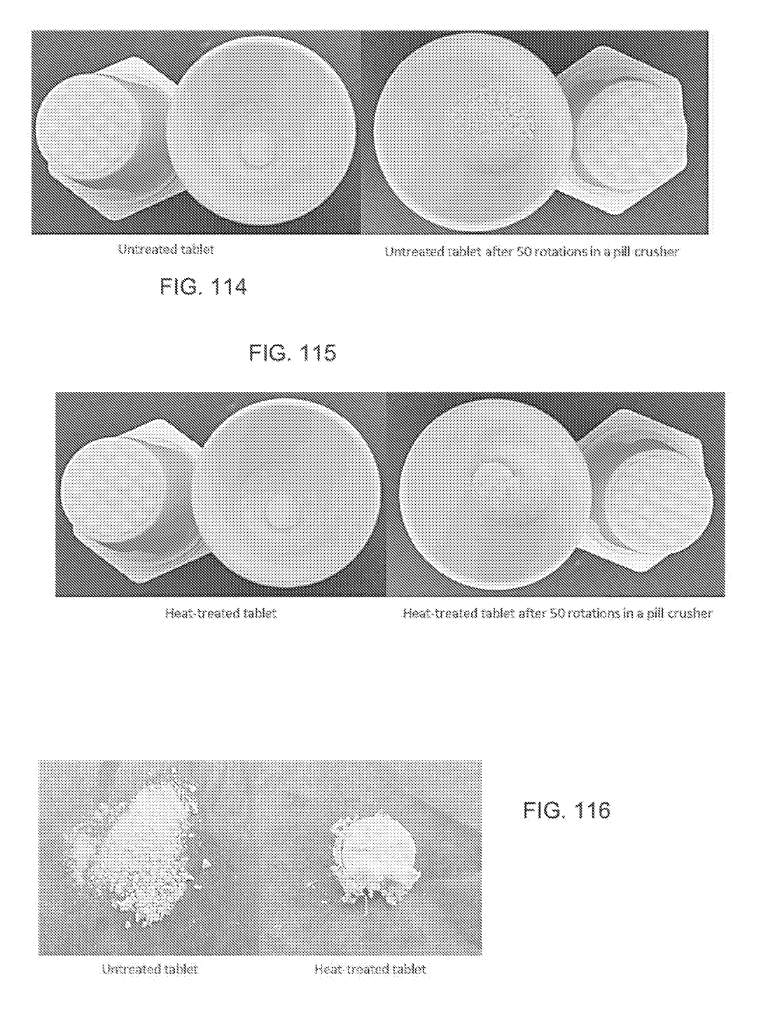

FIGS. 114-116 illustrate results of a pill crusher study, as detailed in the captions.

Ball Mill Study

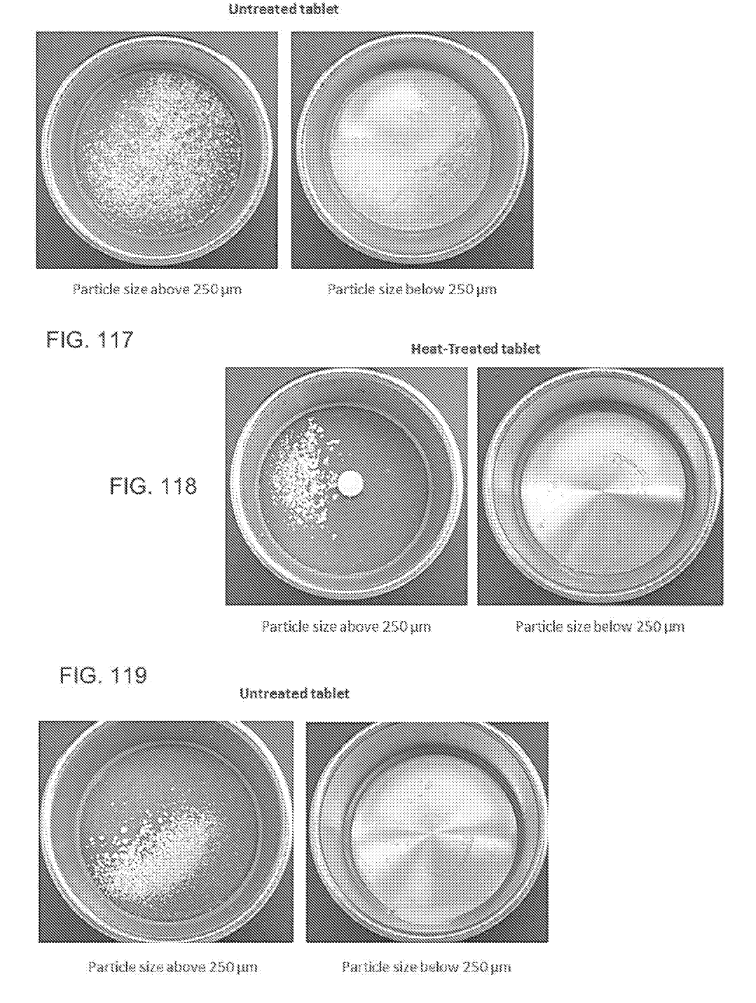

FIG. 117 illustrate results of a ball mill study, as detailed in the captions.

Grind Mill Study

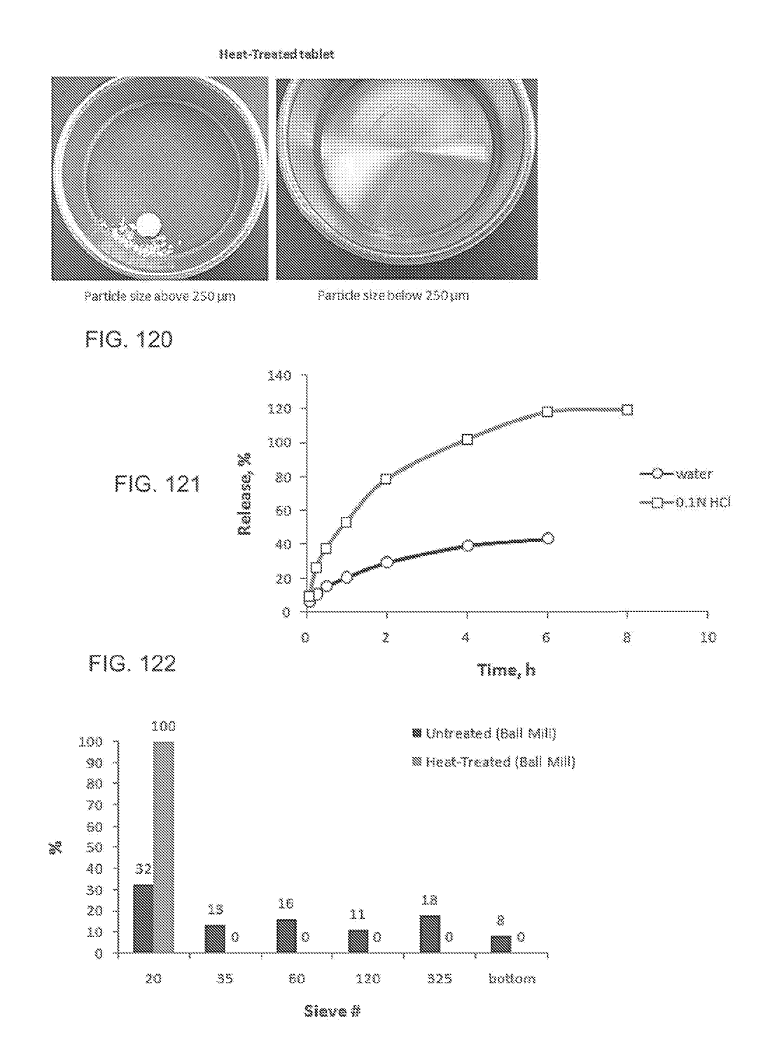

FIGS. 119-120 illustrate results of a grind mill study, as detailed in the captions.

### PEO (100K)-Based Tablet

A release study of a PEO (100K)-based tablet is shown in Tables 74-75, and is illustrated in FIG. 121.

TABLE 74
Drug release in water

| Time, | | | | | Conc. | Amount, | % |
|---|---|---|---|---|---|---|---|
| h | abs 1 | abs 2 | abs 3 | avg abs | μg/mL | mg | Release |
| 0.083 | 0.004 | 0.004 | 0.004 | 0.004 | 1.67 | 1.50 | 6.00 |
| 0.25 | 0.0117 | 0.0118 | 0.0121 | 0.011867 | 2.92 | 2.62 | 10.50 |
| 0.5 | 0.0197 | 0.0198 | 0.0201 | 0.019867 | 4.19 | 3.77 | 15.07 |
| 1 | 0.0292 | 0.0294 | 0.0294 | 0.029333 | 5.69 | 5.12 | 20.48 |
| 2 | 0.0443 | 0.0443 | 0.0455 | 0.0447 | 8.13 | 7.31 | 29.26 |
| 4 | 0.0624 | 0.0623 | 0.0621 | 0.062267 | 10.92 | 9.82 | 39.30 |
| 6 | 0.0697 | 0.0696 | 0.0697 | 0.069667 | 12.09 | 10.88 | 43.52 |

TABLE 75
Drug release in 0.1N HCl

| Time, | | | | | Conc. | Amount, | % |
|---|---|---|---|---|---|---|---|
| h | abs 1 | abs 2 | abs 3 | avg abs | μg/mL | mg | Release |
| 0.083 | 0.0153 | 0.0153 | 0.0154 | 0.015333 | 2.59 | 2.33 | 9.32 |
| 0.25 | 0.0431 | 0.0433 | 0.0431 | 0.043167 | 7.23 | 6.51 | 26.02 |
| 0.5 | 0.063 | 0.063 | 0.0629 | 0.062967 | 10.53 | 9.48 | 37.90 |
| 1 | 0.0889 | 0.0887 | 0.0885 | 0.0887 | 14.82 | 13.34 | 53.34 |
| 2 | 0.131 | 0.1306 | 0.1309 | 0.130833 | 21.84 | 19.66 | 78.62 |
| 4 | 0.1702 | 0.1698 | 0.1698 | 0.169933 | 28.36 | 25.52 | 102.08 |
| 6 | 0.1968 | 0.1968 | 0.1968 | 0.1968 | 32.83 | 29.55 | 118.20 |
| 8 | 0.1991 | 0.1992 | 0.1992 | 0.199167 | 33.23 | 29.91 | 119.62 |

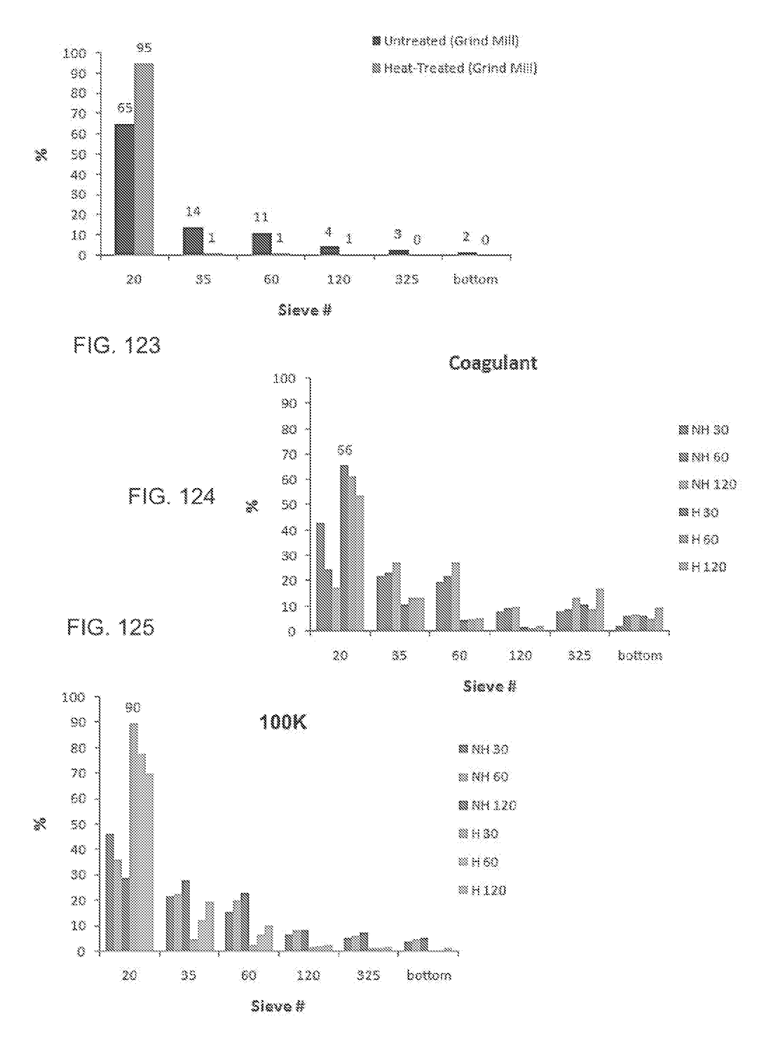

Results of a crush resistance study using a ball mill and a grind mill are illustrated in FIGS. 122 and 123, respectively.

### Comparing Tablets Prepared Using Polyox™ Coagulant and the PEO 100K

Tablet preparation: Physical mixture of 25 mg tramadol HCl, 250 mg PEO (coagulant and PEO 100K) and 250 mg AcDiSol® compressed into tablet at 2000 lb force using a Carver press. Some tablets were also heat treated at 90° C. for 1 hr (NH for no heat treatment and H for heated in the following graphs).

Grind mill studies: Both non treated and heat treated tablets were crushed in a grind mill for 30, 60 and 120 sec, and particle size distribution was determined using sieve analysis, with results illustrated in FIGS. 124-125, which show that tablets made of PEO100K are more resistant to crushing than tablets made of PEO coagulant.

Pill Crusher Study

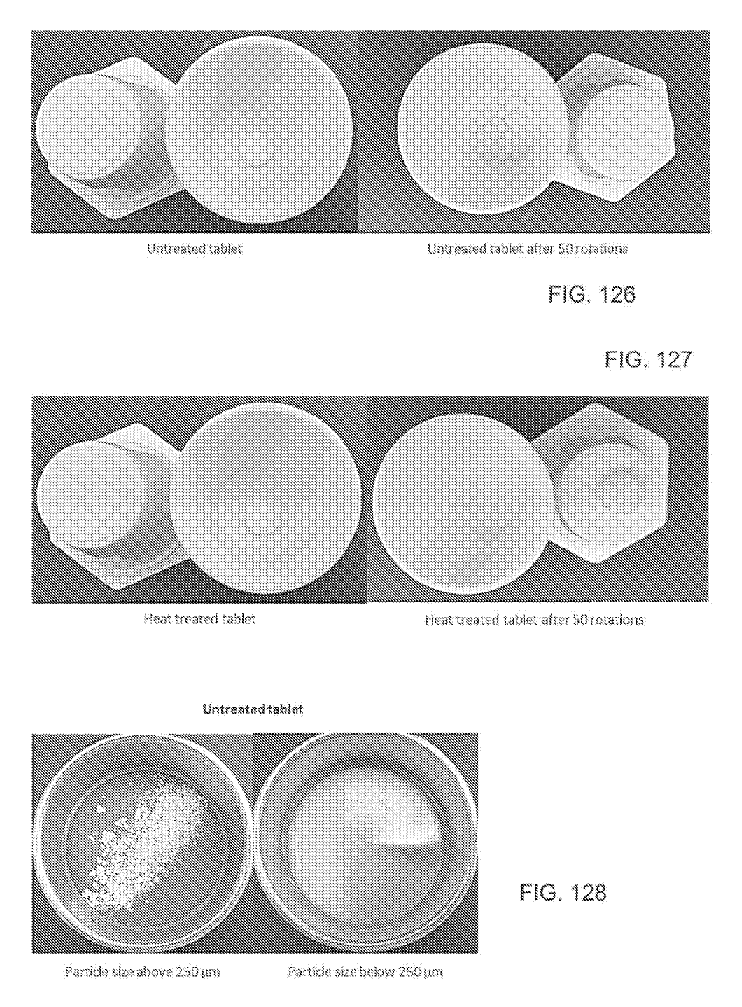

FIGS. 126-127 illustrate the results of a pill crusher study for untreated and treated tablets.

Ball Mill Study

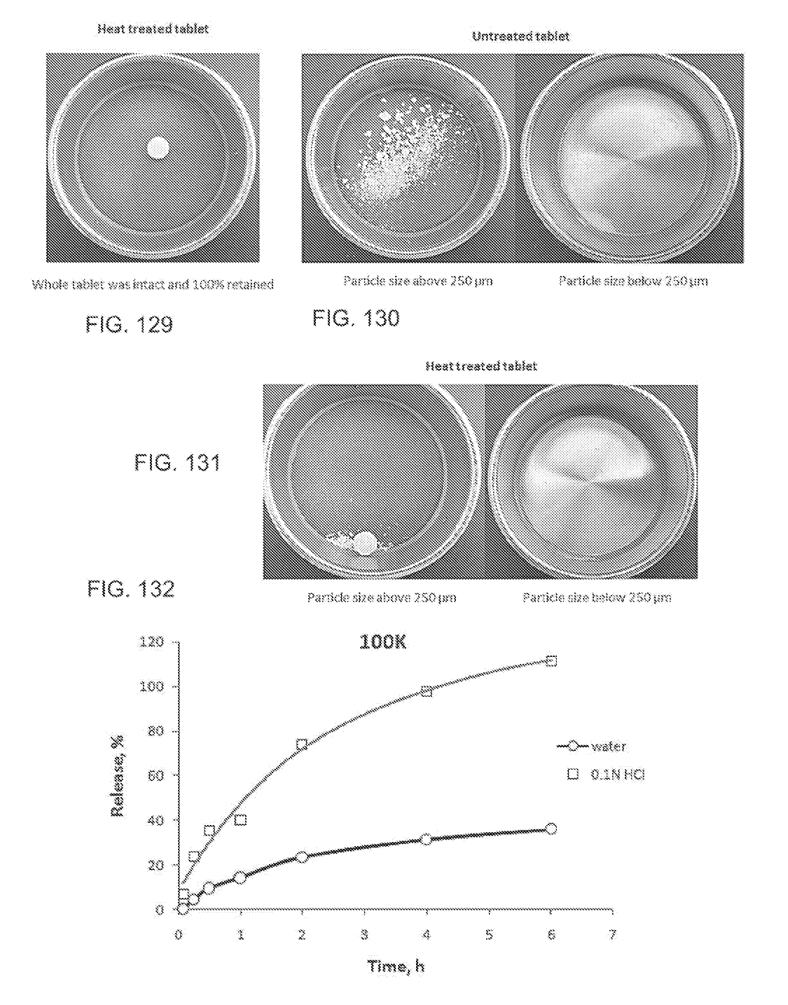

FIGS. 128 and 129 illustrate the results of a ball mill study for treated and untreated tablets.

Grind Mill Study

FIGS. 130 and 131 illustrate the results of a grind mill study for treated and untreated tablets.

### Drug Release Studies for Tablets Based on PEO 100K

Tables 76 and 77 show release data for tablets based on PEO 100K, as illustrated in FIG. 132.

TABLE 76
In water

| Time, | avg abs | blank | adjusted | Conc., | Amount, | % |
|---|---|---|---|---|---|---|
| h | (3 readings) | abs | abs | μg/mL | mg | Release |
| 0.083 | 0.004 | 0.0017 | 0.0023 | 0.101695 | 0.09 | 0.37 |
| 0.25 | 0.011867 | 0.002667 | 0.0092 | 1.271186 | 1.14 | 4.58 |
| 0.5 | 0.019867 | 0.0027 | 0.017167 | 2.621469 | 2.36 | 9.44 |
| 1 | 0.029333 | 0.004233 | 0.0251 | 3.966102 | 3.57 | 14.28 |
| 2 | 0.0447 | 0.004533 | 0.040167 | 6.519774 | 5.87 | 23.47 |
| 4 | 0.062267 | 0.0091 | 0.053167 | 8.723164 | 7.85 | 31.40 |
| 6 | 0.069667 | 0.009133 | 0.060533 | 9.971751 | 8.97 | 35.90 |

TABLE 77
In 0.1N HCl

| time | avg abs | blank | | Conc., | Amount, | % |
|---|---|---|---|---|---|---|
| (h) | (3 readings) | abs | adjusted | μg/mL | mg | Release |
| 0.083 | 0.015333 | 0.0034 | 0.011933 | 1.903955 | 1.71 | 6.85 |
| 0.25 | 0.043167 | 0.0034 | 0.039767 | 6.621469 | 5.96 | 23.84 |
| 0.5 | 0.062967 | 0.003867 | 0.0591 | 9.898305 | 8.91 | 35.63 |
| 1 | 0.0887 | 0.0221 | 0.0666 | 11.16949 | 10.05 | 40.21 |
| 2 | 0.130833 | 0.008933 | 0.1219 | 20.54237 | 18.49 | 73.95 |
| 4 | 0.169933 | 0.0092 | 0.160733 | 27.12429 | 24.41 | 97.65 |
| 6 | 0.1968 | 0.013367 | 0.183433 | 30.97175 | 27.87 | 111.50 |
| 8 | 0.199167 | 0.017033 | 0.182133 | 30.75141 | 27.68 | 110.71 |

### Example 6: Abuse-Deterrent Formulations Having Enhanced Crush Resistance

An abuse-deterrent cage of the invention is composed of a water-swellable polymer that relatively grows or expands in size when exposed to an aqueous medium. It also effectively binds to the active cationic ingredient such as pain medications carrying positive charges in their structure. The drug binding to the water swellable component of the cage is pH sensitive; in other words the binding is effective at normal pHs; however, it becomes ineffective at physiological pHs close to 1. The cage also includes a water-insoluble hydrophobic polymer that possesses a relatively low glass transition temperature preferably ranging 25-60° C. and advantageously ranging between 27 and 40° C. When tablet containing the drug, the water swellable polymer and the hydrophilic polymer is heated above the glass transition temperature of the hydrophobic component of the cage, the tablet becomes integrated or sintered into one strong body that can resist abuse by crushing and extraction. Elsewhere herein it is disclosed that if the tablet is heat treated at 120° C. for 1 hour, its mechanical strength would be sufficiently high to resist ball milling and heavy duty grind milling. For instance, if such tablet is processed in a ball mill (having 2 steel balls each 10 mm in diameter) at a frequency of 25/min for 5 min, the tablet generally stays intact or generates over 80% of large particles (greater than 250 μm). This disclosure relates to using sintering enhancers to lower the temperature at which the tablet is sintered into one integrated piece with sufficient mechanical strength against abuse forces. The disclosure includes the use of Carbowax™ (polyethylene glycol 8000) in combination with a water-swellable binding polymer (AcDiSol®) and a hydrophobic polymer (Kollidon® SR). The tablet is heat treated at generally lower temperature than what was previously reported. According to this invention, a 500 mg tablet composition containing 100 mg of tramadol HCl, 250 mg of Kollidon® SR, 150 mg of Ac-Di-Sol® and 25 mg of PEG8000, heat-treated at 90° C. for 1 hr can offer improved resistance to abuse by crushing and extraction.

### EXAMPLES

A plasticizer has been incorporated into the formulation in accordance with Table 78.

TABLE 78

| Component | Control | Trial 1 | Trial 2 | Trial 3 |
|---|---|---|---|---|
| AcDiSol ®, mg | 150 | 150 | 150 | 150 |
| PEG 8000 NF, mg | 0.0 | 12.5 | 25 | 50 |
| Kollidon ® SR, mg | 250 | 250 | 250 | 250 |

Preparation of Tablets:

Individual excipient was weighed and mixed in a glass mortar. The physical mixture was transferred to Carver press and compressed @ 2000 pound force. Prepared tablets were heat treated @120° C. for 1 hour. Heat treated tablets were evaluated for their crush resistance.

Ball Mill Method:

Each tablet was weighed and transferred to a stainless steel container, and ball milled using two stainless steel balls (10 mm in diameter) at a frequency of 25/sec for 5 minutes.

Sieve Analysis:

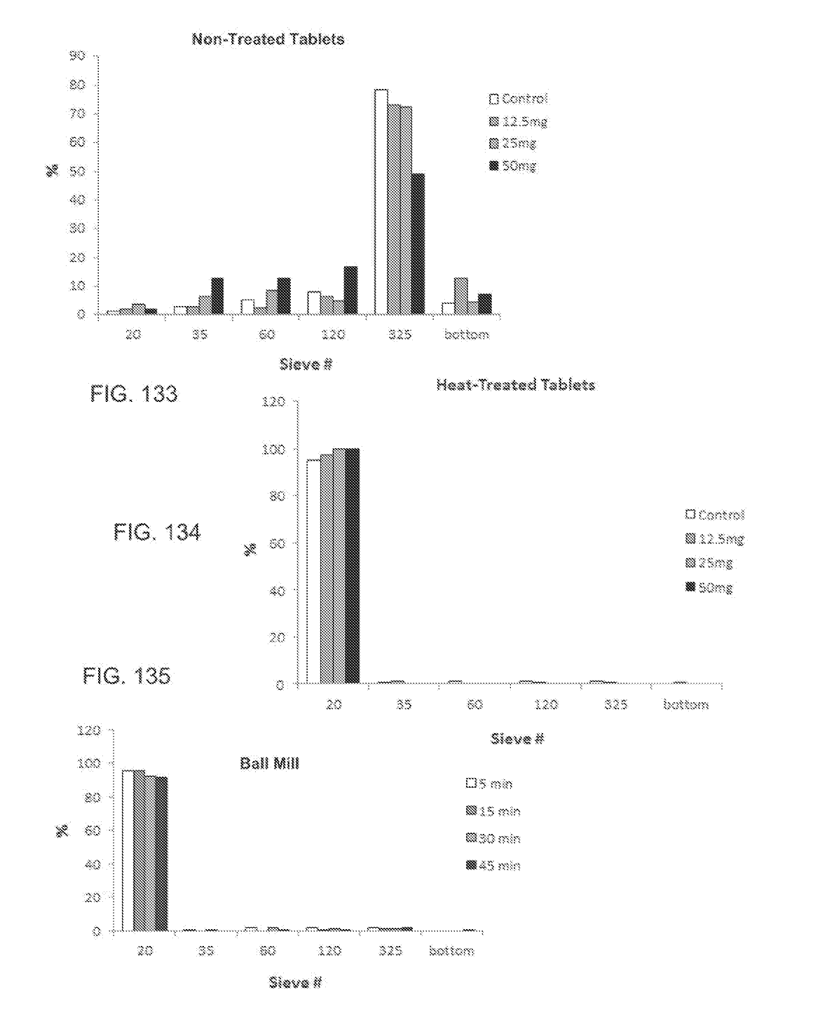

Sieves were placed in a column by their sieve number (20, 35, 60, 120 and 325) and the receiver was placed at the bottom. Top sieve was loaded with the sample and sieves were placed on a sifter (Sieve sifter SS-3CP by Cole Parmer). Tapping was performed for one minute @ 60 tapping per minute. Particles retained on each sieve were separated and weighed according to the formula % retained on sieve# ((powder retained on sieve #/total powder weight)*100), with results shown in Table 79 and FIG. 133. It may be seen in Table 79 and FIG. 133 that the effect of plasticizer concentration up to 25 mg is negligible, whereas the effect becomes significant at 50 mg.

TABLE 79
Non-treated tablets (with no heat treatment)

| Sieve # | Control | 12.5 mg | 25 mg | 50 mg |
|---|---|---|---|---|
| 20 | 1.39 | 2.09 | 3.75 | 2.01 |
| 35 | 2.79 | 2.88 | 6.56 | 12.56 |
| 60 | 5.23 | 2.62 | 8.44 | 12.56 |
| 120 | 8.01 | 6.54 | 4.69 | 16.83 |
| 325 | 78.40 | 73.04 | 72.19 | 48.99 |
| Bottom | 4.18 | 12.83 | 4.37 | 7.04 |

### Heat-Treated Tablets:

All heat-treated tablets were ball-milled under similar conditions, and resisted crushing and generated only large particles under crushing forces. There is a slight effect on crush resistance as PEG concentration increases, however the effect reaches plateau at 25 mg concentration, as shown in Table 80 and FIG. 134.

TABLE 80
Heat-treated tablets

| sieve # | Control | 12.5 mg | 25 mg | 50 mg |
|---|---|---|---|---|
| 20 | 95.22 | 97.61 | 100.00 | 100.00 |
| 35 | 0.56 | 1.06 | 0.00 | 0.00 |
| 60 | 1.40 | 0.00 | 0.00 | 0.00 |
| 120 | 1.40 | 0.27 | 0.00 | 0.00 |
| 325 | 1.40 | 0.53 | 0.00 | 0.00 |
| Bottom | 0.00 | 0.53 | 0.00 | 0.00 |

Since there is no difference in particle size distribution of the 25 mg and the 50 mg heat-treated compositions when processed via ball milling for 5 minutes, the 25 mg heated composition was evaluated for further testing, ball milling for longer period of time, and the use of a heavy duty crusher (grind milling).

### Heat Treated Tablets at 120° C. for 1 hr (Control Versus Plasticized):

TABLE 81

| Component | Control | Trial2 |
|---|---|---|
| AcDiSol ®, mg | 150 | 150 |
| PEG 8000, mg | 0 | 25 |
| Kollidon ® SR, mg | 250 | 250 |

Ball Mill Method:

Each tablet was weighed and transferred to a stainless steel container with two stainless steel balls (10 mm in diameter). The milling was performed for 15, 30 and 45 minutes at the frequency of 25/sec.

Grind Mill Method:

Tablets, after the ball mill experiment, were transferred to a heavy-duty grinder and processed for 10 sec. The ground tablets were analyzed for particle size distribution by sieve analysis.

Sieve Analysis:

Sieves were placed in a column by their sieve number (20, 35, 60, 120 and 325) and the receiver at the bottom. Top sieve was loaded with the sample and sieves were placed on a sifter (Sieve sifter SS-3CP by Cole Parmer). Tapping was performed for one minute @ 60 tapping per minute. Particles retained on each sieve were separated and weighed.

% retained on sieve #: (powder retained on sieve #/total powder weight)*100

Results are shown in Table 82 and FIG. 135.

Control Tablet with No PEG:

TABLE 82

| Sieve # | 5 min | 15 min | 30 min | 45 min |
|---|---|---|---|---|
| 20 | 95.22 | 95.29 | 91.92 | 91.57 |
| 35 | 0.56 | 0.00 | 0.28 | 0.00 |
| 60 | 1.40 | 0.00 | 1.67 | 0.28 |
| 120 | 1.40 | 0.55 | 1.39 | 0.56 |
| 325 | 1.40 | 0.83 | 0.84 | 1.69 |
| bottom | 0.00 | 0.00 | 0.00 | 0.28 |

Heat-Treated Control Tablets with No PEG:

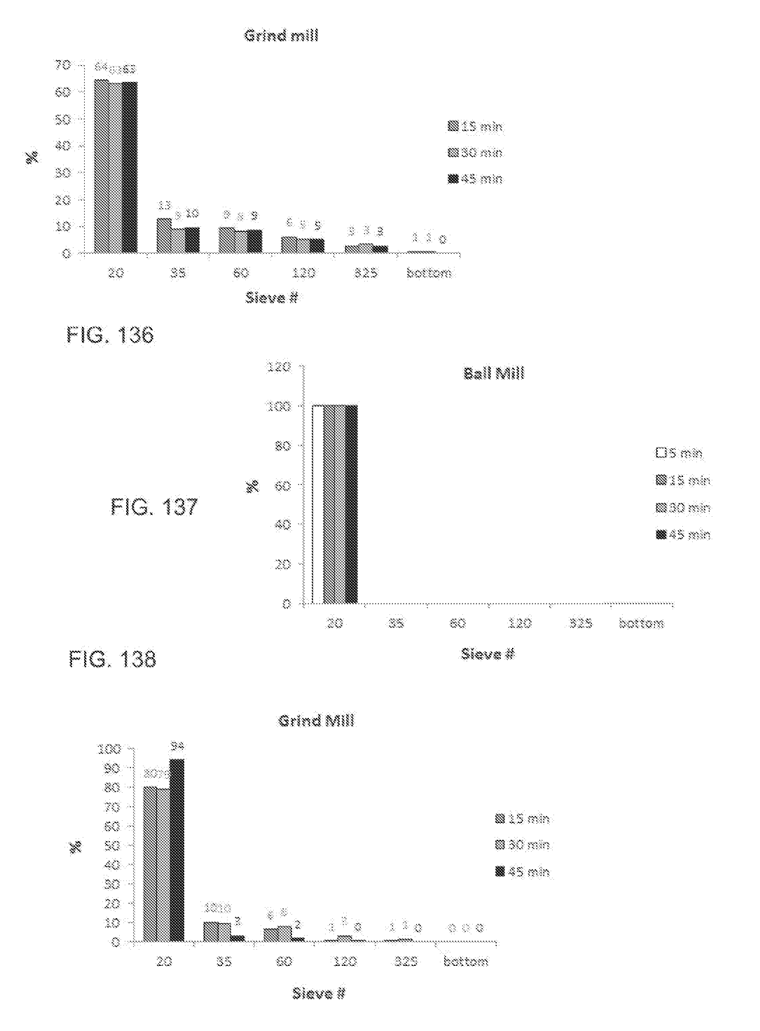

Ball mill duration did not affect particle size distribution, and hence tablets remained crush resistant. Results are shown in Table 83 and FIG. 136.

TABLE 83

| Control | 15 min | 30 min | 45 min |
|---|---|---|---|
| 20 | 64.3 | 62.95 | 63.5 |
| 35 | 12.74 | 8.91 | 9.55 |
| 60 | 9.42 | 8.36 | 8.71 |
| 120 | 6.1 | 5.3 | 5.06 |
| 325 | 2.77 | 3.34 | 2.81 |
| bottom | 0.55 | 0.58 | 0 |

Heat-treated control tablets with no PEG show almost no difference in resistance to grind milling with milling duration. However, comparison of the two graphs shows that grind milling is much more effective than ball milling in crushing the tablets into finer particles.

### Tablets Containing 25 mg PEG8000

TABLE 84
Heat treated plasticized tablets containing 25 mg PEG8000

| Sieve # | 5 min | 15 min | 30 min | 45 min |
|---|---|---|---|---|
| 20 | 100.00 | 100.00 | 100.00 | 100.00 |
| 35 | 0.00 | 0.00 | 0.00 | 0.00 |
| 60 | 0.00 | 0.00 | 0.00 | 0.00 |
| 120 | 0.00 | 0.00 | 0.00 | 0.00 |
| 325 | 0.00 | 0.00 | 0.00 | 0.00 |
| bottom | 0.00 | 0.00 | 0.00 | 0.00 |

As can be seen in Table 84 and FIG. 137, heat-treated plasticized tablets containing 25 mg PEG 8000 completely resist the ball milling regardless of the milling duration.

TABLE 85
Tablets of Table 84 in Grind Mill

| Control | 15 min | 30 min | 45 min |
|---|---|---|---|
| 20 | 79.8 | 79.06 | 93.96 |
| 35 | 10.07 | 9.56 | 3.15 |
| 60 | 6.2 | 7.75 | 1.57 |
| 120 | 0.52 | 3.1 | 0.26 |
| 325 | 0.52 | 1.29 | 0 |
| bottom | 0 | 0 | 0 |

As can be seen in Table 85 and FIG. 138, heat-treated plasticized tablets containing 25 mg PEG 8000 resist grind milling at 15 and 30 min to the same extent, however crush resistance increases as milling process extends to 45 min. This is perhaps due to heat build-up during the extended milling process. Nevertheless, plasticized tablets resist ball and grind millings significantly better than control tablets with no plasticizer.

### Effect of the Milling Process on Control Sample (Ball Versus Grind Milling):

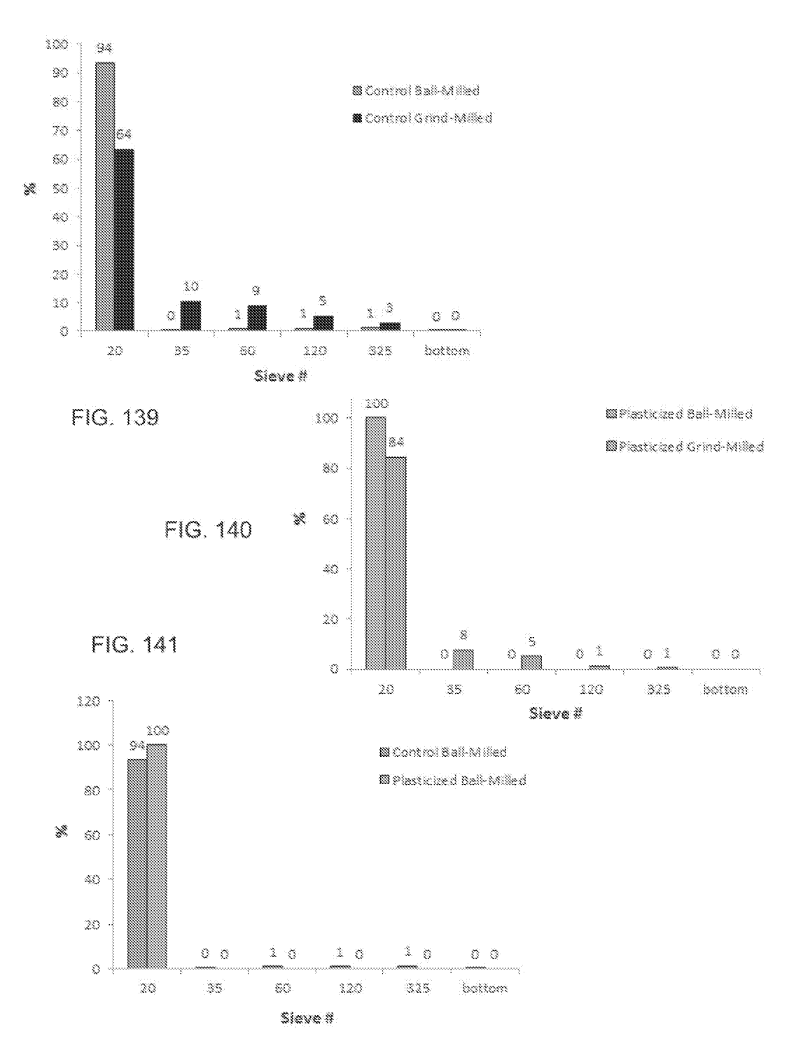

Control tablets with no PEG collectively (over total milling duration) resist ball milling, however when crushed using grind milling, 30% more of finer particles were generated, as shown in FIG. 139.

### Effect of Milling Process on Plasticized Sample (Ball Versus Grind Milling):

Plasticized tablets containing 25 mg PEG 8000 collectively resist ball milling to full extent of 100%, however they showed relatively less resistance to grind milling as 16% finer particles were generated during the process. The effect of crushing process was shown to be significant. Tablets containing 25 mg of PEG 8000 generate 14% less finer particles, as shown in FIG. 140.

### Effect of Plasticization on Tablets Processed Through Ball Milling:

When processed through ball milling, control and plasticized tablets both showed almost same crush resistance. In other words, for plasticized tablets, the effect of crushing process (ball versus grind milling) is much less compared to tablets with no plasticizer, as shown in FIG. 141.

### Effect of Plasticization on Tablets Processed Through Grind Milling:

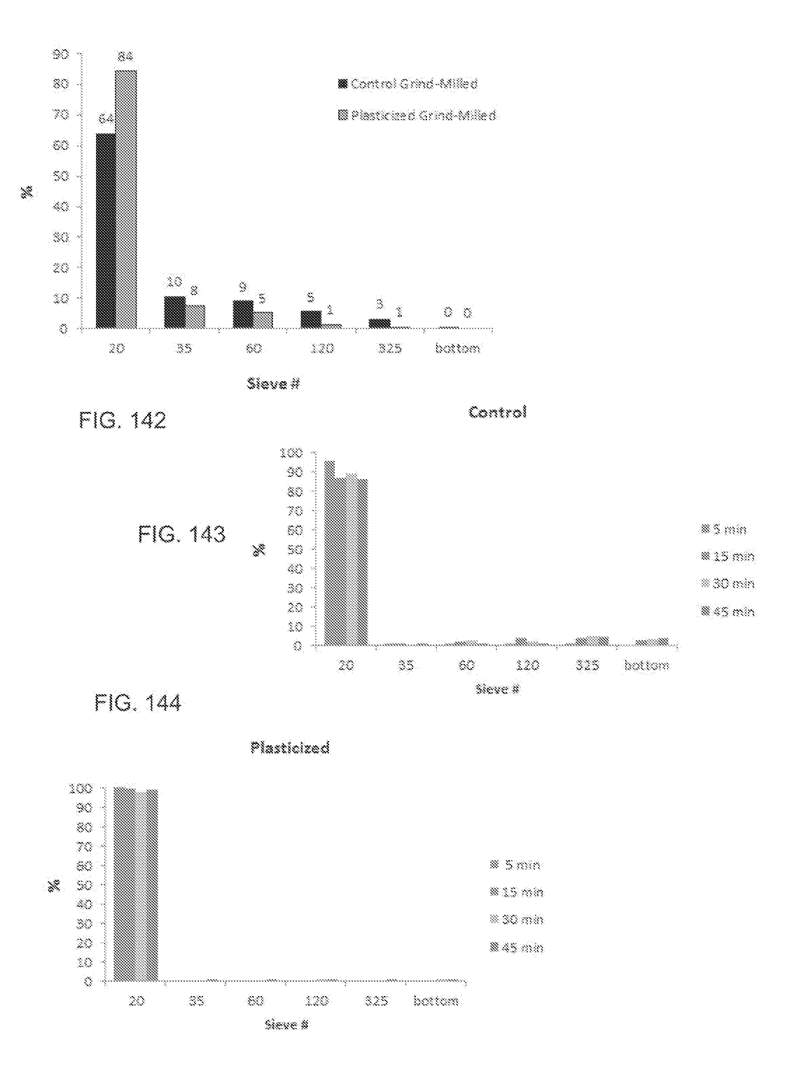

When processed through grind milling, control tablets with no plasticizer showed significantly less resistance to crushing compared to plasticized tablets, as shown in FIG. 142.

### Heat Treated Tablets at 100° C. for 1 hr (Control Versus Plasticized)

The plasticized formulation is shown in Table 86, and the results are shown in Table 87 and FIGS. 143 and 144.

TABLE 86
Formulation

| | Control | Plasticized |
|---|---|---|
| AcDiSol ® | 150 | 150 |
| Kollidon ® SR | 250 | 250 |
| PEG8000 | 0 | 25 |

TABLE 87
Ball mill study

| Control | 5 min | 15 min | 30 min | 45 min | Plasticized | 5 min | 15 min | 30 min | 45 min |
|---|---|---|---|---|---|---|---|---|---|
| 20 | 95.3 | 86.8 | 88.67 | 85.95 | 20 | 100 | 99.24 | 97.5 | 98.73 |
| 35 | 0.27 | 0.82 | 0 | 0.55 | 35 | 0 | 0 | 0 | 0.25 |
| 60 | 0.82 | 2.19 | 2.49 | 0.55 | 60 | 0 | 0 | 0 | 0.76 |
| 120 | 0.82 | 3.57 | 1.66 | 0.83 | 120 | 0 | 0 | 0.25 | 0.51 |
| 325 | 0.82 | 3.85 | 4.7 | 4.43 | 325 | 0 | 0 | 0 | 0.51 |
| bottom | 0 | 2.75 | 3.04 | 3.6 | bottom | 0 | 0 | 0.5 | 0.76 |

### Grind Mill Study:

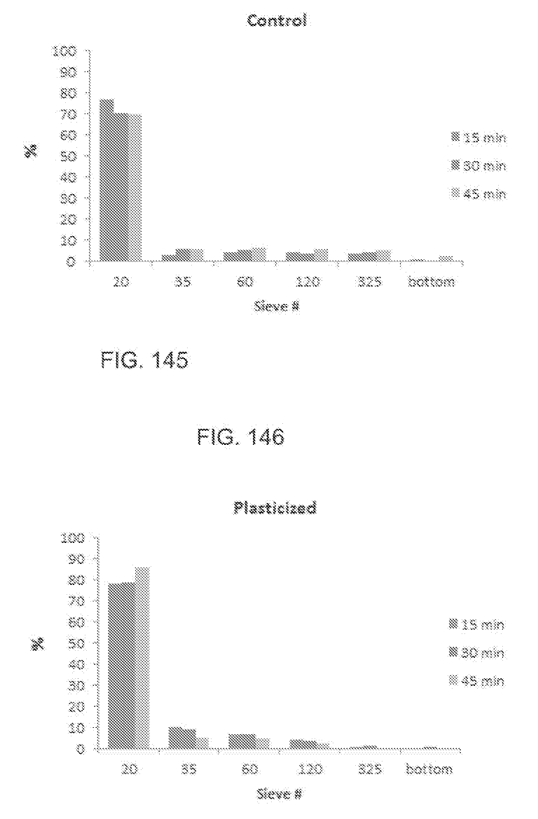

Tablets ball-milled at 15, 30, and 45 min, processed in a grind mill for 10 sec. Results are shown in Table 88, and in FIGS. 145 and 146.

TABLE 88

| Control | 15 min | 30 min | 45 min | Plasticized | 15 min | 30 min | 45 min |
|---|---|---|---|---|---|---|---|
| 20 | 76.67 | 70.44 | 69.52 | 20 | 78.12 | 78.44 | 85.82 |
| 35 | 2.78 | 5.52 | 5.82 | 35 | 9.92 | 9.02 | 5.32 |
| 60 | 3.89 | 5.25 | 6.09 | 60 | 7.12 | 6.67 | 4.81 |
| 120 | 4.17 | 3.31 | 5.54 | 120 | 4.33 | 3.26 | 2.28 |
| 325 | 3.61 | 3.87 | 4.97 | 325 | 0.25 | 1.25 | 0 |
| bottom | 0.83 | 0 | 2.49 | bottom | 0 | 0.75 | 0 |

### Heat Treated Tablets at 80° C. for 1 hr (Control Versus Plasticized)

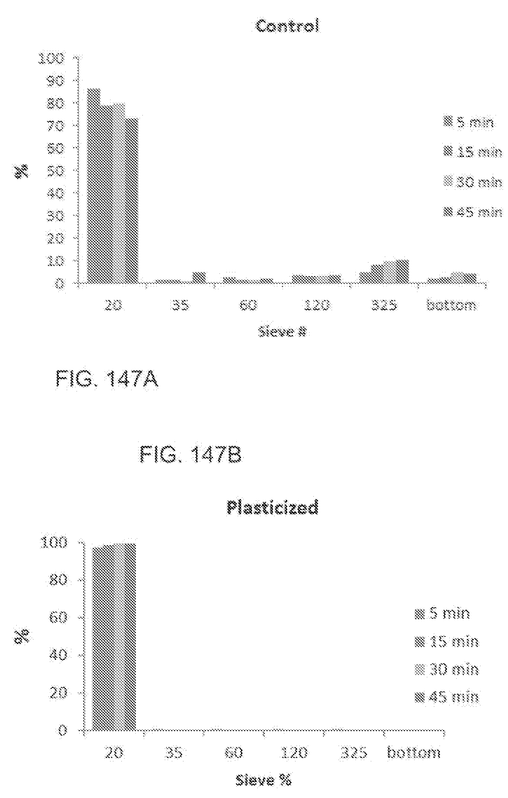

The plasticized formulation is shown in Table 89, and the results are shown in Table 90 and FIGS. 147A and 147B.

TABLE 89
Formulation

| | Control | Plasticized |
|---|---|---|
| AcDiSol ® | 150 | 150 |
| Kollidon ® SR | 250 | 250 |
| PEG8000 | 0 | 25 |

TABLE 90
Ball mill study

| Control | 5 min | 15 min | 30 min | 45 min | Plasticized | 5 min | 15 min | 30 min | 45 min |
|---|---|---|---|---|---|---|---|---|---|
| 20 | 86.2 | 78.44 | 79.51 | 73.12 | 20 | 97.54 | 98.74 | 99.74 | 99.74 |
| 35 | 1.08 | 1.08 | 0.54 | 4.45 | 35 | 0.98 | 0 | 0 | 0 |
| 60 | 2.43 | 1.35 | 1.35 | 1.74 | 60 | 0.49 | 0 | 0 | 0 |
| 120 | 3.78 | 3.23 | 2.96 | 3.49 | 120 | 0.25 | 0 | 0 | 0 |
| 325 | 4.86 | 7.81 | 9.7 | 9.94 | 325 | 0.25 | 0 | 0 | 0 |
| bottom | 1.62 | 2.15 | 4.58 | 4.29 | bottom | 0 | 0 | 0 | 0 |

### Grind Mill Study:

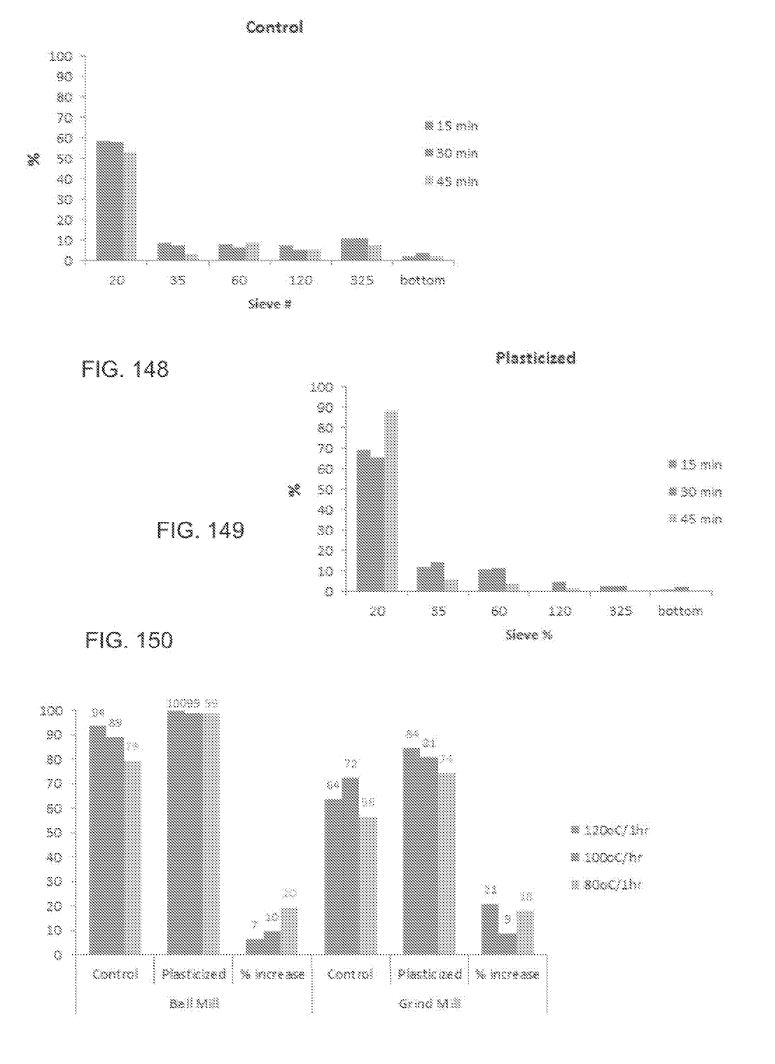

Tablets ball-milled for 15, 30, and 45 min, processed in a grind mill for 10 sec. Results are shown in Table 91, and are illustrated in FIGS. 148-149.

TABLE 91

| Control | 15 min | 30 min | 45 min | Plasticized | 15 min | 30 min | 45 min |
|---|---|---|---|---|---|---|---|
| 20 | 58.49 | 57.82 | 52.81 | 20 | 69.27 | 65.23 | 88.05 |
| 35 | 8.62 | 7.16 | 2.96 | 35 | 11.84 | 14.2 | 5.71 |
| 60 | 7.82 | 6.1 | 8.34 | 60 | 10.83 | 11.17 | 3.64 |
| 120 | 7.55 | 5.31 | 5.24 | 120 | 0 | 4.57 | 1.04 |
| 325 | 10.51 | 10.88 | 7.67 | 325 | 2.52 | 2.54 | 0 |
| bottom | 1.89 | 3.71 | 1.62 | bottom | 0.25 | 1.76 | 0 |

Table 92 and FIG. 150 show the percentage of particles retained on Sieve #20 (particles larger than 850 μm).

TABLE 92

| | Ball Mill | | | Grind Mill | | |
|---|---|---|---|---|---|---|
| | | | % | | | % |
| | Control | Plasticized | increase | Control | Plasticized | increase |
| 120° C.- | 93.5 | 99.95 | 6.5 | 63.58 | 84.3 | 20.7 |
| 1 hr | | | | | | |
| 100° C.- | 89.2 | 98.9 | 9.7 | 72.2 | 80.8 | 8.6 |
| hr | | | | | | |
| 80° C.- | 79.3 | 98.9 | 19.6 | 56.4 | 74.2 | 17.8 |
| 1 hr | | | | | | |

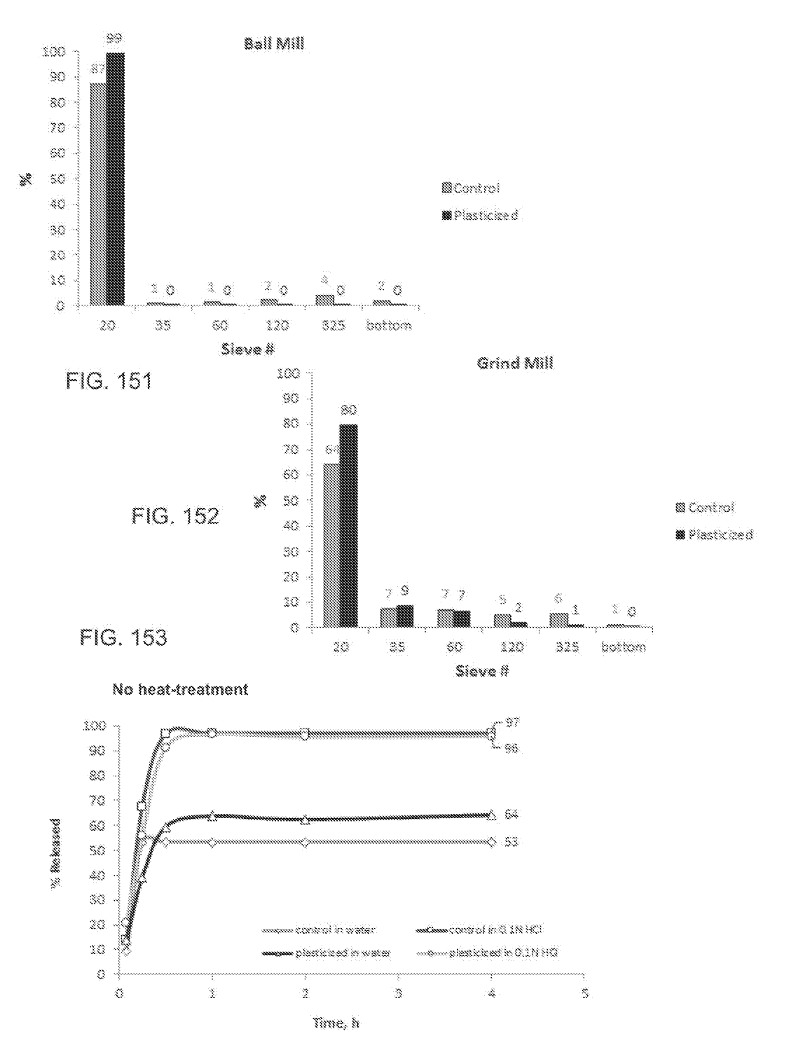

All data for the tablets which have undergone different heat treatments at different grinding times are shown in FIGS. 151-152. Compared to the tablets without sintering enhancers, tablets containing 25 mg of the sintering enhancer show 12% and 16% more resistance to crushing by ball and grind mill, respectively.

### Drug Release Studies:

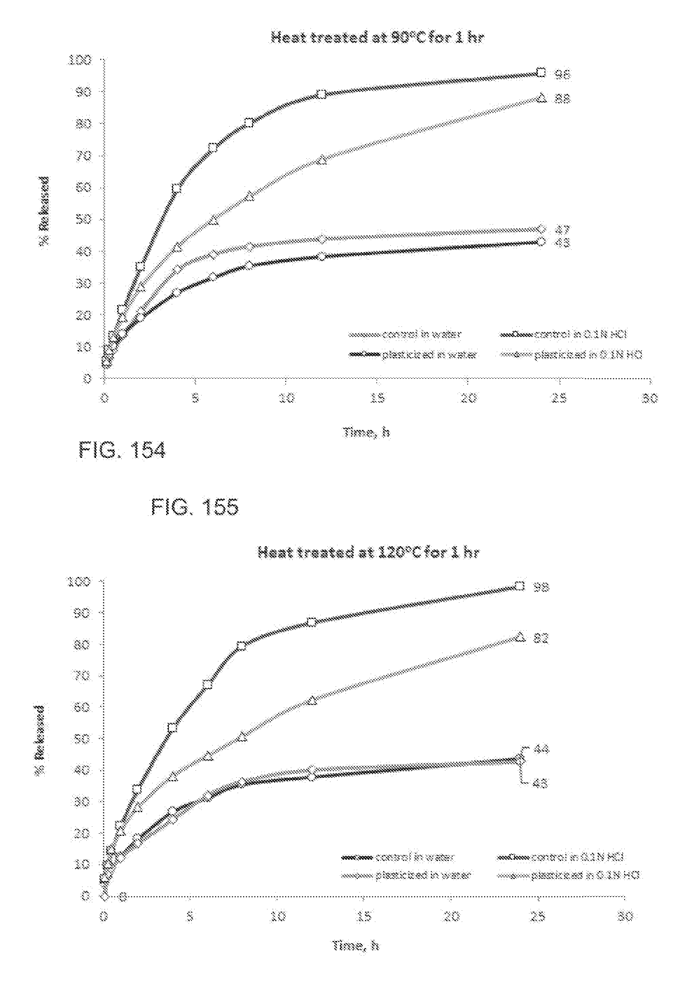

Control and plasticized tablets (with 25 mg PEG8000) to be heat treated are formulated as in Table 93, with release results for no heat-treatment shown in Table 94 and FIG. 153, and for heat-treatment in Table 95 and FIG. 154 at 90° C., and Table 96 and FIG. 155 at 120° C.

TABLE 93

| | Control | Plasticized |
|---|---|---|
| AcDiSol ® | 150 | 150 |
| Kollidon ® SR | 250 | 250 |
| PEG8000 | 0 | 25 |

| | |
|---|---|
| Heat-Treatment | 90° C. and 120° C. for 1 hour |

TABLE 94

| | Control | Control in | Plasticized | Plasticized |
|---|---|---|---|---|
| Time, h | in water | 0.1N HCl | in water | in 0.1N HCl |
| 0 | 0 | 0 | 0 | 0 |
| 0.083 | 9.42 | 13.95 | 14.05238 | 20.69 |
| 0.25 | 53.40 | 67.41 | 39.03571 | 55.795 |
| 0.5 | 53.36 | 96.84 | 59.42143 | 91.055 |
| 1 | 53.36 | 97.10 | 63.8 | 96.615 |
| 2 | 53.36 | 97.10 | 62.43333 | 95.58 |
| 4 | 53.36 | 97.10 | 64.22143 | 95.975 |

TABLE 95
Heat treated at 90° C. for 1 hour

| | control | control in | plasticized | plasticized |
|---|---|---|---|---|
| Time, h | in water | 0.1N HCl | in water | in 0.1N HCl |
| 0 | 0 | 0 | 0 | 0 |
| 0.083 | 5.00 | 5.45 | 4.53 | 5.31 |
| 0.25 | 6.69 | 8.92 | 7.31 | 8.94 |
| 0.5 | 8.90 | 13.35 | 9.93 | 12.88 |
| 1 | 13.48 | 21.28 | 13.83 | 19.42 |
| 2 | 21.24 | 34.96 | 18.97 | 28.85 |
| 4 | 34.17 | 59.42 | 26.95 | 41.55 |
| 6 | 39.05 | 72.17 | 31.69 | 50.08 |
| 8 | 41.46 | 80.17 | 35.33 | 57.24 |
| 12 | 43.80 | 89.06 | 38.22 | 68.90 |
| 24 | 47.00 | 95.65 | 42.88 | 88.23 |

TABLE 96
Heat treated at 120° C. for 1 hour

| | control | control in | plasticized | plasticized |
|---|---|---|---|---|
| Time, h | in water | 0.1N HCl | in water | in 0.1N HCl |
| 0 | 0 | 0 | 0 | 0 |
| 0.083 | 4.20 | 5.34 | n/a | 5.94 |
| 0.25 | 6.33 | 9.45 | 7.19 | 10.36 |
| 0.5 | 8.96 | 14.37 | 8.93 | 14.87 |
| 1 | 12.48 | 22.06 | 12.22 | 20.67 |
| 2 | 18.06 | 33.93 | 16.70 | 28.34 |
| 4 | 26.78 | 53.14 | 24.37 | 38.26 |
| 6 | 31.19 | 66.96 | 32.00 | 44.55 |
| 8 | 35.43 | 79.40 | 36.42 | 50.62 |
| 12 | 37.83 | 86.77 | 40.08 | 62.19 |
| 24 | 43.59 | 98.34 | 42.64 | 82.41 |

### Example 7: High Performing Abuse Deterrent Formulations (ADFs)

It has previously been disclosed, for example in U.S. Patent No. 61/918,880, which is incorporated herein by reference in its entirety, that the addition of bentonite clay into a tablet dosage form containing tramadol HCl will significantly reduce the amount of available drug if the dosage form is tampered. Bentonite clay can directly be added into a solid dosage form containing the controlled substance drug tramadol HCl. Alternatively, the drug can be formulated into a complex with the clay before being formulated into a final dosage form. This drug-clay complex will not readily release drug if an abuser were to sniff the powder from crushed tablets as compared to free drug. Moreover, the clay itself is irritating in powder form; as would occur when tablets are crushed into fine particles. In the case where free clay particles are incorporated into a dosage form, the clay will immediately form a strong complex with the basic drug in solution, preventing the abusable drug from being extracted into solution. The following graph shows the powerful binding effect of bentonite clay with tramadol HCl.

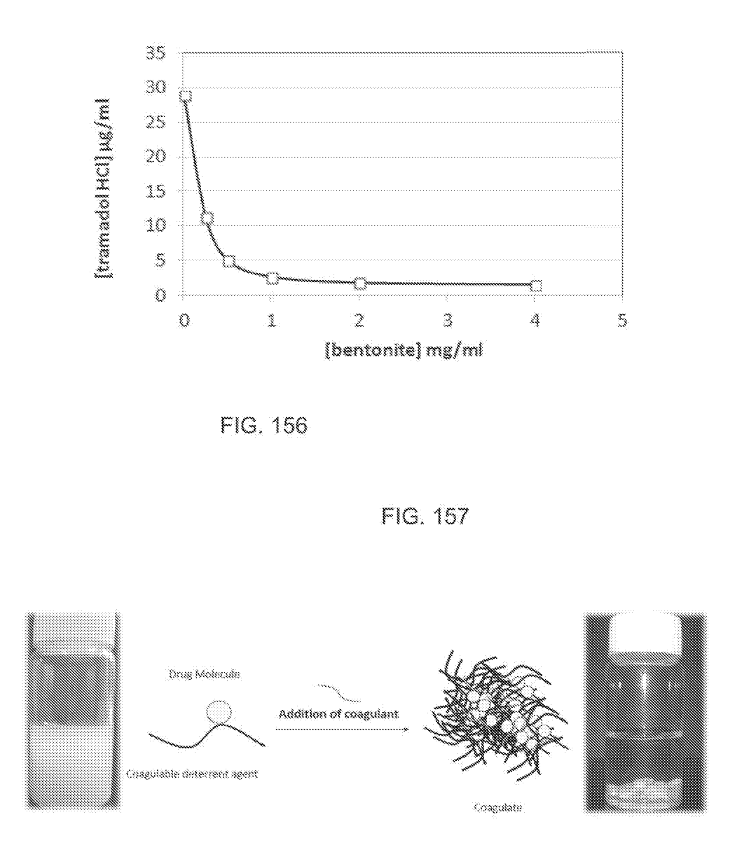

With reference to FIG. 156, different clay amounts added into 10 ml of 25 μg/ml tramadol HCl aqueous solution are illustrated. Dispersions were vortexed for 5 sec and then centrifuged at 1500 rpm for 5 min. Supernatant was then analyzed for tramadol concentration using UV-Visible Spectroscopy at 271 nm (UV-1700, Shimadzu).

For extraction/injection purposes, abusers are likely to disperse crushed tablet(s) in an aqueous medium (<10 mL in volume), then filter the medium to obtain a clear solution for injection. It is known that bentonite particles can strongly bind to tramadol HCl molecules in an aqueous medium, and the objective of this disclosure is to enhance this effect by reducing filtrate volume (volume passing through filter), enhancing binding capacity through molecular entrapment and coagulation, and preventing drug extraction from an aqueous dispersion. This disclosure discloses that 1) addition of a minute amount of very high molecular weight polyethylene oxide (PEO) in solid state can induce coagulation (as shown below) in a dispersion system composed of tramadol HCl and bentonite clay. As shown herein, a tablet formulation containing tramadol HCl, bentonite clay, and minute amounts of a PEO polymer possesses better deterrence capacity than control tablet without PEO. Compared to control tablets containing drug and bentonite clay, new tablets containing addition of PEO can enhance the deterrent capacity by about 4-5% due to molecular entrapment of the drug and 8-10% by reducing the filtrate volume. The new tablets also display aversive properties once in solution; and addition of sodium bentonite to a pharmaceutical compositions containing an abusable medication can enhance drug binding and prevent drug extraction due to its binding and dispersion stability. FIG. 157 illustrates coagulation. It should be understood that other therapeutic drugs may be combined with clay and PEO as described herein, with the same or similar benefits.

### Section 1: Induced Coagulation of Clay Particles

Experimental:

A drug stock solution containing tramadol HCl in deionized water (2.5 mg/mL) was prepared. Different amounts of the bentonite clay were added into 20 mL scintillation vials, to which 10 mL of the drug solution was added. The dispersion was mixed by vortexing for 5 seconds followed by centrifugation at 1500 rpm for 5 minutes. The supernatant was then extracted and filtered through a 0.2 micron syringe filter. Concentration of drug remaining in this filtered extract was then measured by UV-Visible spectroscopy at 271 nm (Shimadzu UV-1700 spectrophotometer).

### Enhanced Drug Entrapment Due to Coagulation:

Sample Preparation:

Control: 0.5 mL of drug solution (2.5 mg/mL) was diluted to 10 mL with DI water to measure drug binding.

Non-coagulated suspension: To three different glass vials containing 10 mL of drug solution (2.5 mg/mL) was added 50 mg, 75 mg and 100 mg of bentonite. The suspensions were then centrifuged for 5 min at 1500 rpm and the supernatants were filtered. A 0.5 mL of filtered supernatants were diluted to 10 mL with DI water to measure drug binding.

Coagulated suspension: To three different glass vials containing 10 mL of drug solution (2.5 mg/mL) was added 50 mg, 75 mg and 100 mg of bentonite, and then 2 mg of polyethylene oxide (PEO). The dispersions were centrifuged for 5 min at 1500 rpm. The 0.5 mL of filtered supernatants were diluted to 10 mL with DI water to measure drug binding.

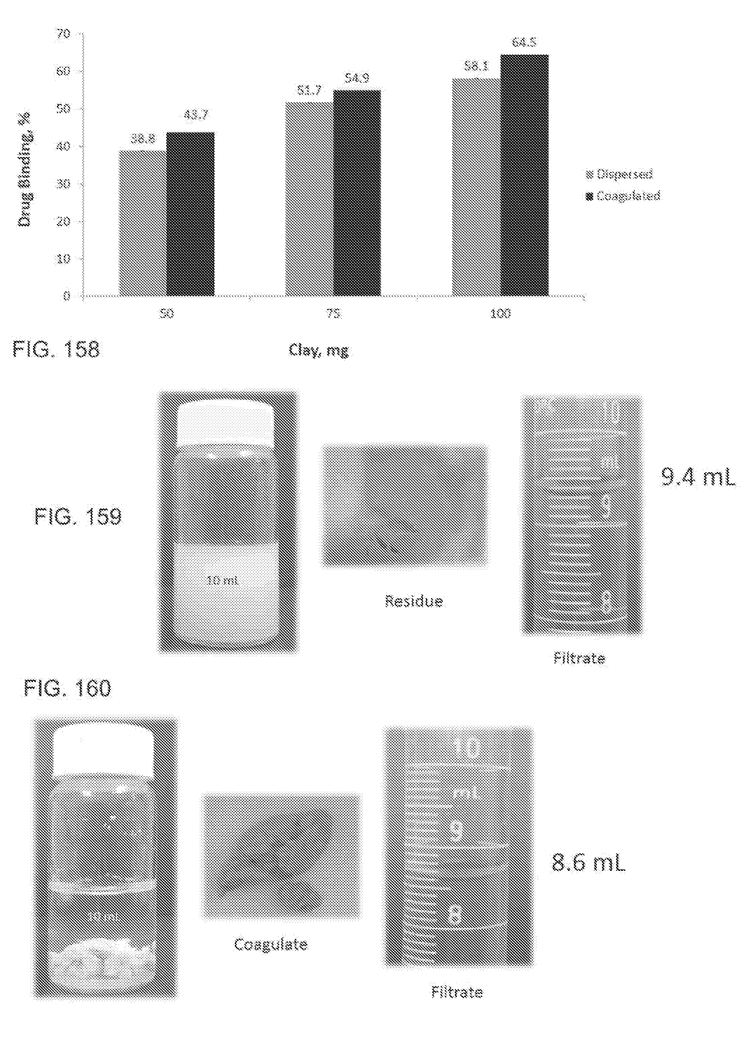

The amount of drug bound (%) to clay particles for the control, non-coagulated, and coagulated dispersion systems are shown in Table 97, and are illustrated in FIG. 158.

TABLE 97
Formulations

| | 50 mg clay | | | 75 mg clay | | | 100 mg clay | | |
|---|---|---|---|---|---|---|---|---|---|
| | | Non | | | Non | | | Non | |
| Expt. | Control | coagulated | Coagulated | Control | coagulated | Coagulated | Control | coagulated | Coagulated |
| 1 | 0.746 | 0.457 | 0.422 | 0.751 | 0.363 | 0.338 | 0.753 | 0.315 | 0.268 |
| 2 | 0.750 | 0.460 | 0.422 | 0.751 | 0.363 | 0.339 | 0.752 | 0.316 | 0.268 |
| 3 | 0.750 | 0.460 | 0.422 | 0.750 | 0.363 | 0.339 | 0.753 | 0.316 | 0.268 |
| 4 | 0.750 | 0.459 | 0.422 | 0.751 | 0.362 | 0.339 | 0.752 | 0.316 | 0.268 |
| 5 | 0.745 | 0.458 | 0.422 | 0.750 | 0.362 | 0.340 | 0.752 | 0.316 | 0.268 |
| Average | 0.750 | 0.458 | 0.422 | 0.750 | 0.363 | 0.339 | 0.753 | 0.316 | 0.268 |
| % Drug Binding | | 38.8 | 43.7 | | 51.7 | 54.9 | | 58.1 | 64.5 |

### Reduced Filtrate Volume Due to Coagulation:

Starting with 10 mL of an initial drug solution, the filtrate recoverable after clay flocculation was determined volumetrically. As shown in FIGS. 159-160, coagulation can lower the volume of filtrate for the coagulated system.

### Section II: Preventing Drug Extraction Using Sodium Bentonite

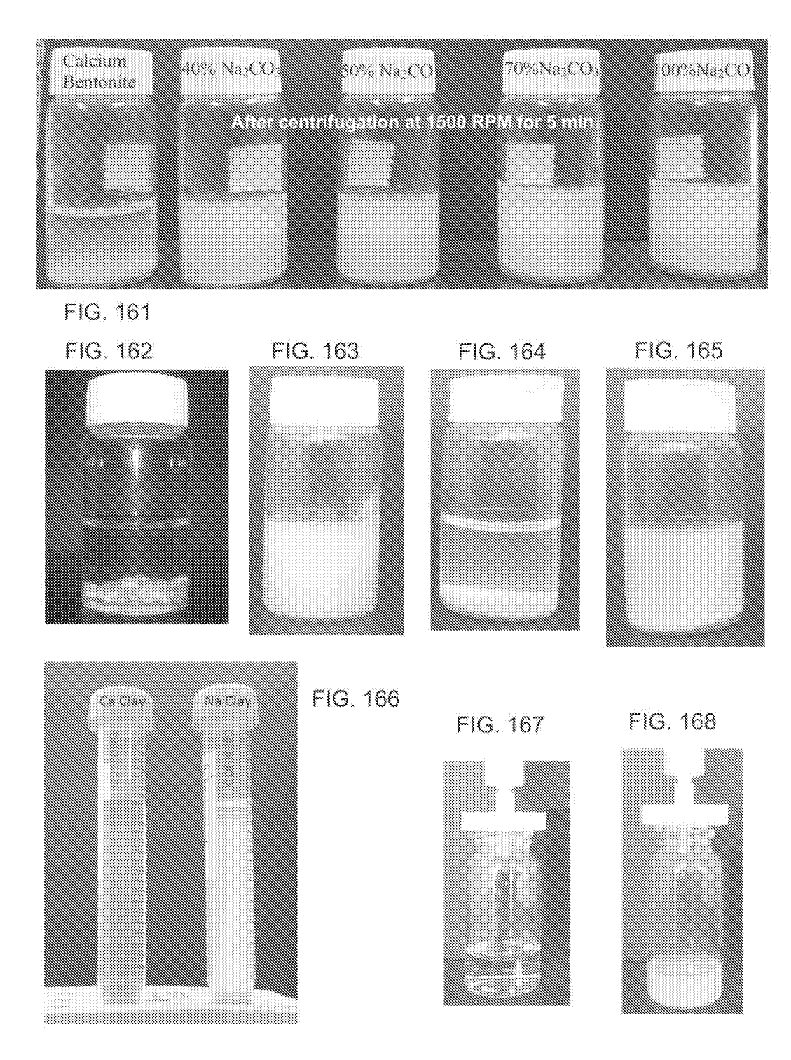

With reference to FIG. 161, sodium bentonite was prepared as follows: Calcium bentonite (1 g) was mixed with different amounts of sodium carbonate (100, 300, 500, 700, and 1000 mg). Each solid mixture was then dispersed in 100 mL of distilled water under stirring. The dispersion was heated to over 80° C. until most water evaporated, then transferred to a hot air-oven and heated again at 100° C. for 1 hr. The mixture was triple washed with ultrapure deionized water and dried again in a hot air oven at 100° C. for 1 hr. The dried mixture was further tested for its suspendability and filterability.

Suspension behavior of different sodium bentonite samples after centrifugation at 1500 RPM for 5 min compared with that of calcium bentonite suspension is described below. Sodium bentonite prepared using 40-50% sodium carbonate displayed maximum stability.

The main characteristic differences observed between sodium bentonite and its calcium form are shown in the Table 98.

TABLE 98

| | | Sodium Bentonite |
|---|---|---|
| | Calcium | (prepared using 50% |
| Sample | Bentonite | sodium carbonate) |
| Reaction with 6.0N HCl | No reaction | Effervescence |
| Reaction to PEO | Effective | No effect even after |
| | coagulation | 5 mg addition |
| | upon 2 mg | FIG. 163 |
| | addition | |
| | FIG. 162 | |
| Dispersion stability | Particles | No Particle settling |
| after centrifugation | settle | FIG. 165 |
| at 1500 RPM for 5 min | FIG. 164 | |
| Dispersion stability | FIG. 166 | |
| after centrifugation | | |
| at 4500 RPM for 5 min | | |
| Filtration through | FIG. 167 | FIG. 168 |
| 0.2 μm filter | | |

### Determination of Filtration Time:

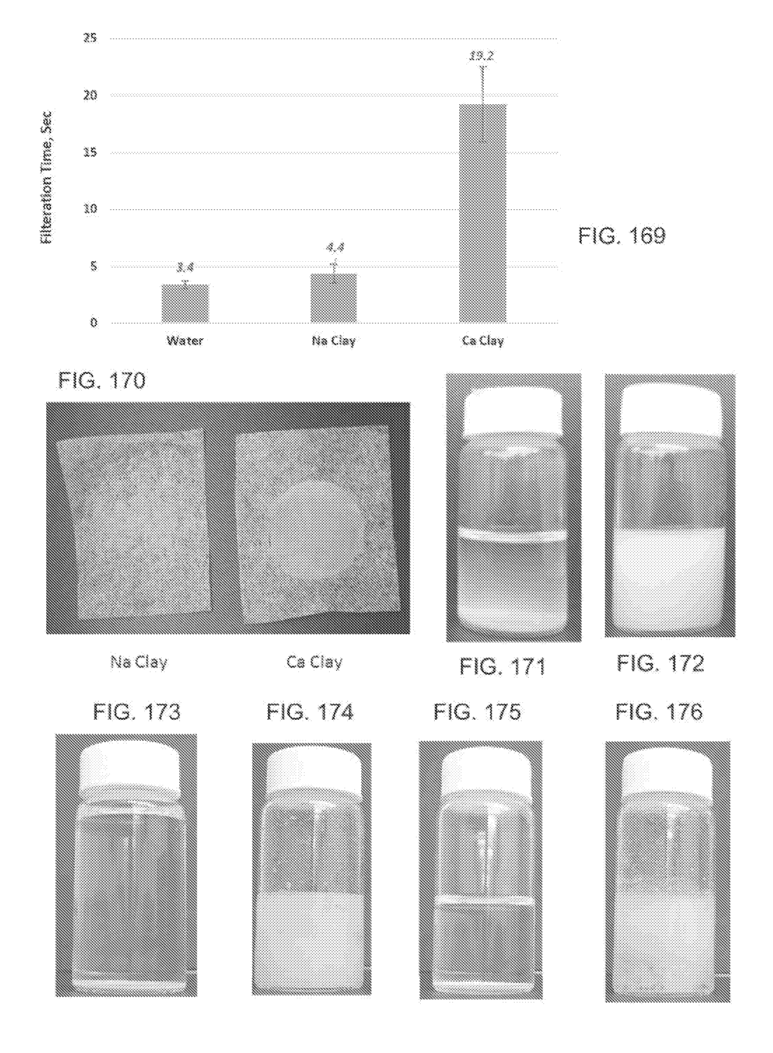

Experimental: A 200 mg of calcium bentonite (Sigma) and sodium bentonite (Alfa Aesar) were dispersed in 10 mL of ultrapure deionized water. The suspension was then filtered under negative vacuum pressure of 200 mmHg through a filter membrane (tea bag). A 20 mL of ultrapure deionized water was passed through the filter after each experiment to clean the filter. For each experiment, the amount of time needed for the dispersion to pass through the filter was recorded, as illustrated in FIG. 169.

Bentonite retention on filter membrane (tea bag) after vacuum filtration can be visualized in FIG. 170. In case of calcium bentonite the sample was fully retained on the membrane whereas with sodium bentonite, all the sample was fully passed through.

### Free Settling:

Suspension stability of sodium and calcium bentonite clays have been examined under normal gravity for 12 hours. Sodium bentonite remained well suspended while calcium clay settled as shown in Table 99.

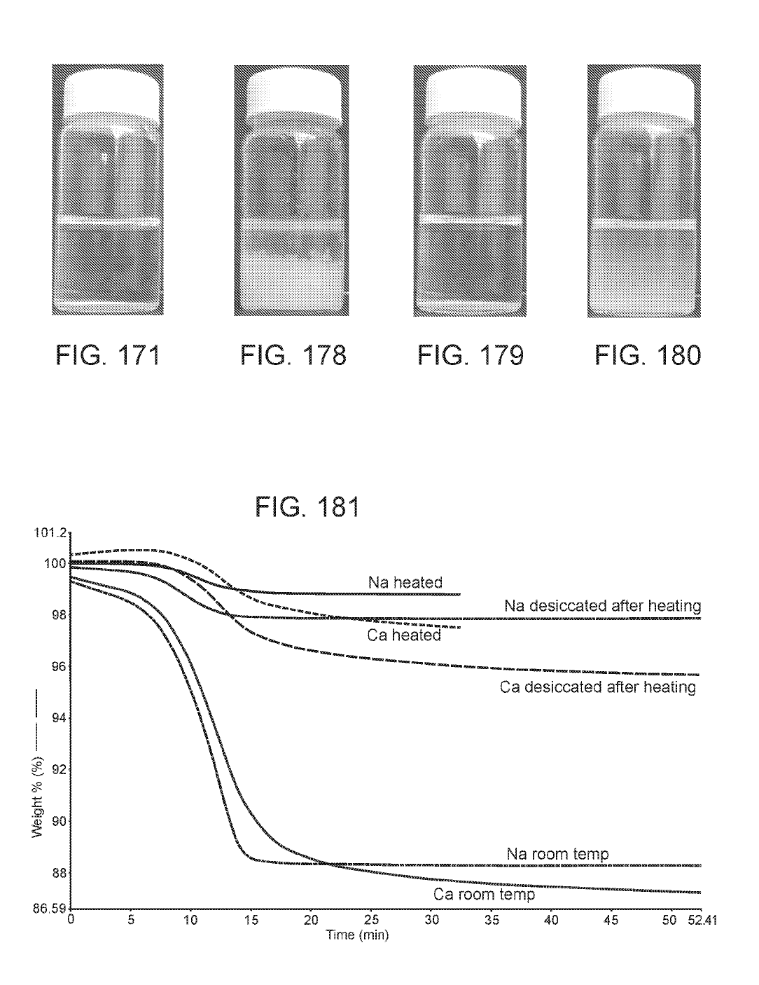

TABLE 99

| Drug Conc. μg/mL | Ca Clay | Na Clay |
|---|---|---|
| No Drug | FIG. 171 | FIG. 172 |
| 1000 | FIG. 173 | FIG. 174 |
| 500 | FIG. 175 | FIG. 176 |
| 200 | FIG. 177 | FIG. 178 |
| 100 | FIG. 179 | FIG. 180 |

TABLE 100
Electrical conductivity of the dispersions

| | Conductivity (mV) | | | | pH | | | |
|---|---|---|---|---|---|---|---|---|
| Sample | Trial 1 | Trial 2 | Trial 3 | Mean | Trial 1 | Trial 2 | Trial 3 | Mean |
| Na Clay | −202.1 | −205.2 | −204.7 | −204 | 10.61 | 10.66 | 10.65 | 10.64 |
| Ca Clay | −76.5 | −77.1 | −79.1 | −77.56 | 8.42 | 8.43 | 8.46 | 8.43 |
| Na₂CO₃ | −247.4 | −248.3 | −247.5 | −247.7 | 11.40 | 11.42 | 11.40 | 11.40 |
| NaCl | 34.7 | 33.6 | 52.6 | 40.3 | 6.48 | 6.50 | 6.16 | 6.38 |
| DW | 25.4 | 22.6 | 21.5 | 23.16 | 6.58 | 6.63 | 6.65 | 6.62 |

### Moisture Determination in Clay Samples:

Thermogravimetric analysis was used to determine the amount of moisture in different bentonite clays, as shown in Table 101, and as illustrated in FIG. 181.

TABLE 101

| | | Wt % loss |
|---|---|---|
| | | due to |
| Sample | Wt % | moisture |
| Na heated | 98.78 | 1.22 |
| Ca heated | 97.51 | 2.49 |
| Na desiccated | 97.84 | 2.16 |
| Ca desiccated | 95.66 | 4.34 |
| Na RT | 88.27 | 11.73 |
| Ca RT | 87.25 | 12.75 |

Binding with Diphenhydramine:

Drug solutions (at different concentrations) were prepared by dissolving the desired amount of diphenhydramine HCl in 200 mL DI water. A 25 mg of desiccated clay sample was added to 10 mL of this solution in a centrifuge tube. The suspension was vortex-mixed for 30 sec., centrifuged at 2500 RPM for 5 min, and the supernatant was filtered through a 0.2 micron filter. A control solution with no clay content was prepared for each drug concentration, and the absorbance of all solutions was determined by UV spectrophotometer at 258 nm.

TABLE 102
1000 μg/mL (100 mg in 100 mL of ultrapure deionized water)

| | Ca RT | | Ca Δ | | Na RT | | Na Δ | |
|---|---|---|---|---|---|---|---|---|
| | % Drug | Cond. | % Drug | Cond. | % Drug | Cond. | % Drug | Cond. |
| Run | binding | (mV) | binding | (mV) | binding | (mV) | binding | (mV) |
| 1 | 40.3 | −3.9 | 38.8 | −21.4 | 58.3 | −85.3 | 57.3 | −83.2 |
| 2 | 35.7 | 3.1 | 37.4 | −9.3 | 54.0 | −90.2 | 44.1 | −94 |
| 3 | 39.3 | 2.0 | 34.7 | 3.7 | 56.1 | −88.2 | 55.7 | −90.7 |
| AVG | 38.4 | 0.4 | 37.0 | −9.0 | 56.2 | −87.9 | 52.3 | −89.3 |

TABLE 103
500 μg/mL (50 mg in 100 mL of ultrapure deionized water)

| | Ca RT | | Ca Δ | | Na RT | | Na Δ | |
|---|---|---|---|---|---|---|---|---|
| | % Drug | Cond. | % Drug | Cond. | % Drug | Cond. | % Drug | Cond. |
| Run | binding | (mV) | binding | (mV) | binding | (mV) | binding | (mV) |
| 1 | 55.6 | −14.5 | 65.1 | 6.0 | 78.0 | −124.3 | 86.2 | −131.6 |
| 2 | 55.7 | 1.0 | 66.5 | −17.5 | 85.5 | −127.6 | 86.8 | −130.2 |
| 3 | 59.3 | −1.3 | 64.1 | −6.8 | 84.8 | −129.4 | 83.7 | −132.4 |
| AVG | 56.9 | −4.9 | 65.2 | −6.1 | 82.7 | −127.1 | 85.5 | −131.4 |

TABLE 104
250 μg/mL (25 mg in 100 mL of ultrapure deionized water)

| | Ca RT | | Ca Δ | | Na RT | | Na Δ | |
|---|---|---|---|---|---|---|---|---|
| | % Drug | Cond. | % Drug | Cond. | % Drug | Cond. | % Drug | Cond. |
| Run | binding | (mV) | binding | (mV) | binding | (mV) | binding | (mV) |
| 1 | 74.9 | 3.1 | 87.4 | 2.9 | 92.8 | −169.7 | 93.7 | −176.3 |
| 2 | 78.4 | −5.3 | 85.7 | 2.5 | 91.5 | −172.3 | 90.5 | −172.9 |
| 3 | 79.0 | −2.0 | 85.4 | −1.5 | 92.9 | −172.1 | 93.8 | −173.2 |
| AVG | 77.4 | −1.4 | 86.2 | 1.3 | 92.4 | −171.3 | 92.7 | −174.13 |

TABLE 105
100 μg/mL (10 mg in 100 mL of ultrapure deionized water)

| | Ca RT | | Ca Δ | | Na RT | | Na Δ | |
|---|---|---|---|---|---|---|---|---|
| | % Drug | Cond. | % Drug | Cond. | % Drug | Cond. | % Drug | Cond, |
| Run | binding | (mV) | binding | (mV) | binding | (mV) | binding | (mV) |
| 1 | 96.2 | −11.4 | 95.4 | −1.7 | −ve | −187.3 | −ve | −188.4 |
| 2 | 96.6 | −21.8 | 97.0 | −3.4 | −ve | −188.8 | −ve | −190.3 |
| 3 | 96.5 | −4.2 | 96.9 | 8.2 | −ve | −188.9 | −ve | −192.2 |
| AVG | 96.5 | −12.46 | 96.5 | 1.03 | | −188.3 | | −190.3 |

Table 106 and FIG. 190 illustrate conductivity of drug solutions in ultrapure deionized water.

TABLE 106

| Drug Conc., μg/mL | Cond. (mV) | pH |
|---|---|---|
| 1000 | 31.3 | 6.56 |
| 500 | 21.3 | 6.73 |
| 250 | 21.3 | 6.73 |
| 200 | 24.5 | 6.68 |
| 100 μg/mL | 28.2 | 6.61 |

TABLE 107
Conductivity of aqueous clay dispersion in
ultrapure deionized water (25 mg in 10 mL)

| Clay | Cond. (mV) | pH |
|---|---|---|
| Ca RT | −77.1 | 8.44 |
| Ca Δ | −65.6 | 8.24 |
| Na RT | −190.3 | 10.43 |
| Na Δ | −190.2 | 10.43 |

TABLE 108
Conductivity of drug solutions in 0.1N HCl

| Sample | Cond. (mV) | pH |
|---|---|---|
| 0.1N HCl | 331.1 | 1.09 |
| 1000 μg/mL | 332.1 | 1.08 |
| 500 μg/mL | 333.7 | 1.05 |
| 250 μg/mL | 333.2 | 1.06 |
| 200 μg/mL | 333.5 | 1.05 |
| 100 μg/mL | 334.4 | 1.04 |

TABLE 109
Conductivity of clay in 0.1N HCl

| Clay | Cond. (mV) | pH |
|---|---|---|
| Ca Δ | 325.6 | 1.19 |
| Na Δ | 330.7 | 1.10 |

TABLE 110
Conductivity of different drug solutions
containing 25 mg clay in 0.1N HCl

| | |
|---|---|
| Ca Δ | Na Δ |

| | Cond. | | Cond. | |
|---|---|---|---|---|
| Conc. | (mV) | pH | (mV) | pH |
| 1000 μg/mL | 331.9 | 1.08 | 333.4 | 1.05 |
| 500 μg/mL | 332.8 | 1.06 | 335.4 | 1.02 |
| 250 μg/mL | 333.0 | 1.06 | 331.2 | 1.09 |
| 200 μg/mL | 332.7 | 1.07 | 330.2 | 1.11 |
| 100 μg/mL | 330.6 | 1.10 | 332.3 | 1.07 |

### Effect on Flocculation

Flocculation behavior of two bentonite samples was studied by adding 2 mg of PEO to 10 mL of the suspension containing 25 mg clay, as shown in Table 111.

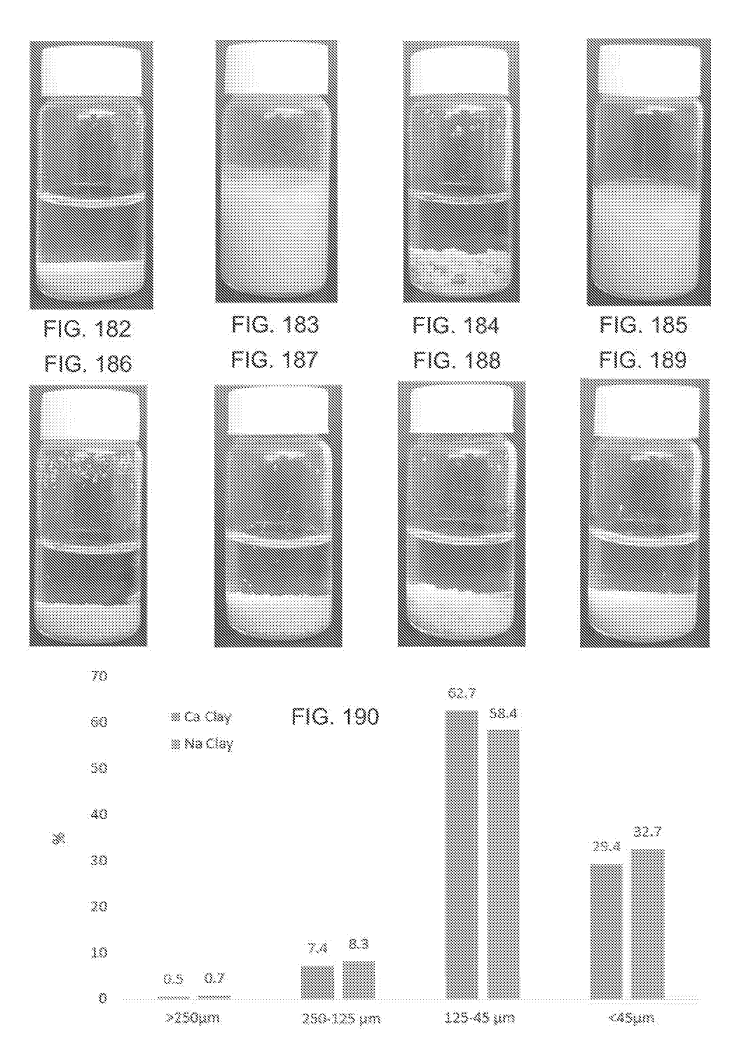

TABLE 111

| | 1000 μg/mL | | 100 μg/mL | |
|---|---|---|---|---|
| | Ca Clay | Na Clay | Ca Clay | Na Clay |
| In Water | FIG. 182 | FIG. 183 | FIG. 184 | FIG. 185 |
| In 0.1N HCl | FIG. 186 | FIG. 187 | FIG. 188 | FIG. 189 |

Particle Size of the Clays Used in this Study:

With reference to FIG. 190, a sieve analysis was carried out to determine particle size distribution of Calcium and Sodium bentonite samples. Sieves were stacked in ascending order of 60, 120, and 325. 1 g sample powder was added on the top sieve, number 60. After standard 60 taps for 1 min, particles retained on sieves were collected and weighed. Also, particles passed through sieve number 325 were collected and weighed.

TABLE 112
Drug binding and clay particle size (25
mg clay and drug solution of 1000 μg/mL)

% Drug binding

| Particle size | Ca Clay | Na Clay |
|---|---|---|
| 125μ-250μ | 27.70 | 45.52 |
| 45μ-125μ | 35.56 | 53.47 |
| Less than 45μ | 45.93 | 57.18 |

### Example 8: Croscarmellose Sodium (CCS)—Dextromethorphan HBR (DEX) Complex

200 mg of Croscarmellose sodium was added to 10 ml solution of 25 mg of Dextromethorphan HBr in water. Mixture was thoroughly mixed by vortexing for 30 seconds, and centrifuged @1500 rpm for 5 minutes. Supernatant solution was separated from the complex and washed with deionized water three times. Washings were collected and drug concentration (UV absorption at 276 nm) was determined in both supernatant and washings, per Tables 113 and 114.

TABLE 113
Pure CCS

| Supernatant | Wash1 | Wash2 | Wash3 |
|---|---|---|---|
| 0.0117 | 0.0068 | 0.0005 | 0.0001 |
| 0.0117 | 0.0065 | 0.0002 | 0.0009 |
| 0.0117 | 0.0067 | 0.0001 | 0.0011 |

TABLE 114
CCS - DEX Complex

| Supernatant | Wash1 | Wash2 | Wash3 |
|---|---|---|---|
| 0.1848 | 0.0127 | 0.0012 | 0.0012 |
| 0.1842 | 0.0135 | 0.0006 | 0.0016 |
| 0.1843 | 0.0127 | 0.0016 | 0.0013 |

### Complex Yield:

In a 20 ml scintillating vial containing 10 ml of 0.1N HCl, 200 mg of the complex was added. Solution was vortexed and centrifuged@1500 rpm for 5 minutes. Supernatant solution was separated and drug concentration was determined at λₘₐₓ of 276 nm, per Table 115.

TABLE 115

| | | | | | Concentration | Amount |
|---|---|---|---|---|---|---|
| Trial | Abs 1 | Abs 2 | Abs 3 | Avg | (μg/mL) | (mg) |
| 1 | 0.4697 | 0.4695 | 0.4694 | 0.470 | 90.28 | 18.06 |
| 2 | 0.9377 | 0.9373 | 0.9373 | 0.937 | 180.26 | 18.03 |

This data shows that 200 mg of sodium croscarmellose can bind to about 18 mg of dextromethorphan HBr.

### Bulk Production of Complex:

10 g of Croscarmellose sodium was added to 500 ml drug solution (2.5 mg/ml). Complex prepared was centrifuged and separated. Prepared complex was washed under vacuum three times with deionized water and dried in a hot air oven @65° C. Dried complex was crushed into fine powder in a ball mill and passed through sieve #60(250 microns). Powdered complex was further used for preparation of tablets, and the physical blend was used as control.

TABLE 116
Preparation of tablets

| | Physical | |
|---|---|---|
| Ingredient | blend (mg) | Complex (mg) |
| Croscarmellose | 250 | NA |
| Dex | 25 | NA |
| Dex-Croscarmellose | | 277 |
| complex | | (equal to 25 mg of drug) |
| MCC (Avicel ®101) | 225 | 223 |

All ingredients were weighed and mixed in a glass mortar and pestle. 500 mg of mixture was transferred to carver press and compressed into a tablet @2000 lb load force. Prepared tablets were studied for their drug release profiles and extraction studies.

TABLE 117
Drug release studies

| Time, | blend in | blend in | complex in | complex in |
|---|---|---|---|---|
| min | water | 0.1N HCl | water | 0.1N HCl |
| 5 | 15.79 | 97.15 | 7.15 | 104.67 |
| 15 | 16.65 | 97.37 | 8.25 | 102.78 |
| 30 | 17.55 | 96.56 | 9.30 | 101.72 |
| 60 | 18.75 | 96.32 | 10.48 | 101.34 |
| 90 | 16.48 | 98.63 | 12.98 | 100.58 |
| 120 | 17.97 | 95.40 | 12.82 | 100.52 |
| 240 | 17.21 | 96.47 | 13.99 | 101.58 |
| 360 | 16.85 | 98.37 | 15.02 | 102.77 |

### Extraction Studies:

200 mg of the complex was added to different extraction solvents including 0.1N hydrochloric acid (0.1N HCl), pH3 solution, normal saline, 40% ethanol-water solution (40% EtOH), 60% ethanol-water solution (60% EtOH), and pure ethanol (100% EtOH). The solution was vortex mixed and left standing for 15 min to reach equilibrium extraction. A physical blend of CCS and DEX (8:1 weight ratio similar to the complex) was also subjected to extraction in above solvents. Both the complex and physical blend solutions were centrifuged @1500 rpm for 5 min, and the supernatants were analyzed by UV spectrophotometry @276 nm to determine the amount of drug extracted into the solvents. 1 mL of the supernatant was diluted 10 times in the corresponding solvent. Table 118 shows the amount of DEX extracted and % extraction in different solvents for physical blend and complex.

TABLE 118

| | |
|---|---|
| Physical blend | Complex |

| | Drug, | % | Drug, | % |
|---|---|---|---|---|
| | mg | extracted | mg | extracted |
| Water | 2.54 | 10.2 | 0.81 | 3.25 |
| pH3 solution | 2.92 | 11.6 | 0.72 | 2.8 |
| Normal Saline | 6.44 | 25.7 | 5.29 | 21.1 |
| 40% EtOH | 6.22 | 24.8 | 0.7 | 2.8 |
| 60% EtOH | 14.39 | 57.5 | 1.5 | 6.3 |
| 100% EtOH | 23.2 | 92.8 | 0.008 | 0.03 |
| 0.1N HCl | 22.04 | 88.1 | 25.03 | 100.1 |

### Complex Yield:

The following experiments show that the yield of complexation increases as drug concentration in solution increases.

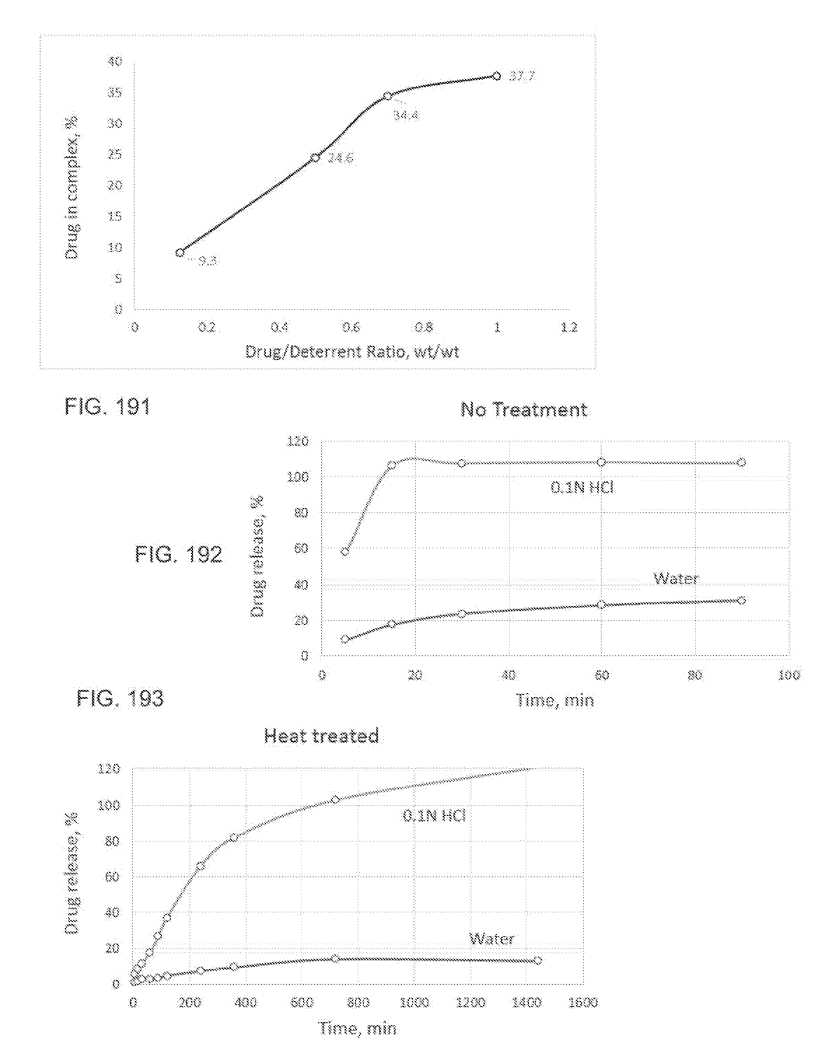

Croscarmellose—DEX Complexes were prepared at ratios of 8:1 (200 mg of CCS and 25 mg DEX), 2:1 (200 mg CCS and 100 mg DEX), 1.4:1 (200 mg CCS and 140 mg DEX), and 1:1 (140 mg CCS and 140 mg DEX). Complexes were washed with DI water three times and dried at 65° C. for 4 hours. The dried complexes were crushed into fine particles using ball mill and passed through 60 # sieve. To compare complexation or entrapment efficiency of these complexes, a fixed weight of the complex was added to 0.1N HCl. For 8:1, 2:1, and 1.4:1 ratios, 200 mg of complex was added to 10 mL of 0.1N HCl and 150 mg of complex was added to 10 mL of 0.1 N HCl for the 1:1 ratio. The solution was vortex mixed and left standing for 15 min to reach equilibrium extraction. After 15 min, solutions were centrifuged @1500 rpm for 5 min and supernatants were collected and analyzed by UV spectrophotometer @276 nm. Table 119 and FIG. 191 illustrate an amount of drug recovered after extraction in 0.1N HCl.

TABLE 119

| Drug/deterrent | Drug recovered, | Drug in complex |
|---|---|---|
| ratio, w/w | mg | (wt/wt %) |
| 0.125 | 18.53 | 9.26 |
| 0.5 | 49.15 | 24.57 |
| 0.7 | 68.85 | 34.42 |
| 1.0 | 56.59 | 37.72 |

### Multiple Administration of Deterrent-Drug Blend and Complex

Blend: 500 mg of croscarmellose sodium and 25 mg of DEX were weighed and added to 300 ml of different solvents (water, pH3 and 0.1N HCl). Samples were pulled at different time points (5, 15 and 30 min) and their drug concentrations were determined using UV. Similar procedure was repeated for 2500 mg of croscarmellose sodium and 125 mg of drug (5 times the initial amounts).

Complex: Complex equivalent to 125 mg (five doses) of DEX was weighed and added to 300 ml of different solvents including water, pH3 solution, and 0.1 N HCl, under magnetic stirring. Samples were pulled at 5, 15 and 30 min form each solvent, and then analyzed for drug content using UV spectrophotometer @ 276 nm. Percentage of drug released was calculated using standard curve equations in each solvent, as shown in Table 120.

TABLE 120

| | Blend | | | Complex | | |
|---|---|---|---|---|---|---|
| | % Drug release | | | | | |
| | 5 min | 15 min | 30 min | 5 min | 15 min | 30 min |
| Water | 10.6 | 11.1 | 10.8 | 1.4 | 2.2 | 1.6 |
| pH 3 | 11.7 | 11.7 | 14.3 | 7.9 | 7.7 | 8.9 |
| 0.1N HCl | 88.8 | 87.9 | 86.7 | 86.5 | 87.0 | 89.1 |

### Crush Resistant Sustained Release Abuse-Deterrent Formulation Containing Complex

TABLE 121
Formulation

| Ingredient | Amount (mg) |
|---|---|
| Kollidon ® SR | 250 |
| Complex (equivalent | 250 |
| to 22.8 mg DEX) | |
| Heat treatment | 90° C. for 1 hr |

All ingredients were weighed and mixed in a glass mortar and pestle. 500 mg of mixture was transferred to carver press and compressed into a tablet @2000 lb load force. Prepared tablets were studied for their drug release profiles and crush resistance studies, as shown in Table 122 and FIGS. 192-193.

TABLE 122
Drug release studies

| Time, | Regular in | Regular in | HT in | HT in |
|---|---|---|---|---|
| min | water | 0.1N HCl | water | 0.1N HCl |
| 5 | 9.01 | 58.00 | 1.20 | 5.32 |
| 15 | 17.50 | 106.22 | 1.80 | 8.34 |
| 30 | 23.48 | 107.55 | 2.59 | 11.19 |
| 60 | 28.27 | 108.24 | 2.67 | 17.31 |
| 90 | 30.83 | 107.88 | 3.28 | 26.31 |
| 120 | | | 4.59 | 36.80 |
| 240 | | | 7.26 | 65.78 |
| 360 | | | 9.39 | 81.72 |
| 720 | | | 13.89 | 102.46 |
| 1440 | | | 12.97 | 120.93 |

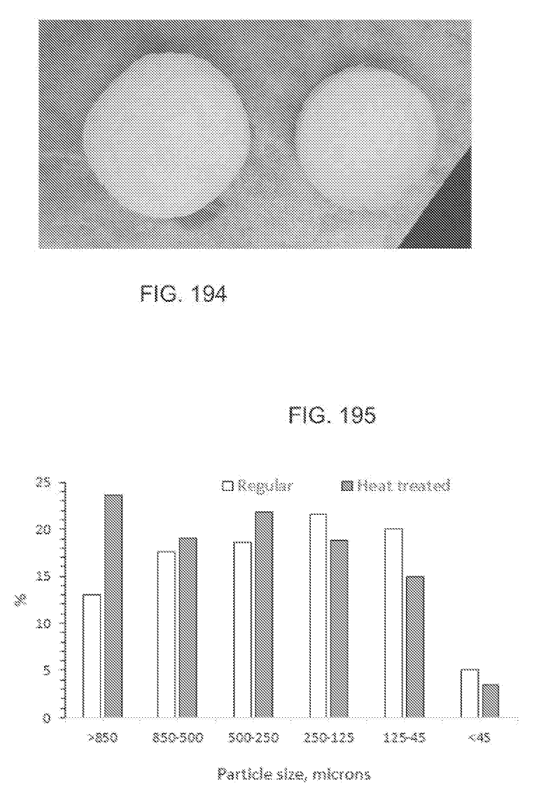

In FIG. 194, from left to right, are heat treated tablets in water and 0.1N HCl, after 24 hrs. Table 123 and FIG. 195 illustrate results of crush resistance studies using a high shear grinder.

TABLE 123

| particle size, μm | Regular | Heat treated |
|---|---|---|
| >850 | 13.00 | 23.59 |
| 850-500 | 17.60 | 19.05 |
| 500-250 | 18.60 | 21.86 |
| 250-125 | 21.60 | 18.83 |
| 125-45 | 20.00 | 14.94 |
| <45 | 5.00 | 3.46 |

### Extraction Study:

Table 124 shows that 22.8 mg (100% added as complex) is being released in 0.1N HCl.

TABLE 124

| | | | | | | |
|---|---|---|---|---|---|---|
| control | 0.0088 | 0.0089 | 0.0089 | 0.008867 | 1.685897 | 0.34 |
| Trial 1 | 0.5851 | 0.5852 | 0.5852 | 0.585167 | 112.5128 | 22.5 |
| Trial 2 | 0.6038 | 0.6038 | 0.6039 | 0.603833 | 116.1026 | 23.2 |
| Trial 3 | 0.5905 | 0.59 | 0.59 | 0.590167 | 113.4744 | 22.7 |

| | |
|---|---|
| Average | 22.8 |

In conclusion, the invention exemplifies a variety of compositions useful for inhibiting and/or preventing the isolation and concentration of drug constituents for misuse. These compositions are anticipated to reduce the incidence of tampering and abuse of pharmaceutical products and alcohol worldwide.

All patents and publications mentioned in this specification are indicative of the levels of those skilled in the art to which the disclosure pertains. All patents and publications are herein incorporated by reference to the same extent as if each individual publication was specifically and individually indicated to be incorporated by reference. It is to be understood that while a certain form of the disclosure is illustrated, it is not intended to be limited to the specific form or arrangement herein described and shown. It will be apparent to those skilled in the art that various changes may be made without departing from the scope of the disclosure and the disclosure is not to be considered limited to what is shown and described in the specification. One skilled in the art will readily appreciate that the present disclosure is well adapted to carry out the objectives and obtain the ends and advantages mentioned, as well as those inherent therein. The compositions, pharmaceutical compositions, methods, procedures, and techniques described herein are presently representative of the preferred embodiments, are intended to be exemplary and are not intended as limitations on the scope. Changes therein and other uses will occur to those skilled in the art which are encompassed within the disclosure. Although the disclosure has been described in connection with specific, preferred embodiments, it should be understood that the disclosure as ultimately claimed should not be unduly limited to such specific embodiments. Indeed various modifications of the described modes for carrying out the disclosure which are obvious to those skilled in the art are intended to be within the scope of the disclosure.

In addition, unless mention was made above to the contrary, it should be noted that all of the accompanying drawings are not to scale. There are many different features to the present invention and it is contemplated that these features may be used together or separately. Thus, the disclosure should not be limited to any particular combination of features or to a particular application of the disclosure. Further, it should be understood that variations and modifications might occur to those skilled in the art to which the disclosure pertains. Accordingly, all expedient modifications readily attainable by one versed in the art from the disclosure set forth herein are to be included as further embodiments of the present invention.

### REFERENCES

1. H Omidian, Y Qiu, D J Kim, S C Yang, H Park, K Park; Hydrogels Having Enhanced Elasticity and Mechanical Strength Properties, U.S. Pat. No. 6,960,617 (Issued Nov. 1, 2005)
2. H Omidian, J G Rocca; Formation of Strong Superporous Hydrogels, U.S. Pat. No. 7,056,957 (Issued Jun. 6, 2006)
3. H Omidian, D Mastropietro: Fighting a New Drug Epidemic, Journal of Developing Drugs, 2(1): Editorial note, e120, 2013
4. H Omidian, D Mastropietro; Abuse-Deterrent Pharmaceutical Compositions, Provisional U.S. Patent Application 61/875,173 (Filed Sep. 9, 2013)
5. H Omidian, D Mastropietro; Abuse Deterrents in Pharmaceutical Compositions, Provisional U.S. Patent Application 61/918,870 (Filed Dec. 20, 2013)
6. H Omidian, D Mastropietro; A Therapeutic Composition for Alcohol Cessation and Abuse, Provisional U.S. Patent Application 61/918,879 (Filed Dec. 20, 2013)
7. H Omidian, D Mastropietro; Powerful Deterrent Agents for Abusable Medications, Provisional U.S. Patent Application 61/918,880 (Filed Dec. 20, 2013)
8. D Mastropietro, H Omidian; An Aversive Super-Deterrent Agent for Abusable Medications, Provisional U.S. Patent Application 61/919,443 (Filed Dec. 20, 2013):
9. Muppalaneni, S. and H. Omidian, Polyvinyl Alcohol in Medicine and Pharmacy. Journal of Developing Drugs, 2013. 2(3): p. 1000112.
10. http://www.emdmillipore.com/ visited Sep. 29, 2013.
11. http://www.sekisui-sc.com/ visited Sep. 29, 2013.
12. Lozinsky, V. I. and F. M. Plieva, Poly(vinyl alcohol) cryogels employed as matrices for cell immobilization. 3. Overview of recent research and developments. Enzyme and Microbial Technology, 1998. 23: p. 227-242.
13. Damshkaln, L. G., I. A. Simenel, and V. I. Lozinsky, Study of cryostructurization of polymer systems. Xv. Freeze-thaw-induced formation of cryoprecipitate matter from low-concentratetd aqueous solutions of poly(vinyl alcohol). Journal of Applied Polymer Science, 1999. 74: p. 1978-1986.
14. Lozinsky, V. I. and L. G. Damshkaln, Study of cryostructurization of polymer systems. Xvii. Poly(vinyl alcohol) cryogels: Dynamics of the cryotropic gel formation. Journal of Applied Polymer Science, 2000. 77: p. 2017-2023.
15. Hassan, C. M. and N. A. Peppas, Cellular pva hydrogels produced by freeze/thawing. Journal of Applied Polymer Science, 2000. 76: p. 2075-2079.
16. Hassan, C. M. and N. A. Peppas, Structure and applications ofpoly(vinyl alcohol) hydrogels produced by conventional crosslinking or by freezing/thawing methods. Advances in Polymer Science 2000. 153: p. 37-65.
17. Hassan, C. M. and N. A. Peppas, Structure and morphology of freeze/thawed pva hydrogels. Macromolecules, 2000. 33: p. 2472-2479.
18. Lozinsky, V. I. and L. G. Damshkaln, Cryotropic gelation of polyvinyl alcohol) solutions. Uspekhi Khimi, 1998. 67: p. 641-655.
19. Lozinsky, V. I., A. L. Zubov, and E. F. Titova, Swelling behavior ofpoly(vinyl alcohol) cryogels employed as matrices for cell immobilization. Enzyme and Microbial Technology 1996. 18: p. 561-569.
20. Lozinsky, V. I., et al., Study of cryostructurization of polymer systems. 9. Poly(vinyl alcohol) cryogels filled with particles of crosslinked dextran gel. Journal of Applied Polymer Science, 1992. 44: p. 1423-1435.
21. Lozinsky, V. I., et al., Study of cryostructurization of polymer systems. 11. The formation of pva cryogels by freezing-thawing the polymer aqueous solutions containing additives of some polyols. Journal of Applied Polymer Science, 1995. 58: p. 171-177.
22. Lozinsky, V. I., et al., Study of cryostructurization of polymer systems. Xiv. Poly(vinyl alcohol) cryogels: Apparent yield of the freeze-thaw-induced gelation of concentrated aqueous solutions of the polymer. Journal of Applied Polymer Science, 2000. 77: p. 1822-1831.
23. Lozinsky, V. I., et al., Study of cryostructurization of polymer systems 0.12. Poly(vinyl alcohol) cryogels: Influence of low-molecular electrolytes. Journal of Applied Polymer Science, 1996. 61: p. 1991-1998.
24. Hassan, C. M., J. E. Stewart, and P. N. A., Diffusional characteristics of freeze/thawed poly(vinyl alcohol) hydrogels: Applications to protein controlled release from multilaminate devices. European Journal of Pharmaceutics and Biopharmaceutics, 2000. 49: p. 161-165
25. Omidian, H., S. Muppalaneni, and D. Mastropietro, A Crush Resistant Vehicle for Abuse-Deterrent Compositions. Disclosure Disclosure NSU 14/02: Filed with the NSU's Office of Reserach and Technology Transfer 2014.
26. Omidian, H. and S. Muppalaneni, Multifunction Deterrents For Abusable Medications. Disclosure Disclosure NSU 14/01: Filed with the NSU's Office of Research and Technology Transfer, 2014.
27. http://msdssearch.dow.com/PublishedLiteratureDOWCOM/dh_0031/0901b80380031a4a.pdf?filepath=/326-00001.pdf&fromPage=GetDoc.
28. Mastropietro, D., et al., Shear-Thinning Rheology of the Abuse-Deterrent Dosage Form Extracts. Journal of Developing Drugs, 2013. 2(3): p. 114.
29. Omidian, H. and S. Muppalaneni, A Deterrent Cage For Immediate and Sustained Release Abuse-Deterrent Medications; Disclosure Disclosure NSU 14/05; Filed with the NSU's Office of Research and Technology Transfer. 2014
30. Baum C, Hsu J P, Nelson R C. The Impact of the Addition of Naloxone on the Use and Abuse of Pentazocine. Public Health Rep. 1987; 102(4):426-9.
31. Strain E C, Harrison J A, Bigelow G E. Induction of opioid-dependent individuals onto buprenorphine and buprenorphine/naloxone soluble-films. Clin Pharmacol Ther. 2011; 89(3):443-9.
32. Farrell J J, inventorTamper-proof narcotic delivery system patent U.S. Pat. No. 7,968,119. 2011.
33. Palermo P J, Colucci R D, Kaiko R F, inventors; Euro-Celtique S. A, assignee. Method of preventing abuse of opioid dosage forms patent U.S. Pat. No. 6,228,863. 2001 May 8.
34. Oshlack B, Wright C, Haddox J D, inventors; Euro-Celtique, S. A., assignee. Tamper-resistant oral opioid agonist formulations patent U.S. Pat. No. 6,696,088. 2004.
35. Oshlack B, Wright C, Haddox J D, inventorsTamper-resistant oral opioid agonist formulations patent U.S. Pat. No. 7,658,939. 2010 Feb. 9.
36. Breder C, Wright C, Oshlack B, inventors; Purdue Pharma L. P., assignee. Opioid agonist formulations with releasable and sequestered antagonist patent U.S. Pat. No. 7,914,818. 2011 Mar. 29.
37. Shaw I F, Berk J, inventors; West Laboratories, Inc, assignee. Orally administered drug composition for therapy in the treatment of narcotic drug addition patent U.S. Pat. No. 3,980,766. 1976 Sep. 14.
38. Hoffmeister F, Hiltmann R, Wollweber H, Kramer H, inventors; Bayer Aktiengesellschaft, assignee. Enteral Pharmaceutical Compositions patent U.S. Pat. No. 4,070,494. 1978 Jan. 24.
39. Bastin R J, Lithgow B H, inventors; Aventis Pharma Limited, assignee. Abuse resistant tablets patent U.S. Pat. No. 6,309,668. 2001 Oct. 30.
40. Kumar V, Dixon D, Tewari D, Wadgaonkar D B, inventors; Acura Pharmaceuticals, Inc, assignee. Methods and compositions for deterring abuse of opioid containing dosage forms patent U.S. Pat. No. 7,201,920. 2007.
41. Kumar V, Dixon D, Tewari D, Wadgaonkar D B, inventors; Acura Pharmaceuticals, Inc, assignee. Methods and compositions for deterring abuse of opioid containing dosage forms patent U.S. Pat. No. 7,476,402. 2009.
42. Kumar V, Dixon D, Tewari D, Wadgaonkar D B, inventors; Acura Pharmaceuticals, Inc, assignee. Methods and compositions for deterring abuse of opioid containing dosage forms. patent U.S. Pat. No. 7,510,726. 2009 Mar. 31.
43. Porter G, inventor Composition and method to prevent accidental and intentional overdosage with psychoactive drugs patent U.S. Pat. No. 4,175,119. 1979.
44. Porter G, inventor; Clear Lake Development Group, assignee. Composition and method of immobilizing emetics and method of treating human beings with emetics patent U.S. Pat. No. 4,459,278. 1984.
45. Ruan X, Chen T, Gudin J, Couch J P, Chiravuri S. Acute opioid withdrawal precipitated by ingestion of crushed embeda (morphine extended release with sequestered naltrexone): case report and the focused review of the literature. Journal of opioid management. 2010; 6(4):300-3.
46. Passik S D. Issues in Long-term Opioid Therapy: Unmet Needs, Risks, and Solutions. Mayo Clin Proc. 2009; 84(7):593-601.
47. Mastropietro D J, Omidian H. Current approaches in tamper-resistant and abuse-deterrent formulations. Drug Dev Ind Pharm. 2013; 39(5):611-24.
48. Mastropietro D J, Omidian H. Commercial Abuse-Deterrent Dosage Forms: Clinical Status. J Develop Drugs. 2013; 2(103):1000103.
49. Passik S D, Hays L, Eisner N, Kirsh K L. Psychiatric and pain characteristics of prescription drug abusers entering drug rehabilitation. J Pain Palliat Care Pharmacother. 2006; 20(2):5-13.
50. McCabe S E, Cranford J A, Morales M, Young A. Simultaneous and concurrent polydrug use of alcohol and prescription drugs: prevalence, correlates, and consequences. J Stud Alcohol. 2006; 67(4):529-37.

What is claimed is:

1. A therapeutic pharmaceutical composition for deterring abuse of drugs or alcohol, the composition comprising:
a. at least one pharmaceutically-active ingredient known to be abused;
b. a multifunction polymer capable of:
i. increasing viscosity of water, water alcohol mixtures, and saline to a level that a 2.5 wt % solution cannot be filtered using filtration methods;
ii. enhancing viscosity of water, water alcohol mixtures, and saline up to maximum extent over a time period of a few minutes;
iii. binding with the pharmaceutically-active ingredient in water and water-alcohol mixtures;
iv. releasing bound pharmaceutically-active ingredient in 0.1N hydrochloric acid (HCl) solution; and
v. providing different release profiles in water and 0.1N HCl solutions;
c. a multifunction gel enhancer capable of:
i. changing a viscous solution of the multifunction polymer into a gel mass through strong physical binding with the multifunction polymer to the level that the gel mass cannot be filtered or drawn into a syringe; and
ii. binding with the pharmaceutically-active ingredient; and
d. a multifunction filler capable of:
i. binding with the pharmaceutically-active ingredient in aqueous solutions and losing binding capacity in 0.1N HCl; and
ii. functioning as a pharmaceutically-acceptable filler.

2. The therapeutic pharmaceutical composition of claim 1, further comprising at least one pharmaceutically-acceptable excipient for preparing a dosage form.

3. The therapeutic pharmaceutical composition of claim 2, wherein the dosage form is a tablet.

4. The therapeutic pharmaceutical composition of claim 1, wherein the pharmaceutically-active ingredient is for treating at least one of anxiety, depression, sleep disorders, pain, lack of energy, attention deficit, cough, and cold.

5. The therapeutic pharmaceutical composition of claim 1, wherein the pharmaceutically-active ingredient is a barbiturate.

6. The therapeutic pharmaceutical composition of claim 5, wherein the barbiturate is at least one of a phenobarbital, a benzodiazepine, codeine, morphine, oxycodone, oxymorphone, hydrocodone, hydromorphone, tramadol, an amphetamine, a methyl phenidate, dextromethorphan, and pseudoephedrine.

7. The therapeutic pharmaceutical composition of claim 1, wherein the pharmaceutically-active ingredient is an abusable drug in a form of a weak base as a salt of organic and inorganic acids.

8. The therapeutic pharmaceutical composition of claim 7, wherein the organic and inorganic acids are hydrochloric acid, hydrosulfuric acid, hydrophosphoric acid, or tartaric acid.

9. The therapeutic pharmaceutical composition of claim 7, wherein the multifunction polymer comprises binding sites for forming an effective and stable complex with the weak base.

10. The therapeutic pharmaceutical composition of claim 1, wherein the multifunction polymer is selected from synthetic or natural polymers carrying anionic groups including carboxymethylcellulose, carboxymethyl starch, and carboxymethyl chitosan.

11. The therapeutic pharmaceutical composition of claim 10, wherein the polymer is carboxymethylcellulose or pre-hydrated carboxymethylcellulose.

12. The therapeutic pharmaceutical composition of claim 1, wherein the composition comprises 1-99% of the multifunction polymer.

13. The therapeutic pharmaceutical composition of claim 1, wherein the multifunction polymer is soluble in water and hydro-alcoholic solutions and precipitates in pH 1 acidic solution.

14. The therapeutic pharmaceutical composition of claim 1, wherein the 2.5 wt % solution of the multifunction polymer can generate a gel with a strength of greater than 50 mN in water, saline, ethanol (EtOH) 20%, and EtOH 40% at room temperature.

15. The therapeutic pharmaceutical composition of claim 1, wherein the multifunction gel enhancer is a salt that releases 1, 2, 3 valence cations or combinations thereof in solution.

16. The therapeutic pharmaceutical composition of claim 1, wherein the multifunction gel enhancer is potassium chloride, calcium chloride, aluminum chloride, iron chloride, aluminum hydroxide, calcium citrate, calcium diphosphate, or zinc acetate.

17. The therapeutic pharmaceutical composition of claim 1, wherein a ratio of the multifunction gel enhancer to multifunction polymer is 1-50 wt %.

18. The therapeutic pharmaceutical composition of claim 17, wherein the ratio of the multifunction gel enhancer to multifunction polymer is 10-20 wt %.

19. The therapeutic pharmaceutical composition of claim 1, wherein the multifunction filler is talc.

20. The therapeutic pharmaceutical composition of claim 19, wherein the composition comprises 1-99% talc.

21. The therapeutic pharmaceutical composition of claim 3, wherein the tablet is a single layer tablet, a multi-layer tablet, or a coated tablet.

22. The therapeutic pharmaceutical composition of claim 3, wherein the multifunction polymer, the multifunction gel enhancer, and the multifunction filler are mixed together in a dry state and compressed into a tablet.

23. The therapeutic pharmaceutical composition of claim 3, wherein the pharmaceutically-active ingredient is mixed with aqueous or hydro-alcoholic solutions of the multifunction polymer, dried out to form a stable complex, and compressed into a tablet along with pharmaceutically-acceptable excipients.

24. The therapeutic pharmaceutical composition of claim 2, wherein the multifunction gel enhancer is coated before mixing with the pharmaceutically-active ingredient and the pharmaceutically-acceptable excipients.

25. The therapeutic pharmaceutical composition of claim 1, further comprising a mixture of two or more multifunction gel enhancers for controlling gel strength and release profile.

26. The therapeutic pharmaceutical composition of claim 1, wherein release of the pharmaceutically-active ingredient is sustained or extended.

27. A therapeutic pharmaceutical composition for deterring abuse of drugs or alcohol, the composition comprising:
a. one or more pharmaceutically-active ingredients known to be abused;
b. a cryogel having;
i. a group of vinyl alcohol-based copolymers with vinyl acetate, vinyl amine, vinyl pyrrolidone, or ethylene glycol;
ii. adhesive force in a range of 0-300 mN;
iii. adhesiveness in a range of 0-0.2mJ;
iv. gumminess in a range of 0-20N;
v. hardness in a range of 0-20mJ; and
vi. water absorption in an amount of 0-100 wt %;
c. and; at least one deterrent agent, the deterrent agent selected from a group of natural-based or synthetic-based polyacid polymers containing free carboxyl groups.

28. The therapeutic pharmaceutical composition of claim 27, wherein the vinyl alcohol-based copolymers contain over 99% vinyl alcohol.

29. The therapeutic pharmaceutical composition of claim 27, wherein the range of the adhesive force is 100-200 mN.

30. The therapeutic pharmaceutical composition of claim 29, wherein the range of the adhesive force is 100-160 mN.

31. The therapeutic pharmaceutical composition of claim 27, wherein the range of adhesiveness is 0.02-0.15 mJ.

32. The therapeutic pharmaceutical composition of claim 31, wherein the range of adhesiveness is 0.07-0.12 mJ.

33. The therapeutic pharmaceutical composition of claim 27, wherein the range of gumminess is 4-15 N.

34. The therapeutic pharmaceutical composition of claim 33, wherein the range of gumminess is 8-14 N.

35. The therapeutic pharmaceutical composition of claim 27, wherein the range of hardness is 8-18 mJ.

36. The therapeutic pharmaceutical composition of claim 35, wherein the range of hardness is 10-14 mJ.

37. The therapeutic pharmaceutical composition of claim 27, wherein the water absorption is in the range of 5-50 wt %.

38. The therapeutic pharmaceutical composition of claim 37, wherein the water absorption is in the range of 10-30 wt %.

39. The therapeutic pharmaceutical composition of claim 27, wherein the natural-based polyacid deterrent agent is one of internally crosslinked carboxymethyl cellulose, internally crosslinked carboxymethyl starch, alginic acid, and xanthan gum.

40. The therapeutic pharmaceutical composition of claim 27, wherein the synthetic-based polyacid deterrent agent is one of crosslinked polymers or copolymers of acrylic acid and salts thereof, methacrylic acid and salts thereof sulfopropyl acrylic acid and salts thereof, 2-acrylamido 2-methyl 1-propane sulfonic acid and salts thereof.

41. The therapeutic pharmaceutical composition of claim 27, further comprising at least one pharmaceutically-acceptable excipient for preparing a dosage form.

42. The therapeutic pharmaceutical composition of claim 41, wherein the dosage form is a tablet, a capsule, or a transdermal patch.

43. The therapeutic pharmaceutical composition of claim 27, wherein the pharmaceutically-active ingredient is for treating at least one of anxiety, depression, sleep disorders, pain, lack of energy, attention deficit, cough, and cold.

44. The therapeutic pharmaceutical composition of claim 27, wherein the pharmaceutically-active ingredient is a barbiturate.

45. The therapeutic pharmaceutical composition of claim 44, wherein the barbiturate is at least one of a phenobarbital, a benzodiazepine, codeine, morphine, oxycodone, oxymorphone, hydrocodone, hydromorphone, tramadol, an amphetamine, a methyl phenidate, dextromethorphan, and pseudoephedrine.

46. The therapeutic pharmaceutical composition of claim 27, wherein the pharmaceutically-active ingredient is an abusable drug in a form of a weak base and a salt of organic and inorganic acids.

47. The therapeutic pharmaceutical composition of claim 46, wherein the organic and inorganic acids are hydrochloric acid, hydrosulfuric acid, hydrophosphoric acid, or tartaric acid.

48. The therapeutic pharmaceutical composition of claim 27, comprising an abusable drug as the pharmaceutically-active ingredient and an aqueous polyvinyl alcohol solution which undergoes one or two cycles of freezing at below 0° C. and thawing at above 0° C.

49. The therapeutic pharmaceutical composition of claim 48, wherein the polyvinyl alcohol is at 5 wt %.

50. The therapeutic pharmaceutical composition of claim 27, wherein an amount of the pharmaceutically-active ingredient in the cryogel is 0.1-50 wt %

51. The therapeutic pharmaceutical composition of claim 50, wherein the amount of the pharmaceutically-active ingredient in the cryogel is 0.1-20 wt %

52. The therapeutic pharmaceutical composition of claim 51, wherein the amount of the pharmaceutically-active ingredient in the cryogel is 0.1-5 wt %

53. The therapeutic pharmaceutical composition of claim 27, comprising an abusable drug as the pharmaceutically-active ingredient and a polyvinyl alcohol cryogel having a smallest dimension in a range of 0-8 mm.

54. The therapeutic pharmaceutical composition of claim 53, wherein the range is 1-5 mm.

55. The therapeutic pharmaceutical composition of claim 54, wherein the range is 1.5-3 mm.

56. The therapeutic pharmaceutical composition of claim 53, wherein an amount of drug released in water measured by USP 2 method is: 0-50% after 5 min; 50-100% after 15 min; and 70-100% after 45 min.

57. The therapeutic pharmaceutical composition of claim 56, wherein an amount of drug released in water measured by USP 2 method is: 35-45% after 5 min; 70-90% after 15 min; and 85-95% after 45 min.

58. The therapeutic pharmaceutical composition of claim 53, wherein an amount of drug released in 0.1 N HCL measured by USP 2 method is: 0-50% after 5 min; 50-100% after 15 min; and 70-100% after 45 min.

59. The therapeutic pharmaceutical composition of claim 58, wherein an amount of drug released in 0.1 N HCL measured by USP 2 method is: 30-40% after 5 min; 60-70% after 15 min; and 80-90% after 45 min.

60. The therapeutic pharmaceutical composition of claim 27, further comprising an internally crosslinked sodium carboxymethyl cellulose as an abuse-deterrent agent.

61. The therapeutic pharmaceutical composition of claim 60, wherein an amount of the abuse-deterrent agent in the cyrogel composition is from 1-30 wt %.

62. The therapeutic pharmaceutical composition of claim 61, wherein an amount of the abuse-deterrent agent in the cyrogel composition is from 5-15 wt %.

63. The therapeutic pharmaceutical composition of claim 62, wherein an amount of the abuse-deterrent agent in the cyrogel composition is from 8-12 wt %.

64. The therapeutic pharmaceutical composition of claim 61, comprising 5 wt % of polyvinyl alcohol (PVOH) and 10 wt % internally crosslinked carboxymethyl cellulose, wherein after 5 minutes in a dissolution medium, absorbs 50-60% water and 20-30% of an ethanol solution 40 v/v %.

65. The therapeutic pharmaceutical composition of claim 64, wherein in a fully-swollen state in water and in 0.1 N HCL has a hardness of 5-10 N.

66. The therapeutic pharmaceutical composition of claim 65, wherein the hardness is 7-8 N in water and 8-9 N in 0.1 N HCL.

67. The therapeutic pharmaceutical composition of claim 65, wherein the composition releases content of the pharmaceutically-active ingredient up to a maximum of 30-50% in water and at least 70% in 0.1 N HCL.

68. The therapeutic pharmaceutical composition of claim 67, wherein a release profile, measured by USP 2 using 900 ml of 0.1N HCl, a stirring rate of 50 rpm, and at 37 C.° is: after 5 min (5-8%), after 15 min (22-33%), after 30 min (32-47%), after 45 min (37-56%), after 60 min (42-64%), after 90 min (50-75%), after 120 min (54-80%), after 180 min (61-92%), after 360 min (69-103%), and after 720 min (73-109%).

69. The therapeutic pharmaceutical composition of claim 27, wherein the composition is manufactured by continuous or batch operations.

70. The therapeutic pharmaceutical composition of claim 69, wherein the continuous or batch operations are extrusion or molding.

71. The therapeutic pharmaceutical composition of claim 69, wherein the composition is manufactured in a form of continuous slab, a continuous film, a continuous sheet, a molded article, granules, or pellets.

72. The therapeutic pharmaceutical composition of claim 27, wherein the composition is encapsulated in an orally-administrable capsule including hydroxypropyl methylcellulose or gelatin.

73. The therapeutic pharmaceutical composition of claim 27, wherein the cryogel is coated with a pharmaceutically-acceptable coating or wax.

74. The therapeutic pharmaceutical composition of claim 27, wherein the pharmaceutically-active agent is bound to the deterrent and the bound deterrent agent is then mixed into polyvinyl alcohol (PVOH) to form the cryogel.

75. The therapeutic pharmaceutical composition of claim 27, wherein the pharmaceutically-acceptable agent is added as a solid powder, a solution, or as a dispersion to form the cryogel.

76. The therapeutic pharmaceutical composition of claim 27, further comprising at least one of preservatives, surfactants, plasticizers, solvents, air, and pore-forming agents.

77. The therapeutic pharmaceutical composition of claim 27, wherein the cryogel is a multi-layer cryogel, in which all layers have a same adhesiveness, adhesive force, gumminess, hardness, and swelling and in which each layer includes one or more different pharmaceutically-active agent.

78. The therapeutic pharmaceutical composition of claim 27, wherein the cryogel is a multi-layer cryogel, in which all layers have a different adhesiveness, adhesive force, gumminess, hardness, and swelling and in which each layer includes same or different pharmaceutically-active agents.

79. The therapeutic pharmaceutical composition of claim 27, wherein the composition has an immediate release profile and a controlled release profile for release of the pharmaceutically-active agent.

80. A therapeutic pharmaceutical composition for deterring abuse of drugs or alcohol, the composition comprising:
a. at least one pharmaceutically-active ingredient, the pharmaceutically-active ingredient:
i. is known to be abusable;
ii. is in a form of weak base as a salt of organic and inorganic acids including hydrochloric acid, hydrosulfuric acid, hydrophosphoric acid, and tartaric acid;
iii. is for treating at least one of anxiety, depression, sleep disorders, pain, lack of energy, attention deficit, cough, and cold; and
iv. is a barbiturate selected from at least one of phenobarbitals, benzodiazepines, codeine, morphine, oxycodone, oxymorphone, hydrocodone, hydromorphone, tramadol, amphetamines, methyl phenidate, dextromethorphan, and pseudoephedrine;
b. and, a deterrent cage, the deterrent cage:
i. if heat treated changes a release profile of its pharmaceutically-active ingredient;
ii. is composed of three polymers;
A. a water-swellable polymer that:
a. expands in size when exposed to an aqueous medium;
b. effectively binds to the pharmaceutically-active ingredient;
c. has pH-sensitive binding to the pharmaceutically-active ingredient;
d. allows for complete drug release in 0.1N HCl solution if the deterrent cage is not heat treated, regardless of concentration;
e. increases an amount of pharmaceutically-active released in 0.1N HCl solution as its concentration increases when the deterrent cage is heat treated; and
f. hinders drug release in water as its concentration increases in both heated and non-heat treated deterrent cages;
B. a water-soluble hydrophilic polymer that rapidly dissolves in an aqueous medium;
C. a water-insoluble hydrophobic polymer that:
a. possesses a glass transition temperature in a range of 25−60° C.;
iii. when heated above the glass transition temperature of its water-insoluble hydrophobic polymer for a sufficient period of time, goes through a thermal transition, and binds particles of the water-swellable polymer;
iv. when first heated and then placed in an aqueous medium:
A. its water swellable polymer grows in size due to water absorption;
B. its water-soluble polymer is immediately or gradually dissolved;
C. its water insoluble polymer stays intact forming a sponge-shape structure after releasing part or all of its water swellable and water soluble components;
v. with pharmaceutically-active ingredient incorporated, when first heated and then placed in water or in 0.1N HCl solution, provides a sustained release profile of the pharmaceutically-active ingredient;
vi. with drug incorporated, if not heated and placed in water or in 0.1N HCl solution, provides an immediate release profile of the pharmaceutically-active ingredient;
vii. with drug incorporated, if not heated and processed in a ball mill for 5 minutes, in presence of 2 steel balls each 10 mm in diameter at a frequency of 25 Hz, generates over 80% fine particles smaller than 250 μm and less than 20% coarse particles larger than 250 μm; and
viii. with drug incorporated, if heat treated at 120° C. for 1 hour, and processed in a ball mill for 5 minutes, in the presence of 2 steel balls each 10 mm in diameter at a frequency of 25 Hz, stays almost intact or generates over 80% of particles larger than 250 μm and less than 20% particles smaller than 250 μm.

81. The therapeutic pharmaceutical composition of claim 80, wherein the glass transition temperature of the water-insoluble hydrophobic polymer is in a range of 27-40° C.

82. The therapeutic pharmaceutical composition of claim 80, further comprising at least one pharmaceutically-acceptable excipient for preparing a dosage form.

83. The therapeutic pharmaceutical composition of claim 82, wherein the dosage form is a tablet.

84. The therapeutic pharmaceutical composition of claim 80, wherein the water-swellable polymer of the deterrent cage is at least one of synthetic, natural, or semi-synthetic crosslinked polymers.

85. The therapeutic pharmaceutical composition of claim 84, wherein the synthetic, natural, or semi-synthetic crosslinked polymer is at least one of crosslinked carboxymethylcellulose, crosslinked carboxymethyl starch, crosslinked carboxymethyl chitosan, crosslinked acrylic (methacrylic) acid and salts thereof, crosslinked alginic acid and salts thereof, and crosslinked xanthan.

86. The therapeutic pharmaceutical composition of claim 85, wherein the synthetic, natural, or semi-synthetic crosslinked polymer is crosslinked carboxymethylcellulose.

87. The therapeutic pharmaceutical composition of claim 80, wherein the water soluble polymer of the deterrent cage is at least one of synthetic, natural, or semi-synthetic linear polymers.

88. The therapeutic pharmaceutical composition of claim 87, wherein the water soluble polymer of the deterrent cage is polyvinylpyrrolidone.

89. The therapeutic pharmaceutical composition of claim 80, wherein the water-insoluble polymer of the deterrent cage is at least one of low glass transition polymers having a glass transition temperature in a range of 25−60° C.;

90. The therapeutic pharmaceutical composition of claim 89, wherein the low glass transition polymer comprises vinyl acetate monomers.

91. The therapeutic pharmaceutical composition of claim 90, wherein the low glass transition polymer is a poly (vinyl acetate) homopolymer.

92. The therapeutic pharmaceutical composition of claim 80, wherein the water soluble and water insoluble components of the deterrent cage comprise a polymer blend of polyvinylpyrrolidone and poly(vinyl acetate).

93. The therapeutic pharmaceutical composition of claim 80, wherein a ratio of the water swellable polymer to a complete dosage form is 0-80%.

94. The therapeutic pharmaceutical composition of claim 93, wherein the ratio of the water swellable polymer to a complete dosage form is 10-50%.

95. The therapeutic pharmaceutical composition of claim 94, wherein the ratio of the water swellable polymer to a complete dosage form is 25-35%.

96. The therapeutic pharmaceutical composition of claim 80, wherein a ratio of the water soluble polymer to a complete dosage form is 0-80%.

97. The therapeutic pharmaceutical composition of claim 96, wherein the ratio of the water soluble polymer to a complete dosage form is 1-30%.

98. The therapeutic pharmaceutical composition of claim 74, wherein the ratio of the water swellable polymer to a complete dosage form is 5-15%.

99. The therapeutic pharmaceutical composition of claim 80, wherein a ratio of the water insoluble hydrophobic polymer to a complete dosage form is 0-80%.

100. The therapeutic pharmaceutical composition of claim 99, wherein the ratio of the water insoluble hydrophobic polymer to a complete dosage form is 20-60%.

101. The therapeutic pharmaceutical composition of claim 100, wherein the ratio of the water insoluble hydrophobic polymer to a complete dosage form is 35-45%.

102. A tablet dosage form comprising 100 mg of tramadol HCl, 150 mg of croscarmellose, 50 mg of polyvinylpyrrolidone, and 200 mg of poly(vinyl acetate).

103. The tablet dosage form of claim 102, wherein, without heat treatment, provides hindered immediate release of tramadol HCl up to 50-60% in water and immediate complete release in 0.1 N HCl.

104. The tablet dosage form of claim 102, wherein, with heat treatment, provides a 24 hr complete sustained release of tramadol HCl, a 24 hr hindered sustained release of tramadol HCl in water up to 40-50%, and a 24 hr hindered sustained release of tramadol HCl in pH 3 solution up to 80-90%.

105. A tablet dosage form of 500 mg comprising a 50% mixture of polyvinylpyrrolidone and poly(vinyl acetate), wherein, without heat treatment, provides sustained release of 78±5% and 74±5% tramadol HCl in water and in 0.1N HCl.

106. A tablet dosage form of 500 mg comprising 20% water-soluble polymer and a 50% mixture of polyvinylpyrrolidone and poly(vinyl acetate), wherein, without heat treatment, provides immediate release of 74±5% and 95±5% tramadol HCl in water and in 0.1N HCl.

107. The tablet dosage form of claim 106, wherein the water-soluble polymer is croscaremellose.

108. A tablet dosage form of 500 mg comprising 20% water-soluble polymer and a 50% mixture of polyvinylpyrrolidone and poly(vinyl acetate), wherein, without heat treatment, provides immediate release of 64±5% and 95±5% tramadol HCl in water and in 0.1N HCl.

109. The tablet dosage form of claim 108, wherein the water-soluble polymer is croscaremellose.

110. A tablet dosage form of 500 mg comprising 30% water-soluble polymer and a 50% mixture of polyvinylpyrrolidone and poly(vinyl acetate), wherein, without heat treatment, provides immediate release of 53±5% and 95±5% tramadol HCl in water and in 0.1N HCl.

111. The tablet dosage form of claim 110, wherein the water-soluble polymer is croscaremellose.

112. A tablet dosage form of 500 mg comprising a 50% mixture of polyvinylpyrrolidone and poly(vinyl acetate), wherein, with heat treatment, provides sustained release of 61±5% and 62±5% tramadol HCl in water and in 0.1N HCl.

113. The tablet dosage form of claim 112, wherein the heat treatment is heating at 120° C. for a time period of one hour.

114. A tablet dosage form of 500 mg comprising 10% water-soluble polymer and a 50% mixture of polyvinylpyrrolidone and poly(vinyl acetate), wherein, with heat treatment, provides sustained release of 62±5% and 79±5% tramadol HCl in water and in 0.1N HCl.

115. The tablet dosage form of claim 114, wherein the water-soluble polymer is croscaremellose.

116. The tablet dosage form of claim 114, wherein the heat treatment is heating at 120° C. for a time period of one hour.

117. A tablet dosage form of 500 mg comprising 20% water-soluble polymer and a 50% mixture of polyvinylpyrrolidone and poly(vinyl acetate), wherein, with heat treatment, provides sustained release of 52±5% and 88±5% tramadol HCl in water and in 0.1N HCl.

118. The tablet dosage form of claim 117, wherein the water-soluble polymer is croscaremellose.

119. The tablet dosage form of claim 117, wherein the heat treatment is heating at 120° C. for a time period of one hour.

120. A tablet dosage form of 500 mg comprising 30% water-soluble polymer and a 50% mixture of polyvinylpyrrolidone and poly(vinyl acetate), wherein, with heat treatment, provides sustained release of 44±5% and 95±5% tramadol HCl in water and in 0.1N HCl.

121. The tablet dosage form of claim 120, wherein the water-soluble polymer is croscaremellose.

122. The tablet dosage form of claim 120, wherein the heat treatment is heating at 120° C. for a time period of one hour.

123. The therapeutic pharmaceutical composition of claim 83, wherein the tablet is a single-layer tablet, a multi-layer tablet, or a coated tablet.

124. The therapeutic pharmaceutical composition of claim 80, wherein the three polymers of the deterrent cage are mixed with the pharmaceutically-active ingredient in a dry form and then compressed into a tablet form.

125. The therapeutic pharmaceutical composition of claim 80, wherein the pharmaceutically-active ingredient is mixed with an aqueous or hydro-alcoholic dispersion of the water-swellable polymer and dried to form a stable complex.

126. A therapeutic pharmaceutical composition for deterring abuse of drugs or alcohol, the composition comprising:
a. at least one pharmaceutical active ingredient, the pharmaceutically-active ingredient:
i. is known to be abusable;
ii. is in a form of weak base as a salt of organic and inorganic acids including hydrochloric acid, hydrosulfuric acid, hydrophosphoric acid, and tartaric acid;
iii. is for treating at least one of anxiety, depression, sleep disorders, pain, lack of energy, attention deficit, cough, and cold; and
iv. is a barbiturate selected from at least one of phenobarbitals, benzodiazepines, codeine, morphine, oxycodone, oxymorphone, hydrocodone, hydromorphone, tramadol, amphetamines, methyl phenidate, dextromethorphan, and pseudoephedrine;
b. at least one polymeric binder:
i. that is freely soluble in water and in alcohol-rich hydroalcoholic solutions;
ii. that effectively covers a surface of the pharmaceutically-active ingredient as well as deterrent agent particles;
iii. that if heated from 25 to 150° C. at a heating rate of 10° C./min,
A. shows a melting point in a range of 65-80° C. if no attempt is made to erase its thermal history;
B. shows a heat of fusion in a range of 170-220 J/g if no attempt is made to erase its thermal history;
C. shows a melting point in a range of 60-75° C. if its thermal history is erased;
D. shows a heat of fusion in a range of 125-160 J/g if its thermal history is erased;
E. shows that over a molecular weight range of 100,000-7,000,000 g/mol, the melting point only varies about 5±2° C. in presence of thermal history;
F. shows that over a molecular weight range of 100,000-7,000,000 g/mol, the heat of fusion only varies about 28±2 J/g in presence of thermal history;
G. shows that over a molecular weight range of 100,000-7,000,000 g/mol, the melting point only varies about 5±2° C. in absence of thermal history;
H. shows that over a molecular weight range of 100,000-7,000,000 g/mol, the heat of fusion only varies about 21±2 J/g in absence of thermal history; and
I. is an ethylene oxide homopolymer with a molecular weight below 200,000 g/mol, which its melting point in the presence of thermal history, heat of fusion in the presence of thermal history, melting point in the absence of thermal history, and heat of fusion in the absence of thermal history are about 71±2° C., 182±2 J/g, 65±2° C., and 132±2 J/g, respectively;
c. at least one deterrent agent:
i. that binds to or traps the pharmaceutically-active ingredient in an aqueous, saline, or hydroalcoholic medium;
ii. either has ionic structure enabling the deterrent agent to bind to the pharmaceutically-active ingredient, or a porous functional structure accommodating molecules within; and
A. is organic in origin;
B. is inorganic in origin; or
C. is either organic or inorganic in origin;
iii. a crosslinked carboxymethylcellulose, which when used at a drug/deterrent ratio of about 8 wt %, binds with the pharmaceutically-active ingredient in water, in saline, in 40% ethanol (aq), in a pH 3 solution, and in 0.1N HCl to about 69, 22, 67, 77, and 8% respectively;
d. that provides different release profiles in water and in 0.1N HCl; and
e. that if heat treated, its mechanical strength is significantly improved, while its drug release profile will remain almost unchanged;

127. The therapeutic pharmaceutical composition of claim 126, wherein, the polymeric binder, if heated from 25 to 150° C. at a heating rate of 10° C./min shows a melting point in a range of 67-76° C. if no attempt is made to erase its thermal history.

128. The therapeutic pharmaceutical composition of claim 127, wherein, the polymeric binder, if heated from 25 to 150° C. at a heating rate of 10° C./min shows a melting point in a range of 68-72° C. if no attempt is made to erase its thermal history.

129. The therapeutic pharmaceutical composition of claim 126, wherein, the polymeric binder, if heated from 25 to 150° C. at a heating rate of 10° C./min shows a heat of fusion in a range of 175-200 J/g if no attempt is made to erase its thermal history.

130. The therapeutic pharmaceutical composition of claim 129, wherein, the polymeric binder, if heated from 25 to 150° C. at a heating rate of 10° C./min shows a heat of fusion in a range of 175-190 J/g if no attempt is made to erase its thermal history.

131. The therapeutic pharmaceutical composition of claim 126, wherein, the polymeric binder, if heated from 25 to 150° C. at a heating rate of 10° C./min shows a melting point in a range of 62-70° C. if its thermal history is erased.

132. The therapeutic pharmaceutical composition of claim 131, wherein, the polymeric binder, if heated from 25 to 150° C. at a heating rate of 10° C./min shows a melting point in a range of 63-66° C. if its thermal history is erased.

133. The therapeutic pharmaceutical composition of claim 126, wherein, the polymeric binder, if heated from 25 to 150° C. at a heating rate of 10° C./min shows a heat of fusion in a range of 125-145 J/g if its thermal history is erased.

134. The therapeutic pharmaceutical composition of claim 133, wherein, the polymeric binder, if heated from 25 to 150° C. at a heating rate of 10° C./min shows a heat of fusion in a range of 125-135 J/g if its thermal history is erased.

135. The therapeutic pharmaceutical composition of claim 126, wherein the ethylene oxide homopolymer is a polyethylene oxide with a molecular weight of about 100,000 g/mol.

136. The therapeutic pharmaceutical composition of claim 126, wherein the deterrent agent has an anionic structure.

137. The therapeutic pharmaceutical composition of claim 126, wherein the deterrent agent is organic in origin and is based on starch or cellulose.

138. The therapeutic pharmaceutical composition of claim 137, wherein the deterrent agent is organic in origin and is carboxymethyl derivative of starch or cellulose.

139. The therapeutic pharmaceutical composition of claim 126, wherein the deterrent agent is inorganic in origin and is based on inorganic clays.

140. The therapeutic pharmaceutical composition of claim 139, wherein the deterrent agent is inorganic in origin and is based on bentonite clay.

141. The therapeutic pharmaceutical composition of claim 126, wherein the deterrent agent is organic or inorganic in origin, is processed into a porous functional structure and is a silicate or carbon-based material.

142. The therapeutic pharmaceutical composition of claim 141, wherein the deterrent agent is organic or inorganic in origin, is processed into a porous functional structure and is a medicinal charcoal.

143. The therapeutic pharmaceutical composition of claims 1, 27, 80 and 126, wherein the pharmaceutically-active agent is Tramadol HCl.

144. The therapeutic pharmaceutical composition of claim 126, wherein the deterrent agent provides a limited release profile in water and almost complete release profile in 0.1 N HCl.

145. The therapeutic pharmaceutical composition of claim 126, wherein a dosage form is a tablet.

146. The therapeutic pharmaceutical composition of claim 145, wherein a ratio of the polymeric binder and the deterrent agent determines mechanical strength and drug release profile of the tablet.

147. The therapeutic pharmaceutical composition of claim 145, wherein higher tablet strength and sustained drug release profile are achieved at higher polymeric binder to deterrent agent ratio.

148. The therapeutic pharmaceutical composition of claim 145, wherein an amount of polymeric binder to total tablet weight is 30-100%.

149. The therapeutic pharmaceutical composition of claim 148, wherein an amount of polymeric binder to total tablet weight is 40-70%.

150. The therapeutic pharmaceutical composition of claim 149, wherein an amount of polymeric binder to total tablet weight is 45-55%.

151. The therapeutic pharmaceutical composition of claim 145, wherein an amount of deterrent agent to total tablet weight is below 70%.

152. The therapeutic pharmaceutical composition of claim 151, wherein an amount of deterrent agent to total tablet weight is below 60%.

153. The therapeutic pharmaceutical composition of claim 152, wherein an amount of deterrent agent to total tablet weight is below 50%.

154. The therapeutic pharmaceutical composition of claim 126, wherein a ratio of deterrent agent to polymeric binder varies in a range of 0.0-1.2.

155. The therapeutic pharmaceutical composition of claim 154, wherein a ratio of deterrent agent to polymeric binder varies in a range of 0.6-1.1.

156. The therapeutic pharmaceutical composition of claim 155, wherein a ratio of deterrent agent to polymeric binder varies in a range of 0.85-1.0.

157. The therapeutic pharmaceutical composition of claim 126, comprising 25 mg of Tramadol HCl, 250 mg of a deterrent agent, and 250 mg of any polyethylene oxide (PEO) grade with a molecular weight ranging 100,000 to 7,000,000 g/mol, compressed into a tablet, and heated at a temperature in a range of 60-120° C. for a time period of 1 hour.

158. The therapeutic pharmaceutical composition of claim 157, wherein the temperature is in a range 80-100° C.

159. The therapeutic pharmaceutical composition of claim 158, wherein the temperature is in a range 85-95° C.

160. The therapeutic pharmaceutical composition of claim 126, comprising 25 mg of Tramadol HCl, 250 mg of a deterrent agent, and 250 mg of polyethylene oxide having a molecular weight of 100,000 g/mol, compressed into a tablet, and heated at 90° C. for a time period of 1 hour.

161. The therapeutic pharmaceutical composition of claim 160, wherein a ratio of Tramadol release in water and in 0.1 N HCl is 25±5% after a time period of 0.5 hour and is about 33±5% after time periods of 1 hour, 2 hours, and 4 hours.

162. The therapeutic pharmaceutical composition of claim 160, wherein a maximum amount of drug release in water is 35±5%.

163. The therapeutic pharmaceutical composition of claim 160, wherein a maximum amount of drug release in 0.1 N HCl is 95±5%.

164. The therapeutic pharmaceutical composition of claim 160, wherein the tablet is completely resistant to crushing by a pill crusher and to crushing by a ball mill having two steel balls of 1 cm each in diameter when the ball mill is run at a frequency of 25 Hz for a time period of 5 minutes.

165. The therapeutic pharmaceutical composition of claim 160, wherein the tablet breaks down into large particles when processed for a time period of 30 seconds to 1 minute in a heavy duty grinder in which 85-95% of the large particles generated are larger than 850 μm.

166. The therapeutic pharmaceutical composition of claim 126, wherein a weight percentage of the pharmaceutically-active ingredient to the total composition is 0.01-50%.

167. The therapeutic pharmaceutical composition of claim 166, wherein a weight percentage of the pharmaceutically-active ingredient to the total composition is 0.1-20%.

168. The therapeutic pharmaceutical composition of claim 167, wherein a weight percentage of the pharmaceutically-active ingredient to the total composition is 1-10%.

169. The therapeutic pharmaceutical composition of claim 126, further comprising at least one pharmaceutically-acceptable excipient for preparing a dosage form.

170. The therapeutic pharmaceutical composition of claim 169, wherein the dosage form is a tablet, a capsule, or a transdermal patch.

171. The therapeutic pharmaceutical composition of claim 126, wherein the deterrent agent is selected from natural crosslinked polymers, synthetic crosslinked polymers, and semi-synthetic crosslinked polymers.

172. The therapeutic pharmaceutical composition of claim 171, wherein the natural crosslinked polymers, synthetic crosslinked polymers, and semi-synthetic crosslinked polymers are selected from crosslinked carboxymethyl chitosan, crosslinked acrylic (methacrylic) acid (salts), crosslinked alginic acid (salts), and crosslinked xanthan

173. The therapeutic pharmaceutical composition of claim 126, wherein the composition is a single layer tablet, a multi-layer tablet, or a coated tablet.

174. The therapeutic pharmaceutical composition of claim 126, wherein the pharmaceutically-active ingredient, the polymeric binder, and the deterrent agent are mixed a dry state and then compressed into a tablet.

175. The therapeutic pharmaceutical composition of claim 126, wherein a mixture of the pharmaceutically-active ingredient, the polymeric binder, and the deterrent agent are processed into a dosage form using a thermal processing technique.

176. The therapeutic pharmaceutical composition of claim 174, wherein the thermal processing technique is hot-melt extrusion, heated-compression molding, or injection molding.

177. The therapeutic pharmaceutical composition of claim 174, wherein the mixture is further processed to reduce size and shape of the dosage form.

178. The therapeutic pharmaceutical composition of claim 126, wherein the pharmaceutically-active ingredient is mixed with an aqueous or hydro-alcoholic dispersion of the polymeric binder, dried to form a stable complex, and compressed into a tablet.

179. A therapeutic pharmaceutical composition for deterring abuse of drugs or alcohol, the composition comprising:
a. at least one pharmaceutically-active ingredient known to be abusable by crushing and liquid extraction;
b. a water-swellable polymer that binds to the abusable pharmaceutically-active ingredient in an aqueous or hydroalcoholic medium, the binding enhancing resistance to abuse by liquid extraction;
c. a sintering agent comprising a water-insoluble hydrophobic polymer, the sintering agent providing mechanical strength to the composition, enhancing resistance to abuse by crushing; and
d. a sintering enhancer comprising a water-soluble low molecular weight polymer, the sintering enhancer lowering a temperature at which the hydrophobic polymer is sintered.

180. The therapeutic pharmaceutical composition of claim 179, wherein the pharmaceutically-active ingredient is for treating at least one of anxiety, depression, sleep disorders, pain, lack of energy, attention deficit, cough, and cold.

181. The therapeutic pharmaceutical composition of claim 179, wherein the pharmaceutically-active ingredient is a barbiturate.

182. The therapeutic pharmaceutical composition of claim 181, wherein the barbiturate is at least one of a phenobarbital, a benzodiazepine, codeine, morphine, oxycodone, oxymorphone, hydrocodone, hydromorphone, tramadol, an amphetamine, a methyl phenidate, dextromethorphan, and pseudoephedrine.

183. The therapeutic pharmaceutical composition of claim 179, further comprising at least one pharmaceutically-acceptable excipient for preparing a dosage form.

184. The therapeutic pharmaceutical composition of claim 183, wherein the dosage form is a tablet.

185. The therapeutic pharmaceutical composition of claim 179, wherein the water-swellable polymer is at least one of synthetic, natural, or semi-synthetic crosslinked polymers.

186. The therapeutic pharmaceutical composition of claim 185, wherein the synthetic, natural, or semi-synthetic crosslinked polymer is at least one of crosslinked carboxymethylcellulose, crosslinked carboxymethyl starch, crosslinked carboxymethyl chitosan, crosslinked acrylic (methacrylic) acid and salts thereof, crosslinked alginic acid and salts thereof, and crosslinked xanthan.

187. The therapeutic pharmaceutical composition of claim 186, wherein the synthetic, natural, or semi-synthetic crosslinked polymer is crosslinked carboxymethylcellulose.

188. The therapeutic pharmaceutical composition of claim 179, wherein the sintering agent is a low glass transition polymer.

189. The therapeutic pharmaceutical composition of claim 188, wherein the low glass transition polymer contains vinyl acetate monomers.

190. The therapeutic pharmaceutical composition of claim 179, wherein the sintering agent is a poly (vinyl acetate) homopolymer.

191. The therapeutic pharmaceutical composition of claim 179, wherein a ratio of the water-swellable polymer to a final dosage form is 25-35%.

192. The therapeutic pharmaceutical composition of claim 179, wherein a ratio of the sintering agent to a final dosage form is 45-55%.

193. The therapeutic pharmaceutical composition of claim 179, wherein a ratio of the sintering enhancer is 2-8%.

194. A tablet dosage form comprising 100 mg of Tramadol HCl, 150 mg crosslinked carboxymethylcellulose, 50 mg of polyvinylpyrrolidone, 200 mg of poly(vinyl acetate), and 25 mg polyethylene glycol (PEG).

195. The tablet dosage form of claim 194, wherein, without heat treatment, provides hindered immediate release in water up to 64% and immediate complete release in 0.1 N HCl.

196. The tablet dosage form of claim 194, wherein, with heat treatment at 90° C. for 1 hour, provides 24 hour sustained release of Tramadol HCl in 0.1N HCl at 37° C. up to 90% and 24 hour hindered sustained release of Tramadol HCl in water up to 43%.

197. The tablet dosage form of claim 194, wherein, with heat treatment at 120° C. for 1 hour, provides 24 hour sustained release of Tramadol HCl in 0.1N HCl at 37° C. up to 82% and 24 hour hindered sustained release of Tramadol HCl in water up to 43%.

198. The tablet dosage form of claim 194, wherein, with heat treatment at 8-120° C. for 1 hour, provides a more sustained release Tramadol HCl over 24 hours compared to the table dosage without sintering enhancer when tested using USP apparatus II at 37° C., 0.1 N HCl, 50 rpm.

199. The tablet dosage form of claim 194, wherein, with heat treatment at 8-120° C. for 1 hour crushed in a ball mill, generates over 99% particles larger than 850 μm and the same tablet dosage without the sintering enhancer generates up to 87% particles larger than 850 μm.

200. The tablet dosage form of claim 194, wherein, with heat treatment at 8-120° C. for 1 hour crushed in a ball mill followed by a grind mill, generates over 80% particles larger than 850 μm and the same tablet dosage without the sintering enhancer generates up to 64% particles larger than 850 μm.

201. The therapeutic pharmaceutical composition of claim 184, wherein the tablet is a single layer tablet, a multi-layer tablet, or a coated tablet.

202. The therapeutic pharmaceutical composition of claim 179, wherein the pharmaceutically-active ingredient, the water-swellable polymer, the sintering agent, and the sintering enhancer are mixed a dry state and then compressed into a tablet.

203. The therapeutic pharmaceutical composition of claim 179, wherein the pharmaceutically-active ingredient is mixed with an aqueous or hydro-alcoholic dispersion of the water-swellable polymer, dried to form a stable complex, and compressed into a tablet.

204. A therapeutic pharmaceutical composition for deterring abuse of drugs or alcohol, the composition comprising:
at least one pharmaceutically-active ingredient known to be abusable; and
at least one inorganic clay;
i. wherein the inorganic clay has sufficient binding sites to form an effective and stable complex with the pharmaceutically-active ingredient;
ii. wherein the inorganic clay can be used as fine particles or as coarse aggregates; and
iii. wherein the clay fine particles or clay coarse aggregates in a dosage form are either coated with a water-insoluble coating material, or physically separated from the pharmaceutically-active ingredient.

205. The therapeutic pharmaceutical composition of claim 204, further comprising at least one pharmaceutically-acceptable excipient for preparing a dosage form.

206. The therapeutic pharmaceutical composition of claim 204, wherein the dosage form is a tablet, a capsule, or a transdermal patch.

207. The therapeutic pharmaceutical composition of claim 204, wherein the pharmaceutically-active ingredient is for treating at least one of anxiety, depression, sleep disorders, pain, lack of energy, attention deficit, cough, and cold.

208. The therapeutic pharmaceutical composition of claim 204, wherein the pharmaceutically-active ingredient is a barbiturate.

209. The therapeutic pharmaceutical composition of claim 208, wherein the barbiturate is at least one of a phenobarbital, a benzodiazepine, codeine, morphine, oxycodone, oxymorphone, hydrocodone, hydromorphone, tramadol, an amphetamine, a methyl phenidate, dextromethorphan, and pseudoephedrine.

210. The therapeutic pharmaceutical composition of claim 204, wherein the pharmaceutically-active ingredient is an abusable drug in a form of a weak base as a salt of organic and inorganic acids.

211. The therapeutic pharmaceutical composition of claim 210, wherein the organic and inorganic acids are hydrochloric acid, hydrosulfuric acid, and hydrophosphoric acid.

212. The therapeutic pharmaceutical composition of claim 204, wherein the inorganic clay is a phyllosilicate composed of aluminum silicate sheets.

213. The therapeutic pharmaceutical composition of claim 212, wherein the phyllosilicate is at least one of halloysite, kaolinite, illite, montmorillonite, vermiculite, talc, palygorskite, pyrophyllite, and zeolite.

214. The therapeutic pharmaceutical composition of claim 212, wherein the aluminum silicate sheets further comprise cations of at least one of potassium, sodium, ammonium, magnesium, lithium, calcium, and iron.

215. The therapeutic pharmaceutical composition of claim 204, wherein the inorganic clay is bentonite.

216. The therapeutic pharmaceutical composition of claim 204, wherein the coarse aggregates of clay are produced using conventional wet granulation or hot melt extrusion techniques.

217. The therapeutic pharmaceutical composition of claim 216, wherein a binder is used to produce the coarse clay aggregates.

218. The therapeutic pharmaceutical composition of claim 217, wherein the binder is a water-soluble or water-dispersible polymer.

219. The therapeutic pharmaceutical composition of claim 218, wherein the water-soluble or water-dispersible polymer is selected from the group consisting of polyacrylic acid, polyvinyl alcohol, polyethylene glycol, polyvinyl pyrrolidone, polyethylene oxide, alginic acid and its salts, chitosan, carrageenan, gum Arabic, guar gum, agar agar, gelatin, xanthan, locust bean gum, methyl cellulose, carboxymethyl cellulose, hydroxyethyl cellulose, hydroxypropyl cellulose, hydroxypropyl methylcellulose, starches, and combinations thereof.

220. The therapeutic pharmaceutical composition of claim 217, wherein the binder is hydroxypropyl methylcellulose.

221. The therapeutic pharmaceutical composition of claim 204, wherein the water-insoluble coating material is selected from the group consisting of ethyl cellulose, cellulose acetate, cellulose acetate phthalate, cellulose acetate butyrate, shellac, methacrylate and acrylate copolymers (enteric and non-enteric), poly(lactic acid), poly(lactide-co-glycolide), hydroxypropyl methylcellulose phthalate, hydroxypropyl methylcellulose acetate succinate, poly(vinyl acetate), and combinations thereof.

222. The therapeutic pharmaceutical composition of claim 221, wherein the water-insoluble coating material is a methacrylic acid ethyl acrylate copolymer.

223. The therapeutic pharmaceutical composition of claim 222, wherein a solid form or a dispersion form of methacrylic acid ethyl acrylate copolymer is used.

224. The therapeutic pharmaceutical composition of claim 223, wherein the solid form or the dispersion form is used as a coating base.

225. The therapeutic pharmaceutical composition of claim 204, wherein the water-insoluble coating material is selected from the group consisting of animal waxes, plant waxes, petroleum waxes, water-insoluble waxes of any origin, stearic acid, magnesium stearate, and combinations thereof.

226. The therapeutic pharmaceutical composition of claim 204, comprising 1-99 wt % of clay particles or clay aggregates.

227. The therapeutic pharmaceutical composition of claim 204, wherein the pharmaceutically-active ingredient and the coated clay particles or aggregates are mixed together and compressed into a tablet.

228. The therapeutic pharmaceutical composition of claim 204, wherein a pharmaceutically-active ingredient-clay complex is prepared and compressed into a tablet.

229. The therapeutic pharmaceutical composition of claim 206, wherein the dosage form is a bilayer or a multi-layer tablet.

230. The therapeutic pharmaceutical composition of claim 229, wherein the clay particles or clay aggregates are separated from the pharmaceutically-active ingredient within the tablet.

231. The therapeutic pharmaceutical composition of claim 204, wherein a process to make clay aggregates and a process to coat the clay aggregates can be done separately or can be done simultaneously in a continuous process.

232. The therapeutic pharmaceutical composition of claim 231, wherein the continuous process is hot melt extrusion.

233. The therapeutic pharmaceutical composition of claim 206, wherein the pharmaceutically-active ingredient is wet granulated and incorporated into a capsule with coated clay aggregates.

234. The therapeutic pharmaceutical composition of claim 204, wherein the composition can be manufactured as a gel, suppository, suspension, emulsion, micro-sized dispersion, nano-sized dispersion, semi-solid, paste, ointment, lozenge, strip, film, or rod.

235. The therapeutic pharmaceutical composition of claim 204, further comprising a coagulating agent that physically binds the inorganic clay or inorganic clay-pharmaceutically-active ingredient complex particles in a form of coagulates, the coagulates physically trapping the pharmaceutically-active ingredient in solution or in a volume of extracting solution.

236. The therapeutic pharmaceutical composition of claim 235, wherein the inorganic clay is bentonite clay.

237. The therapeutic pharmaceutical composition of claim 235, wherein the coagulating agent is selected from the group consisting of high molecular weight polymers based on ethylene oxide and derivatives thereof, acrylamide and derivatives thereof, acrylic acid and derivatives thereof, and methacrylic acid and derivatives thereof.

238. The therapeutic pharmaceutical composition of claim 235, wherein the coagulating agent is non-ionic and is at least one of very high molecular weight polyethylene oxide and polyacrylamide polymers having molecular weights equal or greater than 5,000,000 Da.

239. The therapeutic pharmaceutical composition of claim 236, wherein a weight ratio of the coagulating agent to the bentonite clay is 0.5-25%.

240. The therapeutic pharmaceutical composition of claim 239, wherein a weight ratio of the coagulating agent to the bentonite clay is 0.5-10%.

241. The therapeutic pharmaceutical composition of claim 240, wherein a weight ratio of the coagulating agent to the bentonite clay is 1-3%.

242. A therapeutic pharmaceutical composition comprising 25 mg of a pharmaceutically-active ingredient, 200 mg of bentonite clay, and 2 mg solid polyethylene oxide (PEO) having a molecular weight greater than 5,000,000) in 10 mL of extracting solution, wherein the composition enhances an amount of entrapped drug to as high as 5% compared to a control composition without PEO.

243. A therapeutic pharmaceutical composition comprising 25 mg of a pharmaceutically-active ingredient, 200 mg of bentonite clay, and 2 mg solid polyethylene oxide (PEO) having a molecular weight greater than 5,000,000) in 10 mL of extracting solution, wherein the composition enhances an amount of entrapped extraction solution to as high as 9% compared to a control composition without PEO.

244. The therapeutic pharmaceutical composition of claim 236, wherein the bentonite clay is sodium bentonite.

245. The therapeutic pharmaceutical composition of claim 244, wherein the sodium bentonite functions as a binding agent forming a stable suspension system in water at low to high concentration of the pharmaceutically-active ingredient

246. The therapeutic pharmaceutical composition of claim 245, wherein the sodium bentonite does not coagulate upon addition of a coagulating agent to the suspension system.

247. The therapeutic pharmaceutical composition of claim 245, wherein binding of the sodium bentonite to the pharmaceutically-active ingredient is increased if the system is heated at a temperature greater than 100° C. for a period of time.

248. The therapeutic pharmaceutical composition of claim 245, wherein the system remains stable after centrifugation at 4500 rpm for 5 minutes.

249. The therapeutic pharmaceutical composition of claim 245, wherein the system does not resist filtration.

250. The therapeutic pharmaceutical composition of claim 249, wherein, upon filtration, a time period that it takes the system to fully pass through a 0.2 μm filter is similar to the time period of water.

251. The therapeutic pharmaceutical composition of claim 244, wherein the composition comprises 100-1000 μg/mL of the pharmaceutically-active ingredient, remains stable, and does not settle after a time period of 12 hours.

252. A 10 mL solution in water comprising 1000 μg/ml of a pharmaceutically-active ingredient and 25 mg of heated sodium bentonite, wherein the solution has about 52% binding to the pharmaceutically-active agent.

253. A 10 mL solution in water comprising 500 μg/ml of a pharmaceutically-active ingredient and 25 mg of heated sodium bentonite, wherein the solution has about 85% binding to the pharmaceutically-active agent.

254. A 10 mL solution in water comprising 250 μg/ml of a pharmaceutically-active ingredient and 25 mg of heated sodium bentonite, wherein the solution has about 93% binding to the pharmaceutically-active agent.

255. A 10 mL solution in water comprising 100 μg/ml of a pharmaceutically-active ingredient and 25 mg of heated sodium bentonite, wherein the solution has about 100% binding to the pharmaceutically-active agent.

256. A 10 mL aqueous solution comprising 1000 μg/ml of a pharmaceutically-active ingredient and 25 mg of sodium bentonite, wherein the solution has a conductivity value of about −90 mV.

257. A 10 mL aqueous solution comprising 500 μg/ml of a pharmaceutically-active ingredient and 25 mg of sodium bentonite, wherein the solution has a conductivity value of about −130 mV.

258. A 10 mL aqueous solution comprising 250 μg/ml of a pharmaceutically-active ingredient and 25 mg of sodium bentonite, wherein the solution has a conductivity value of about −174 mV.

259. A 10 mL aqueous solution comprising 100 μg/ml of a pharmaceutically-active ingredient and 25 mg of sodium bentonite, wherein the solution has a conductivity value of about −190 mV.

260. A 10 mL aqueous solution comprising 100 μg/ml of a pharmaceutically-active ingredient and 25 mg of sodium bentonite, wherein the solution remains stable at a high pH and coagulates at a low pH of 1.0.

261. A 10 mL aqueous solution comprising 1000 μg/ml of a pharmaceutically-active ingredient and 25 mg of sodium bentonite, wherein the solution remains stable at a high pH and coagulates at a low pH of 1.0.

262. The therapeutic pharmaceutical composition of claim 244, wherein the sodium bentonite is coated with a water-insoluble coating material selected from the group consisting of ethyl cellulose, cellulose acetate, cellulose acetate phthalate, cellulose acetate butyrate, shellac, methacrylate and acrylate copolymers (enteric and non-enteric), poly(lactic acid), poly(lactide-co-glycolide), hydroxypropyl methylcellulose phthalate, hydroxypropyl methylcellulose acetate succinate, poly(vinyl acetate), and combinations thereof.

263. The therapeutic pharmaceutical composition of claim 262, wherein the water-insoluble coating material is a methacrylic acid ethyl acrylate copolymer.

264. The therapeutic pharmaceutical composition of claim 263, wherein a solid form or a dispersion form of methacrylic acid ethyl acrylate copolymer is used.

265. The therapeutic pharmaceutical composition of claim 264, wherein the solid form or the dispersion form is used as a coating base.

266. The therapeutic pharmaceutical composition of claim 244, wherein the sodium bentonite is coated with a water-insoluble coating material selected from the group consisting of animal waxes, plant waxes, petroleum waxes, water-insoluble waxes of any origin, stearic acid, magnesium stearate, and combinations thereof.

267. An abuse-deterrent composition comprising:
a crosslinked polyacid;
a weak base medication known to be abusable; and
a drug/polyacid complex.

268. The abuse-deterrent composition of claim 267, wherein the crosslinked polyacid is crosslinked sodium carboxymethylcellulose and the weak base medication is dextromethorphan HBr.

269. The abuse-deterrent composition of claim 268, wherein the crosslinked sodium carboxymethylcellulose is from 1-99 wt % of a dosage form.

270. The abuse-deterrent composition of claim 267, wherein the drug/polyacid complex is formed by adding the polyacid to an aqueous solution of the drug.

271. The abuse-deterrent composition of claim 267, wherein a complex containing a higher drug concentration is formed when drug concentration in solution increases.

272. The abuse-deterrent composition of claim 267, wherein an amount of drug within the drug-polyacid complex increases from an average 9 wt % to an average of 37 wt % over a drug/polyacid weight ratio of 0.1-1.

273. The abuse-deterrent composition of claim 267, wherein the complex between the drug and polyacid is retained in water, normal saline, and alcoholic solutions at any concentration.

274. The abuse-deterrent composition of claim 267, wherein the complex between the drug and polyacid breaks apart in full in 0.1N HCl.

275. The abuse-deterrent composition of claim 267, wherein the drug/polyacid complex formed at a 0.125 drug/polyacid weight ratio maintains over 90% of drug content in water, in pH 3 solution, in 40% EtOH, in 60% EtOH 60%, and in 100% EtOH.

276. The abuse-deterrent composition of claim 275, wherein the drug/polyacid complex formed at a 0.125 drug/polyacid weight ratio maintains over 96% of drug content in water.

277. The abuse-deterrent composition of claim 275, wherein the drug/polyacid complex formed at a 0.125 drug/polyacid weight ratio maintains over 97% of drug content in pH 3 solution.

278. The abuse-deterrent composition of claim 275, wherein the drug/polyacid complex formed at a 0.125 drug/polyacid weight ratio maintains over 97% of drug content in 40% EtOH.

279. The abuse-deterrent composition of claim 275, wherein the drug/polyacid complex formed at a 0.125 drug/polyacid weight ratio maintains over 93% of drug content in 60% EtOH.

280. The abuse-deterrent composition of claim 275, wherein the drug/polyacid complex formed at a 0.125 drug/polyacid weight ratio maintains over 99% of drug content in 100% EtOH.

281. The abuse-deterrent composition of claim 275, wherein the drug/polyacid complex formed at a 0.125 drug/polyacid weight ratio releases all drug content in 0.1 N HCL.

282. The abuse-deterrent composition of claim 267, wherein a dosage form is a tablet and can be administered orally in a single dose or in multiple doses.

283. The abuse-deterrent composition of claim 267, comprising 500 mg drug-polyacid complex, when abused by multiple administration to five times as much, can hold over 98% of drug content in water, over 90% of drug content in pH 3, and release not less than 85% of drug content in 0.1N HCl solution.

284. The abuse-deterrent composition of claim 283, wherein the drug-polyacid complex contains about 25 mg of drug.

285. The abuse-deterrent composition of claim 267, further comprising at least one of a clay or a charcoal.

286. The abuse-deterrent composition of claim 267, wherein the composition is formulated into a crush resistant abuse-deterrent formulation using hydrophilic and hydrophobic thermoplastic polymers.

287. The abuse-deterrent composition of claim 286, wherein the hydrophilic and hydrophobic thermoplastic polymers are low to high molecular weight poly(ethylene oxide) and a polymer blend of polyvinyl acetate and poly(vinyl pyrrolidone).

288. The abuse-deterrent composition of claim 267, further comprising at least one of pH-regulating agents, acid-neutralizing agents, and buffering agents.
